SEQUENCE LISTING (103 sequences)

SEQ ID NO: 1 - protein, 7 aa, Simian virus 40
PKKKRKV

SEQ ID NO: 2 - protein, 10 aa, Simian virus 40
PKKKRKVEAS

SEQ ID NO: 3 - protein, 16 aa, Artificial Sequence
  Synthetic
KRPAATKKAGQAKKKK

SEQ ID NO: 4 - protein, 9 aa, Homo sapiens
PAAKRVKLD

SEQ ID NO: 5 - protein, 11 aa, Homo sapiens
RQRRNELKRSP

SEQ ID NO: 6 - protein, 38 aa, Homo sapiens
NQSSNFGPMKGGNFGGRSSGPYGGGGQYFAKPRNQGGY

SEQ ID NO: 7 - protein, 42 aa, Homo sapiens
RMRIZFKNKGKDTAELRRRRVEVSVELRKAKKDEQILKRRNV

SEQ ID NO: 8 - protein, 8 aa, Homo sapiens
VSRKRPRP

SEQ ID NO: 9 - protein, 8 aa, Homo sapiens
PPKKARED

SEQ ID NO: 10 - protein, 8 aa, Homo sapiens
PQPKKKPL

SEQ ID NO: 11 - protein, 12 aa, Mus sp.
SALIKKKKKMAP

SEQ ID NO: 12 - protein, 5 aa, Influenza virus
DRLRR

SEQ ID NO: 13 - protein, 7 aa, Influenza virus
PKQKKRK

SEQ ID NO: 14 - protein, 10 aa, Hepatitis D virus
RKLKKKIKKL

SEQ ID NO: 15 - protein, 10 aa, Mus sp.
REKKKFLKRR

SEQ ID NO: 16 - protein, 20 aa, Homo sapiens
KRKGDEVDGVDEVAKKKSKK

SEQ ID NO: 17 - protein, 17 aa, Homo sapiens
RKCLQAGMNLEARKTKK

SEQ ID NO: 18 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 20 - DNA, 20 bp, Artificial Sequence
  Synthetic
tgagttggacgtgtgttttc

SEQ ID NO: 21 - DNA, 20 bp, Artificial Sequence
  Synthetic
tgattttggggtccattatc

SEQ ID NO: 22 - DNA, 25 bp, Artificial Sequence
  Synthetic
caccgtccccatttgaacactgcca

SEQ ID NO: 23 - DNA, 20 bp, Artificial Sequence
  Synthetic
ttgacgtgcctctgataaga

SEQ ID NO: 24 - DNA, 20 bp, Artificial Sequence
  Synthetic
aacgtaatgcggggtgcatt

SEQ ID NO: 25 - DNA, 25 bp, Artificial Sequence
  Synthetic
aaactggcagtgttcaaatggggac

SEQ ID NO: 26 - DNA, 20 bp, Artificial Sequence
  Synthetic
ccacaagtgccatctttaag

SEQ ID NO: 27 - DNA, 20 bp, Artificial Sequence
  Synthetic
agttgaatctgagggtccac

SEQ ID NO: 28 - DNA, 25 bp, Artificial Sequence
  Synthetic
caccgctactcacaagactcttcga

SEQ ID NO: 29 - DNA, 20 bp, Artificial Sequence
  Synthetic
catagggctgttcaagttga

SEQ ID NO: 30 - DNA, 20 bp, Artificial Sequence
  Synthetic
gttctccattctggttactg

SEQ ID NO: 31 - DNA, 25 bp, Artificial Sequence
  Synthetic
aaactcgaagagtcttgtgagtagc

SEQ ID NO: 32 - DNA, 20 bp, Artificial Sequence
  Synthetic
ccataacatgaccatgaggt

SEQ ID NO: 33 - DNA, 20 bp, Artificial Sequence
  Synthetic
cgacgttgttttgatcgcgc

SEQ ID NO: 34 - DNA, 20 bp, Artificial Sequence
  Synthetic
tactgaatgccttcgagttc

SEQ ID NO: 35 - DNA, 20 bp, Artificial Sequence
  Synthetic
agacgcagtattattgggta

SEQ ID NO: 36 - DNA, 20 bp, Artificial Sequence
  Synthetic
cctttattaccgttcttacg

SEQ ID NO: 37 - DNA, 20 bp, Artificial Sequence
  Synthetic
tgttgagtgagagcggtgaa

SEQ ID NO: 38 - DNA, 20 bp, Artificial Sequence
  Synthetic
tgctatgtttagtgttccag

SEQ ID NO: 39 - DNA, 20 bp, Artificial Sequence
  Synthetic
gggaatttaaggtcttcctt

SEQ ID NO: 40 - DNA, 20 bp, Artificial Sequence
  Synthetic
gttatcgacatagcgagtgt

SEQ ID NO: 41 - DNA, 20 bp, Artificial Sequence
  Synthetic
aattggaacgccttgtcctc

SEQ ID NO: 42 - DNA, 20 bp, Artificial Sequence
  Synthetic
ccagttgttcggacaaagtg

SEQ ID NO: 43 - DNA, 20 bp, Artificial Sequence
  Synthetic
catctggactgctattggtg

SEQ ID NO: 44 - DNA, 20 bp, Artificial Sequence
  Synthetic
tttcagaacgttccgtgtac

SEQ ID NO: 45 - DNA, 20 bp, Artificial Sequence
  Synthetic
cttcggtagtagccaatttg

SEQ ID NO: 46 - DNA, 20 bp, Artificial Sequence
  Synthetic
tctacccataaagccatcaa

SEQ ID NO: 47 - DNA, 20 bp, Artificial Sequence
  Synthetic
accacgaattcgtctggtag

SEQ ID NO: 48 - DNA, 20 bp, Artificial Sequence
  Synthetic
gaaaggcacatttggttgca

SEQ ID NO: 49 - DNA, 20 bp, Artificial Sequence
  Synthetic
gatctttcattttaccgtca

SEQ ID NO: 50 - DNA, 20 bp, Artificial Sequence
  Synthetic
tggctttaacaaaatcgccc

SEQ ID NO: 51 - DNA, 20 bp, Artificial Sequence
  Synthetic
tttgttagcaccatagggaa

SEQ ID NO: 52 - DNA, 20 bp, Artificial Sequence
  Synthetic
agtaaccacaagtagtggca

SEQ ID NO: 53 - DNA, 20 bp, Artificial Sequence
  Synthetic
cagttgcaacccatatgatg

SEQ ID NO: 54 - DNA, 20 bp, Artificial Sequence
  Synthetic
tcaggtcctacttctgaatt

SEQ ID NO: 55 - DNA, 20 bp, Artificial Sequence
  Synthetic
gattgcagcattgttagcag

SEQ ID NO: 56 - DNA, 20 bp, Artificial Sequence
  Synthetic
acaacctatgttagcgctag

SEQ ID NO: 57 - DNA, 20 bp, Artificial Sequence
  Synthetic
tagaagccttttggcaatgt

SEQ ID NO: 58 - DNA, 20 bp, Artificial Sequence
  Synthetic
ttctccaacaacacctgtat

SEQ ID NO: 59 - DNA, 20 bp, Artificial Sequence
  Synthetic
acgagaagaggcttgactgc

SEQ ID NO: 60 - DNA, 20 bp, Artificial Sequence
  Synthetic
ggttgtcattaagaccttcg

SEQ ID NO: 61 - DNA, 20 bp, Artificial Sequence
  Synthetic
tgttgcgactacgtgatgag

SEQ ID NO: 62 - DNA, 20 bp, Artificial Sequence
  Synthetic
caaacctttcacagtttcca

SEQ ID NO: 63 - DNA, 20 bp, Artificial Sequence
  Synthetic
gcctggagttgaatttcttg

SEQ ID NO: 64 - DNA, 20 bp, Artificial Sequence
  Synthetic
ttatagcggccttctgtaaa

SEQ ID NO: 65 - DNA, 20 bp, Artificial Sequence
  Synthetic
caggagaagttcccctactg

SEQ ID NO: 66 - DNA, 20 bp, Artificial Sequence
  Synthetic
actgaacaacaccacctgta

SEQ ID NO: 67 - DNA, 20 bp, Artificial Sequence
  Synthetic
cagcaaagcaagagcagcat

SEQ ID NO: 68 - DNA, 20 bp, Artificial Sequence
  Synthetic
tcaaggacgggtttgagttt

SEQ ID NO: 69 - DNA, 20 bp, Artificial Sequence
  Synthetic
agctggttcaatctgtcaag

SEQ ID NO: 70 - DNA, 20 bp, Artificial Sequence
  Synthetic
atttgtccaccgacaatttc

SEQ ID NO: 71 - DNA, 20 bp, Artificial Sequence
  Synthetic
tttaccagacattttgctct

SEQ ID NO: 72 - DNA, 20 bp, Artificial Sequence
  Synthetic
gaatgtctgaacactctcct

SEQ ID NO: 73 - DNA, 20 bp, Artificial Sequence
  Synthetic
tagtggcagtacgtttttgc

SEQ ID NO: 74 - DNA, 20 bp, Artificial Sequence
  Synthetic
ctttctgtacaatccctttg

SEQ ID NO: 75 - DNA, 20 bp, Artificial Sequence
  Synthetic
gcttgtgttacattgtatgc

SEQ ID NO: 76 - DNA, 20 bp, Artificial Sequence
  Synthetic
acttctgtgggaagtgtttc

SEQ ID NO: 77 - DNA, 20 bp, Artificial Sequence
  Synthetic
tttgttctggaccacgtctg

SEQ ID NO: 78 - DNA, 20 bp, Artificial Sequence
  Synthetic
acagcttcactagtaggttg

SEQ ID NO: 79 - DNA, 20 bp, Artificial Sequence
  Synthetic
tggtccccaaaatttccttg

SEQ ID NO: 80 - DNA, 20 bp, Artificial Sequence
  Synthetic
acaaactggtgtaccaacca

SEQ ID NO: 81 - DNA, 20 bp, Artificial Sequence
  Synthetic
agttccttgtctgattagtt

SEQ ID NO: 82 - DNA, 20 bp, Artificial Sequence
  Synthetic
atcatattaggtgcaagggc

SEQ ID NO: 83 - DNA, 20 bp, Artificial Sequence
  Synthetic
caatttgcggccaatgtttg

SEQ ID NO: 84 - DNA, 20 bp, Artificial Sequence
  Synthetic
aagtaacctttgttggtgca

SEQ ID NO: 85 - DNA, 20 bp, Artificial Sequence
  Synthetic
gaagcgctgggggcaaattg

SEQ ID NO: 86 - DNA, 20 bp, Artificial Sequence
  Synthetic
cttgcacttctatcacagtg

SEQ ID NO: 87 - DNA, 20 bp, Artificial Sequence
  Synthetic
atgcgcgacattccgaagaa

SEQ ID NO: 88 - DNA, 20 bp, Artificial Sequence
  Synthetic
ctcatttacttctgtaccga

SEQ ID NO: 89 - DNA, 20 bp, Artificial Sequence
  Synthetic
cgaaggtgtgacttccatgc

SEQ ID NO: 90 - DNA, 20 bp, Artificial Sequence
  Synthetic
tctaaatcaatgcccagtgg

SEQ ID NO: 91 - DNA, 20 bp, Artificial Sequence
  Synthetic
ctgtgtaggtcaaccacgtt

SEQ ID NO: 92 - DNA, 20 bp, Artificial Sequence
  Synthetic
catcttcagtaccatactca

SEQ ID NO: 93 - DNA, 20 bp, Artificial Sequence
  Synthetic
atttggatctttgtcatcca

SEQ ID NO: 94 - DNA, 20 bp, Artificial Sequence
  Synthetic
gtcttgttgaccaacagttt

SEQ ID NO: 95 - DNA, 20 bp, Artificial Sequence
  Synthetic
ttgtatgcgtcaatatgctt

SEQ ID NO: 96 - DNA, 20 bp, Artificial Sequence
  Synthetic
gttccatctctaattgaggt

SEQ ID NO: 97 - DNA, 20 bp, Artificial Sequence
  Synthetic
ggtaaggcttgagtttcatc

SEQ ID NO: 98 - DNA, 20 bp, Artificial Sequence
  Synthetic
ttggctgcattaacaaccac

SEQ ID NO: 99 - DNA, 20 bp, Artificial Sequence
  Synthetic
gaagagtcacagtttgctgt

SEQ ID NO: 100 - DNA, 20 bp, Artificial Sequence
  Synthetic
cttatttaaggctcctgcaa

SEQ ID NO: 101 - DNA, 20 bp, Artificial Sequence
  Synthetic
atcatccaaatctgcagcag

SEQ ID NO: 102 - DNA, 20 bp, Artificial Sequence
  Synthetic
acctttgttaacatttgggc

SEQ ID NO: 103 - DNA, 20 bp, Artificial Sequence
  Synthetic
ggattgttgcaattgtttgg

(19) United States
(12) Patent Application Publication
(10) Pub. No.: US 2024/0041851 A1
(43) Pub. Date: Feb. 8, 2024

(54) METHOD OF TREATING AND PREVENTING VIRAL INFECTION

(71) Applicants: THE GENERAL HOSPITAL CORPORATION, Boston, MA (US); THE BRIGHAM AND WOMEN'S HOSPITAL, INC., Boston, MA (US)

(72) Inventors: Vamsi Mootha, Boston, MA (US); Benjamin Gewurz, Boston, MA (US)

(21) Appl. No.: 18/265,030

(22) PCT Filed: Dec. 3, 2021
(86) PCT No.: PCT/US2021/061847

§ 371 Date: Jun. 2, 2023

Related U.S. Application Data
(60) Provisional application No. 63/120,847, filed on Dec. 3, 2020.

(51) Int. Cl. A61K 31/4412; A61K 31/4162; A61K 31/4439; A61K 31/454; A61K 31/426; A61K 31/427; A61K 31/198; A61K 31/4418; A61K 31/505; A61K 45/06; A61P 31/14

(52) U.S. Cl. CPC A61K 31/4412; A61K 31/4162; A61K 31/4439; A61K 31/454; A61K 31/426; A61K 31/427; A61K 31/198; A61K 31/4418; A61K 31/505; A61K 45/06; A61P 31/14

(57) ABSTRACT

A method of treating and preventing viral infection in a subject comprising administering an effective amount of one or more inhibitors of folate or one-carbon metabolism pathways to the subject.

### CROSS-REFERENCE TO RELATED APPLICATIONS

This application claims the benefit of U.S. Provisional Application No. 63/120,847, filed Dec. 3, 2020. The entire contents of the above-identified application are hereby fully incorporated herein by reference.

### REFERENCE TO AN ELECTRONIC SEQUENCE LISTING

The contents of the electronic sequence listing (“BROD-5310WP ST25.txt”; Size is 23,118 bytes (25 KB on disk) and it was created on Dec. 3, 2021) is herein incorporated by reference in its entirety.

### STATEMENT REGARDING FEDERALLY SPONSORED RESEARCH

This invention was made with government support under Grant Nos. AI137337 and CA228700, and GM122455 awarded by the National Institutes of Health. The government has certain rights in the invention.

### TECHNICAL FIELD

The subject matter disclosed herein is generally directed to methods for treating and preventing viral injection.

### BACKGROUND

The recently identified Severe Acute Respiratory Syndrome Coronavirus-2 (SARS-CoV-2) is an enveloped, single-stranded positive-sense RNA coronavirus responsible for COVID-19. Prior to vaccine or antiviral strategies approval for clinical use, SARS-CoV-2 rapidly spread and caused nearly a million deaths worldwide in less than one year. Therefore, there is a great need for the identification of novel antiviral targets and therapeutic agents.

Citation or identification of any document in this application is not an admission that such document is available as prior art to the present invention.

### SUMMARY

In one aspect, the present disclosure provides a method of inhibiting reproduction of a virus, comprising administering to a subject in need thereof infected by the virus, an effective amount of one or more inhibitors of folate metabolism or one-carbon metabolism in one or more cells of the subject.

In some embodiments, the inhibitor of folate or one-carbon metabolism is an inhibitor of cytosolic and/or mitochondrial isoforms of serine hydroxymethyltransferase (SHMT1 and SHMT2, respectively).

In some embodiments, the SHMT1/SHMT2 inhibitor is a compound of Formula (I),

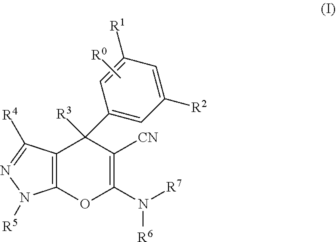

wherein:
R⁰, R¹ and R² are each independently selected from the group consisting of —H, halogen, hydroxyl, nitro, nitrile, —SOR¹¹, —S(O)₂R¹¹, —S(O)₂NR¹⁰R¹², OR¹¹, —OC(O)R¹², C(O)OR¹², —C(O)R¹¹, —C(O)NR¹⁰R¹², —NR¹⁰R¹², —N(R¹²)C(O)R¹¹, NS(O)₂R¹², substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl, and substituted or unsubstituted haloalkoxy; provided that at least one of R⁰, R¹ and R² is selected from the group consisting of substituted or unsubstituted alkenyl, and substituted or unsubstituted alkynyl;
R³, is selected from the group consisting of —H, halogen, hydroxyl, nitro, nitrile, SOR¹¹, —S(O)₂R¹¹, —S(O)₂NR¹⁰R¹², —OR¹¹, —OC(O)R¹², —C(O)OR¹², —C(O)R¹¹, C(O)NR¹⁰R¹², —NR¹⁰R¹², N(R¹²)C(O)R¹¹, NS(O)₂R¹², substituted or unsubstituted alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl, and substituted or unsubstituted haloalkoxy;
R⁴ is selected from the group consisting of —H, substituted or unsubstituted alkyl, substituted or unsubstituted cycloalkyl, substituted or unsubstituted aryl, substituted or unsubstituted heteroaryl, substituted or unsubstituted arylalkyl, and substituted or unsubstituted heteroarylalkyl;
R⁵, R⁶, and R⁷ are each independently selected from the group consisting —H, C(O)R¹¹, substituted or unsubstituted alkyl, substituted or unsubstituted cycloalkyl, substituted or unsubstituted aryl, substituted or unsubstituted heteroaryl, substituted or unsubstituted arylalkyl, and substituted or unsubstituted heteroarylalkyl; or R⁵ is selected from any of the foregoing and R⁶ and R⁷ taken together with the nitrogen atom to which they are attached form a substituted or unsubstituted 3-6 membered ring;
each occurrence of R¹¹ is independently selected from the group consisting of substituted or unsubstituted alkyl, substituted or unsubstituted cycloalkyl, substituted or unsubstituted heterocyclyl, substituted or unsubstituted aryl, and substituted or unsubstituted heteroaryl; and
each occurrence of R¹⁰ and R¹² is independently selected from the group consisting of —H, substituted or unsubstituted alkyl, substituted or unsubstituted cycloalkyl, substituted or unsubstituted heterocyclyl, substituted or unsubstituted aryl, and substituted or unsubstituted heteroaryl. In some embodiments, the SHMT1/SHMT2 inhibitor is a compound selected from Table 2.

In some embodiments, the SHMT1/SHMT2 inhibitor is a compound of Formula Formula (II),

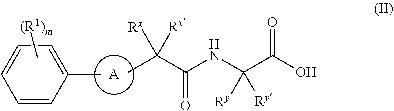

wherein:
Ring A is an optionally substituted bivalent ring;
each R¹ is independently halogen, —CN, —NO₂, —OR, -Cy, or an optionally substituted C₁₋₆ aliphatic group; or
two R¹ groups taken together with their intervening atoms are a 5-8 membered partially unsaturated or aryl fused ring having 0-3 heteroatoms independently selected from nitrogen, oxygen or sulfur; or
each R is independently hydrogen or an optionally substituted group selected from C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
Cy is an optionally substituted group selected from a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
R^{x} and R^{y} are independently hydrogen or an optionally substituted group selected from C₁₋₆ aliphatic or phenyl;
R^{x′} and R^{y′} are independently hydrogen or C₁₋₄ alkyl; or
R^{y} and R^{y′} can be taken together with their intervening atoms to form a 3-6 membered saturated ring having 0-3 heteroatoms independently selected from nitrogen, oxygen or sulfur; and m is 1, 2, 3, 4 or 5. In some embodiments, the SHMT1/SHMT2 inhibitor is a compound selected from Table 3. In some embodiments, the inhibitor of folate or one-carbon metabolism is a antifolate agent.

In another aspect, the present disclosure provides a method of inhibiting reproduction of a virus, comprising administering to a subject in need thereof, an effective amount of one or more inhibitors of glucose metabolism.

In another aspect, the present disclosure provides a method of inhibiting reproduction of a virus, comprising administering to a subject in need thereof, an effective amount of one or more inhibitors of purine synthesis. In another aspect, the present disclosure provides a method of inhibiting reproduction of a virus, comprising administering to a subject in need thereof, an effective amount of one or more inhibitors of serine synthesis. In some embodiments, the one or more inhibitors of serine synthesis is one or more inhibitors of phosphoglycerate dehydrogenase (PHGDH).

In another aspect, the present disclosure provides a method of inhibiting reproduction of a virus, comprising administering to a subject in need thereof, an effective amount of one or more inhibitors of oxidative phosphorylation. In some embodiments, the one or more inhibitors of oxidative phosphorylation is an inhibitor of electron transport chain complex I. In some embodiments, the inhibitor of electron transport chain complex I is Piericidin A (PierA). In some embodiments, the one or more inhibitors of oxidative phosphorylation is metformin, phenformin, atovaquone, chloramphenicol, meclizine, or S-meclizine.

In some embodiments, the virus is an RNA virus. In some embodiments, the RNA virus is a positive-sense single strand RNA virus. In some embodiments, the positive-sense single strand RNA virus is a coronavirus, flavivirus, enterovirus, or rhinovirus. In some embodiments, the coronavirus is SARS-CoV-1, MERS-CoV or SARS-CoV-2. In some embodiments, the flavivirus is hepatitis C virus, West Nile virus, or dengue virus. In some embodiments, the RNA virus is a negative-sense single strand RNA virus. In some embodiments, the negative-sense single strand RNA virus is an influenza, paramyxovirus, respiratory syncytial virus, lyssavirus, hantavirus, Lassa virus, or Ebola virus. In some embodiments, the paramyxovirus is the measles virus or the mumps virus. In some embodiments, the lyssavirus is the rabies virus.

1. In some embodiments, the one or more inhibitors is administered in combination with one or more anti-viral therapeutics. In some embodiments, the one or more anti-viral therapeutics comprises remdesivir, favipiravir, ribavirin, lopinavir/ritonavir, tocilizumab, leronlimab, IFNλ, ivermectin, chloroquine, hydroxychloroquine, colchicine, dexamethasone, prednisone, methylprednisolone, nicotine, vitamin D, spironolactone, casirivimab/imdevimab, bamlanivimab/etesevimab, sotrovimab, convalescent plasma, an mRNA vaccine, a viral vector vaccine, or any combination thereof. In some embodiments, the one or more inhibitors is administered prophylactically.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1G—Metabolic changes induced by early SARS-CoV-2 infection. FIG. 1A, Schematic of the metabolic profiling approach. Vero E6 T1VIPRSS2+ cells were infected with concentrated SARS-CoV-2 at a MOI=2 or mock-infected with virus-depleted flow-through for 8 hours and profiled by LC/MS and RNAseq in parallel. FIG. 1B, Immunofluorescence of nucleoprotein (IF-Np), fluorescence in situ hybridization (FISH) for +strand viral genomic RNA (FISH-gRNA) and merge with Hoeschst stained nuclei in infected vs mock-infected cells. FIG. 1C, Volcano plot visualization of SARS-CoV-2 versus mock infected cell RNAseq at 8 hours post infection/mock-infection from n=3 datasets. Values of selected inflammatory and metabolism pathway genes are shown. P-value generated with DESeq under default setting. FIG. 1D, Box plot visualization of RNAseq reads in SARS-CoV-2 versus mock infected cells. 1-way ANOVA with multiple comparison using the Sidak method. FIG. 1E, PCA of 104 intracellular metabolites and 25 spent media metabolites, as determined by LC-MS in SARS-CoV-2 infected or mock-infected cells, n=6 replicates. FIG. 1F, Volcano plot visualization of loge fold change (x-axis) and −log 10(P value) (y-axis) of intracellular metabolites measured by LC-MS. Significantly increased or decreased metabolites related to glycolysis, de novo purine synthesis, 1C metabolism/transsulfuration pathway, amino acids, histidine catabolism, and urea cycle/polyamine metabolism are labeled. n=6 biologically independent replicates, P-values were generated with two-tailed Student's t-test. FIG. 1G, Intracellular LC-MS metabolite levels at 8 hours of SARS-CoV-2 infection, normalized to levels detected in mock-infected. Druggable targets are labeled. All barplots show mean±SD. *P<0.05, **P<0.01, or ***P<0.001 from Student's two-tailed t-test.

FIG. 2A-2I—SARS-CoV-2 induced glycolysis and one-carbon metabolism supported viral RNA and protein expression, replication and cytopathic effect. FIG. 2A, SARS-CoV-2+strand gRNA FISH, nucleoprotein IF and merge with Hoeschst stained nuclei at 48 hpi of cells cultured in media with 25 mM glucose versus galactose as the sugar source. FIG. 2B, Fold change of live cell number and median tissue culture infectious dose (TCID50) per ml of culture supernatant at 48 hours post-infection of cells cultured in glucose versus galactose. Mean±SD values from n=3 biologically independent replicates. FIG. 2C, FISH analysis of +strand gRNA, IF for Np and merge with Hoeschst stained nuclei in SARS-CoV-2 infected cells treated DMSO or 100 nM piericidin A for 48 hours. FIG. 2D, Mean±SD fold change live cell number from n=3 biologically independent replicates. FIG. 2E, Mean±SD TCID50 values from n=3 biologically independent replicates, as in FIG. 2C-2D. FIG. 2F, Phase microscopic images of SARS-CoV-2 versus mock infected cells cultured for 48 hours with DMSO, 1 μM of methotrexate (MTX), 30 μM hypoxanthine (hypo), 100 μM thymidine, or 1 mM formate, as indicated. Scale bar indicates 100 μm. FIG. 2G, Fold change live cell number and TCID50/ml from samples collected as in f. Bar chart values show mean±SD values from n=3 biologically independent replicates. FIG. 211, FISH microscopic analysis of viral+strand gRNA, IF of Np and merge with Hoeschst stained nuclei in SARS-CoV-2 infected cells treated for 48 hours. Arrows indicate representative cells with high gRNA but low Np signal. FIG. 2I, Ratios of +strand gRNA FISH versus Np IF signals from 500 cells from twenty random fields for each condition in (FIG. 2H). In all panels, cells were infected at MOI=0.1 for 48 hours. Microscopy images are representative of at least n=3 biologically independent values. P-values in this figure were calculated by one-way ANOVA with multiple comparisons using Sidak method.

FIG. 3A-3F—SARS-CoV-2 induced serine one-carbon metabolism supported viral RNA and protein expression, replication and cytopathic effect. FIG. 3A, Phase microscopic images of SARS-CoV-2 versus mock infected cells cultured for 48 hours with DMSO, 10 μM of the dual SHMT1/2 inhibitor SHIN1 or 10 μM SHIN1+1 mM formate, as indicated. White scale bar indicates 100 μm. FIG. 3B, Fold change live cell (left) and TCID50 (right), as in a. Mean±SD values from n=3 biologically independent replicates are shown with one-way ANOVA, Sidak post-test. FIG. 3C, IF of Np, FISH for +strand gRNA and merge with Hoeschst stained nuclei in SARS-CoV-2 infected or mock-infected cells treated with DMSO, 10 μM SHIN1, or 10 μM SHIN1 and 1 mM formate for 48 hours. FIG. 3D, Immunoblot analysis of whole cell lysates from Cas9+T1VIPRSS2+Vero E6 expressing control, SHMT1 or SHMT2 sgRNAs. FIG. 3E Fold change live cell (left) and TCID50 (right) in cells with control, SHMT1 or SHMT2 targeting sgRNAs infected by SARS-CoV-2 for 48 hours. Mean±SD values from n=3 biologically independent replicates are shown with one-way ANOVA, Sidak post-test. FIG. 3F, FISH of subgenomic Np RNA, IF of Np, FISH for +strand gRNA and merge with Hoeschst stained nuclei in cells with control, SHMT1 or SHMT2 targeting sgRNAs infected by SARS-CoV-2 for 48 hours. In all panels, cells were infected at MOI=0.1 for 48 hours. Microscopy images are representative of at least n=3 biologically independent values. P-values in this figure were calculated by one-way ANOVA with multiple comparisons using Sidak method.

FIG. 4: Schematic of SARS-CoV-2 induced one-carbon metabolism in support of viral replication. SARS-CoV-2 induced glycolysis and one-carbon metabolism at the post-transcriptional level in newly infected cells. Serine metabolism, particularly by cytosolic SHMT1 produces carbon units for de novo purine synthesis in support of massive viral subgenomic RNA synthesis, non-structural protein expression and viral replication.

FIG. 5. Transcriptome analysis of mock or SARS-CoV-1 infected Vero-E6 cells at 7 hpi. Volcano plot visualization of −Log 10 (p-value) statistical significance (y-axis) and Log 2 fold-change of mRNA abundance in SARS-CoV-1 versus mock-infected cells (x-axis) from triplicate microarray datasets. Values for selected SARS-CoV-1 induced genes labeled.

FIG. 6A-6B—Media metabolite profiling of mock or SARS-CoV-2 infected Vero-E6 cells at 8 hpi. FIG. 6A, PCA of 25 spent media metabolites, as determined by LC-MS of n=6 biologically independent replicates. FIG. 6B, Quantitative analyses of fold-change of select media metabolites in SARS-CoV-2 infected versus mock-infected cells from n=6 biologically independent replicates.

FIG. 7A-7D—Analysis of ISR inhibitor, sugar source and glutathione synthesis enzyme GCL inhibitor effects on SARS-CoV-2 infection. FIG. 7A, IF of Np, FISH for +strand gRNA and merge with Hoeschst stained nuclei in mock or SARS-CoV-2 infected cells treated with DMSO or 1004 of ISRIB, a small molecule integrated stress response inhibitor for 48 hours. FIG. 7B, IF of Np, FISH for +strand gRNA and merge with Hoeschst stained nuclei in mock or SARS-CoV-2 infected cells cultured in media with 25 mM glucose or galactose for 48 hours. FIG. 7C, Fold change live cell (left) and TCID50 (right), of mock or SARS-CoV-2 infected cells treated for 48 hours with DMSO or 1004 of BSO, an inhibitor of glutathione synthesis. Mean±SD values from n=3 biologically independent replicates are shown with one-way ANOVA, Sidak post-test. FIG. 7D, IF-Np, FISH for +strand gRNA and merge with Hoeschst stained nuclei in mock or SARS-CoV-2 infected cells treated with DMSO or 1004 BSO for 48 hours. In all panels, cells were infected at MOI=0.1 for 48 hours. Microscopy images are representative of at least n=3 biologically independent values.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

A protein or nucleic acid derived from a species means that the protein or nucleic acid has a sequence identical to an endogenous protein or nucleic acid or a portion thereof in the species. The protein or nucleic acid derived from the species may be directly obtained from an organism of the species (e.g., by isolation), or may be produced, e.g., by recombination production or chemical synthesis.

As used herein, when an protein (e.g., an enzyme) is mentioned, the term also includes a functional domain of the protein (e.g., enzyme). For example, a reverse transcriptase may refer to a reverse transcriptase protein or a reverse transcriptase domain.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides for methods and compositions for treating and preventing viral infection and related health problems in a subject. In general, the methods may comprise modulating metabolic pathways in host cells that are involved in viral reproduction. In some embodiments, the methods comprise administering to a subject an effective amount of one or more inhibitors of folate and/or one-carbon metabolism pathways in the cells of the subject infected by a virus (e.g., SARS-CoV-2). In some examples, the inhibitors of folate metabolism (e.g., folate-mediated one-carbon metabolism) may be inhibitors of dihydrofolate reductase (e.g., methotrexate or analog thereof) or inhibitors of one or more isoforms of serine hydroxymethyltransferase (e.g., SHMT1 (cytosol isoform) and SHMT2 (mitochondrial isoform)).

### Methods of Inhibiting Viral Reproduction

In an aspect, the present disclosure provides methods for inhibiting viral reproduction. The methods may comprise administering to cells, tissues, organs or a subject (e.g., a subject in need thereof) one or more modulating agents that inhibit the reproduction of a virus. For example, the modulating agents may modulate (e.g., inhibit) one or more metabolic pathways involved in virus reproduction in the host.

A virus is an infectious agent composed of a protein sheath surrounding a nucleic acid core, which is capable of infecting a plant, animal or bacteria. Viruses are characterized by having a total dependence on a living host cell for reproduction, and by a lack of independent metabolism. After passing into the host cell, during an early stage of viral replication, the virion (i.e., the complete, mature virus particle) disintegrates, freeing elements that direct the host cell's metabolism in the process of reproducing the virus. Specifically, the nucleic acid core (RNA or DNA) is released as the basic infectious material of the virus. Generally, virions containing DNA are referred to by the common term viruses, or more properly DNA viruses. Those containing RNA in the virion core, and whose genomic RNA is copied to form DNA which is integrated into the host DNA, are referred to as retroviruses or RNA viruses.

When DNA from a DNA virus is released in the host cell, this DNA is integrated into the cellular DNA for later reproduction of the virion which, often after destroying the host cell is expelled to continue the infection in other cells. If positive strand RNA is the genetic material of the virus, this RNA is copied to DNA by one of the viral proteins (reverse transcriptase) and the resulting viral DNA is integrated and reproduced by the reproductive mechanism of the host cell as described in the preceding sentence. For RNA viruses in which the genomic material does not integrate into the host DNA, both positive and negative stranded viral RNA are ultimately translated into structural proteins and copied into host cell genomic RNA by a viral RNA-dependent RNA polymerase for packaging into virions with no requirement for integration into cellular DNA.

As used herein, “viral reproduction” may refer to one or more or all of the stages of the viral life cycle, including binding, cell entry, release of viral genetic material into the cell, replication of viral genetic material and synthesis of viral proteins, assembly and packaging of viral genetic materials and proteins into virions, and release of virions from the cell. As used herein, “inhibition of viral reproduction” may comprise inhibition of one or more or all stages of the viral life cycle.

As used herein, “inhibiting viral reproduction “inhibit” or “inhibition” may refer to preventing a viral infection, or reducing the efficiency of viral replication at one or more stages of the viral replication cycle. Inhibition of viral reproduction may be measured, for example, by an assay for viral titer. Assays for viral titer may include, for example, a measurement over time, e.g. a fluorescent-focus assay, to measure number of infected cells as described in methods herein. A fluorescent-focus assay measurement can be in fluorescent-focus units per milliliter (FFU/ml) and can be measured in a patient, cells, or some population thereof over a period of time, e.g. before and after treatment periods, etc. Alternative assays for viral titer may include nucleic acid amplification tests (NAATs) for quantification of viruses in a sample such that over time inhibition of viral reproduction is shown by a decrease in quantity of viral nucleic acid measured. Inhibition of viral infection may be measured, for example, by an assay for viral genomic material, e.g., positive strand genomic RNA or subgenomic RNA. Assays for viral genomic material may include, for example, a Stellaris' RNA fluorescence in situ hybridization (RNA-FISH) measurement as described in methods herein. Inhibition of viral infection may be measured, for example, by an assay for viral proteins, e.g., nucleocapsid protein. Assays for viral proteins may include, for example, immunofluorescence measurement as described in methods herein. Assays for viral genomic material may be performed simultaneously with assays for viral proteins, such as a simultaneous Stellaris™ RNA-FISH/Immunofluorescence assay.

The method may include administering a therapeutically effective amount of a modulating agent. The term “therapeutically effective amount” refers to an amount effective, at dosages and for periods of time necessary, to achieve the desired therapeutic result, e.g., inhibition of viral reproduction. A therapeutically effective amount of a modulating agent can vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects. In some embodiments, a therapeutically effective amount is an “effective amount”, which as used herein, refers to the amount of modulating agent and/or therapeutic agents to alleviate at least one or some of the symptoms of the disease or disorder. An “effective amount” for purposes herein is thus determined by such considerations as are known in the art and is the amount to achieve improvement including, but not limited to, improved survival rate or more rapid recovery, or improvement or elimination of at least one symptom and other indicator of a viral infection which are appropriate measures by those skilled in the art. It should be noted that a modulating agent as disclosed herein can be administered as a pharmaceutically acceptable salt and can be administered alone or as an active ingredient in combination with pharmaceutically acceptable carriers, diluents, adjuvants and vehicles.

The treatment method may include administering a prophylactically effective amount of modulating agent(s). The term “prophylactically effective amount” refers to an amount of a modulating agent which is effective, at dosages and for periods of time necessary, to achieve the desired prophylactic result, e.g., the amount of a target gene or gene product modulator. Typically, since a prophylactic dose of a modulating agent is administered to a subject prior to or at an earlier stage of a disease, and in some embodiments, a prophylactically effective amount is less than the therapeutically effective amount. A prophylactically effective amount of a modulating agent is also one in which any toxic or detrimental effects of the compound are outweighed by the beneficial effects.

In some embodiments, the one or more modulating agents herein may be administered as prophylactic agent(s) in response to a potential exposure to a virus, e.g., for subjects at high risk of contracting the viral infection. In some cases, the one or more modulating agents may be administered prophylactically to humans deemed to have underlying conditions, seniors (e.g., those above 65 years old), front-line healthcare workers, emergency responders, etc.

As used herein, the terms “prevent”, “preventing” and “prevention” refer to the avoidance or delay in manifestation of one or more symptoms or measurable markers of a disease or disorder. A delay in the manifestation of a symptom or marker is a delay relative to the time at which such symptom or marker manifests in a control or untreated subject with a similar likelihood or susceptibility of developing the disease or disorder. The terms “prevent” “preventing” and “prevention” include not only the avoidance or prevention of a symptom or marker of the disease, but also a reduced severity or degree of any one of the symptoms or markers of the disease, relative to those symptoms or markers in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., by at least 15%, at least 20%, at least 30%, at least 40%, at least 50%, at least 60%, at least 70%, at least 80%, at least 90%, at least 95%, at least 99% or even 100% (i.e., no symptoms or measurable markers).

As used herein, the terms “administering” and “introducing” are used interchangeably herein and refer to the placement of modulating agents of the present invention into a subject by a method or route which results in at least partial localization of the modulating agent at a desired site, e.g. a site of viral infection. The modulating agent(s) can be administered by any appropriate route which results in an effective treatment in the subject. In some embodiments, administering is not systemic administration.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration”, “administered systemically”, “peripheral administration” and “administered peripherally” as used herein mean the administration of a modulator such that it enters the animal's system and, thus, is subject to metabolism and other like processes, for example, subcutaneous administration.

### Modulating Agents

Modulating the one or more pathways herein in the cells herein may be performed by administering one or more modulating agents to the cells or the subject. In some embodiments, the methods herein include administering one or more agents that modulate the expression and/or activity of gene(s) or protein(s).

For example, the methods may include administering at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, or at least 100 modulating agents.

Modulating a gene may include modulating the expression of the gene. Modulating a gene may also include modulating the expression, the level, and/or the activity of a product encoded by the gene, e.g., a RNA or a protein. As will be clear to the skilled person, “modulating” can also involve affecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

The modulating agents may alter the function of a protein, e.g., an enzyme. For example, the modulating agents may block the interacting with the protein with its substrate or another protein. In certain examples, the modulating agents may cause conformation change that resulting in altered activity of the protein. In some examples, the modulating agents may cause degradation of the protein.

“Altered expression” as intended herein may encompass modulating the activity of one or more endogenous gene products. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar. As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

Modulation herein may include increasing, decreasing, abolishing, expression and/or activity of the one or more genes or proteins. The terms “increased” or “increase” or “upregulated” or “upregulate” as used herein generally mean an increase by a statically significant amount compared to a reference. For avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein.

The term “reduced” or “reduce” or “decrease” or “decreased” or “downregulate” or “downregulated” as used herein generally means a decrease by a statistically significant amount relative to a reference. For avoidance of doubt, “reduced” means statistically significant decrease of at least 10% as compared to a reference level, for example a decrease by at least 20%, at least 30%, at least 40%, at least t 50%, or least 60%, or least 70%, or least 80%, at least 90% or more, up to and including a 100% decrease (i.e., absent level as compared to a reference sample), or any decrease between 10-100% as compared to a reference level, as that term is defined herein. The term “abolish” or “abolished” may in particular refer to a decrease by 100%, i.e., absent level as compared to a reference sample.

The term “agent” as used herein generally refers to any substance or composition, such as a chemical entity or biological product, or combination of chemical entities or biological products, capable of achieving a desired effect in a system, more particularly in a biological system, e.g., in a cell, tissue, organ, or an organism. In the present context, an agent may be exposed to, contacted with or introduced into an immune cell to modify at least one characteristic of the immune cell, such as to (inducibly) alter the expression or activity of the one or more genes or gene products as taught herein by the immune cell. Further in the present context, an agent may be administered to a subject to treat or prevent or control a disease or condition, for example by (inducibly) altering the expression or activity of the one or more genes or gene products as taught herein by immune cells of the subject.

In some embodiments, agents useful in the methods as disclosed herein are proteins and/or peptides or fragment thereof, which inhibit the gene expression of a target gene or gene product, or the function of a target protein. Such agents include, for example, but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. As disclosed herein, a protein which inhibits the function of a target protein may be a soluble dominant negative form of the target protein or a functional fragment or variant thereof which inhibits wild-type full length target protein function.

In certain embodiments, the agents may be small molecules, antibodies, therapeutic antibody, antibody fragment, antibody-like protein scaffold, aptamer, protein, genetic modifying agent or small molecule. The chemical entity or biological product may be, but not necessarily a low molecular weight compound, but may also be a larger compound, or any organic or inorganic molecule effective in the given situation, including modified and unmodified nucleic acids such as antisense nucleic acids, RNAi, such as siRNA or shRNA, CRISPR-Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to, mutated proteins; therapeutic proteins and truncated proteins, wherein the protein is normally absent or expressed at lower levels in the cell. Proteins can also be selected from a group comprising mutated proteins, genetically engineered proteins, peptides, synthetic peptides, recombinant proteins, chimeric proteins, antibodies, minibodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments the agent is a small molecule having a chemical moiety. Agents can be known to have a desired activity and/or property, or can be selected from a library of diverse compounds.

In some embodiments, the one or more modulating agents may be small molecules. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

In certain embodiments, the modulating agents may be protein-binding agents that permit modulation or activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof.

As used herein, a “blocking” antibody” or an antibody “antagonist” is one which inhibits or reduces biological activity of the antigen(s) it binds. For example, an antagonist antibody may bind a surface receptor or ligand and inhibit the ability of the receptor and ligand to induce an ILC class 2 inflammatory response. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (e.g., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

In some embodiments, the present disclosure also features receptor-specific antibodies which both prevent ligand binding and receptor activation as well as antibodies that recognize the receptor-ligand complex. Likewise, encompassed by the present disclosure are neutralizing antibodies which bind the ligand and prevent binding of the ligand to the receptor, as well as antibodies which bind the ligand, thereby preventing receptor activation, but do not prevent the ligand from binding the receptor. Further included in the present disclosure are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., International Patent Publication No. WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present disclosure include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Methods for administering antibodies for therapeutic use is well known to one skilled in the art. In certain embodiments, small particle aerosols of antibodies or fragments thereof may be administered, preferably for treating a respiratory inflammatory disease (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies are administered in metered-dose propellant driven aerosols. In preferred embodiments, antibodies are used as inhibitors or antagonists to depress inflammatory diseases or allergen-induced asthmatic responses. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In some embodiments, the modulating agents may be nucleic acid molecules. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos e.g., the nucleic acid molecule is an RNAi molecule, i.e., RNA interference molecule. Example RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

In certain embodiments, an modulating agent may be a hormone, a cytokine, a lymphokine, a growth factor, a chemokine, a cell surface receptor ligand such as a cell surface receptor agonist or antagonist, or a mitogen.

Non-limiting examples of hormones include growth hormone (GH), adrenocorticotropic hormone (ACTH), dehydroepiandrosterone (DHEA), cortisol, epinephrine, thyroid hormone, estrogen, progesterone, testosterone, or combinations thereof.

Non-limiting examples of cytokines include lymphokines (e.g., interferon-γ, IL-2, IL-3, IL-4, IL-6, granulocyte-macrophage colony-stimulating factor (GM-CSF), interferon-γ, leukocyte migration inhibitory factors (T-LIF, B-LIF), lymphotoxin-alpha, macrophage-activating factor (MAF), macrophage migration-inhibitory factor (MIF), neuroleukin, immunologic suppressor factors, transfer factors, or combinations thereof), monokines (e.g., IL-1, TNF-alpha, interferon-α, interferon-β, colony stimulating factors, e.g., CSF2, CSF3, macrophage CSF or GM-CSF, or combinations thereof), chemokines (e.g., beta-thromboglobulin, C chemokines, CC chemokines, CXC chemokines, CX3C chemokines, macrophage inflammatory protein (MIP), or combinations thereof), interleukins (e.g., IL-1, IL-2, IL-3, IL-4, IL-5, IL-6, IL-7, IL-8, IL-9, IL-10, IL-11, IL-12, IL-13, IL-14, IL-15, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or combinations thereof), and several related signaling molecules, such as tumor necrosis factor (TNF) and interferons (e.g., interferon-α, interferon-β, interferon-γ, interferon-λ, or combinations thereof).

Non-limiting examples of growth factors include those of fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, or combinations thereof.

Non-limiting examples of mitogens include phytohaemagglutinin (PHA), concanavalin A (conA), lipopolysaccharide (LPS), pokeweed mitogen (PWM), phorbol ester such as phorbol myristate acetate (PMA) with or without ionomycin, or combinations thereof.

Non-limiting examples of cell surface receptors the ligands of which may act as agents include Toll-like receptors (TLRs) (e.g., TLR1, TLR2, TLR3, TLR4, TLR5, TLR6, TLR7, TLR8, TLR9, TLR10, TLR11, TLR12 or TLR13), CD80, CD86, CD40, CCR7, or C-type lectin receptors.

Inhibitors of Folate and/or One-Carbon Metabolism

In some embodiments, the modulating agents may be an inhibitor of folate metabolism and/or one carbon metabolism. The term “signaling pathway” or “pathway” refers to a series of cellular components involved in the intracellular or intercellular communication or transfer of information, including cell surface receptors, nuclear receptors, signal regulatory proteins, and intracellular signaling components.

### Folate and One-Carbon Metabolism Pathways

In some embodiments, the modulating agents may be antifolates, which include antimetabolite agents that antagonize the actions of folic acid. Examples of antifolates include methotrexate, pemetrexed, proguanil, pyrimethamine, trimethoprim, anticonvulsants (e.g., valproic acid, carbamazepine, phenobarbital, phenytoin, and primidone), and lamotrigine. In some examples, the antifolate may be an inhibitor of dihydrofolate reductase (DHFR). In some examples, the modulating agents may be a folate analog. The folate analog may modulate the interaction between folate and enzyme of which folate is a substrate. In some examples, the modulating agent may be a folate analog inhibiting may be dihydrofolate reductase (DHFR). In a particular example, the folate analog is methotrexate (MTX) or analog thereof.

In some examples, the modulating agent may be an inhibitor of one or more isoforms of serine hydroxymethyltransferase (e.g., SHMT1 (cytosol isoform) and SHMT2 (mitochondrial isoform)). In some cases the inhibitor may be a dual inhibitor for SHMT1 and SHMT2. An example of such an inhibitor is SHIN1:

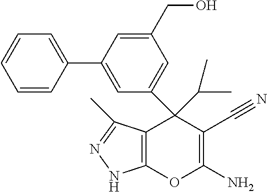 [SMILES: Cc1n[nH]c2c1C(c1cc(CO)cc(-c3ccccc3)c1)(C(C)C)C(C#N)=C(N)O2]

### SHIN, CAS No. 2146095-85-2 PGP-23,C1

In other cases, the modulating agent may be an inhibitor of a specific isoform of serine hydroxymethyltransferase, e.g., a specific inhibitor of SHMT1, or a specific inhibitor of SHMT2.

In some examples, the serine hydroxymethyltransferase inhibitor may be a compound of Formula (I)

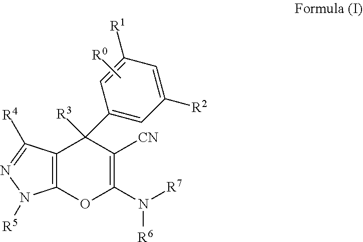

wherein:
R⁰, R¹ and R² are each independently selected from the group consisting of —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, NS(O)₂R¹², substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl, and substituted or unsubstituted haloalkoxy; provided that at least one of R⁰, R¹ and R² is selected from the group consisting of substituted or unsubstituted alkenyl, and substituted or unsubstituted alkynyl;
R³ is selected from the group consisting of —H, halogen, hydroxyl, nitro, nitrile, SOR¹¹, —S(O)₂R¹¹, —S(O)₂NR¹⁰R¹², —OR¹¹, —OC(O)R¹², —C(O)OR¹², —C(O)Rⁱⁱ, C(O)NR¹⁰R¹², —NR¹⁰R¹², N(R¹²)C(O)R¹¹, NS(O)₂R¹², substituted or unsubstituted alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl, and substituted or unsubstituted haloalkoxy;
R⁴ is selected from the group consisting of —H, substituted or unsubstituted alkyl, substituted or unsubstituted cycloalkyl, substituted or unsubstituted aryl, substituted or unsubstituted heteroaryl, substituted or unsubstituted arylalkyl, and substituted or unsubstituted heteroarylalkyl;
R⁵, R⁶ and R⁷ are each independently selected from the group consisting of —H, C(O)R¹¹, substituted or unsubstituted alkyl, substituted or unsubstituted cycloalkyl, substituted or unsubstituted aryl, substituted or unsubstituted heteroaryl, substituted or unsubstituted arylalkyl, and substituted or unsubstituted heteroarylalkyl; or R⁵ is selected from any of the foregoing and R⁶ and R⁷ taken together with the nitrogen atom to which they are attached form a substituted or unsubstituted 3-6 membered ring;
each occurrence of R¹¹is independently selected from the group consisting of substituted or unsubstituted alkyl, substituted or unsubstituted cycloalkyl, substituted or unsubstituted heterocyclyl, substituted or unsubstituted aryl, and substituted or unsubstituted heteroaryl; and
each occurrence of R¹⁰ and R¹² is independently selected from the group consisting of —H, substituted or unsubstituted alkyl, substituted or unsubstituted cycloalkyl, substituted or unsubstituted heterocyclyl, substituted or unsubstituted aryl, and substituted or unsubstituted heteroaryl.

The serine hydroxymethyltransferase inhibitor may be a 1,4-dihydropyrano[2,3-c]pyrazole derivative. Example of such inhibitors include those in Table 2 below.

TABLE 2
SHMT inhibitors having 1,4-dihydropyrano[2,3-c]pyrazole structures.

| | |
|---|---|
| 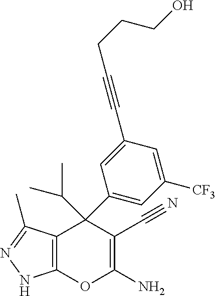 [SMILES: Cc1n[nH]c2c1C(c1cc(C#CCCCO)cc(C(F)(F)F)c1)(C(C)C)C(C#N)=C(N)O2] | 1 |
| 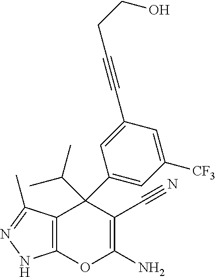 [SMILES: Cc1n[nH]c2c1C(c1cc(C#CCCO)cc(C(F)(F)F)c1)(C(C)C)C(C#N)=C(N)O2] | 2 |
| 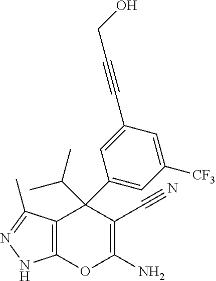 [SMILES: Cc1n[nH]c2c1C(c1cc(C#CCO)cc(C(F)(F)F)c1)(C(C)C)C(C#N)=C(N)O2] | 3 |
| 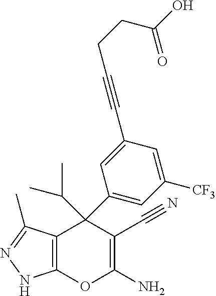 [SMILES: Cc1n[nH]c2c1C(c1cc(C#CCCC(=O)O)cc(C(F)(F)F)c1)(C(C)C)C(C#N)=C(N)O2] | 4 |
| 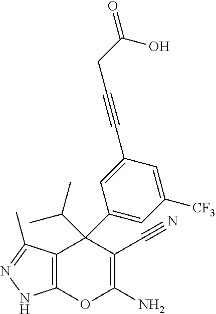 [SMILES: Cc1n[nH]c2c1C(c1cc(C#CCC(=O)O)cc(C(F)(F)F)c1)(C(C)C)C(C#N)=C(N)O2] | 5 |
| 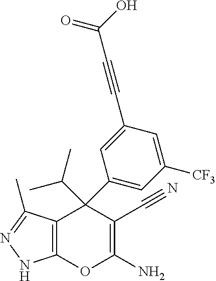 [SMILES: Cc1n[nH]c2c1C(c1cc(C#CC(=O)O)cc(C(F)(F)F)c1)(C(C)C)C(C#N)=C(N)O2] | 6 |
| 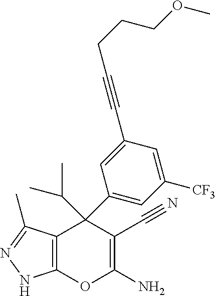 [SMILES: COCCCC#Cc1cc(C(F)(F)F)cc(C2(C(C)C)C(C#N)=C(N)Oc3[nH]nc(C)c32)c1] | 7 |
| 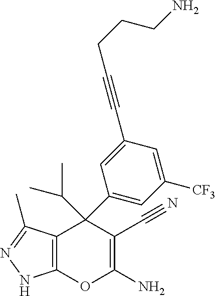 [SMILES: Cc1n[nH]c2c1C(c1cc(C#CCCCN)cc(C(F)(F)F)c1)(C(C)C)C(C#N)=C(N)O2] | 8 |
| 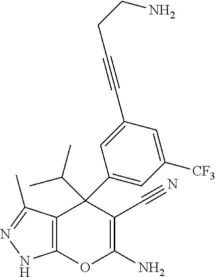 [SMILES: Cc1n[nH]c2c1C(c1cc(C#CCCN)cc(C(F)(F)F)c1)(C(C)C)C(C#N)=C(N)O2] | 9 |
| 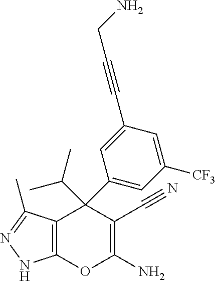 [SMILES: Cc1n[nH]c2c1C(c1cc(C#CCN)cc(C(F)(F)F)c1)(C(C)C)C(C#N)=C(N)O2] | 10 |
| 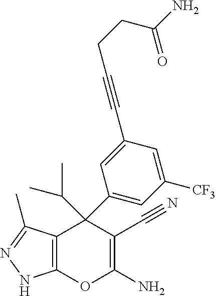 [SMILES: Cc1n[nH]c2c1C(c1cc(C#CCCC(N)=O)cc(C(F)(F)F)c1)(C(C)C)C(C#N)=C(N)O2] | 11 |
| 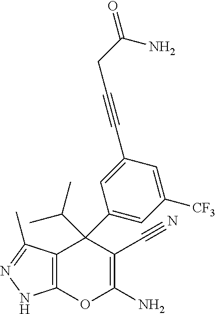 [SMILES: Cc1n[nH]c2c1C(c1cc(C#CCC(N)=O)cc(C(F)(F)F)c1)(C(C)C)C(C#N)=C(N)O2] | 12 |
| 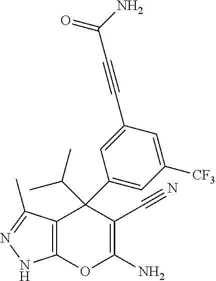 [SMILES: Cc1n[nH]c2c1C(c1cc(C#CC(N)=O)cc(C(F)(F)F)c1)(C(C)C)C(C#N)=C(N)O2] | 13 |
| 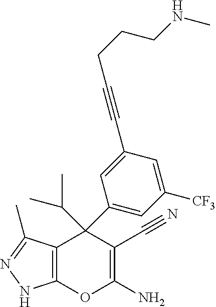 [SMILES: CNCCCC#Cc1cc(C(F)(F)F)cc(C2(C(C)C)C(C#N)=C(N)Oc3[nH]nc(C)c32)c1] | 14 |
| 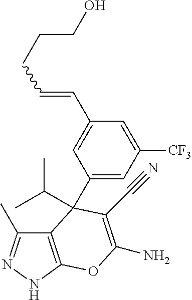 [SMILES: Cc1n[nH]c2c1C(c1cc(C=CCCCO)cc(C(F)(F)F)c1)(C(C)C)C(C#N)=C(N)O2] | 15 |
| 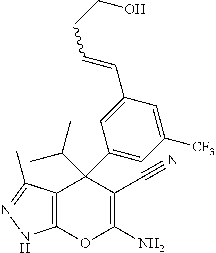 [SMILES: Cc1n[nH]c2c1C(c1cc(C=CCCO)cc(C(F)(F)F)c1)(C(C)C)C(C#N)=C(N)O2] | 16 |
| 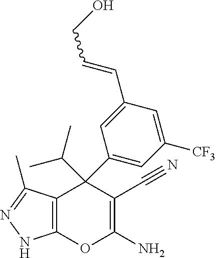 [SMILES: Cc1n[nH]c2c1C(c1cc(C=CCO)cc(C(F)(F)F)c1)(C(C)C)C(C#N)=C(N)O2] | 17 |
| 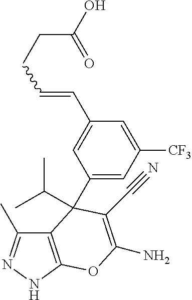 [SMILES: Cc1n[nH]c2c1C(c1cc(C=CCCC(=O)O)cc(C(F)(F)F)c1)(C(C)C)C(C#N)=C(N)O2] | 18 |
| 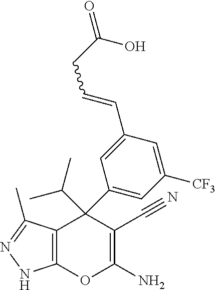 [SMILES: Cc1n[nH]c2c1C(c1cc(C=CCC(=O)O)cc(C(F)(F)F)c1)(C(C)C)C(C#N)=C(N)O2] | 19 |
| 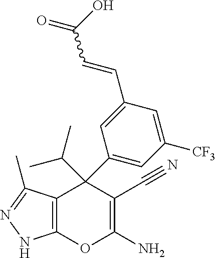 [SMILES: Cc1n[nH]c2c1C(c1cc(C=CC(=O)O)cc(C(F)(F)F)c1)(C(C)C)C(C#N)=C(N)O2] | 20 |
| 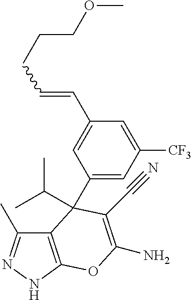 [SMILES: COCCCC=Cc1cc(C(F)(F)F)cc(C2(C(C)C)C(C#N)=C(N)Oc3[nH]nc(C)c32)c1] | 21 |
| 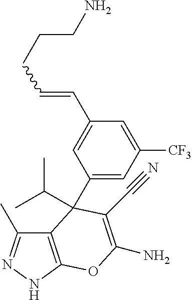 [SMILES: Cc1n[nH]c2c1C(c1cc(C=CCCCN)cc(C(F)(F)F)c1)(C(C)C)C(C#N)=C(N)O2] | 22 |
| 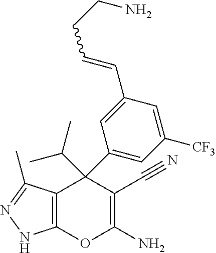 [SMILES: Cc1n[nH]c2c1C(c1cc(C=CCCN)cc(C(F)(F)F)c1)(C(C)C)C(C#N)=C(N)O2] | 23 |
| 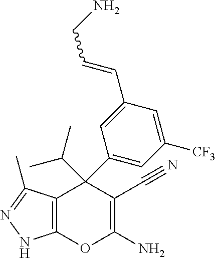 [SMILES: Cc1n[nH]c2c1C(c1cc(C=CCN)cc(C(F)(F)F)c1)(C(C)C)C(C#N)=C(N)O2] | 24 |
| 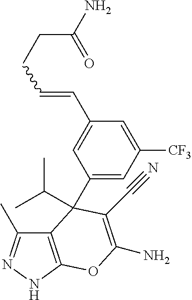 [SMILES: Cc1n[nH]c2c1C(c1cc(C=CCCC(N)=O)cc(C(F)(F)F)c1)(C(C)C)C(C#N)=C(N)O2] | 25 |
| 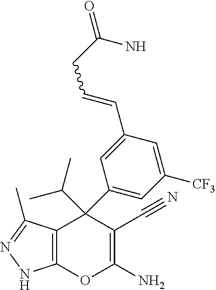 [SMILES: Cc1n[nH]c2c1C(c1cc(C=CCC(N)=O)cc(C(F)(F)F)c1)(C(C)C)C(C#N)=C(N)O2] | 26 |
| 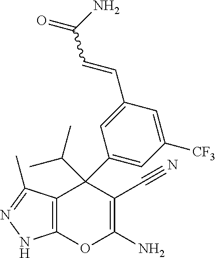 [SMILES: Cc1n[nH]c2c1C(c1cc(C=CC(N)=O)cc(C(F)(F)F)c1)(C(C)C)C(C#N)=C(N)O2] | 27 |
| 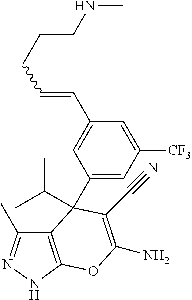 [SMILES: CNCCCC=Cc1cc(C(F)(F)F)cc(C2(C(C)C)C(C#N)=C(N)Oc3[nH]nc(C)c32)c1] | 28 |
| 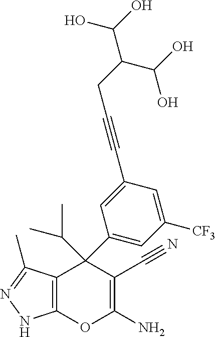 [SMILES: Cc1n[nH]c2c1C(c1cc(C#CCC(C(O)O)C(O)O)cc(C(F)(F)F)c1)(C(C)C)C(C#N)=C(N)O2] | 29 |
| 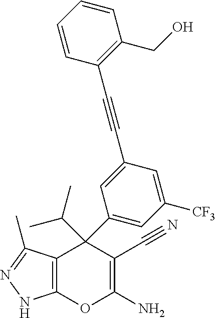 [SMILES: Cc1n[nH]c2c1C(c1cc(C#Cc3ccccc3CO)cc(C(F)(F)F)c1)(C(C)C)C(C#N)=C(N)O2] | 30 |
| 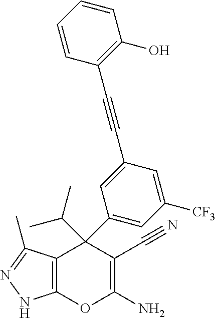 [SMILES: Cc1n[nH]c2c1C(c1cc(C#Cc3ccccc3O)cc(C(F)(F)F)c1)(C(C)C)C(C#N)=C(N)O2] | 31 |
| 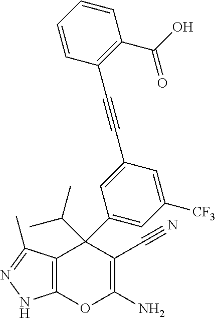 [SMILES: Cc1n[nH]c2c1C(c1cc(C#Cc3ccccc3C(=O)O)cc(C(F)(F)F)c1)(C(C)C)C(C#N)=C(N)O2] | 32 |
| 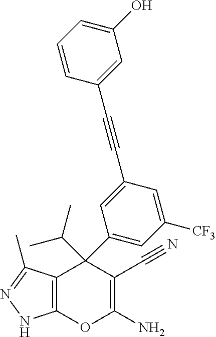 [SMILES: Cc1n[nH]c2c1C(c1cc(C#Cc3cccc(O)c3)cc(C(F)(F)F)c1)(C(C)C)C(C#N)=C(N)O2] | 33 |
| 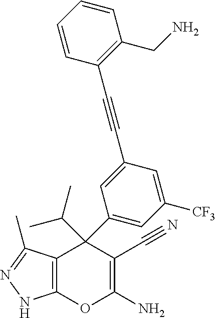 [SMILES: Cc1n[nH]c2c1C(c1cc(C#Cc3ccccc3CN)cc(C(F)(F)F)c1)(C(C)C)C(C#N)=C(N)O2] | 34 |
| 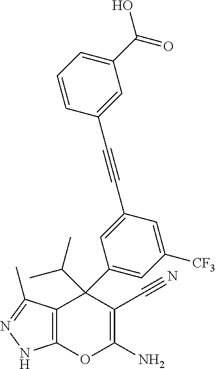 [SMILES: Cc1n[nH]c2c1C(c1cc(C#Cc3cccc(C(=O)O)c3)cc(C(F)(F)F)c1)(C(C)C)C(C#N)=C(N)O2] | 35 |
| 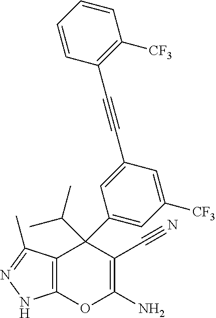 [SMILES: Cc1n[nH]c2c1C(c1cc(C#Cc3ccccc3C(F)(F)F)cc(C(F)(F)F)c1)(C(C)C)C(C#N)=C(N)O2] | 36 |
| 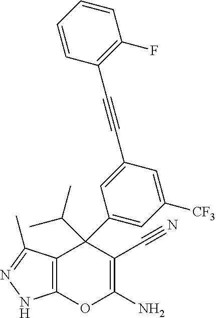 [SMILES: Cc1n[nH]c2c1C(c1cc(C#Cc3ccccc3F)cc(C(F)(F)F)c1)(C(C)C)C(C#N)=C(N)O2] | 37 |
| 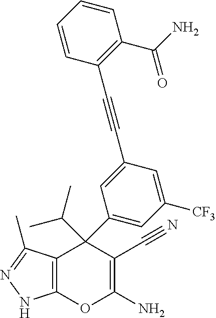 [SMILES: Cc1n[nH]c2c1C(c1cc(C#Cc3ccccc3C(N)=O)cc(C(F)(F)F)c1)(C(C)C)C(C#N)=C(N)O2] | 38 |
| 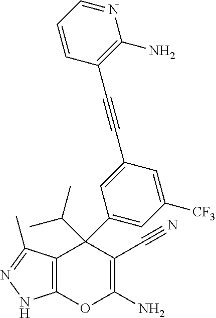 [SMILES: Cc1n[nH]c2c1C(c1cc(C#Cc3cccnc3N)cc(C(F)(F)F)c1)(C(C)C)C(C#N)=C(N)O2] | 39 |
| 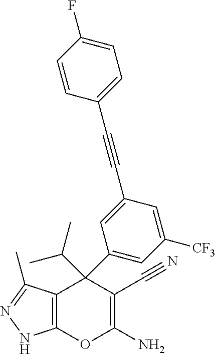 [SMILES: Cc1n[nH]c2c1C(c1cc(C#Cc3ccc(F)cc3)cc(C(F)(F)F)c1)(C(C)C)C(C#N)=C(N)O2] | 40 |
| 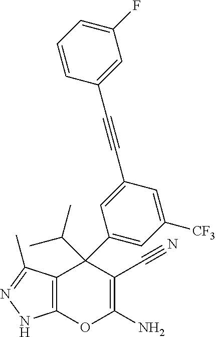 [SMILES: Cc1n[nH]c2c1C(c1cc(C#Cc3cccc(F)c3)cc(C(F)(F)F)c1)(C(C)C)C(C#N)=C(N)O2] | 41 |
| 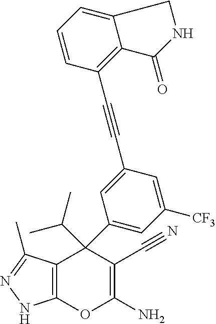 [SMILES: Cc1n[nH]c2c1C(c1cc(C#Cc3cccc4c3C(=O)NC4)cc(C(F)(F)F)c1)(C(C)C)C(C#N)=C(N)O2] | 42 |
| 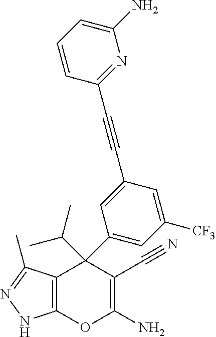 [SMILES: Cc1n[nH]c2c1C(c1cc(C#Cc3cccc(N)n3)cc(C(F)(F)F)c1)(C(C)C)C(C#N)=C(N)O2] | 43 |
| 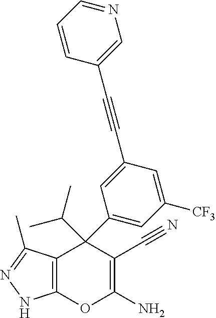 [SMILES: Cc1n[nH]c2c1C(c1cc(C#Cc3cccnc3)cc(C(F)(F)F)c1)(C(C)C)C(C#N)=C(N)O2] | 44 |
| 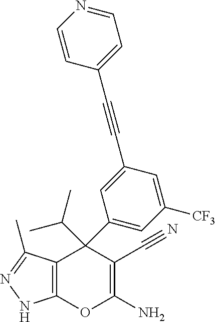 [SMILES: Cc1n[nH]c2c1C(c1cc(C#Cc3ccncc3)cc(C(F)(F)F)c1)(C(C)C)C(C#N)=C(N)O2] | 45 |
| 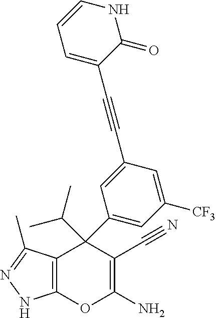 [SMILES: Cc1n[nH]c2c1C(c1cc(C#Cc3ccc[nH]c3=O)cc(C(F)(F)F)c1)(C(C)C)C(C#N)=C(N)O2] | 46 |
| 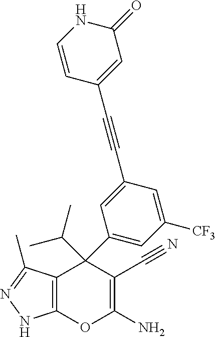 [SMILES: Cc1n[nH]c2c1C(c1cc(C#Cc3cc[nH]c(=O)c3)cc(C(F)(F)F)c1)(C(C)C)C(C#N)=C(N)O2] | 47 |
| 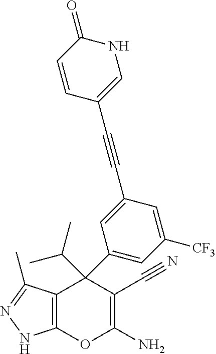 [SMILES: Cc1n[nH]c2c1C(c1cc(C#Cc3ccc(=O)[nH]c3)cc(C(F)(F)F)c1)(C(C)C)C(C#N)=C(N)O2] | 48 |
| 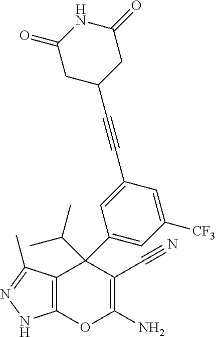 [SMILES: Cc1n[nH]c2c1C(c1cc(C#CC3CC(=O)NC(=O)C3)cc(C(F)(F)F)c1)(C(C)C)C(C#N)=C(N)O2] | 49 |
| 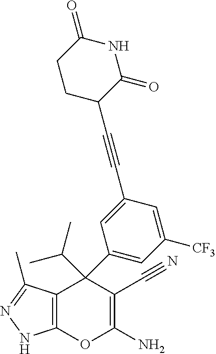 [SMILES: Cc1n[nH]c2c1C(c1cc(C#CC3CCC(=O)NC3=O)cc(C(F)(F)F)c1)(C(C)C)C(C#N)=C(N)O2] | 50 |
| 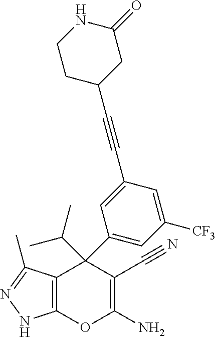 [SMILES: Cc1n[nH]c2c1C(c1cc(C#CC3CCNC(=O)C3)cc(C(F)(F)F)c1)(C(C)C)C(C#N)=C(N)O2] | 51 |
| 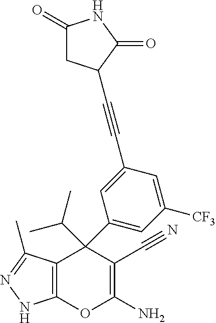 [SMILES: Cc1n[nH]c2c1C(c1cc(C#CC3CC(=O)NC3=O)cc(C(F)(F)F)c1)(C(C)C)C(C#N)=C(N)O2] | 52 |
| 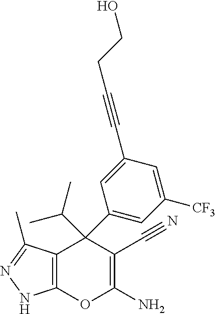 [SMILES: Cc1n[nH]c2c1C(c1cc(C#CCCO)cc(C(F)(F)F)c1)(C(C)C)C(C#N)=C(N)O2] | 53 |
| 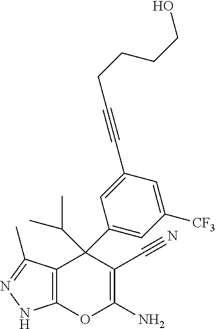 [SMILES: Cc1n[nH]c2c1C(c1cc(C#CCCCCO)cc(C(F)(F)F)c1)(C(C)C)C(C#N)=C(N)O2] | 54 |
| 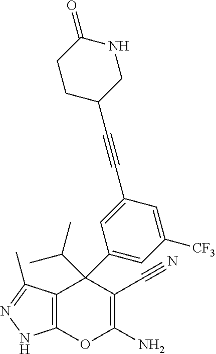 [SMILES: Cc1n[nH]c2c1C(c1cc(C#CC3CCC(=O)NC3)cc(C(F)(F)F)c1)(C(C)C)C(C#N)=C(N)O2] | 55 |
| 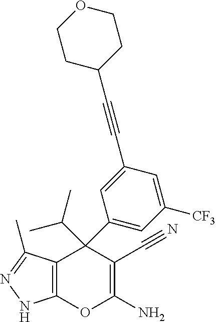 [SMILES: Cc1n[nH]c2c1C(c1cc(C#CC3CCOCC3)cc(C(F)(F)F)c1)(C(C)C)C(C#N)=C(N)O2] | 56 |
| 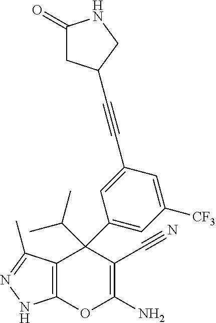 [SMILES: Cc1n[nH]c2c1C(c1cc(C#CC3CNC(=O)C3)cc(C(F)(F)F)c1)(C(C)C)C(C#N)=C(N)O2] | 57 |
| 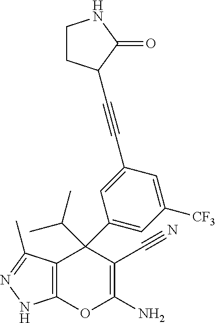 [SMILES: Cc1n[nH]c2c1C(c1cc(C#CC3CCNC3=O)cc(C(F)(F)F)c1)(C(C)C)C(C#N)=C(N)O2] | 58 |
| 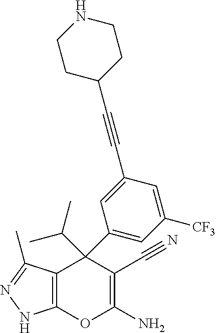 [SMILES: Cc1n[nH]c2c1C(c1cc(C#CC3CCNCC3)cc(C(F)(F)F)c1)(C(C)C)C(C#N)=C(N)O2] | 59 |
| 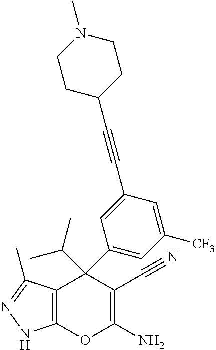 [SMILES: Cc1n[nH]c2c1C(c1cc(C#CC3CCN(C)CC3)cc(C(F)(F)F)c1)(C(C)C)C(C#N)=C(N)O2] | 60 |
| 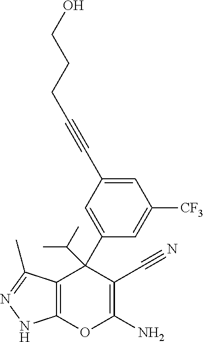 [SMILES: Cc1n[nH]c2c1C(c1cc(C#CCCCO)cc(C(F)(F)F)c1)(C(C)C)C(C#N)=C(N)O2] | 61 |
| 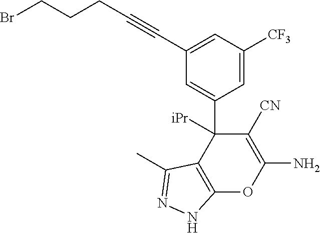 [SMILES: Cc1n[nH]c2c1C(c1cc(C#CCCCBr)cc(C(F)(F)F)c1)(C(C)C)C(C#N)=C(N)O2] | 62 |
| 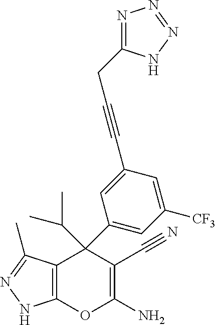 [SMILES: Cc1n[nH]c2c1C(c1cc(C#CCc3nnn[nH]3)cc(C(F)(F)F)c1)(C(C)C)C(C#N)=C(N)O2] | 63 |
| 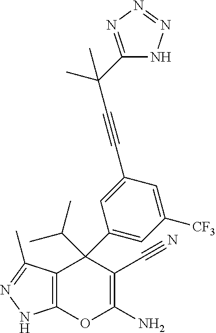 [SMILES: Cc1n[nH]c2c1C(c1cc(C#CC(C)(C)c3nnn[nH]3)cc(C(F)(F)F)c1)(C(C)C)C(C#N)=C(N)O2] | 64 |
| 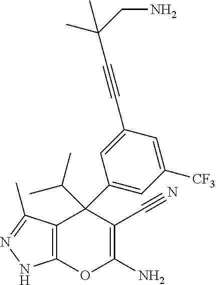 [SMILES: Cc1n[nH]c2c1C(c1cc(C#CC(C)(C)CN)cc(C(F)(F)F)c1)(C(C)C)C(C#N)=C(N)O2] | 65 |
| 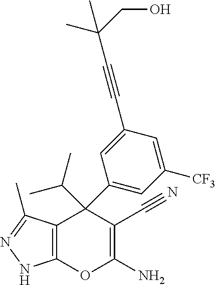 [SMILES: Cc1n[nH]c2c1C(c1cc(C#CC(C)(C)CO)cc(C(F)(F)F)c1)(C(C)C)C(C#N)=C(N)O2] | 66 |
| 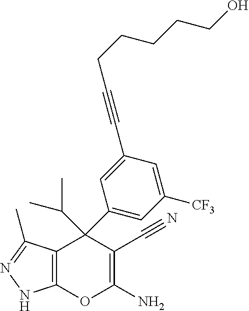 [SMILES: Cc1n[nH]c2c1C(c1cc(C#CCCCCCO)cc(C(F)(F)F)c1)(C(C)C)C(C#N)=C(N)O2] | 67 |
| 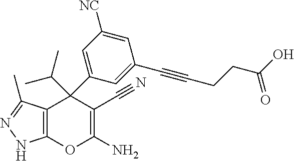 [SMILES: Cc1n[nH]c2c1C(c1cc(C#N)cc(C#CCCC(=O)O)c1)(C(C)C)C(C#N)=C(N)O2] | 68 |
| 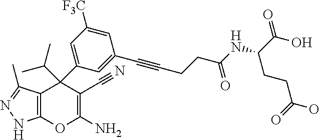 [SMILES: Cc1n[nH]c2c1C(c1cc(C#CCCC(=O)N[C@@H](CCC(=O)O)C(=O)O)cc(C(F)(F)F)c1)(C(C)C)C(C#N)=C(N)O2] | 69 |
| 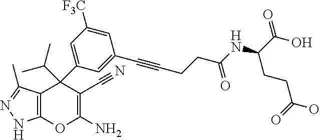 [SMILES: Cc1n[nH]c2c1C(c1cc(C#CCCC(=O)N[C@H](CCC(=O)O)C(=O)O)cc(C(F)(F)F)c1)(C(C)C)C(C#N)=C(N)O2] | 70 |
| 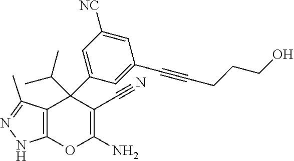 [SMILES: Cc1n[nH]c2c1C(c1cc(C#N)cc(C#CCCCO)c1)(C(C)C)C(C#N)=C(N)O2] | 71 |
| 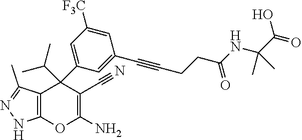 [SMILES: Cc1n[nH]c2c1C(c1cc(C#CCCC(=O)NC(C)(C)C(=O)O)cc(C(F)(F)F)c1)(C(C)C)C(C#N)=C(N)O2] | 72 |
| 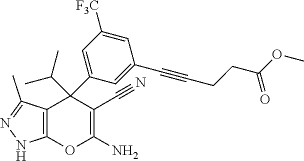 [SMILES: COC(=O)CCC#Cc1cc(C(F)(F)F)cc(C2(C(C)C)C(C#N)=C(N)Oc3[nH]nc(C)c32)c1] | 73 |
| 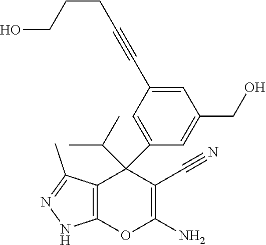 [SMILES: Cc1n[nH]c2c1C(c1cc(C#CCCCO)cc(CO)c1)(C(C)C)C(C#N)=C(N)O2] | 74 |

In a particular example, the serine hydroxymethyltransferase inhibitor is a compound with the structure of

In some examples, the serine hydroxymethyltransferase inhibitor is a compound of Formula (II):

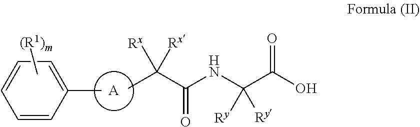

wherein:
Ring A is an optionally substituted bivalent ring optionally selected from

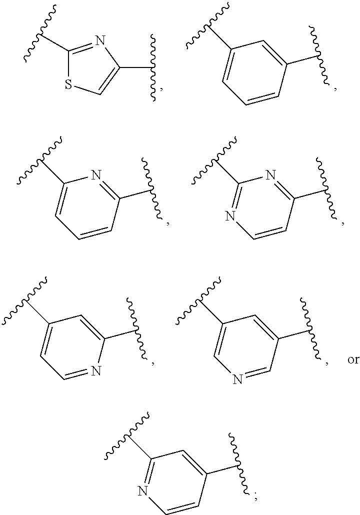

each R¹ is independently halogen, —CN, —NO₂, —OR, -Cy, or an optionally substituted C₁₋₆ aliphatic group; or
two R¹ groups can be taken together with their intervening atoms to form a 5-8 membered partially unsaturated or aryl fused ring having 0-3 heteroatoms independently selected from nitrogen, oxygen or sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
Cy is an optionally substituted group selected from a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
R^{x} and R^{y} are independently hydrogen or an optionally substituted group selected from C₁₋₆ aliphatic or phenyl;
R^{x′} and R^{y′} are independently hydrogen or C₁₋₄ alkyl; or
R^{y} and R^{y′} can be taken together with their intervening atoms to form a 3-6 membered saturated ring having 0-3 heteroatoms independently selected from nitrogen, oxygen or sulfur; and
m is 1, 2, 3, 4 or 5.

Further examples of serine hydroxymethyltransferase inhibitors include those in Table 3 below.

TABLE 3
SHMT inhibitors having structures according to Formula (II)

| | |
|---|---|
| 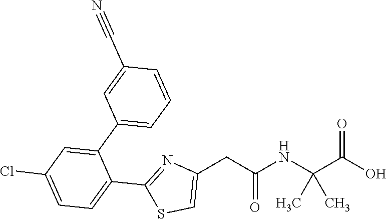 [SMILES: CC(C)(NC(=O)Cc1csc(-c2ccc(Cl)cc2-c2cccc(C#N)c2)n1)C(=O)O] | I-1 |
| 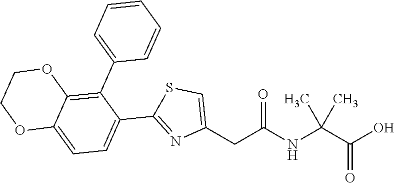 [SMILES: CC(C)(NC(=O)Cc1csc(-c2ccc3c(c2-c2ccccc2)OCCO3)n1)C(=O)O] | I-2 |
| 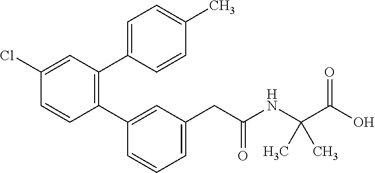 [SMILES: Cc1ccc(-c2cc(Cl)ccc2-c2cccc(CC(=O)NC(C)(C)C(=O)O)c2)cc1] | I-3 |
| 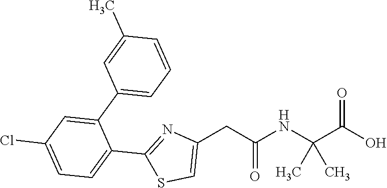 [SMILES: Cc1cccc(-c2cc(Cl)ccc2-c2nc(CC(=O)NC(C)(C)C(=O)O)cs2)c1] | I-4 |
| 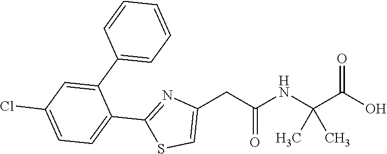 [SMILES: CC(C)(NC(=O)Cc1csc(-c2ccc(Cl)cc2-c2ccccc2)n1)C(=O)O] | I-5 |
| 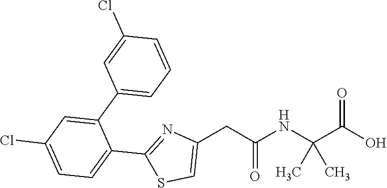 [SMILES: CC(C)(NC(=O)Cc1csc(-c2ccc(Cl)cc2-c2cccc(Cl)c2)n1)C(=O)O] | I-6 |
| 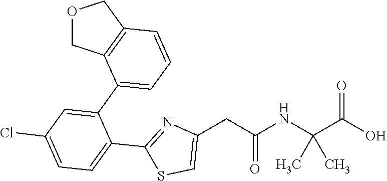 [SMILES: CC(C)(NC(=O)Cc1csc(-c2ccc(Cl)cc2-c2cccc3c2COC3)n1)C(=O)O] | I-7 |
| 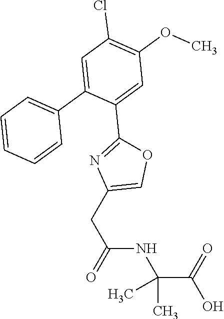 [SMILES: COc1cc(-c2nc(CC(=O)NC(C)(C)C(=O)O)co2)c(-c2ccccc2)cc1Cl] | I-8 |
| 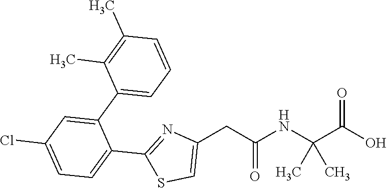 [SMILES: Cc1cccc(-c2cc(Cl)ccc2-c2nc(CC(=O)NC(C)(C)C(=O)O)cs2)c1C] | I-9 |
| 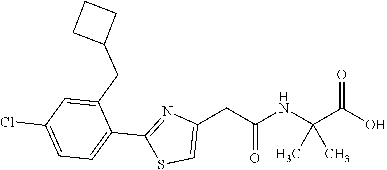 [SMILES: CC(C)(NC(=O)Cc1csc(-c2ccc(Cl)cc2CC2CCC2)n1)C(=O)O] | I-10 |
| 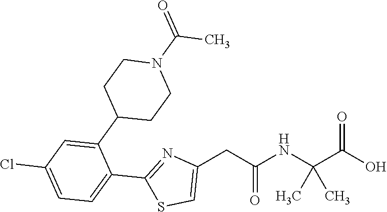 [SMILES: CC(=O)N1CCC(c2cc(Cl)ccc2-c2nc(CC(=O)NC(C)(C)C(=O)O)cs2)CC1] | I-11 |
| 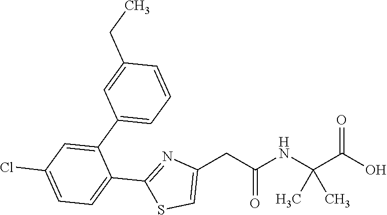 [SMILES: CCc1cccc(-c2cc(Cl)ccc2-c2nc(CC(=O)NC(C)(C)C(=O)O)cs2)c1] | I-12 |
| 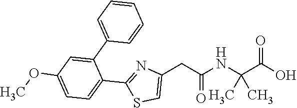 [SMILES: COc1ccc(-c2nc(CC(=O)NC(C)(C)C(=O)O)cs2)c(-c2ccccc2)c1] | I-13 |
| 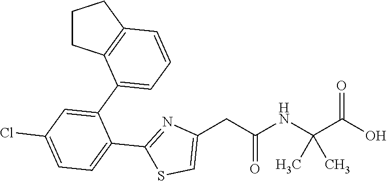 [SMILES: CC(C)(NC(=O)Cc1csc(-c2ccc(Cl)cc2-c2cccc3c2CCC3)n1)C(=O)O] | I-14 |
| 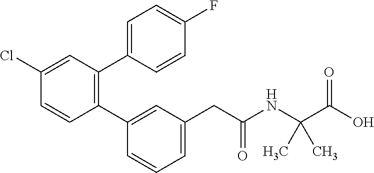 [SMILES: CC(C)(NC(=O)Cc1cccc(-c2ccc(Cl)cc2-c2ccc(F)cc2)c1)C(=O)O] | I-15 |
| 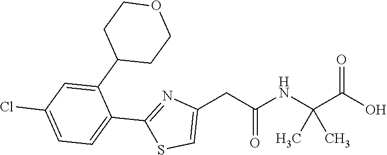 [SMILES: CC(C)(NC(=O)Cc1csc(-c2ccc(Cl)cc2C2CCOCC2)n1)C(=O)O] | I-16 |
| 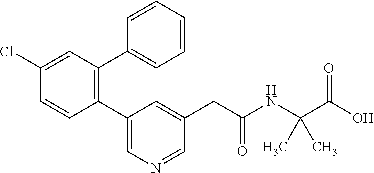 [SMILES: CC(C)(NC(=O)Cc1cncc(-c2ccc(Cl)cc2-c2ccccc2)c1)C(=O)O] | I-17 |
| 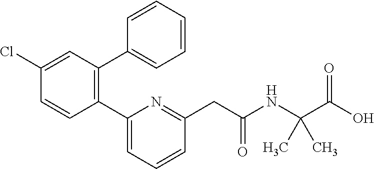 [SMILES: CC(C)(NC(=O)Cc1cccc(-c2ccc(Cl)cc2-c2ccccc2)n1)C(=O)O] | I-18 |
| 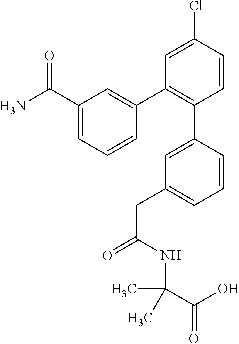 [SMILES: CC(C)(NC(=O)Cc1cccc(-c2ccc(Cl)cc2-c2cccc(C(N)=O)c2)c1)C(=O)O] | I-19 |
| 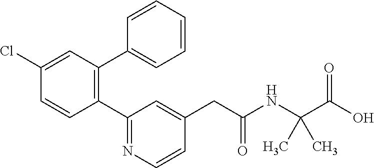 [SMILES: CC(C)(NC(=O)Cc1ccnc(-c2ccc(Cl)cc2-c2ccccc2)c1)C(=O)O] | I-20 |
| 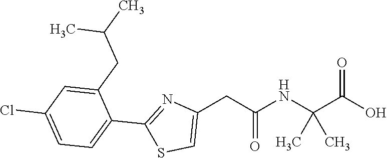 [SMILES: CC(C)Cc1cc(Cl)ccc1-c1nc(CC(=O)NC(C)(C)C(=O)O)cs1] | I-21 |
| 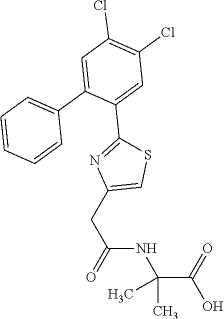 [SMILES: CC(C)(NC(=O)Cc1csc(-c2cc(Cl)c(Cl)cc2-c2ccccc2)n1)C(=O)O] | I-22 |
| 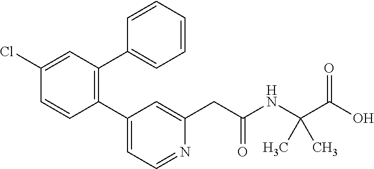 [SMILES: CC(C)(NC(=O)Cc1cc(-c2ccc(Cl)cc2-c2ccccc2)ccn1)C(=O)O] | I-23 |
| 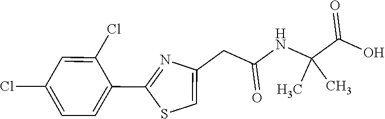 [SMILES: CC(C)(NC(=O)Cc1csc(-c2ccc(Cl)cc2Cl)n1)C(=O)O] | I-24 |
| 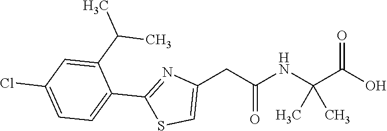 [SMILES: CC(C)c1cc(Cl)ccc1-c1nc(CC(=O)NC(C)(C)C(=O)O)cs1] | I-25 |
| 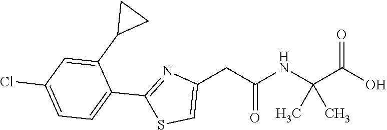 [SMILES: CC(C)(NC(=O)Cc1csc(-c2ccc(Cl)cc2C2CC2)n1)C(=O)O] | I-26 |
| 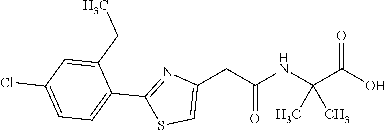 [SMILES: CCc1cc(Cl)ccc1-c1nc(CC(=O)NC(C)(C)C(=O)O)cs1] | I-27 |
| 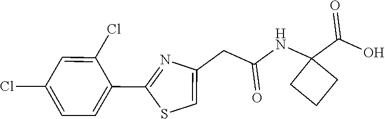 [SMILES: O=C(Cc1csc(-c2ccc(Cl)cc2Cl)n1)NC1(C(=O)O)CCC1] | I-28 |
| 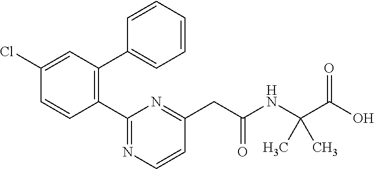 [SMILES: CC(C)(NC(=O)Cc1ccnc(-c2ccc(Cl)cc2-c2ccccc2)n1)C(=O)O] | I-29 |
| 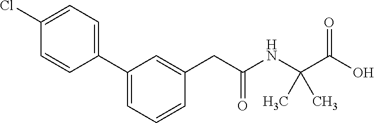 [SMILES: CC(C)(NC(=O)Cc1cccc(-c2ccc(Cl)cc2)c1)C(=O)O] | I-30 |
| 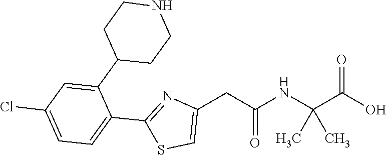 [SMILES: CC(C)(NC(=O)Cc1csc(-c2ccc(Cl)cc2C2CCNCC2)n1)C(=O)O] | I-31 |
| 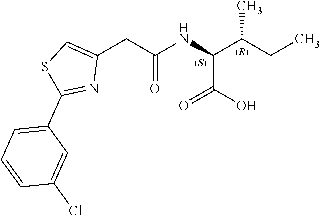 | I-32 |
| 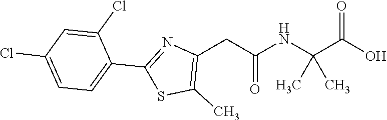 [SMILES: Cc1sc(-c2ccc(Cl)cc2Cl)nc1CC(=O)NC(C)(C)C(=O)O] | I-33 |
| 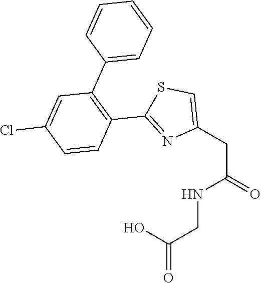 [SMILES: O=C(O)CNC(=O)Cc1csc(-c2ccc(Cl)cc2-c2ccccc2)n1] | I-34 |
| 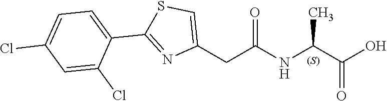 | I-35 |
| 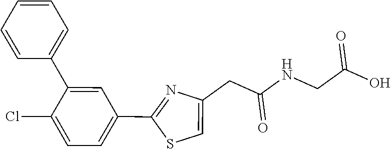 [SMILES: O=C(O)CNC(=O)Cc1csc(-c2ccc(Cl)c(-c3ccccc3)c2)n1] | I-36 |
| 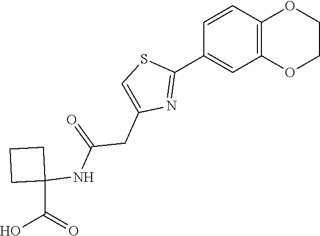 [SMILES: O=C(Cc1csc(-c2ccc3c(c2)OCCO3)n1)NC1(C(=O)O)CCC1] | I-37 |
| 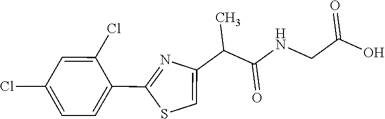 [SMILES: CC(C(=O)NCC(=O)O)c1csc(-c2ccc(Cl)cc2Cl)n1] | I-38 |
| 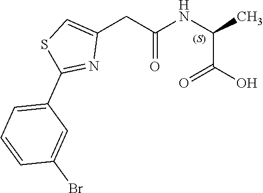 | I-39 |
| 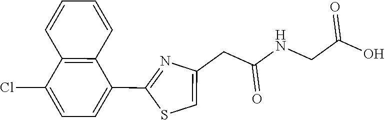 [SMILES: O=C(O)CNC(=O)Cc1csc(-c2ccc(Cl)c3ccccc23)n1] | I-40 |
| 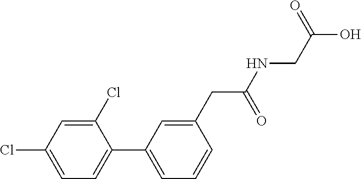 [SMILES: O=C(O)CNC(=O)Cc1cccc(-c2ccc(Cl)cc2Cl)c1] | I-41 |
| 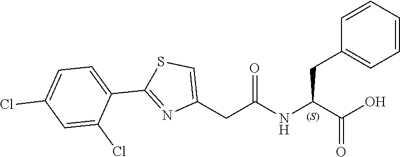 | I-42 |
| 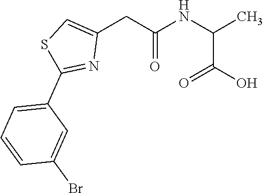 [SMILES: CC(NC(=O)Cc1csc(-c2cccc(Br)c2)n1)C(=O)O] | I-43 |
| 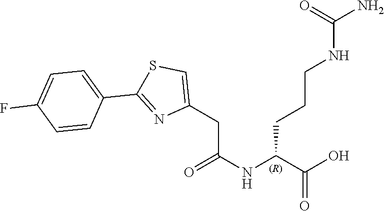 | I-44 |
| 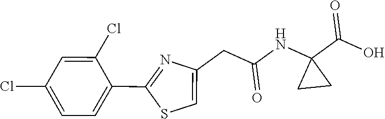 [SMILES: O=C(Cc1csc(-c2ccc(Cl)cc2Cl)n1)NC1(C(=O)O)CC1] | I-45 |
| 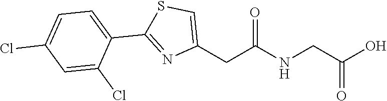 [SMILES: O=C(O)CNC(=O)Cc1csc(-c2ccc(Cl)cc2Cl)n1] | I-46 |
| 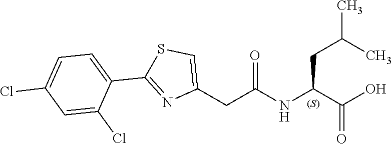 | I-47 |
| 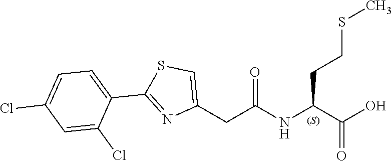 | I-48 |
| 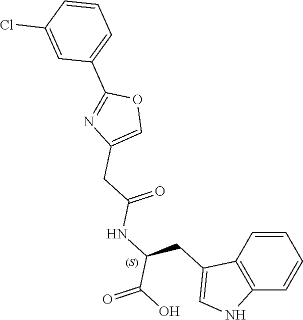 | I-49 |
| 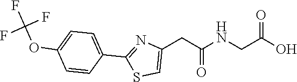 [SMILES: O=C(O)CNC(=O)Cc1csc(-c2ccc(OC(F)(F)F)cc2)n1] | I-50 |
| 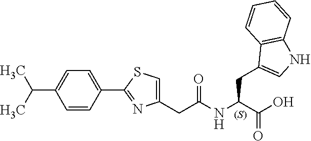 | I-51 |
| 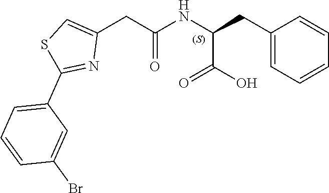 | I-52 |
| 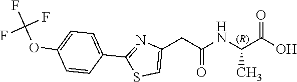 | I-53 |
| 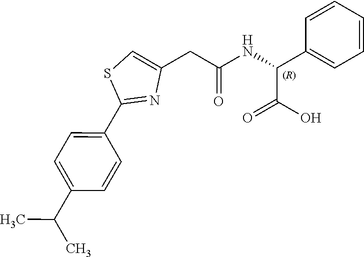 | I-54 |
| 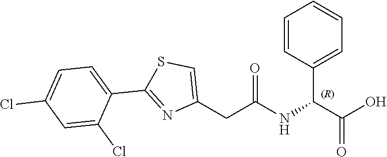 | I-55 |
| 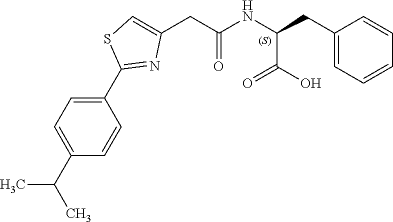 | I-56 |
| 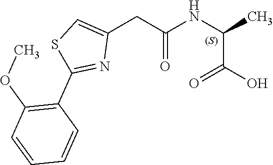 | I-57 |
| 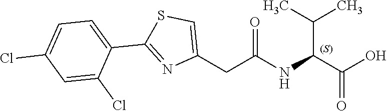 | I-58 |
| 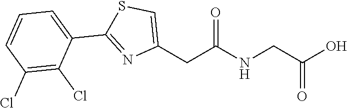 [SMILES: O=C(O)CNC(=O)Cc1csc(-c2cccc(Cl)c2Cl)n1] | I-59 |
| 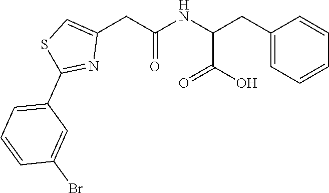 [SMILES: O=C(Cc1csc(-c2cccc(Br)c2)n1)NC(Cc1ccccc1)C(=O)O] | I-60 |
| 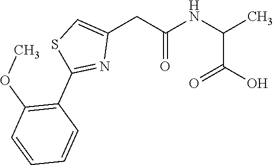 [SMILES: COc1ccccc1-c1nc(CC(=O)NC(C)C(=O)O)cs1] | I-61 |
| 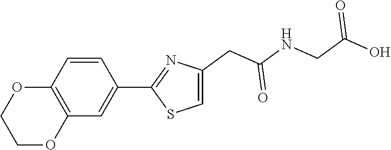 [SMILES: O=C(O)CNC(=O)Cc1csc(-c2ccc3c(c2)OCCO3)n1] | I-62 |
| 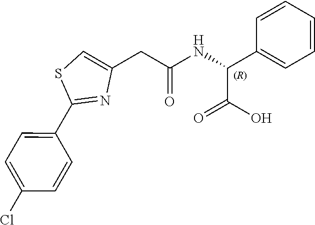 | I-63 |
| 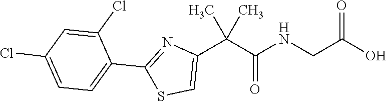 [SMILES: CC(C)(C(=O)NCC(=O)O)c1csc(-c2ccc(Cl)cc2Cl)n1] | I-64 |
| 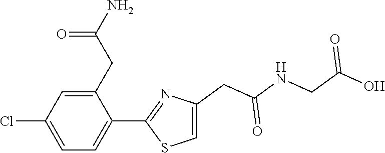 [SMILES: NC(=O)Cc1cc(Cl)ccc1-c1nc(CC(=O)NCC(=O)O)cs1] | I-65 |
| 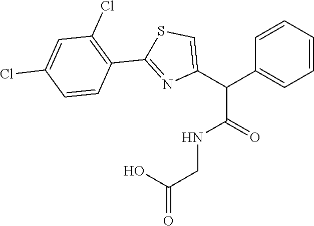 [SMILES: O=C(O)CNC(=O)C(c1ccccc1)c1csc(-c2ccc(Cl)cc2Cl)n1] | I-66 |
| 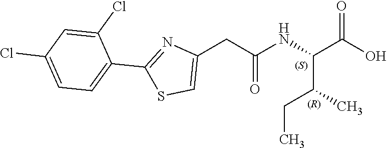 | I-67 |
| 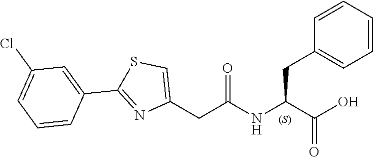 | I-68 |
| 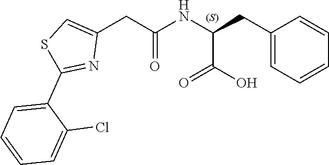 | I-69 |
| 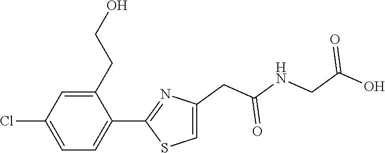 [SMILES: O=C(O)CNC(=O)Cc1csc(-c2ccc(Cl)cc2CCO)n1] | I-70 |
| 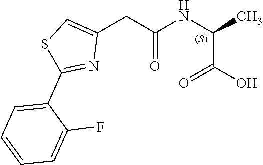 | I-71 |

Examples of serine hydroxymethyltransferase inhibitors also include compounds described in Dabinowitz J D et al., US20200239488A1 and Mainolfi N, US20190345119A1, which are incorporated herein by their entireties.

Example genes and proteins in the folate metabolism and one-carbon metabolism pathways include MTR (methionine synthase), MTHFR (methylenetetrahydrofolate reductase), TYMS (thymidylate synthetase), DHFR (dihydrofolate reductase), SHMT1 (serine hydroxymethyl transferase, cytosolic (1)), MTHFD1 (m ethylenetetrahydrofolate dehydrogenase, cyclohydrolase, and formyltetrahydrofolate synthetase 1), GART (phosphoribosylglycinamide formyltransferase), ATIC (5-aminoimidazole-4-carboxamide ribonucleotide formyltransferase/IMP cyclohydrolase), ALDH1L1 (cytosolic (1) 10-formyltetrahydrofolate dehydrogenase), MTHFD1L (monofunctional tetrahydrofolate synthase, Mitochondria), MTHFD2/L (methylenetetrahydrofolate dehydrogenase 2/2-like), SHMT2 (serine hydroxymethyl transferase, mitochondrial (2)), MTFMT (mitochondrial methionyl-tRNA formyltransferase), ALDH1L2 (mitochondrial (2) 10-formyltetrahydrofolate dehydrogenase), and TYMS (thymidylate synthetase). Examples targets for the modulating agents herein include substrates, ligands, receptors, genes and proteins described in Ducker G S et al., One-Carbon Metabolism in Health and Disease, Cell Metab. 2017 Jan. 10; 25(1):27-42, which is incorporated herein in its entirety.

### Glucose Metabolism and Glycolysis

In some embodiments, the one or more modulating agents may modulate (e.g., inhibit) the substrates, ligands, receptors, genes or proteins in glucose metabolism and/or glycolysis pathways.

Example genes and proteins in the glucose metabolism and/or glycolysis pathways include glucose-6-phosphate dehydrogenase, 6-phosphogluconolactonase, 6-phosphogluconate dehydrogenase, hexokinase, glucose-6-phosphate isomerase, phosphofructokinase-1, fructose-bisphosphate aldolase, triosephosphate isomerase, glyceraldehyde-3-phosphate dehydrogenase, phosphoglycerate kinase, phosphoglycerate mutase, phosphopyruvate hydratase (enolase), and pyruvate kinase.

In some embodiments, the one or more modulating agents comprises one or more inhibitors of glucose-6-phosphate dehydrogenase (G6PD), e.g., 6-aminonicotinamide (6-AN) and dehydroepiandrosterone (DHEA), and dantrolene.

### Oxidative Phosphorylation Pathways

In some embodiments, the one or more modulating agents may modulate (e.g., inhibit) the substrates, ligands, receptors, genes or proteins in oxidative phosphorylation pathways. Example genes and proteins in the oxidative phosphorylation pathways include components of the NADH dehydrogenase, Succinate dehydrogenase, Cytochrome bcl complex, and mitochondrial complex IV. In some examples, the modulating agents may be modulators (e.g., inhibitors) of the electron transport chain complex I. In a particular example, the modulating agent is Piericidin A (PierA). In some examples, the modulating agent is metformin. Other examples of inhibitors of the oxidative phosphorylation pathways include phenformin, atovaquone, chloramphenicol, meclizine, and S-meclizine.

### Amino Acids

In some embodiments, the one or more modulating agents may modulate (e.g., inhibit) the levels of certain amino acids, e.g., aspartate and asparagine. In some examples, the one or more modulating agents may be aspartate, asparagine, and/or analogs thereof.

### Purine Synthesis Pathways

In some embodiments, the one or more modulating agents may modulate (e.g., inhibit) purine synthesis (e.g., de novo purine synthesis). The one or more modulating agents may be inhibitors of purine synthesis, e.g., Azathioprine, Mycophenolate mofetil, Allopurinol, mercaptopurine, and agents described in Christopherson R I et al., Acc Chem Res. 2002 Nov;35(11):961-71. Inhibitors of de novo nucleotide biosynthesis as drugs, which is incorporated by reference herein in its entirety. In some examples, the one or more modulating agents may be purine synthesis intermediates, including 5-phosphoribosyl-1-pyrophosphate (PRPP), N-formylglycinamide ribonucleotide (FGAR), aminoimidazole ribonucleotide (AIR), and succinylaminoimidazolecarboxamide ribonucleotide (SAICAR), or analogs thereof.

### Serine Synthesis Pathways

In some embodiments, the one or more modulating agents may modulate (e.g., inhibit) serine synthesis (e.g., de novo serine synthesis). The one or more modulating agents may be inhibitors of serine synthesis, e.g., inhibitors of Phosphoglycerate dehydrogenase (PHGDH). Examples of PHGDH inhibitors include CBR-5884, and those described in Edouard Mullarky et al., Identification of a small molecule inhibitor of 3-phosphoglycerate dehydrogenase to target serine biosynthesis in cancers, PNAS Feb. 16, 2016 113 (7) 1778-1783; and EdouardMullarky et al., Inhibition of 3-phosphoglycerate dehydrogenase (PHGDH) by indole amides abrogates de novo serine synthesis in cancer cells, Bioorganic & Medicinal Chemistry Letters, Volume 29, Issue 17, 1 Sep. 2019, Pages 2503-2510, which are incorporated by reference in their entireties.

### Other Example Targets

Other examples of targets for the modulating agents include genes and proteins in Table 4A identified by RNAseq analysis in Example 1. In some embodiments, examples of targets for the modulating agents include genes and proteins in Table 4B identified by RNAseq analysis in Example 1. Additional examples of targets for the modulating agents include the metabolites in Table 6. In an example embodiment, the metabolite is selected from Glutamate, N6,N6,N6-Trimethyl-L-lysine. Glutamine, Taurine, Fumarate, FGAR, Malate, 2-Hydroxyglutarate, Creatine, S-Adenosylhomocysteine, Succinate, Nicotinamide, UDP, Glucuronate, Valine, Glycerol-3-Phosphate, Glycine, 5-Aminoimidazole ribotide, Phosphocreatine, Mannose-6-Phosphate/Fructose-1-phosphate, Arginine, Serine, Isoleucine, Lactate, Glucosamine-6-Phosphate, Saccharate/Galactarate_01, Saccharate/Galactarate_02, Pyruvate, Threonine, Glucose, N-Acetylaspartate, Cystathionine, Pyridoxine, Galactitol/Mannitol/Sorbitol, Y-Aminobutyric acid, Proline, S-Adenosylmethionine, Alpha-ketoglutarate, 5-Aminovaleric acid, Putrescine, L-Glutathione reduced, Aspartate, Asparagine, Phosphorylcholine, Inosine monophosphate, methionine sulfoxide, Choline, Citrulline, Citrate, L-Glutathione oxidized, UDP-N-acetylglucosamine/UDP-N-acetylgalactosamine, Methionine, Alanine, Folate, Glucose-6-Phosphate, S-adnosyolhomocysteine, 3-Phosphoglycerate/2-Phosphoglycerate, N-Acetylglutamate, Pyroglutamate/Oxoproline, Leucine, L-Histidine, Lysine, Tryptophan, N,N-Dimethyl-L-arginine, SAICAR, Cysteine, Allantoin, PRPP, Argininosuccinic acid, Ornithine, Fructose 1,6-bisphosphate, Phenylalanine, Kynurenine, L-Carnitine, Aminoadipate, Creatinine, 3-Hydroxymethylglutarate, Tyrosine, Deoxycarnitine, GDP-Glucose, Ribose-5-Phosphate/Xylulose-5-phosphate, Xylitol/Ribitol/Arabitol, 1-Aminocyclopropanecarboxylate, Phosphoenolpyruvate, CDP-ethanolamine, Betaine, Acetyl-L-carnitine, 1-Methyl-L-Histidine, Hypotaurine, UDP Glucose/UDP Galactose, D-Gluconate, Cytidine, Hippurate, Isociitrate, 2-Hydroxybutyrate/Alpha-hydroxyisobutyrate, 2-Quinolinecarboxylate/4-Quinolinecarboxylate, Ureidosuccinic acid, N-Acetylcysteine, L-Cysteinesulfinic acid, Glyceraldehyde 3-phosphate, Dihydroxyacetone Phosphate, Urocanic acid, 5-Formimino-tetrahydrofol ate, L-Cysteine-gutathione Disulfide, and Spermidine. For example, the modulating agents may increase or inhibit production of the metabolites. In some examples, the modulating agents may be the metabolites in Table 6. In some examples, the modulating agents may be analogs of the metabolites in Table 6.

### Nutritional Manipulation

In some embodiments, the methods may comprise nutritional manipulation, e.g., providing a suitable diet to a subject for treating and/or preventing viral infection. For example, the method may comprise providing a subject with a serine/glycine deficient diet. Such diet may nutritionally inhibit one carbon metabolism.

### Regulatory Sequences

When the modulating agents comprise nucleic acids, the nucleic acid may be operably linked to one or more regulatory sequences. In some cases, the regulatory sequences may direct the expression of the nucleic acids in specific types. The term “operably linked” as used herein refers to linkage of a regulatory sequence to from a DNA sequence such that the regulatory sequence regulates the mediates transcription of the DNA sequence. Regulatory sequences include transcription control sequences, e.g., sequences which control the initiation, elongation and termination of transcription. In some cases, regulatory sequences include those control transcriptions. Examples of such regulatory sequences include promoters, enhancers, operators, repressor, transcription terminator sequences.

### Promoters

In some examples, the regulatory sequences are promoters. A promoter refers to a nucleic acid sequence that directs the transcription of a operably linked sequence into mRNA. The promoter or promoter region may provide a recognition site for RNA polymerase and the other factors necessary for proper initiation of transcription. When a sequence operably linked to a promoter is controlled or driven by the promoter. A promoter may include at least the Core promoter, e.g., a sequence for initiating transcription. The promoter may further at least the Proximal promoter, e.g., a proximal sequence upstream of the gene that tends to contain primary regulatory elements. The promoter may also include the Distal promoter, e.g., the distal sequence upstream of the gene that may contain additional regulatory elements.

The promoters may be from about 50 to about 2000 base pairs (bp), from about 100 to about 1000, from about 50 to about 150, from about 100 to about 200, from about 150 to about 250, from about 200 to about 300, from about 250 to about 350, from about 300 to about 400, from about 350 to about 450, from about 400 to about 500, from about 450 to about 550, from about 500 to about 600, from about 550 to about 650, from about 600 to about 700, from about 650 to about 750, from about 700 to about 800, from about 750 to about 850, from about 800 to about 900, from about 850 to about 950, from about 900 to about 1000, from about 950 to about 1050, from about 1000 to about 1100 bp in length.

The promoters may include sequences that bind to regulatory proteins. In some examples, the regulatory sequences may be sequences that bind to transcription activators. In certain examples, the regulatory sequences may be sequences that bind to transcription repressors.

In some cases, the promoter may be a constitutive promoter, e.g., U6 and H1 promoters, retroviral Rous sarcoma virus (RSV) LTR promoter, cytomegalovirus (CMV) promoter, SV40 promoter, dihydrofolate reductase promoter, (3-actin promoter, phosphoglycerol kinase (PGK) promoter, ubiquitin C, U5 snRNA, U7 snRNA, tRNA promoters or EF1α promoter. In certain cases, the promoter may be a tissue-specific promoter may direct expression primarily in a desired tissue of interest, such as muscle, neuron, bone, skin, blood, specific organs (e.g. liver, pancreas), or particular cell types (e.g. lymphocytes). Examples of tissue-specific promoters include Ick, myogenin, or thy1 promoters. In some embodiments, the promoter may direct expression in a temporal-dependent manner, such as in a cell-cycle dependent or developmental stage-dependent manner, which may or may not also be tissue or cell-type specific. In certain cases, the promoter may be an inducible promoter, e.g., can be activated by a chemical such as doxycycline.

In some cases, a promoter is specific to one or more genes. For examples, the promoter may only regulate (e.g., activates) transcription of the one or more genes, not other genes.

In some cases, the promoters may be cell-specific, tissue-specific, or organ-specific promoters. In some examples, the promoters may be CD+4 T cell specific promoters, monocyte specific promoters, cytotoxic lymphocyte specific promoters, natural killer (NK) cell specific promoters, proliferating T cell specific promoters, resting monocyte specific promoters, inflammatory monocyte specific promoters, CD 16+monocyte specific promoters, anti-viral monocyte specific promoters, anti-viral/inflammatory monocyte specific promoters, CD1C+dendric cell specific promoters, plasmacytoid dendric cell specific promoters, B cell specific promoters, plasmablast specific promoters, dendric cell specific promoters, or any combination thereof. Examples of the cell-specific promoters include B29 promoters (for B cells), CD14 prooters (for monocytes), CD43 promoters (leukocytes and platelets), CD68 promoters (for macrophages). Other examples of tissue-specific promoters for lymphocytes include the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′transcriptional enhancer, and the human NK and T cell specific activation (NKG5) promoter.

Example of cell-specific, tissue-specific, or organ-specific promoters include promoter for creatine kinase, (for expression in muscle and cardiac tissue), immunoglobulin heavy or light chain promoters (for expression in B cells), smooth muscle alpha-actin promoter. Exemplary tissue-specific promoters for the liver include HMG-COA reductase promoter, sterol regulatory element 1, phosphoenol pyruvate carboxy kinase (PEPCK) promoter, human C-reactive protein (CRP) promoter, human glucokinase promoter, cholesterol 7-alpha hydroylase (CYP-7) promoter, beta-galactosidase alpha-2,6 sialyltransferase promoter, insulin-like growth factor binding protein (IGFBP-1) promoter, aldolase B promoter, human transferrin promoter, and collagen type I promoter. Exemplary tissue-specific promoters for the prostate include the prostatic acid phosphatase (PAP) promoter, prostatic secretory protein of 94 (PSP 94) promoter, prostate specific antigen complex promoter, and human glandular kallikrein gene promoter (hgt-1). Exemplary tissue-specific promoters for gastric tissue include H+/K+-ATPase alpha subunit promoter. Exemplary tissue-specific expression elements for the pancreas include pancreatitis associated protein promoter (PAP), elastase 1 transcriptional enhancer, pancreas specific amylase and elastase enhancer promoter, and pancreatic cholesterol esterase gene promoter. Exemplary tissue-specific promoters for the endometrium include, the uteroglobin promoter. Exemplary tissue-specific promoters for adrenal cells include cholesterol side-chain cleavage (SCC) promoter. Exemplary tissue-specific promoters for the general nervous system include gamma-gamma enolase (neuron-specific enolase, NSE) promoter. Exemplary tissue-specific promoters for the brain include the neurofilament heavy chain (NF-H) promoter. Exemplary tissue-specific promoters for the colon include pp60c-src tyrosine kinase promoter, organ-specific neoantigens (OSNs) promoter, and colon specific antigen-P promoter. Exemplary tissue-specific promoters for breast cells include the human alpha-lactalbumin promoter. Exemplary tissue-specific promoters for the lung include the cystic fibrosis transmembrane conductance regulator (CFTR) gene promoter.

Examples of cell-specific, tissue-specific, or organ-specific promoters may also include those used for expressing the barcode or other transcripts within a particular plant tissue (See e.g., International Patent Publication No. WO 2001/098480A2, “Promoters for regulation of plant gene expression”). Examples of such promoters include the lectin (Vodkin, Prog. Clinc. Biol. Res., 138:87-98 (1983); and Lindstrom et al., Dev. Genet., 11:160-167 (1990)), corn alcohol dehydrogenase 1 (Dennis et al., Nucleic Acids Res., 12:3983-4000 (1984)), corn light harvesting complex (Becker, Plant Mol Biol., 20(1): 49-60 (1992); and Bansal et al., Proc. Natl. Acad. Sci. U.S.A., 89:3654-3658 (1992)), corn heat shock protein (Odell et al., Nature (1985) 313:810-812; and Marrs et al., Dev. Genet., 14(1):27-41 (1993)), small subunit RuBP carboxylase (Waksman et al., Nucleic Acids Res., 15(17):7181 (1987); and Berry-Lowe et al., J. Mol. Appl. Genet., 1(6):483-498 (1982)), Ti plasmid mannopine synthase (Ni et al., Plant Mol. Biol., 30(1):77-96 (1996)), Ti plasmid nopaline synthase (Bevan, Nucleic Acids Res., 11(2):369-385 (1983)), petunia chalcone isomerase (Van Tunen et al., EMBO J., 7:1257-1263 (1988)), bean glycine rich protein 1 (Keller et al., Genes Dev., 3:1639-1646 (1989)), truncated CaMV 35s (Odell et al., Nature (1985) 313:810-812), potato patatin (Wenzler et al., Plant Mol. Biol., 13:347-354 (1989)), root cell (Yamamoto et al., Nucleic Acids Res., 18:7449 (1990)), maize zein (Reina et al., Nucleic Acids Res., 18:6425 (1990); Kriz et al., Mol. Gen. Genet., 207:90-98 1987; Wandelt and Feix, Nucleic Acids Res., 17:2354 (1989); Langridge and Feix, Cell, 34:1015-1022 (1983); and Reina et al., Nucleic Acids Res., 18:7449 (1990)), globulin-1 (Belanger et al., Genetics, 129:863-872 (1991)), α-tubulin, cab (Sullivan et al., Mol. Gen. Genet.,215:431-440 (1989)), PEPCase (Cushman et al., Plant Cell, 1(7):715-25 (1989)), R gene complex-associated promoters (Chandler et al., Plant Cell, 1: 1175-1183 (1989)), and chalcone synthase promoters (Franken et al., EMBO J., 10:2605-2612, 1991)). Examples of tissue-specific promoters also include those described in the following references: Yamamoto et al., Plant J (1997) 12(2):255-265; Kawamata et al., Plant Cell Physiol. (1997) 38(7):792-803; Hansen et al., Mol. Gen Genet. (1997) 254(3):337); Russell et al., Transgenic Res. (1997) 6(2):157-168; Rinehart et al., Plant Physiol. (1996) 112(3):1331; Van Camp et al., Plant Physiol. (1996) 112(2):525-535; Canevascini et al., Plant Physiol. (1996) 112(2):513-524; Yamamoto et al., Plant Cell Pkysiol. (1994) 35(5):773-778; Lam, Results Probl. Cell Differ. (1994) 20:181-196; Orozco et al., Plant Mol. Biol. (1993) 23(6):1129-1138; Matsuoka et al., Proc Natl. Acad. Sci. USA (1993) 90(20):9586-9590; and Guevara-Garcia et al., Plant J. (1993) 4(3):495-505; maize phosphoenol carboxylase (PEPC) has been described by Hudspeth & Grula (Plant Molec Biol 12: 579-589 (1989)); leaf-specific promoters such as those described in Yamamoto et al., Plant J. (1997) 12(2):255-265; Kwon et al., Plant Physiol. (1994) 105:357-367; Yamamoto et al., Plant Cell Physiol. (1994) 35(5):773-778; Gotor et al., Plant J. (1993) 3:509-518; Orozco et al., Plant Mol. Biol. (1993) 23(6):1129-1138; and Matsuoka et al., Proc. Natl. Acad. Sci. USA (1993) 90(20):9586-9590.

### Gene Editing Systems

In certain embodiments, the one or more modulating agents may be one or more components of a gene editing system. Examples of gene editing systems include a CRISPR-Cas system, a zinc finger nuclease system, a TALEN, and a meganuclease system.

### CRISPR-Cas System

In some embodiments, the one or more modulating agents may be one or more components of a CRISPR-Cas system. In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

### Class 1 Systems

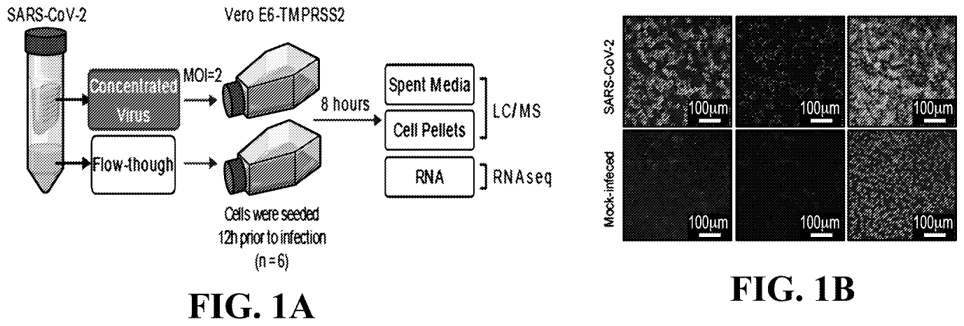

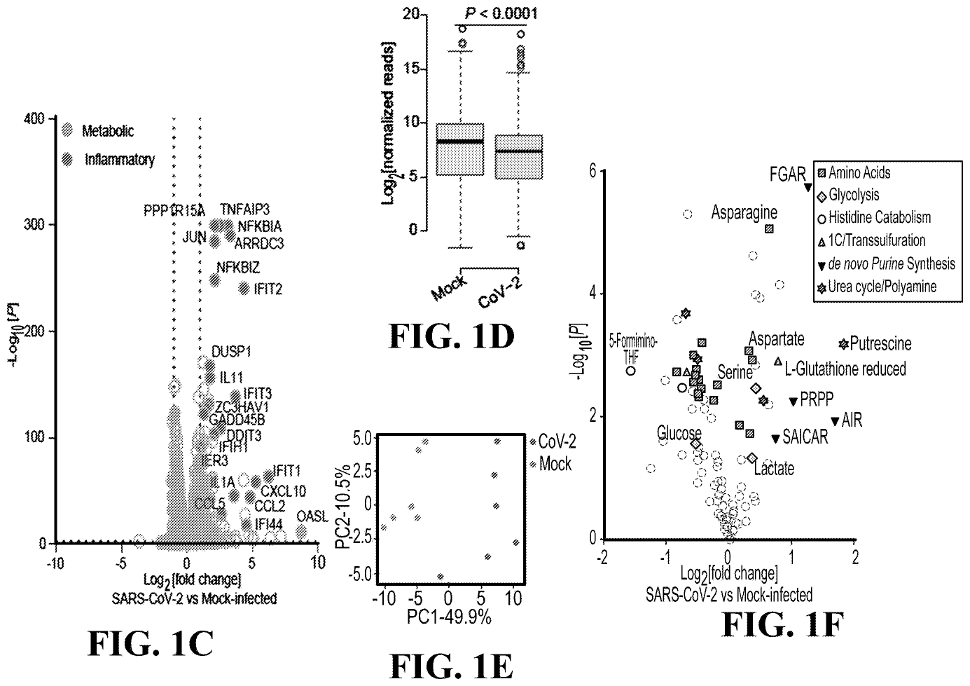

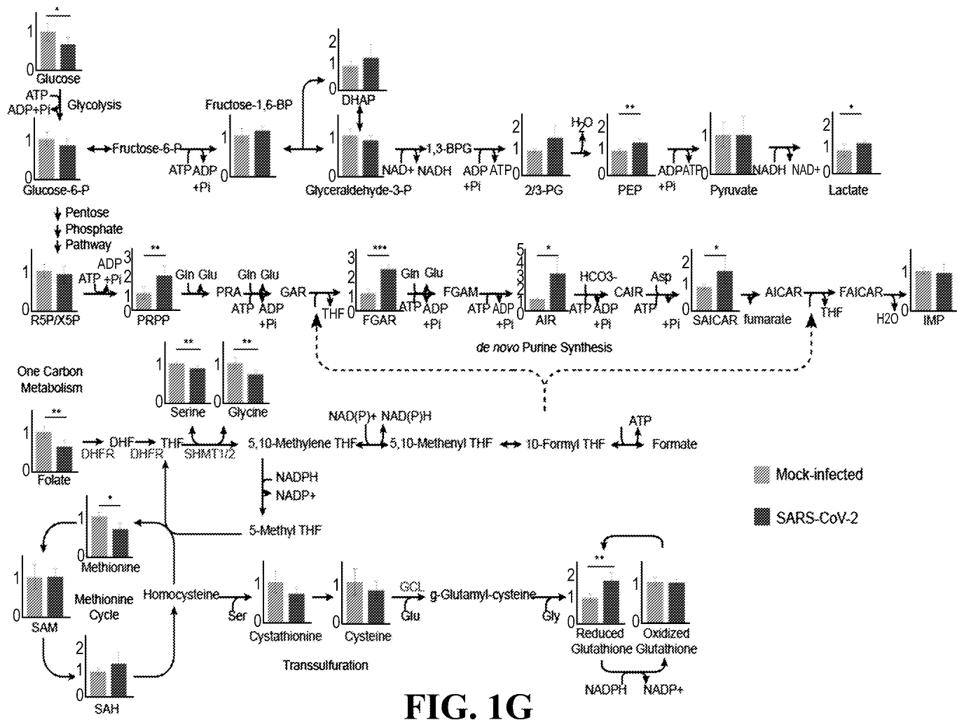

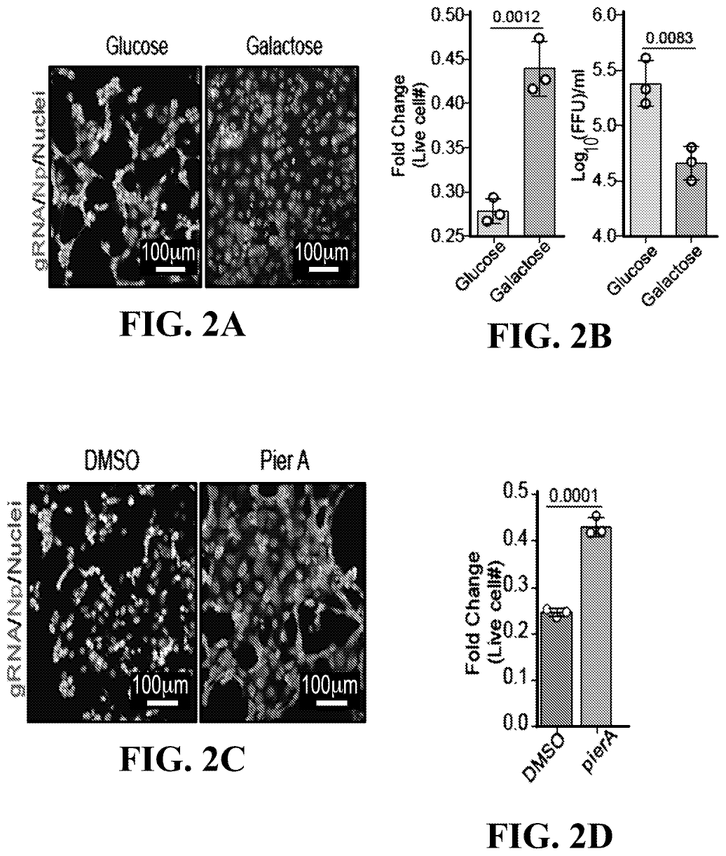

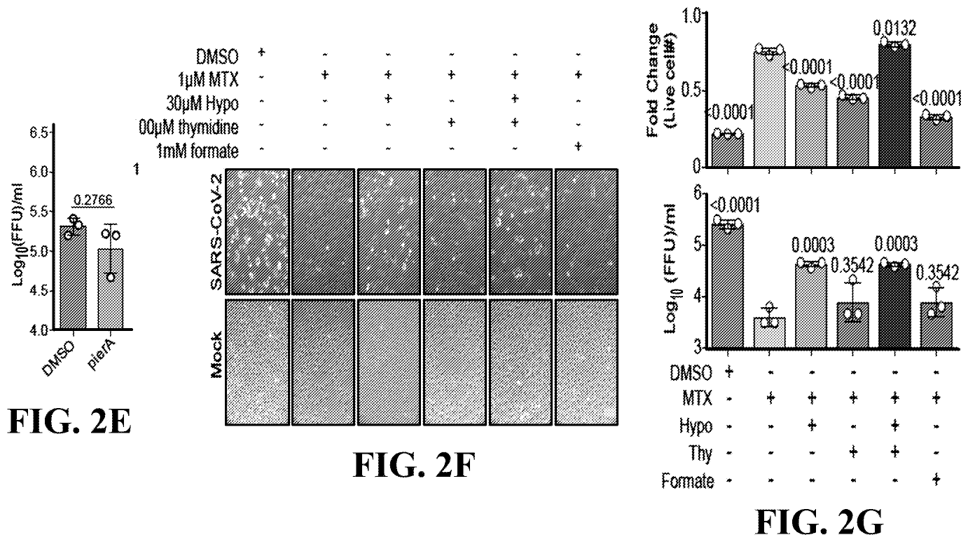

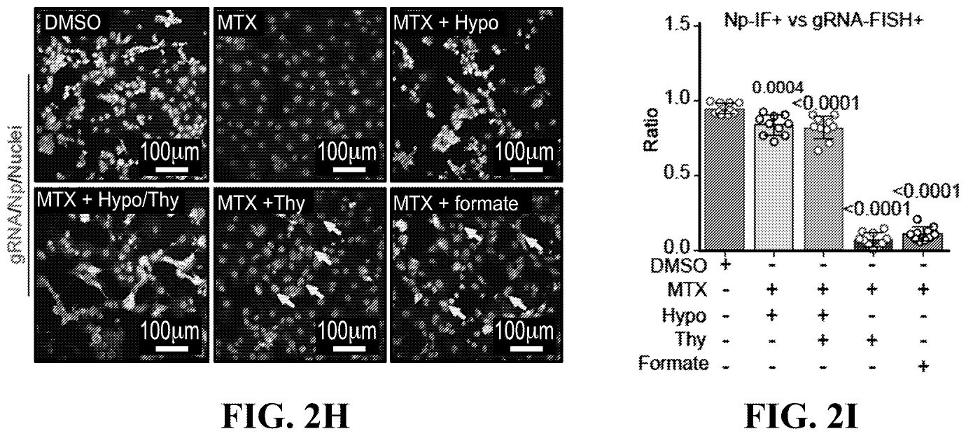

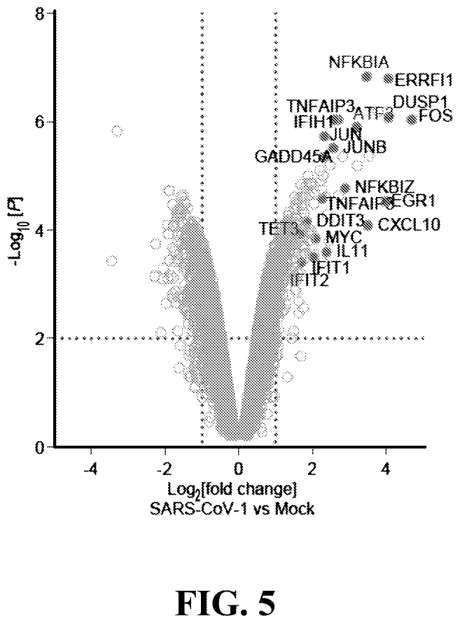

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cast, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g. Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade, in particular Class1 proteins, can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprising one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas 13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B 1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B 1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas 13 c, and/or Cas 13 d.

In some embodiments, the gene editing system (e.g., a class 2, Type VI systems herein) may modify a target RNA. Such systems may knock down target RNA molecules (e.g., transcripts of target genes herein) without permanent modification of the DNA sequences of the genes. This approach may provide temporal control in modulating the expression of target genes.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication No. WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, the Cas split domains (e.g., RuvC and HNH domains in the case of Cas9) can be simultaneously or sequentially introduced into the cell such that the split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018.Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

Examples of base editing systems include those described in International Patent Publication NOs. WO 2019/071048 (e.g. paragraphs [0933]-0938]), WO 2019/084063 (e.g., paragraphs [0173]-[0186], [0323]-[0475], [0893]-[1094]), WO 2019/126716 (e.g., paragraphs [0290]-[0425], [1077]-[1084]), WO 2019/126709 (e.g., paragraphs [0294]-[0453]), WO2019126762 (e.g., paragraphs [0309]-[0438]), WO 2019/126774 (e.g., paragraphs [0511][0670]), Cox DBT, et al., RNA editing with CRISPR-Cas13, Science. 2017 Nov. 24; 358(6366):1019-1027; Abudayyeh 00, et al., A cytosine deaminase for programmable single-base RNA editing, Science 26 Jul. 2019: Vol. 365, Issue 6451, pp. 382-386; Gaudelli N M et al., Programmable base editing of A⋅T to G⋅C in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19;533(7603):420-4; Jordan L. Doman et al., Evaluation and minimization of Cas9-independent off-target DNA editing by cytosine base editors, Nat Biotechnol (2020). doi.org/10.1038/s41587-020-0414-6; and Richter M F et al., Phage-assisted evolution of an adenine base editor with improved Cas domain compatibility and activity, Nat Biotechnol (2020). doi.org/10.1038/s41587-020-0453-z, which are incorporated by reference herein in their entireties.

### Prime Editors

In some embodiments, a polynucleotide of the present invention described elsewhere herein can be modified using a prime editing system. See e.g. Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. In some examples, a pegRNA is a sgRNA comprising a primer binding sequence (PBS) and a template containing a desired RNA sequence (e.g., added at the 3′ end). Embodiments that can be used with the present invention include these and variants thereof. Prime editing can have the advantage of lower off-target activity than traditional CRISPR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′ hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

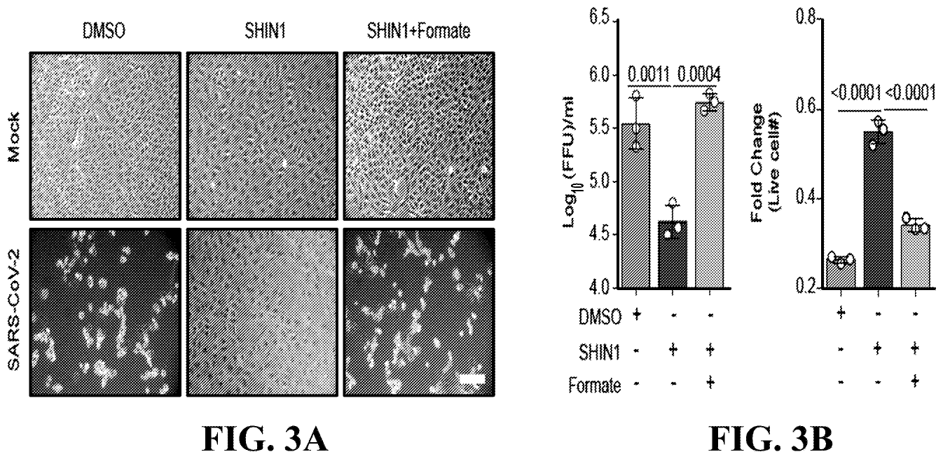

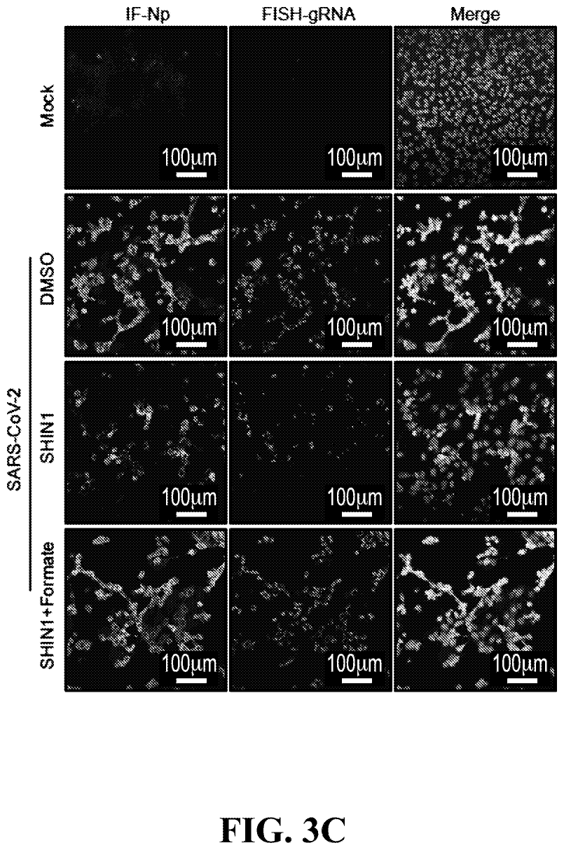

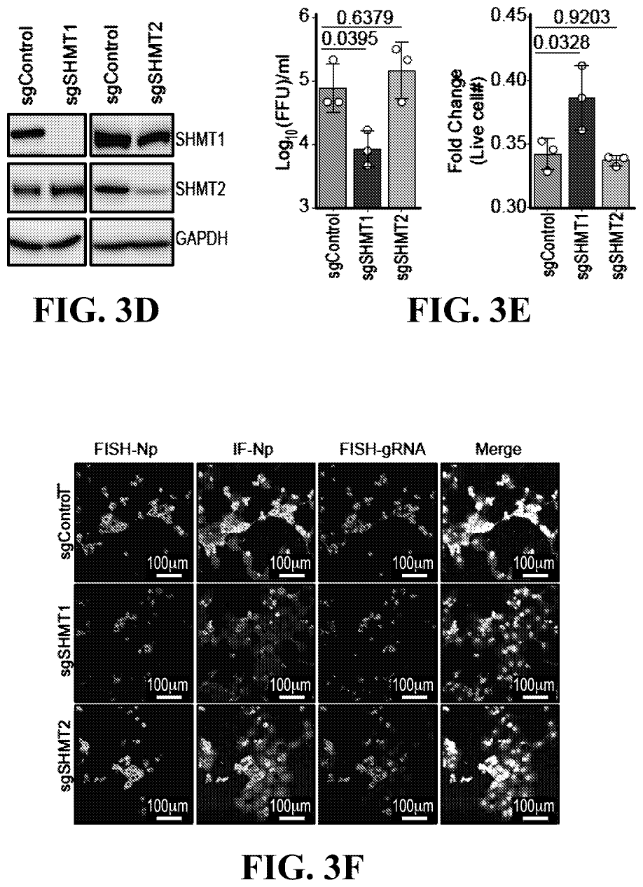

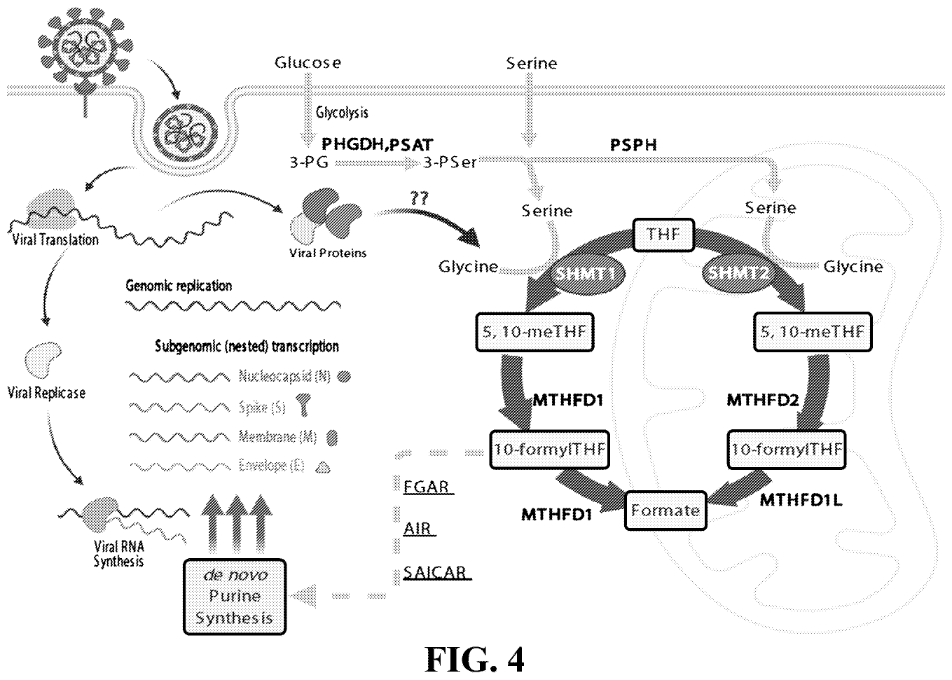

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq. sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 1 (from Gleditzsch et al. 2019) below shows several Cas polypeptides and the PAM sequence they recognize.

TABLE 1
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including LbCpf1 and AsCpf1) | TTTV |
| Cas12b (C2c1) | TTT, TTA, and TTC |
| Cas12c (C2c3) | TA |
| Cas12d (CasY) | TA |
| Cas12e (CasX) | 5′-TTCN-3′ |

In some embodiments, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 1) or PKKKRKVEAS (SEQ ID NO: 2); the NLS from nucleoplasmin (e.g., the nucleoplasmin bipartite NLS with the sequence KRPAATKKAGQAKKKK (SEQ ID NO: 3)); the c-myc NLS having the amino acid sequence PAAKRVKLD (SEQ ID NO: 4) or RQRRNELKRSP (SEQ ID NO: 5); the hRNPA1 M9 NLS having the sequence NQSSNFGPMKGGNFGGRSSGPYGGGGQYFAKPRNQGGY (SEQ ID NO: 6); the sequence RMRIZFKNKGKDTAELRRRRVEVSVELRKAKKDEQILKRRNV (SEQ ID NO: 7) of the IBB domain from importin-alpha; the sequences VSRKRPRP (SEQ ID NO: 8) and PPKKARED (SEQ ID NO: 9) of the myoma T protein; the sequence PQPKKKPL (SEQ ID NO: 10 of human p53; the sequence SALIKKKKKMAP (SEQ ID NO: 11) of mouse c-abl IV; the sequences DRLRR (SEQ ID NO: 12) and PKQKKRK (SEQ ID NO: 13) of the influenza virus NS 1; the sequence RKLKKKIKKL (SEQ ID NO: 14) of the Hepatitis virus delta antigen; the sequence REKKKFLKRR (SEQ ID NO: 15) of the mouse Mx1 protein; the sequence KRKGDEVDGVDEVAKKKSKK (SEQ ID NO: 16) of the human poly(ADP-ribose) polymerase; and the sequence RKCLQAGMNLEARKTKK (SEQ ID NO: 17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, e.g., C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include a sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### TALE Systems

The modulating agents may comprise one or more components of a TALE system. The modulating agents may also comprise nucleotide sequences that are or encode one or more components of a TALE system. As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle EL. Christian M. Wang L. Zhang Y. Schmidt C, et al. Efficient design and assembly of custom TALEN and other TAL effector-based constructs for DNA targeting. Nucleic Acids Res. 2011;39:e82; Zhang F. Cong L. Lodato S. Kosuri S. Church GM. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In some embodiments, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 18)
M D P I R S R T P S P A R E L L S G P Q P D G V Q P T A D R G V S P
P A G G P L D G L P A R R T M S R T R L P S P P A P S P A F S A D S
F S D L L R Q F D P S L E N T S L F D S L P P F G A H H T E A A T G
E W D E V Q S G L R A A D A P P P T M R V A V T A A R P P R A K P A
P R R R A A Q P S D A S P A A Q V D L R T L G Y S Q Q Q Q E K I K P
K V R S T V A Q H H E A L V G H G F T H A H I V A L S Q H P A A L G
T V A V K Y Q D M I A A L P E A T H E A I V G V G K Q W S G A R A L
E A L L T V A G E L R G P P L Q L D T G Q L L K I A K R G G V T A V
E A V H A W R N A L T G A P L N.

An exemplary amino acid sequence of a C-terminal capping region is:

(SEQ ID NO: 19)
R P A L E S I V A Q L S R P D P A L A A L T N D H L V A L A C L G
G R P A L D A V K K G L P H A P A L I K R T N R R I P E R T S H R
V A D H A Q V V R V L G F F Q C H S H P A Q A F D D A M T Q F G M
S R H G L L Q L F R R V G V T E L E A R S G T L P P A S Q R W D R
I L Q A S G M K R A K P S P T S T Q T P D Q A S L H A F A D S L E
R D L D A P S P M H E G D Q T R A S.

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, at least 60%, at least 70%, at least 80%, at least 85%, at least 90%, at least 91%, at least 92%, at least 93%, at least 94%, at least 95%, at least 96%, at least 97%, at least 98% or at least 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### Zn-Finger Nucleases

The modulating agents may comprise one or more Zn-finger nucleases or nucleic acids encoding thereof. In some cases, the nucleotide sequences may comprise coding sequences for Zn-Finger nucleases. Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated herein by reference.

### Meganucleases

The modulating agents may comprise one or more meganucleases or nucleic acids encoding thereof. As disclosed herein editing can be made by way of meganucleases, which are endodeoxyribonucleases characterized by a large recognition site (double-stranded DNA sequences of 12 to 40 base pairs). In some cases, the nucleotide sequences may comprise coding sequences for meganucleases. Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, any of the nucleases, including the modified nucleases as described herein, may be used in the methods, compositions, and kits according to the invention. In particular embodiments, nuclease activity of an unmodified nuclease may be compared with nuclease activity of any of the modified nucleases as described herein, e.g. to compare for instance off-target or on-target effects. Alternatively, nuclease activity (or a modified activity as described herein) of different modified nucleases may be compared, e.g. to compare for instance off-target or on-target effects.

### Interfering RNA

In some embodiments, the modulating agents may be interfering RNAs. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agents may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

In further embodiments, agents useful in the methods as disclosed herein are proteins and/or peptides or fragment thereof, which inhibit the gene expression of a target gene or gene product, or the function of a target protein. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. As disclosed herein, a protein which inhibits the function of a target protein may be a soluble dominant negative form of the target protein or a functional fragment or variant thereof which inhibits wild-type full length target protein function.

In certain embodiments, the agents may be small molecules, antibodies, therapeutic antibody, antibody fragment, antibody-like protein scaffold, aptamer, protein, genetic modifying agent or small molecule. The chemical entity or biological product is preferably, but not necessarily a low molecular weight compound, but may also be a larger compound, or any organic or inorganic molecule effective in the given situation, including modified and unmodified nucleic acids such as antisense nucleic acids, RNAi, such as siRNA or shRNA, CRISPR-Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, minibodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments the agent is a small molecule having a chemical moiety. Agents can be known to have a desired activity and/or property, or can be selected from a library of diverse compounds.

### Exogenous Genes

In some embodiments, the modulating agents are exogenous genes or the coded products, e.g., RNA or proteins. Such exogenous genes may be any genes described herein. In certain cases, the exogenous genes may be delivered on a vector (e.g., plasmid). The expression level of the exogenous genes may be different (e.g., higher) than an endogenous gene. The exogenous gene may comprise one or more mutations or truncations compared to an endogenous counterpart gene. In certain cases, the exogenous genes may be a fusion product of multiple genes or functional fragments thereof.

### Pharmaceutical Compositions

The present disclosure also provides for pharmaceutical compositions comprising the one or more modulating agents. In certain cases, the methods of treatment comprise administering the pharmaceutical composition(s) to a subject in need thereof. A “pharmaceutical composition” refers to a composition that usually contains an excipient, such as a pharmaceutically acceptable carrier that is conventional in the art and that is suitable for administration to cells or to a subject.

In certain embodiments, the methods of the disclosure include administering to a subject in need thereof an effective amount (e.g., therapeutically effective amount or prophylactically effective amount) of the treatments provided herein. Such treatment may be supplemented with other known treatments, such as surgery on the subject. In certain embodiments, the surgery is strictureplasty, resection (e.g., bowel resection, colon resection), colectomy, surgery for abscesses and fistulas, proctocolectomy, restorative proctocolectomy, vaginal surgery, cataract surgery, or a combination thereof.

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. Each of the cells or active components (e.g., modulants, immunomodulants, antigens) as discussed herein may be administered by the same route or may be administered by a different route. By means of example, and without limitation, cells may be administered parenterally, and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

The pharmaceutical compositions may comprise one or more pharmaceutically acceptable salts. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agents, cells, agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

### Delivery of Modulating Agents and Pharmaceutical Compositions

Various delivery systems are known and can be used to administer the agents and pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

In some embodiments, the present disclosure provides compositions, systems, devices, and methods for delivering the modulating agents to a desired tissue or organ in a subject. In some examples, the present disclosure provides compositions, systems, devices, and methods for delivering the modulating agents to the lungs. For examples, the modulating agents may be delivered by devices like inhalers. In some cases, the modulating agents may be formulated to be suitable for delivery to lungs and other part of the respiratory systems. For examples, the modulating agents may be formulated in liposomes, nanoparticles (e.g., solid lipid nanoparticles, polymeric nanoparticles, inorganic nanoparticles), large porous microparticles, or nanosegregates. Such formulations may be inhalable and administered to a subject with an inhaler. Examples of approaches for delivery the modulating agents include those described in Newman SP, Drug delivery to the lungs: challenges and opportunities, Ther Deliv. 2017 July;8(8):647-661; Kurt Nikander, Challenges and opportunities in respiratory drug delivery devices, Expert Opin Drug Deliv. 2010 November; 7(11):1235-8, which are incorporated by reference herein in their entireties.

Delivery of Modulating Agents that are Polynucleotides

In cases the modulating agents are polynucleotides, they may be delivered to cell using suitable methods. In some embodiments, the polynucleotides may be packaged in viruses or particles, or conjugated to a vehicle for delivering into cells.

In some embodiments, the methods include packaging the polynucleotides in viruses and transducing cell with the viruses. Transduction or transducing herein refers to the delivery of a polynucleotide molecule to a recipient cell either in vivo or in vitro, by infecting the cells with a virus carrying that polynucleotide molecule. The virus may be a replication-defective viral vector. In some examples, the viruses may be virus (e.g. retroviruses, replication defective retroviruses, adenoviruses, replication defective adenoviruses, and adeno-associated viruses (AAVs)).

In some examples, the viruses are lentiviruses. Lentiviruses are complex retroviruses that have the ability to infect and express their genes in both mitotic and post-mitotic cells. Examples of lentiviruses include human immunodeficiency virus (HIV) (e.g., strain 1 and strain 2), simian immunodeficiency virus (SIV), feline immunodeficiency virus (FIV), BLV, EIAV, CEV, and visna virus. Lentiviruses may be used for nondividing or terminally differentiated cells such as neurons, macrophages, hematopoietic stem cells, retinal photoreceptors, and muscle and liver cells, cell types for which previous gene therapy methods could not be used. A vector containing such a lentivirus core (e.g. gag gene) can transduce both dividing and non-dividing cells.

In certain embodiments, the viruses are adeno-associated viruses (AAVs). AAVs are naturally occurring defective viruses that require helper viruses to produce infectious particles (Muzyczka, N., Curr. Topics in Microbiol. Immunol. 158:97 (1992)). It is also one of the few viruses that can integrate its DNA into nondividing cells. Vectors containing as little as 300 base pairs of AAV can be packaged and can integrate, but space for exogenous DNA is limited to about 4.5 kb. In some cases, an AAV vector may include all the sequences necessary for DNA replication, encapsidation, and host-cell integration. The recombinant AAV vector can be transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of Felgner, and International Patent Publication Nos. WO 91/17424 and WO 91/16024. Delivery can be to cells (e.g. in vitro or ex vivo administration) or target tissues (e.g. in vivo administration). Physical methods of introducing polynucleotides may also be used. Examples of such methods include injection of a solution containing the polynucleotides, bombardment by particles covered by the polynucleotides, soaking a cell, tissue sample or organism in a solution of the polynucleotides, or electroporation of cell membranes in the presence of the polynucleotides.

Examples of delivery methods and vehicles include viruses, nanoparticles, exosomes, nanoclews, liposomes, lipids (e.g., LNPs), supercharged proteins, cell permeabilizing peptides, and implantable devices. The nucleic acids, proteins and other molecules, as well as cells described herein may be delivered to cells, tissues, organs, or subjects using methods described in paragraphs [00117] to [00278] of Feng Zhang et al., (International Patent Publication No. WO 2016/106236A1), which is incorporated by reference herein in its entirety.

In some cases, the methods include delivering the barcode construct and/or another element (e.g., a perturbation element) to cells. In such cases, the barcode construct and/or another element (e.g., a perturbation element) may be RNA molecules.

Combination Therapy with Other Therapeutic Agents

In some embodiments, the methods may further comprise administering to a subject (e.g., a subject in need thereof) one or more therapeutic agents in addition to the modulating agent(s). The one or more therapeutic agents may be administered at the same time when the modulating agent(s) is administered. Alternatively or additionally, the one or more therapeutic agents may be administered at a different time from when the modulating agent(s) is administered. In some examples, the one or more therapeutic agents are prophylactic therapeutic agents which are administered prior to a viral infection.

The therapeutic agents may be for treating virus infection and related health problems. In some cases, the therapeutic agent(s) are drug(s) for treating SARS-CoV-2 and related diseases. Examples of such therapeutic agents include nucleoside analogues (e.g., Remdesivir, Favipiravir, Ribavirin), HIV protease inhibitors (e.g., Kaletra (lopinavir/ritonavir)), agents targeting proinflammatory hypercytokinemia (e.g., Tocilizumab and leronlimab), IFNλ, Antiparasitics (e.g., Ivermectin), antimalarial drugs (e.g., Chloroquine and hydroxychloroquine), agents targeting cardioprotective derivatives (e.g., Colchicine), agents targeting angiotensin-converting enzyme 2 (ACE2), corticosteroids (e.g., Dexamethasone, Prednisone, Methylprednisolone), Nicotine, Vitamin D, and Spironolactone. Additional examples of therapeutic agents include those described in Konstantinidou S K et al., Repurposing current therapeutic regimens against SARS-CoV-2 (Review), Exp Ther Med. 2020 September;20(3):1845-1855, which is incorporated herein in its entirety. In some cases, the therapeutic agents are monoclonal antibodies for treating SARS-CoV-2 and related diseases. Examples of such therapeutic agents include a combination of casirivimab and imdevimab, called REGN-COV, made by Regeneron; a combination of bamlanivimab and etesevimab, made by Eli Lilly; and sotrovimab, made by GlaxoSmithKline. In some cases, the therapeutic agent is convalescent plasma derived from patients who have recovered from SARS-CoV-2 and related diseases. In some cases, the therapeutic agents are prophylactic therapeutic agents administered prior to a viral infection. Examples of such prophylactic therapeutic agents include mRNA vaccines (e.g., made by Pfizer-BioNTech or Moderna) and viral vector vaccines (e.g., made by Johnson & Johnson's Janssen).

### Example Viral Agents

The methods and modulating agents disclosed herein may be used to inhibit viral reproduction. In some embodiments, the virus is an RNA virus. In some embodiments, the RNA virus is a positive-sense single strand RNA virus. In some embodiments, the positive-sense single strand RNA virus is a coronavirus, flavivirus, or enterovirus. In some embodiments, the RNA virus is a negative-sense single strand RNA virus. In some embodiments, the negative-sense single strand RNA virus is an influenza, paramyxovirus, respiratory syncytial virus, measles, rabies, or Ebola. The diseases and conditions include infections caused by coronaviruses. Coronaviruses include positive-sense single stranded RNA family of viruses, infecting a variety of animals and humans. Example of coronaviruses include Coronaviridae (e.g. SARS-CoV is one type of coronavirus infection, as well as MERS-CoV). In some examples, the coronavirus is Severe acute respiratory syndrome coronavirus 2 (SARS-CoV-2 or COVID-19). SARS-CoV-2 are available at GISAID accession no. EPI_ISL_402124 and EPI_ISL_402127-402130, and described in DOI: 10.1101/2020.01.22.914952. Further deposits of the SARS-CoV2 are deposited in the GISAID platform include EP_ISL_402119-402121 and EP_ISL 402123-402124; see also GenBank Accession No. MN908947.3.

Examples of other types of viruses that cause infection, the viruses including Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A (e.g., H1N1, H1N2, H2N1, H3N1, H3N2, and H2N3) or influenza B, Swine Influenza Virus (SIV) strains which include influenza C. An HIV may include HIV 1 or HIV 2.

In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus, California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus ZIO, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-I, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MS5121.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, 0 ‘nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Pow assan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence.

In certain example embodiments, the virus may be an RNA viruse that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zorter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others.

### EXAMPLES

### Example 1

The recently identified Severe Acute Respiratory Syndrome Coronavirus 2 (SARS-CoV-2) is the cause of the COVID-19 pandemic. How this novel beta-coronavirus virus, and coronaviruses more generally, alter cellular metabolism to support massive production of ˜30 kB viral genomes and abundant subgenomic viral RNAs remains largely unknown. To gain insights, transcriptional and metabolomic analyses were performed 8 hours after SARS-CoV-2 infection, an early timepoint where the viral lifecycle was completed but prior to overt effects on host cell growth or survival. Here, Applicants showed that SARS-CoV-2 post-transcriptionally hijacked host folate and one-carbon metabolism to support de novo purine synthesis. Intracellular glucose and folate were significantly depleted in SARS-CoV-2-infected cells, and viral replication was exquisitely sensitive to inhibitors of folate and one-carbon metabolism, notably methotrexate. Anti-viral therapy targeting host metabolism could add to the armamentarium against future coronavirus outbreaks and circumvent resistance to direct acting antivirals.

Upon host cell infection, the SARS-CoV-2 replication/transcription complex synthesizes ˜30 kilobase viral genomes and highly abundant subgenomic RNAs that serve as templates for viral structural proteins. Subgenomic RNAs are co-terminal, nested mRNAs encoded by a common 5’ leader sequence that is spliced to regions from −10 kilobases at the 3′ end of the genome. Viral genomes are first produced as negative-strand RNA, which serve as templates for the production of the positive strand virion genomes. Coronavirus positive strand genomes exist at 50- to 100-fold excess of their minus strand counterparts (4) and are coated by the viral nucleocapsid protein (Np) prior to virion assembly (5).

Between 1 and 5 hours post-infection by the model beta-coronavirus murine hepatitis virus, the percentage of virus encoded to total cellular protein translation increases by as much as 20,000-fold. Over this time period, the fraction of viral to cellular RNA reached 90%, much of which was subgenomic RNA (6), underscoring a major transformation in the metabolism of newly infected cells. Recent profiling studies indicate that high levels of SARS-CoV-2 transcripts likewise enable newly infected cell translation to be dominated by production of viral proteins (7). SARS-CoV-2 encoded proteins assemble a network of double membrane vesicles, leading to the production and secretion of abundant infectious virion (8, 9). Yet, host metabolic pathways that are rapidly subverted by SARS-CoV-2 to support this biosynthesis remain largely unknown.

To gain insights into SARS-CoV-2 remodeling of key host metabolic pathways, Vero E6 TMPRSS2+ cells were mock infected, or infected at a multiplicity of infection (MOI) of 2. Vero E6 were used to enable high percentage target cell infection, at an early timepoint (8 hours post infection, hpi) prior to the onset of viral cytostatic or cytopathic effects that confound measurements at later timepoints. To minimize unwanted effects of lactate and other metabolites in the virus stock, parallel Vero cultures were either infected by SARS-CoV-2 that had been concentrated by membrane filtration, or mock-infected with an equal volume of the virus-depleted flow-through (FIG. 1A). Robust production of viral genomic RNA (gRNA) and nucleocapsid protein (Np) was evident by 8 hours post-infection (hpi) in most cells (FIG. 1B), consistent with prior estimates of the eclipse period time from SARS-CoV adsorption to release of infectious progeny (10).

RNA-seq analysis at 8 hpi revealed robust induction of anti-viral genes (e.g. IFIT1, ZC3HAV1), NF-kappa B targets (e.g., CCL5, CXCL10) and ER stress response (e.g. DDIT3, PPP1R15A, GADD45B). Yet, surprisingly few changes were observed in the abundances of mRNAs encoding metabolic enzymes (FIG. 1C, Table 4A), despite a global decrease in host mRNAs in SARS-CoV-2 infected cells (FIG. 1D). Re-analysis of SARS-CoV-1-infected Vero E6 cell mRNA abundance (12) showed strikingly similar results (Table 4B), with minimal remodeling of metabolism pathways evident at the mRNA level at 8 hpi, suggesting that this property may be conserved across SARS coronaviruses (FIG. 5). Curiously, nearly all of the mitochondrial DNA (mtDNA) encoded transcripts related to oxidative phosphorylation (OXPHOS) were elevated, while nuclear genome encoded OXPHOS transcripts were all decreased, a gene expression pattern that has been previously been associated with ATP depletion (Table 4) (13).

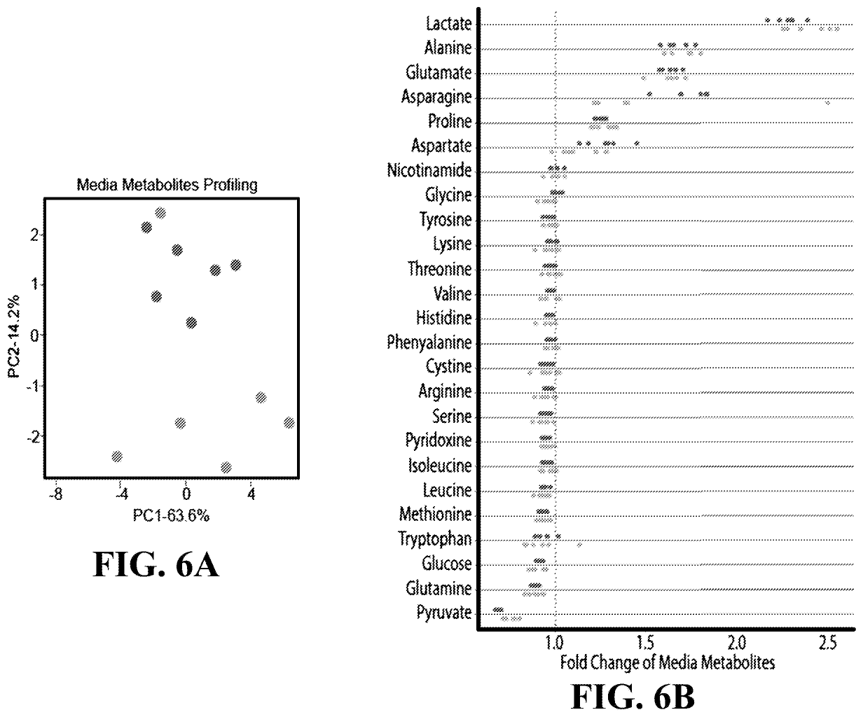

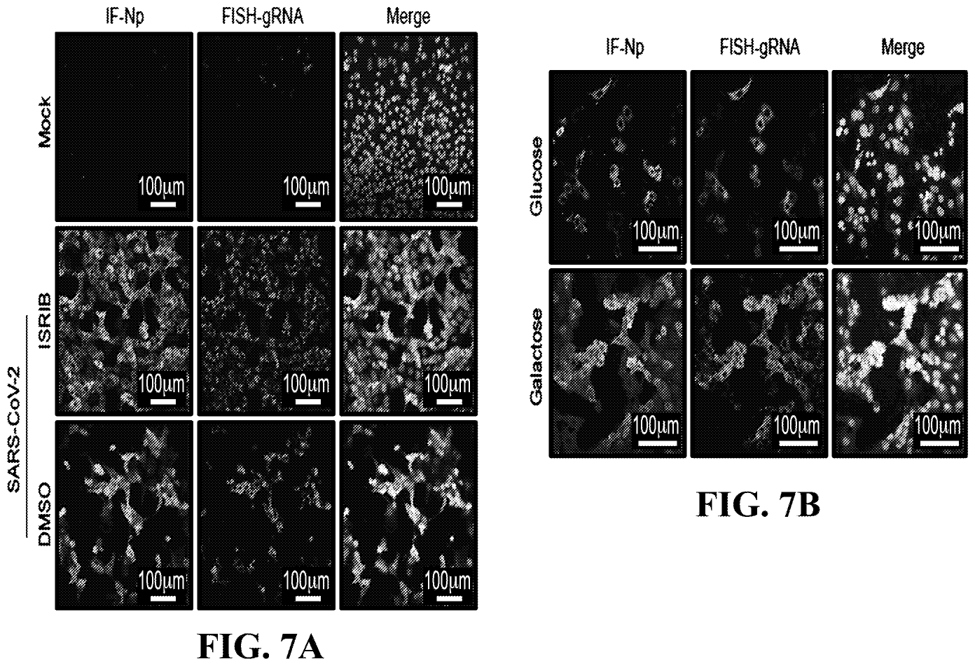

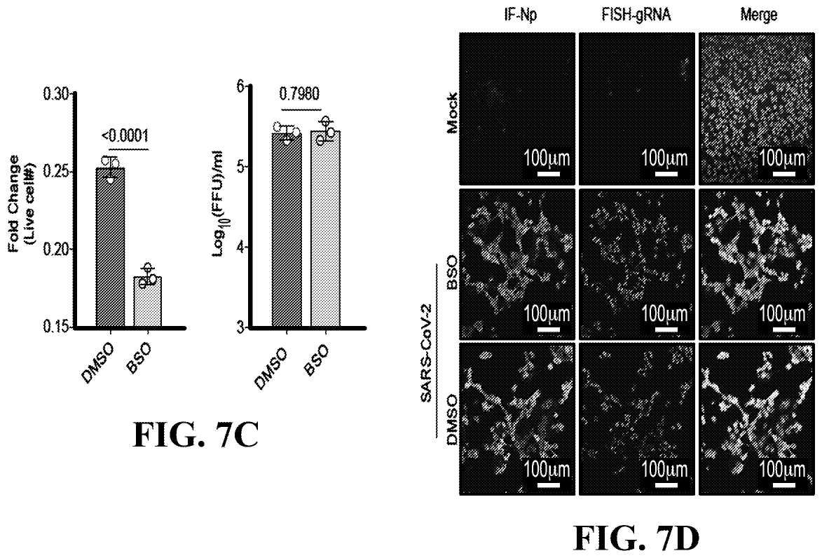

To cross-compare SARS-CoV-2-mediated transcriptional and metabolism changes within the same cells, metabolomic analyses of spent media and cell pellets were performed in parallel (FIG. 1A). Quantitative production and consumption analyses of spent media metabolites found subtle, mostly non-significant changes between infected and mock-infected cells at this early timepoint (FIG. 6A-6B, Table 5). By contrast, pronounced differences in intracellular metabolites were already observed at this early timepoint (FIG. 1E-1F, Table 6), suggesting that SARS-CoV-2 rewired intracellular metabolism to support viral replication. Of note, most amino acids were depleted by SARS-CoV-2 infection, although several including aspartate and asparagine were upregulated (FIG. 1F, FIG. 6B). Amino acids might have been largely consumed for viral protein synthesis, while simultaneous activation of cellular integrated stress response (ISR) may lead to selective increase in aspartate and asparagine (14). Interestingly, the small molecule ISR inhibitor ISRIB (15) increased the number of live cells at 48 hours post-infection, even though it did not appreciably diminish viral gRNA or Np expression (FIG. 7A).

One of the most striking changes in intracellular metabolomics was accumulation of de novo purine synthesis intermediates, including 5-phosphoribosyl-1-pyrophosphate (PRPP), N-formylglycinamide ribonucleotide (FGAR), aminoimidazole ribonucleotide (AIR), and succinylaminoimidazolecarboxamide ribonucleotide (SAICAR) in virus-infected cells (FIG. 1F-1G, Table 3). Ribonucleotide synthesis involved ribose derived from glucose as well as one-carbon (1C) units carried by folate species. Intracellular glucose and folate were significantly depleted in SARS-CoV-2-infected cells, raising the possibility that host glucose and folate metabolism were hijacked to meet the demand for viral subgenomic RNA replication. Interestingly, this occurred concomitantly with a significant decrease in host mRNA abundance (FIG. 1D), which may be due to destabilization by the host shut-off activity of SARS-CoV-2 to salvage host nucleotide supply for viral biosynthesis (16). Of note, inosine monophosphate (IMP) was not significantly increased (FIG. 1F), suggestive of a balance between production and consumption at this regulated step in purine synthesis.

Glycolysis provides ATP as well as building blocks for de novo serine and nucleotide synthesis. In support of a key glucose metabolism role in support of viral replication, shifting the media sugar source from glucose to galactose just prior to infection, which allowed continued operation of OXPHOS but not glycolysis, strongly impaired production of viral+sense genomic RNA (gRNA), nucleoprotein and infectious virion and increased live cell number at 48 hpi, suggesting reduction of viral cytopathic effect (CPE) (FIG. 2A-2B and FIG.¶7B). Treatment of cells with the electron transport chain complex I inhibitor Piericidin A (PierA), which blunted bioenergetics by blocking OXPHOS also reduced viral CPE, but did not have a major impact on viral gRNA, Np or secreted virus levels (FIG. 2C-2E). Taken together, these results suggest that impairment of glucose-derived precursors for anabolic reactions likely underlies the observed decrease in viral replication upon glucose starvation.

Folate metabolism is critical for transfer of 1C units for nucleotide synthesis and is also closely linked to glutathione biosynthesis via the transsulfuration pathway (FIG. 1G) (17). Depletion of folate in SARS-CoV-2-infected cells was coupled to increased glutathione abundance, raising the possibility that 1C metabolism might support SARS-CoV-2 replication through roles in antioxidant defense (FIG. 1F-1G). However, the glutamylcysteine synthetase inhibitor L-buthionine-sulfoximine (BSO), which blocked synthesis of reduced and oxidized glutathione (GSH, GSSG, FIG. 1G), did not have any apparent effect on viral gRNA and Np levels (FIG. 7D). This result suggests that the increase in SARS-CoV-2 infected cell glutathione pool is likely an epiphenomenon of increased 1C metabolism. Collectively, these observations suggest that SARS-CoV-2 activates glucose and folate metabolism at the post-transcriptional level in newly-infected cells to supply the massive need for ribonucleotide synthesis.

Guided by these early post-infection metabolomic changes, Applicants asked whether inhibition of folate metabolism could blunt viral RNA expression and virion production. The widely used drug methotrexate (MTX), a folate analog which competitively inhibits the enzyme dihydrofolate reductase (DHFR) as well as several additional steps in 1C metabolism and nucleotide synthesis (18-20), significantly blocked virus-induced CPE, reduced viral gRNA and Np levels, and diminished secretion of infectious virion by nearly 2-log (FIG. 2F-2I). All of the mentioned effects of 1 μM MTX could be reversed by 30 μM hypoxanthine, an intermediate of purine salvage pathway and an alternate source of purines (21). Interestingly, addition of 10011M of the pyrimidine deoxynucleotide thymidine or 1 mM of the one-carbon group donor formate could only partially restore viral gRNA levels, but were unable to rescue translation of viral Np, virion production or CPE (FIG. 2F-2I). These results suggest that the sensitivity of viral replication to methotrexate is closely related to the critical role of folate metabolism in supporting de novo purine synthesis.

To further isolate the roles of folate species generation and interconversion in viral replication, Applicants inhibited cytosolic and mitochondrial isoforms of serine hydroxymethyltransferase (SHMT1 and SHMT2, respectively) (FIG. 1G) (17). Treatment of Vero E6 cells just prior to infection with the highly-specific SHMT1/2 dual inhibitor SHIN1 (22) reduced infectious virus titer by ˜1-log at 48hpi, diminished+strand gRNA and nucleoprotein levels, and induced resistance to viral CPE (FIG. 3A-3C). SHIN1 effects were fully reversed by the addition of formate, demonstrating an on-target mechanism of action of the inhibitor (FIG. 3A-3C). As SHIN1 is a dual SHMT1/2 antagonist, Applicants next used CRISPR to test whether SARS-CoV-2 replication was specifically dependent on a SHMT1- or SHMT2-mediated one-carbon metabolism pathway. CRISPR SHMT1 targeting reduced expression of +strand gRNA, Np RNA and protein, diminished infectious virus titer by nearly 1-log and enhanced cell survival (FIG. 3D-3F). By contrast, CRISPR SHMT2 targeting had minimal effect on these parameters of viral infection (FIG. 3D-3F). Although Applicants note that there is some residual SHMT2 expression in this experiment, treatment with PierA is also known to indirectly block the mitochondrial 1C pathway (21), but also did not reduce viral replication (FIG. 2E). Taken together, these results suggested that host cytosolic 1C metabolism is important for virion production, potentially at the level of viral subgenomic RNA expression (FIG. 4).

These parallel transcriptomic and metabolomic analyses of SARS-CoV-2-infected epithelial cells demonstrated rapid rewiring of host glycolysis and cytosolic folate/one-carbon metabolism at the post-transcriptional level to support viral RNA, protein and virion synthesis. These studies therefore highlighted an interesting difference between SARS-CoV-2 infected epithelial cell and monocytes, where glycolytic flux also supports viral replication (23). However, in monocytes, SARS-CoV-2 replication increased mitochondrial reactive oxygen species to trigger a hypoxia-inducible factor-1a dependent pathway that upregulates glycolysis genes at the transcriptional level by 24 hours post-infection (24).

While many host/virus metabolism studies have focused on later timepoints of infection (26), these analyses point to an unappreciated role for host one-carbon metabolism early in the viral lifecycle. Interestingly, viral NSP14 interacts with the host enzyme IMPDH2 (27), which catalyzes inosine 5′-phosphate conversion to xanthosine 5′-phosphate in the first committed and rate-limiting step of de novo guanine synthesis. IMPDH inhibition reduces viral replication (28, 29), raising the possibility that NSP14 may activate de novo purine synthesis to support massive viral RNA production. Alternatively, global phosphoproteomic analysis has also revealed that host kinase signaling is altered at these early timepoints of Vero-E6 SARS-CoV-2 infection, including in pathways such as AKT that can activate metabolism responses (25).

Murine hepatitis virus (MHV) and SARS-CoV-2 studies indicate that high levels of transcription enables massive production of coronavirus structural proteins, in particular Np (7, 25). Indeed, by five hours post-(MHV) infection, positive-sense virus RNA accounts for 80-90% of total virus cellular RNA (6). These results, taken together with the data herein, suggest a model in which de novo purine synthesis is particularly important for vast subgenomic RNAs production within the first 8 hours of SARS-CoV-2 infection (7) (FIG. 4).

Antifolate roles in COVID-19 prophylaxis versus treatment approaches would be of significant interest. Antifolates may be useful in counteracting COVID-19 via two distinct mechanisms: synergistic antiviral activity and anti-inflammatory action. Methotrexate effects on the cellular purine nucleotide pool likewise impair replication of the RNA flavivirus Zika (33). The work suggested important clinical indications that may be rapidly and practically actionable, given that long-term methotrexate therapy is already FDA approved for inflammatory disorders such as rheumatoid arthritis and psoriasis, and that there is ample precedent for modulating host one-carbon pools via dietary modulation (34, 35). Methotrexate or SHMT inhibition may be synergistic with the antiviral nucleotide analog remdesivir, which competes with ATP for incorporation by the viral RNA polymerase (36, 37).

It is estimated that there may be thousands of coronaviruses in bat reservoirs with potential for human transmission. Given that there have been three recent spillovers of pathogenic coronavirus into the human population during the past two decades, there is great need for preparedness for a future coronavirus outbreak. As it is difficult to predict which strain might next emerge, host-targeted antiviral therapy offers the promise of conferring broad protection against future zoonotic coronaviruses with pandemic potential.

### Methods

Cells and viruses: The African Green Monkey Vero E6 with stable TMPRSS2 expression was a gift from Drs. Matteo Gentili and Nir HaCohen. It was constructed by lentiviral transduction of Vero E6 (ATCC) using the construct pTRIP-SFFV-Hygro-2A-TMPRSS2 and selection at 500 μg/ml hygromycin. Vero E6 TMPRSS2+cells were maintained in Dulbecco's Modified Eagle's Medium (DMEM, 25 mM glucose, 4 mM glutamine, 1 mM sodium pyruvate, Gibco, #11965118) supplemented with 10% fetal bovine serum (FBS), 100U/mL penicillin/streptomycin and 250 μg/ml hygromycin at 37° C. with 5% CO₂. Cell lines with stable Streptococcus pyogenes Cas9 expression were generated by lentiviral transduction using pXPR_BRD111 (Addgene plasmid #78166, a gift from Dr. William Hahn) and blasticidin selection (5 ug/ml). Cells were routinely tested and certified as mycoplasma-free using the MycoAlert kit (Lonza, LT07-318). SARS-CoV-2 isolate from USA-WA1/2020 (GenBank accession #MN985325) was obtained from BEI Resources (#NR-52281) and worked with exclusively in the Broad Institute BSL-3 laboratory with approval from the Broad Environmental Health and Safety Office. The virus was propagated as described previously (38). Vero E6 cells were used for virus propagation and titration. For virus stock preparation, 100 μL of passage 0 (P0) virus was mixed with 5 ml of trypsinized cell suspension (0.25×10\hat{ }6/ml) and seeded into a T25 flask. The cell culture supernatant was harvested at 3 dpi and used as the P1 virus stock. Applicants then mixed 100 μl of P1 virus with 10 ml of Vero cells suspension (0.25×10\hat{ }6/ml) and seeded the mixture into a T75 flask. The cell culture supernatant was again harvested at 3 dpi and used as the P2 virus stock for all the experiments in this study. Applicants used same procedure to grow a large amount of P2 viruses, which was carefully titered with a fluorescent-focus assay.

To acquire highly concentrated virus stock, the viral culture supernatant was concentrated up to 25× by using the Microsep Advance Centrifugal Devices with Omega Membrane 30K (Pall Corporation, MAP030C38). The flow-through of the centrifugal devices was collected as the mock infection control. Viral stock titer was performed with a fluorescent-focus assay. For CRISPR editing, sequences of sgRNAs against African Green Monkey SHMT1 and SHMT2 listed in Table 7 were cloned into pLentiguide Puro (Addgene #52963, a gift from Dr. Feng Zhang). Transduced Vero E6 TMPRSS2 cells were selected with puromycin 3 μg/ml at 48 hours post-transduction for 5 days.

### Fluorescent-Focus Assay:

Virus titer in the cell culture supernatant was determined by a fluorescent-focus assay (39). Briefly, Applicants aliquoted 90 μL of serum-free DMEM into columns 1-12 of a 96-well tissue culture plate, then pipetted 10 μL of viruses supernatant into column 1 and serially diluted 10-fold across the plate. Applicants then trypsinized and resuspended Vero cells in DMEM containing 10% FBS, at a density of 5×10⁵ cells/mL. Applicants added 50 μL of cell suspension directly to each well and mixed gently by pipetting. Applicants then inoculated cultures in a 37° C. incubator with 5% CO2 for 2 days. The titration plates were fixed with 4% paraformaldehyde (PFA) overnight. Fixed cells were stained with SARS-CoV-2 N protein-specific monoclonal antibody SA-46 (MAb) and Alexa Fluor 488-conjugated goat anti-mouse IgG was used as a secondary antibody. Fluorescent foci of infected cells were observed and counted using a phase-contrast fluorescence microscope. Virus titers were presented in number of fluorescent-focus units per ml (FFU/ml).

Inhibitors and special media conditions: The small molecules methotrexate (Selleckchem, S1210), hypoxanthine (Sigma, H9636), thymidine (Sigma, T1895), and sodium formate ((Fisher Scientific, S648-500) were used. The dual SHMT1/2 inhibitor SHIN1 (Tocris, 6998) was used at the concentration of 10 μM. 1 mM of sodium formate was used to demonstrate on-target effects of SHIN1. Glutamylcysteine synthetase inhibitor L-buthionine-sulfoximine (BSO) (Sigma, 83730-53-4) was used at the concentration of 10 μM. Mitochondrial complex I inhibitor Piericidin A (Cayman, 15379) was used at the concentration of 0.1 μM. Glucose-free media containing galactose was prepared by supplementing 25 mM galactose (Sigma, G5388) into glucose-free DMEM (#11966025, ThermoFisher) with 10% dialyzed FBS (#26400044, ThermoFisher). Cells were always treated with small molecules or special media 12 hour prior to infection. Samples were harvested at 48 hpi.

Simultaneous Stellaris FISH and Immunofluorescence: Two sets of Stellaris FISH probes, targeting either SARS-CoV-2 ORF pp1a positive strand genomic RNA or the subgenomic RNA encoding ORF9 (nucleocapsid protein) were obtained from Biosearch Technologies. Nucleocapsid protein was detected by using the mouse monoclonal antibody SA46, kindly provided by Dr. Ying Fang. Cells grown on chambered dishes (Mattek) were fixed with 4% paraformaldehyde buffered with PBS overnight in the BSL3 laboratory. Cells were then permeabilized with 70% ethanol for at least 2 hours and subjected to FISH and immunofluorescence following the product manual from Stellaris RNA-FISH. Briefly, cells were incubated with primary antibodies against nucleocapsid (the monoclonal antibody SA46, 1:1000), genomic RNA FISH probes (1:1000) or sub-genomic RNA FISH probes (1:1000) in Hybridization Buffer (#SMF-HB1-10, Biosearch Technologies) containing 10% formamide for at least 4 hours at 37° C. Slides were then washed three times with Buffer A and then incubated with secondary antibodies (Alexa Fluor 488-conjugated goat antimouse 1:250 in Buffer A (#SMF-WA1-60, Biosearch Technologies) for 1 hour at 37° C. Finally, slides were washed three times in Buffer B (#SMF-WB1-20, Biosearch Technologies) and incubated with 100 uL of Hoechst 33258 staining buffer (10 μg/mL in PBS) for 10 minutes to stain nuclear DNA. Cells were then washed two times with Buffer B. ProLong Gold anti-fade reagent (P36935, ThermoFisher) was applied to the slide, which was then sealed with a No. 1.5 coverslip. Image acquisition was performed with the Zeiss LSM 800 instrument. Image analysis was performed with the Zeiss ZEN Blue software.

### Live Cell Number (Cytopathic Effect) Quantification Assay

Cell number was quantified by Viral Tox-Glo (VTG, G8943, Promega) live cell assay as described previously (40). Normalized cytopathic effect was calculated by normalizing VTG values of the infected samples to the VTG values of the corresponding uninfected samples. To normalize the ATP suppressing effects caused by media change, the fold change of live cell number was calculated dividing the luminescence signal of the SARS-CoV-2 infected cells by the signal of mock infected cells with same treatment.

### Immunoblot Analysis

Immunoblot analysis was performed as previously described (40). In brief, whole cell lysates (WCL) were separated by SDS-PAGE electrophoresis, transferred onto the nitrocellulose membranes, blocked with 5% milk in TBST buffer and then probed with relevant primary antibodies at 4° C. overnight, followed by secondary antibody (Cell Signaling Technology) incubation for 1 h at room temperature. Blots were then developed by incubation with ECL chemiluminescence for 1 min (Millipore) and images were captured by Licor Fc platform. The SHMT1 (D3B3J), SHMT2 (E7F4Q) and GAPDH (D16H11) polyclonal antibodies were purchased from Cell Signaling Technology.

RNAseq Analysis

Total RNA from mock infected or SARS-CoV-2 infected cells were isolated using TRIzol Reagent (ThermoFisher, 15596026) following the product manual. To construct indexed libraries, 1 μg of total RNA was used for polyA mRNA-selection, using NEBNext Poly(A) mRNA Magnetic Isolation Module (New England Biolabs), followed by library construction via NEBNext Ultra RNA Library Prep Kit for Illumina (New England Biolabs). Each experimental treatment was performed in triplicate. Libraries were multi-indexed, pooled and sequenced on an Illumina NextSeq 500 sequencer using single-end 75 bp reads (Illunima). Adaptor-trimmed Illumina reads for each individual library were mapped back to the Chlorocebus sabaeus ChlSab1.1 transcriptome assembly using STAR2.5.2b (41). Feature Counts was used to estimate the number of reads mapped to each contig (42). Only transcripts with at least 5 cumulative mapping counts were used in this analysis. DESeq2 was used to evaluate differential expression (DE) (43). DESeq2 uses a negative binomial distribution to account for overdispersion in transcriptome datasets. It is conservative and uses a heuristic approach to detect outliers while avoiding false positives. Each DE analysis was composed of a pairwise comparison between experimental group and the control group. Differentially expressed genes were identified after a correction for false discovery rate (FDR). For more stringent analyses, Applicants set the cutoff for truly differentially expressed genes as adjusted p value (FDR corrected)<0.05 and absolute fold change>2. The volcano plots were built based on the log 2(foldchange) at x-axis and −log 10(p-Value) at y-axis with Graphpad Prism7.

Cell culture for intracellular and media metabolites profiling: 3.5×106 Vero E6 cells were seeded in T25 flask with 10 mL of fresh media (DMEM, 25 mM glucose, 4 mM glutamine, 1 mM sodium pyruvate, Gibco, #11965118) supplemented with 10% fetal bovine serum (FBS) and 100U/mL penicillin/streptomycin. Twelve hours after seeding, cells were infected with either 500 μL of concentrated SARS-CoV-2 (MOI=2) virus or 500 μL of virus-depleted flow-through. Media controls without cells were maintained in parallel throughout the experiment for quantitative media consumption and production analysis.

Intracellular metabolite profiling: At 8hpi, media was collected for quantitative metabolite consumption and production analyses, as described below. After removing all media and washing cells with 5 mL of room temperature PBS, 1 mL of dry ice-cold 80% methanol was added to cell monolayer to quench metabolism. Cells were incubated at −80° C. for 30 mins, harvested with cell scraper, and centrifuged at 21,000×g for 5 mins to precipitate proteins. The supernatant was collected in pre-chilled tubes and stored at −80° C. On the day of analysis, the supernatant was incubated on ice for 20 mins and centrifuged at 21,000×g at 4° C. to clarify. The supernatant was dried down in a speed vacuum concentrator (Savant SPD 1010, Thermofisher Scientific) and re-suspended in 1004, of 60/40 acetonitrile/water. The samples were then vortexed, sonicated in ice-cold water for 1 min, and incubated on ice for 20 mins. Supernatant was collected in an autosampler vial after centrifugation at 21,000×g for 20 mins at 4° C. Pooled QC samples were generated by combining 154, of each sample. Metabolite profiling was performed using Dionex Ultimate 3000 UHPLC system coupled to Q-Exactive plus orbitrap mass spectrometer (ThermoFisher Scientific, Waltham, MA) with an Ion Max source and HESI II probe operating in switch polarity mode. Zwitterionic Sequent zic philic column (150×2.1 mm, 5 μm polymer, part #150460, MilliporeSigma, Burlington, MA) was used for polar metabolite separation. Mobile phase A (MPA) was 20 mM ammonium carbonate in water, pH9.6 (adjusted with ammonium hydroxide) and MPB was acetonitrile. The column was held at 27° C., injection volume 5 μL, autosampler temperature 4° C. and LC conditions at flow rate of 0.15 mL/min were: 0 min: 80% B, 0.5 min: 80% B, 20.5 min: 20% B, 21.3 min: 20% B, 21.5 min: 80% B with 7.5 min of column equilibration time. MS parameters were: sheath gas flow 30, aux gas flow 7, sweep gas flow 2, spray voltage 2.80 kV for negative & 3.80 kV for positive, capillary temperature 310° C., S-lens RF level 50 and aux gas heater temp 370° C. Data acquisition was done using Xcalibur 4.1 (ThermoFisher Scientific) and performed in full scan mode with a range of 70-1000m/z, resolution 70,000, AGC target 1e6 and maximum injection time of 80 ms. Data analysis was performed in Compound Discoverer 3.1 and Tracefinder 4.1. Samples were injected in randomized order and pooled QC samples were injected regularly throughout the analytical batch. Metabolite annotation was done base on accurate mass (±5 ppm) and matching retention time (±0.5 min) as well as MS/MS fragmentation pattern from the pooled QC samples against in-house retention time+MSMS library of reference chemical standards. Metabolites with CV<30% in pooledQC, were used for the statistical analysis. The quality of integration for each metabolite peak was reviewed. Polyamines and sulfur-containing metabolites were detected using the method described in quantitative media analysis.

Media quantitative analysis: At 8hpi, spent media was collected, centrifuged at 300×g for 4 mins and kept on ice. 30 μL of spent media was extracted with 120 μL of ice-cold acetonitrile containing metabolomics amino acid mix standard from Cambridge Isotope (MSK-A2-1.2), ¹³C6-glucose, ¹³C3-pyruvate, ¹³C3-lactate, ¹³C5-glutamine, ¹³C4-pyridoxine as internal standards and incubated on ice for 20 mins to precipitate proteins. After centrifugation at 21,000×g for 5 mins, 90 μL of supernatant was collected and stored at −80° C. On the day of analysis, samples were incubated on ice for 20 mins and the supernatant was collected in an autosampler vial after centrifugation at 21,000×g at 4° C. for 20 mins. Calibration curves were prepared in water at varying concentrations depending on the amino acid/metabolite level in DMEM media. Metabolite separation was done using XBridge BEH amide (2.1×100 mm, 2.5 μm, part #186006091, Waters Corporation, MA). Mobile phase A was 90/5/5 water/acetonitrile/methanol, 20 mM ammonium acetate, 0.2% acetic acid and mobile phase B was 90/10 acetonitrile/water, 10 mM ammonium acetate, 0.2% acetic acid. The column temperature was 40° C., injection volume 54, and the flow rate was 0.3 mL/min. The chromatographic gradient was 0 min: 95% B, 5 min: 70% B, 5.5 min: 40% B, 6 min: 40% B, 6.5 min: 30% B, 7 min: 30% B, 7.1 min: 20% B, 8.6 min: 20% B, 8.7 min: 95% B, 12.5 min: 95% B. MS parameters were same as those mentioned in the intracellular metabolite profiling method. Samples were injected in randomized order and pooled QC samples were injected regularly throughout the analytical batch. Data analysis was performed using Tracefinder 4.1 and respective internal standards were used to calculate the absolute concentration. External calibration curve was used for the quantification of asparagine, nicotinamide and tryptophan.

Virus inactivation assay: Applicants tested whether solvents used for cell pellet and spent media metabolomics (dry ice-cold 80% methanol and ice-cold acetonitrile, respectively) inactivated the virus for safe future experimentation. 5×10⁴/well of Vero E6 cells in a 96 well plate were infected with 10uL of samples treated with the solvents. Each sample were validated in triplicates. After culturing for 72 hrs, Applicants confirmed absence of viral protein, indicated by negative signal in nucleocapsid immunofluorescence assay.

### Statistical Analysis

Unless otherwise indicated, all bar graphs represent the arithmetic mean of three independent experiments (n=3), with error bars denoting standard deviations. Data were analyzed using two-tailed paired Student t test or analysis of variance (ANOVA) with the appropriate post-test using GraphPad Prism7 software. P values correlate with symbols as follows, ns=not significant, p>0.05; *p<0.05; **p<0.01; ***p<0.001, ***p<0.0001.

Graphics

Figures were drawn with GraphPad, Biorender, and ggplot2 in R.
1 Coronaviridae Study Group of the International Committee on Taxonomy of, V. The species Severe acute respiratory syndrome-related coronavirus: classifying 2019-nCoV and naming it SARS-CoV-2. Nat Microbiol 5, 536-544, doi:10.1038/s41564-020-0695-z (2020).
2 Viglione, G. How many people has the coronavirus killed? Nature 585, 22-24, doi: 10.1038/d41586-020-02497-w (2020).
3 Zhu, N. et al. A Novel Coronavirus from Patients with Pneumonia in China, 2019. N Engl J Med 382, 727-733, doi:10.1056/NEJMoa2001017 (2020).
4 Sawicki, S. G., Sawicki, D. L. & Siddell, S. G. A contemporary view of coronavirus transcription. Journal of virology 81, 20-29, doi:10.1128/JVI.01358-06 (2007).
5 de Wit, E., van Doremalen, N., Falzarano, D. & Munster, V. J. SARS and MERS: recent insights into emerging coronaviruses. Nat Rev Microbiol 14, 523-534, doi:10.1038/nrmicro.2016.81 (2016).
6 Irigoyen, N. et al. High-Resolution Analysis of Coronavirus Gene Expression by RNA Sequencing and Ribosome Profiling. PLoS pathogens 12, e1005473, doi:10.1371/journal.ppat.1005473 (2016).
7 Finkel, Y. et al. The coding capacity of SARS-CoV-2. Nature, doi: 10.1038/s41586-020-2739-1 (2020).
8 Snijder, E. J. et al. A unifying structural and functional model of the coronavirus replication organelle: Tracking down RNA synthesis. PLoS biology 18, e3000715, doi:10.1371/journal.pbio.3000715 (2020).
9 Hartenian, E. et al. The molecular virology of Coronaviruses. The Journal of biological chemistry, doi:10.1074/jbc.REV120.013930 (2020).
10 Schneider, M. et al. Severe acute respiratory syndrome coronavirus replication is severely impaired by MG132 due to proteasome-independent inhibition of M-calpain. Journal of virology 86, 10112-10122, doi:10.1128/jvi.01001-12 (2012).
11 Blanco-Melo, D. et al. Imbalanced Host Response to SARS-CoV-2 Drives Development of COVID-19. Cell 181, 1036-1045 e1039, doi:10.1016/j.ce11.2020.04.026 (2020).
12 DeDiego, M. L. et al. Severe acute respiratory syndrome coronavirus envelope protein regulates cell stress response and apoptosis. PLoS pathogens 7, e1002315, doi:10.1371/journal.ppat.1002315 (2011).
13 Wagner, B. K. et al. Large-scale chemical dissection of mitochondrial function. Nat Biotechnol 26, 343-351, doi:10.1038/nbt1387 (2008).
14 Mick, E. et al. Distinct mitochondrial defects trigger the integrated stress response depending on the metabolic state of the cell. Elife 9, doi:10.7554/eLife.49178 (2020).
15 Rabouw, H. H. et al. Small molecule ISRIB suppresses the integrated stress response within a defined window of activation. Proc Natl Acad Sci USA 116, 2097-2102, doi:10.1073/pnas.1815767116 (2019).
16 Kamitani, W. et al. Severe acute respiratory syndrome coronavirus nspl protein suppresses host gene expression by promoting host mRNA degradation. Proc Natl Acad Sci U S A 103, 12885-12890, doi:10.1073/pnas.0603144103 (2006).
17 Ducker, G. S. & Rabinowitz, J. D. One-Carbon Metabolism in Health and Disease. Cell metabolism 25, 27-42, doi:10.1016/j.cmet.2016.08.009 (2017).
18 Allegra, C. J. et al. Enhanced inhibition of thymidylate synthase by methotrexate polyglutamates. The Journal of biological chemistry 260, 9720-9726 (1985).
19 Allegra, C. J., Drake, J. C., Jolivet, J. & Chabner, B. A. Inhibition of phosphoribosylaminoimidazolecarboxamide transformylase by methotrexate and dihydrofolic acid polyglutamates. Proc Natl Acad Sci USA 82, 4881-4885, doi:10.1073/pnas.82.15.4881 (1985).
20 Chabner, B. A. et al. Polyglutamation of methotrexate. Is methotrexate a prodrug? J Clin Invest 76, 907-912, doi:10.1172/JCI112088 (1985).
21 Bao, X. R. et al. Mitochondria) dysfunction remodels one-carbon metabolism in human cells. eLife 5, e10575, doi:10.7554/eLife.10575 (2016).
22 Ducker, G. S. et al. Human SHMT inhibitors reveal defective glycine import as a targetable metabolic vulnerability of diffuse large B-cell lymphoma. Proceedings of the National Academy of Sciences 114, 11404, doi:10.1073/pnas.1706617114 (2017).
23 Codo, A. C. et al. Elevated Glucose Levels Favor SARS-CoV-2 Infection and Monocyte Response through a HIF-1alpha/Glycolysis-Dependent Axis. Cell metabolism 32, 498-499, doi:10.1016/j.cmet.2020.07.015 (2020).
24 Codo, A. C. et al. Elevated Glucose Levels Favor SARS-CoV-2 Infection and Monocyte Response through a HIF-1alpha/Glycolysis-Dependent Axis. Cell metabolism 32, 437-446 e435, doi:10.1016/j.cmet.2020.07.007 (2020).
25 Bouhaddou, M. et al. The Global Phosphorylation Landscape of SARS-CoV-2 Infection. Cell 182, 685-712 e619, doi:10.1016/j.ce11.2020.06.034 (2020).
26 Thaker, S. K., Ch'ng, J. & Christofk, H. R. Viral hijacking of cellular metabolism. BMC Biol 17, 59, doi:10.1186/s12915-019-0678-9 (2019).
27 Gordon, D. E. et al. A SARS-CoV-2 protein interaction map reveals targets for drug repurposing. Nature 583, 459-468, doi:10.1038/s41586-020-2286-9 (2020).
28 Bukreyeva, N. et al. The IMPDH inhibitor merimepodib suppresses SARS-CoV-2 replication &lt;em&gt;in vitro&lt;/em&gt. bioRxiv, 2020.2004.2007.028589, doi:10.1101/2020.04.07.028589 (2020).
29 Tong, X. et al. Merimepodib, an IMPDH inhibitor, suppresses replication of Zika virus and other emerging viral pathogens. Antiviral Research 149, 34-40, doi: 10.1016/j.antiviral.2017.11.004 (2018).
30 Garcia-Canaveras, J. C. et al. SHMT inhibition is effective and synergizes with methotrexate in T-cell acute lymphoblastic leukemia. Leukemia, doi:10.1038/s41375-020-0845-6 (2020).
31 Stegmann, K. M. et al. The folate antagonist methotrexate diminishes replication of the coronavirus SARS-CoV-2 and enhances the antiviral efficacy of remdesivir in cell culture models. bioRxiv, 2020.2007.2018.210013, doi:10.1101/2020.07.18.210013 (2020).
32 Frohman, E. M. et al. Part II. high-dose methotrexate with leucovorin rescue for severe COVID-19: An immune stabilization strategy for SARS-CoV-2 induced ‘PANIC’ attack. Journal of the neurological sciences 415, 116935, doi:10.1016/j.jns.2020.116935 (2020).
33 Beck, S. et al. Mechanism of Action of Methotrexate Against Zika Virus. Viruses 11, doi:10.3390/v11040338 (2019).
34 Muthusamy, T. et al. Serine restriction alters sphingolipid diversity to constrain tumour growth. Nature, doi: 10.1038/s41586-020-2609-x (2020).
35 Gao, X. et al. Dietary methionine influences therapy in mouse cancer models and alters human metabolism. Nature 572, 397-401, doi: 10.1038/s41586-019-1437-3 (2019).
36 Gordon, C. J. et al. Remdesivir is a direct-acting antiviral that inhibits RNA-dependent RNA polymerase from severe acute respiratory syndrome coronavirus 2 with high potency. The Journal of biological chemistry 295, 6785-6797, doi:10.1074/jbc.RA120.013679 (2020).
37 Agostini, M. L. et al. Coronavirus Susceptibility to the Antiviral Remdesivir (GS-5734) Is Mediated by the Viral Polymerase and the Proofreading Exoribonuclease. mBio 9, doi:10.1128/mBio.00221-18 (2018).
38 Harcourt, J. et al. Severe Acute Respiratory Syndrome Coronavirus 2 from Patient with Coronavirus Disease, United States. Emerging infectious diseases 26, 1266-1273, doi: 10.3201/eid2606.200516 (2020).
39 Sun, Z., Li, Y., Ransburgh, R., Snijder, E. J. & Fang, Y. Nonstructural protein 2 of porcine reproductive and respiratory syndrome virus inhibits the antiviral function of interferon-stimulated gene 15. Journal of virology 86, 3839-3850, doi:10.1128/JVI.06466-11 (2012).
40 Ma, Y. et al. CRISPR/Cas9 Screens Reveal Epstein-Barr Virus-Transformed B Cell Host Dependency Factors. Cell Host Microbe 21, 580-591 e587, doi:10.1016/j.chom.2017.04.005 (2017).
41 Dobin, A. et al. STAR: ultrafast universal RNA-seq aligner. Bioinformatics (Oxford, England) 29, 15-21, doi:10.1093/bioinformatics/bts635 (2013).
42 Liao, Y., Smyth, G. K. & Shi, W. featureCounts: an efficient general purpose program for assigning sequence reads to genomic features. Bioinformatics (Oxford, England) 30, 923-930, doi:10.1093/bioinformatics/btt656 (2014).
43 Love, M. I., Huber, W. & Anders, S. Moderated estimation of fold change and dispersion for RNA-seq data with DESeq2. Genome biology 15, 550, doi:10.1186/s 13059-014-0550-8 (2014).

TABLE 4A
Genes and Proteins from RNAseq Analysis.

NFKBIA, PPP1R15A, TNFAIP3, ARRDC3, JUN, NFKBIZ, IFIT2, HSPA5, DUSP1, IL11, EEF1A1, TIMP2, KANSL1, IFIT3, SQSTM1,
TIPARP, ZC3HAV1, KLF6, ENO1, GADD45B, NPC2, ANXA1, IGFBP3, DDIT3, PSAP, SPP1, CSRNP1, IFIH1, ADAMTS1, GPNMB,
CTSL, BHLHE40, ARID5B, PRDM1, TPT1, TUBA1B, IER3, TMBIM6, HES1, OAZ1, EGR1, GHITM, COX3, APLP2, KPNA2, VIM,
HNRNPA1, YWHAQ, NUAK2, AMOTL2, GRN, PRKDC, CCNL1, CHD2, MYC, ALDH1A1, CALM2, PFKFB3, LGMN, COX1,
CALM1, ACTR2, DYRK1A, PLK2, SUPT16H, PDGFB, TPI1, CCN1, SLC39A1, COX2, IFIT1, STMN1, PFN1, CHRDL1, DUSP8,
CST3, NOCT, ATF3, SPEN, NEURL3, RHOB, RGS2, CRYZ, PHGDH, IRF1, CLK1, CXCL10, LAPTM4A, RUNX1, MYH9, SMAD7,
EPC1, AEN, AP2M1, TUBA1A, CDC42EP3, RPS4X, ZFAND5, MT-ND4, CTNNB1, FOS, POGLUT2, MSN, RRM1, GANAB,
FAM83G, TXNIP, HSP90AB1, BRD2, CYTB, EPRS1, PDZD8, ARHGEF28, LGALS1, FOSL2, HMGB2, HSPA8, CD63, ARL6IP5,
TMED9, CHD7, ND3, IER2, EIF1, DHX58, MMP1, TUFT1, IL1A, PDZK1, UQCRC1, HNRNPM, PABPC1, CCN2, ATP6, HIVEP2,
SPRY2, CCL2, ZFP36L2, LGALS3, ATP5F1A, ERRFI1, EPHA2, SLC12A7, LRPAP1, SETD5, PPT1, AKAP17A, EEF2, ND4L,
PDIA3, NHSL1, PPARG, ATPIA1, DUSP5, SLC25A25, ZNF503, HSPD1, XRCC5, SBNO2, TNFRSF10B, PCNA, PTTG1IP, PELI1,
TPX2, PGRMC1, STARD13, HNRNPU, CALU, CLIC4, PPP1R18, PPM1D, MAP3K1, OAT, TSC22D2, TPM1, WSB1, EPCAM, OS9,
EDN1, MIDN, ND1, KLF3, S100A4, NPM1, KMT2C, CLDN2, RPN2, EPB41L2, CD9, PIM1, AKR1B1, MDH1, URB1, SERPINE1,
NARS1, NDUFA4, ZMPSTE24, NCKAP5L, SKP1, PPRC1, MIF, PSMB1, ATP6AP2, CTNNA1, GSTP1, SPCS2, ND5, PAIP2, ZFP36,
DDB1, FRS2, ANP32A, MCFD2, STK35, PSMA5, MMP10, BCL10, IDH3B, MCM3, ATP5MC1, DDX1, SFSWAP, CETN2, BCAR1,
CALR, MT-ND2, ATP6VOE1, MYL12A, EPHA7, SERPINH1, UQCR10, NOLC1, CAP1, KAT6A, NCL, PRC1, PSMB7, CERK, SND1,
ARHGAP23, NR1D1, ANP32E, YTHDF1, FAM107B, PALM2AKAP2, PIM3, TMED2, SEPTIN2, PDIA6, IGFBP6, PTPN14, PRDX3,
INTS5, TNRC6A, SH3BP4, ZBTB7A, MYL12B, THBS1, TAGLN2, BCAP31, CSDE1, RPS12, NDUFA13, COL4A1, UQCRQ, GPC4,
EDARADD, HMGXB3, CRIP2, LAMA5, FASN, POLR2L, PSMB3, UGDH, MDH2, ABL2, ACO2, JUND, PSMD1, SOX4, PSME2,
NUAK1, TENT4A, DYNC112, ZNF608, SDC2, KLF10, COL5A2, STIP1, WARS1, GDF15, NDUFA5, RPS6, PARP1, MDM2, NOP56,
CCL5, ZNF592, PGD, JUNB, MYH10, GADD45A, ERH, ZNF217, DUSP6, EIF4G2, FAM71A, WDR45B, RBM3, CREBBP, DLD,
RPS19, BUB1, NCOA6, MARS1, NACC1, LMTK2, JPT2, NUP98, ACADM, CD44, ANKRD50, TRIO, SRRM2, COX5B, TMED10,
USP36, FRMD6, ELL2, DUSP16, SPAG5, IREB2, G3BP2, KDM6B, TK1, ENY2, MTMR10, AGO2, POLR2B, COX8A, PELO,
FBXO42, STAT1, CSNK2A2, KMT2E, TET3, KMT2D, COX4I1, PGRMC2, KIF20A, POLD2, STOM, CYP3A5, MRPL13, SETD2,
ATN1, MED13, HELZ2, ASXL1, KMT2B, GPATCH8, DLGAP5, FOXN2, MORF4L2, RIPK4, OTUD1, VDAC3, NOP10, COPE,
FAM193A, MAP2K3, XRCC6, DSTN, TMEM59, IFFO2, RUSC2, ATP6AP1, NCOR2, NUTF2, RNF111, PARD6B, FANCD2, EWSR1,
ITGB1, RIC1, RPL30, ATP5F1E, ITPRID2, PSMB2, SERF2, URB2, INHBA, SAMD4A, BTF3, PSMA4, C16orf72, SRP14, TMCC1,
B4GALT5, MAGED2, AP1S1, DAZAP2, PCGF2, CIRBP, ZWINT, CEP85L, HNRNPH2, ARIDIA, RNF41, SOCS5, PPP4R3A, IK,
SSBP1, PHF12, SRSF3, LARS1, PLK1, TNFRSF1A, GBX2, PSMB6, DDX5, DIDO1, TLK2, NFIL3, CCNB1, MARS2, UGCG, VPS29,
EIF2AK4, FGF5, PPP1R3C, ARAP2, SEC24A, ZNF316, SPATS2L, ISG15, SCAF4, ETFA, SLC7A5, TMEM123, SEMA7A, TUBB4B,
RBBP6, PREPL, REXO1, ARHGAP1, DLG5, ETS2, PSMB5, DPP3, RAD54L2, GARS1, ALCAM, ABL1, POLA1, POGZ, ARID2,
NDUFB5, CSE1L, CIB1, CKAP2, NDUFA8, YTHDC1, CTSA, CAPZB, ACTN4, KPNA4, SPRED2, HNRNPC, SSR2, HBEGF, AHCY,
PRPF31, ECHS1, TRAF1, CRAMP1, COPA, RLF, KCNJ8, GPX1, NFKB2, RPLP1, FBXO11, API5, BRPF1, ZNF462, EXPH5, UBA2,
SNRPF, DUT, MGST3, TAF1D, ARPC5, EAF1, CAMSAP1, SSH2, NAA20, PDHB, ANLN, CDH17, IRF2BP2, CDH16, MLLT10,
ZCCHC14, CAPG, SDC4, COPS5, MAP3K14, SKI, SUPV3L1, NCAPG2, USP42, BDH2, BANF1, MSL2, ATP1B3, CDC20, DUSP3,
DDOST, HS3ST1, MCM6, PRKAG2, AIFMI, PBK, BEX3, COG3, AMMECR1L, IVD, NDUFB3, RASSF3, LDLR, FRMD4B, DAD1,
POLR2G, RGL1, ACAA2, NDUFA9, N4BP1, NUCKS1, PSMA7, VCP, TPP1, SRP54, G3BP1, MEF2D, TET2, ATP5PB, MITF, BSG,
ATP10D, CD81, RBM33, HSPA4, ANKRD17, CNN3, ID1, CETN3, GLUL, NUCB1, ZNF398, APOE, PSMD10, SPARC, TRAF4,
POMP, IFI44, BCCIP, CHMP5, SCARB2, NDUFS2, HEG1, GALK2, VCAN, SERBP1, PRDX5, TECR, HLTF, TES, S100A1, IPO8,
SLIRP, KIAA1522, UBN1, VPS26A, RREB1, CCNB1IP1, TSPAN3, OCIAD2, PLEKHM2, HERPUD1, NEDD9, HSPB1, SHMT2,
KDM2A, BRPF3, SMURF1, TMEM109, FOXJ3, ANXA5, ATP6VIA, PTPRF, SNRPB, ND6, RPS3, RERE, DHX15, AFF1, TRIB1,
YOD1, GATAD2B, CLUH, UBL5, UIMC1, PCYOX1, NUF2, SETDB1, DAPKI, CYB5B, KLF11, UQCRB, COPS6, BTBD7, TTC7B,
IER5, FNBP4, PLEKHG3, PPFIA1, ST3GAL1, MCM2, PMPCB, ITM2C, GNS, FEM1C, SCP2, RAB10, ZKSCAN5, RUVBL1,
HECTD1, SLC30A9, ABTB2, RBM39, PSMA6, SLC39A10, PPP1R9B, CRKL, PSMD2, USP5, NME1, RBAK, LAMA4, SLC25A37,
STARD7, TANC1, DSG2, RBM8A, MARCHF6, ZCCHC2, POLE4, CDK12, ZNF7, SHCBP1, CBX3, MB21D2, IFNAR1, PPP1R15B,
RBBP7, PSMD14, EFTUD2, DDX60, ROMO1, MRPL15, G6PD, HADHB, AKAP13, MIDEAS, DYNLT1, INO80, ERN1, NDRG1,
FBXO28, BUB3, TM9SF3, CTTN, RAB12, ARPC2, LASP1, PRKCSH, FSTL3, CPSF7, NPNT, FBXW11, EIF4A1, RUBCN, CLDN4,
CNBP, NPTN, NRIP1, MARK2, TRPS1, BCAS2, E2F4, RACK1, ZDHHC6, RSPO3, MYO10, CHD4, RBM15B, DOCK5, REV1, SRI,
PPP2R2A, CYBA, FOXA1, BAZ1B, PIGN, NUP107, MRPS9, DOCK10, PIBF1, PSIP1, VRK1, PLD3, PCF11, RANBP1, UPF1,
SRBD1, SUN1, RPS2, HNRNPDL, BRD4, TGFBRAP1, RAB7A, ARID3B, EVPL, YWHAG, LMNB1, CLK4, PCBP1, SERPINE2,
ZZEF1, ZFHX3, ZNF267, CHD1, APP, CCT2, ZBTB4, ZFY, IMMT, C1QBP, KIFBP, ETFB, NOTCH1, EP300, AKAP8L, DYNLL1,
ADNP2, CLIC1, MGST1, TM7SF3, ZSCAN12, MICALL2, GET3, BNIP3L, CAPNS1, MAGOH, C11orf58, MAPRE1, CCDC88C,
TBC1D10B, CANX, PTPRJ, ZNF746, MRPL27, ZNF655, ZNF574, ZNF853, EIF1B, SAT1, FANCI, POLR2K, PER2, KIF11, CNOT4,
IVNS1ABP, ZBTB43, PIEZO1, UBIAD1, SDC1, RAB11FIP5, MRTFA, PTBP2, MCM5, RAB8A, NRBP1, CD164, GPAT3, NOC2L,
POLR2E, ZNF787, TICAM1, COL4A2, VEGFA, LRIG2, CMAS, ATP5MPL, LIF, KLHL21, TGIF2, CREG1, SLC12A1, PIN1, IP6K3,
MYSM1, TOP2A, PCNT, ARHGDIA, EXOSC5, ADGRL2, HIRIP3, FSTL5, DGKD, DNAJA3, SYNCRIP, RFX7, RFNG, CDC25A,
PITRM1, CHCHD7, VTA1, XBP1, MFHAS1, RBM47, GLO1, ACTN1, BZW1, RGS3, PSMC2, LAS1L, CDCA3, CDK13, GZF1,
ZDHHC8, DHX37, PARK7, CHURCH, ACAD9, SMG1, SNIP1, UBR2, EMC2, H2AC6, DAGLA, TPM3, PAK2, RASSF8, ATP13A3,
PSMA3, SLC1A4, EIF2AK3, CHAF1A, ARID4B, SAE1, BECN1, ATXN10, PLIN1, GOLIM4, ZNF131, ENSA, TFRC, RAI1, CRLS1,
FBXW7, PPP1CA, PIGS, ASNS, ZNF142, GEMIN4, TBC1D9, CYC1, CSRP1, PKD2, SLC25A33, LGALS3BP, PAXIP1, ZNHIT1,
ELOA, NAV2, SARNP, SUPT6H, LHX1, RAPGEF2, CAPZA1, FAM83H, ZNF335, RBM5, MACF1, COMMD2, IARS2, JADE1,
NDUFB6, IDH3G, ZNF827, CHPF, EIF4A2, SSB, NDC80, SH2B3, RPA2, S100A16, PLS3, ZC3H4, SNAPC4, RSAD2, SHMT1, RCN2,
LEPROT, ZNF3, RTF2, ITGA6, IFI6, SZT2, VPS35, KIDINS220, PAPOLA, ATXN2L, FOXO1, FUS, PAPSS2, CALD1, MAGT1,
CDKN1B, RAB5C, MRPL46, UBA1, AURKA, NHP2, PTHIR, TAXIBP1, COX5A, NDUFA6, INTS6, CD46, RAD23B, ARID1B,
EBNA1BP2, POLR1E, ZSCAN29, PHACTR4, POLR2F, PCMT1, C11orf68, SLC25A3, DMAC2, RSU1, GNA13, DDIT4, SELENOW,
RNF128, EP400, DPCD, PPP6C, EIF6, TPRN, PRMT5, SERPINI1, ZSCAN25, AXIN1, PPP1R10, MAST4, PPP4R1, RNF139, LMAN2,
LIMK1, ZBTB18, PRAG1, OASL, SERTAD2, LOXL1, SEC16A, MTA1, HNRNPD, UTP15, TMEM43, AFF4, DHFR, ACTL6A, BCL6,
DTYMK, ICE1, EHMT1, SCIN, ATP8B2, FOXK2, TRIM4, CLTC, H2AZ2, GLIS2, ZNF778, ECHDC1, ABCC1, BMP2, CEMIP2,
EAPP, OST4, KRT81, LRRC59, PRKAR1A, RBL2, VIRMA, UCK2, SCPEP1, MRPS35, LUC7L, CCNB2, CUEDC2, TAF15, ARL6IP6,
RRM2, STK40, UGP2, RNPEP, ERP44, PNRC1, DMAC1, CAMTA1, AZIN1, TARS1, MIS18A, DCXR, CWC15, HIPK3, NUP37,
RAD21, EIF4E, CPE, NEU1, TOP3B, PTER, NOP58, TXNDC12, HSD17B12, GSS, PHRF1, MAF1, ATP8B1, FAM193B, GTF3C6,
RPS21, BEX1, CRIM1, RSRC2, MPDU1, CCNJ, PML, HERC6, CLDND1, XPO1, ZMAT2, ZNF385B, FLCN, HNRNPL, UBN2,
EIF4H, ATP6V1C1, RNF213, DNAJB5, ATAD2, CCNT1, USPL1, SYNJ1, RPS13, ARHGAP17, HNRNPR, TBX18, SLC16A3, RPS15,
ERBIN, COMMD4, TAFA4, MAP11, MRPL11, SAP18, MRPS11, TNFRSF10D, BPNT1, DHRS2, PSPH, DARS2, OGFR, SPC25,
RASA3, TRIM24, PDCD4, NSUN2, GGA3, SETD3, UBE2D1, RARS1, TCEAL8, YME1L1, USP38, UBAP2, ZBTB10, TNKS2,
LMAN1, SPECC1, SAR1A, GRAMD1B, ZNF79, CTDSPL, CDC25C, ZBTB2, CDC14A, RPS5, CDK5RAP3, KIAA0232, LRP12,
DDX18, RELN, TMEM106C, MCM3AP, NUP93, AGTRAP, TIMP1, G2E3, ATRAID, PSMC3, MICAL3, NCOA4, ARF4, COG4,
BCKDHB, DECR1, MARCHF9, DTL, MCM4, PFAS, MLF2, GNG10, RUVBL2, CRK, ZNF48, MRPL58, HPS6, EXOSC8, DNAJC10,
UBE2A, SF3A3, SMARCC1, LYZ, MFSD14A, PSEN1, CTSF, SCCPDH, LSM7, SPINDOC, CEBPZ, NCLN, NUP210, C15orf39,
YWHAZ, MSMO1, PRR13, EPDR1, ESCO2, PARL, NAXE, NCAPG, ZNF408, BCL9, SYBU, EMC4, MFAP1, SRPRB, SMC2, REL,
DLG2, HLX, RPS8, SF3B6, AARS1, ATP6V1E1, TIGD5, TRIB3, CEP350, MYO1E, NR3C1, SHROOM3, HTATSF1, SNX2, RFC2,
EDIL3, ACADSB, SMG5, POT1, SPTSSA, RNF216, S100A2, CPSF3, PLEKHH3, OTUD4, PRDX1, PLP2, PAPOLG, PDE4D,
MACROH2A1, PTBP1, ARL14EP, PLAGL2, DMWD, ACVR1, WDR46, CCT7, PPP2R2D, IARS1, RANGAP1, EID1, ANKLE2,
ZFAND6, HSD17B2, DIP2C, AKIRIN1, TMEM131, RC3H1, THOC3, EIF4ENIF1, TSN, TFPI, TTC1, WIZ, RTL8C, SPRED1, PAM,
ABCB1, PPPIR3B, SEC61G, CENPM, ADAM17, ITGB5, AREG, VDAC2, CISD2, LRFN1, ZBED4, GRAMD1A, ZNF384, ACSL5,
LRRC17, MRPL42, MEPCE, MRPL37, NSD1, TM7SF2, AKR7L, YWHAH, RAN, ARL2, MXDI, CAT, MALSU1, COPB2, AIDA,
PRR12, TGIF1, CYB5R3, PMAIP1, DUSP4, CDK17, BRD1, ORC1, REST, MRPL28, LTBR, UBE20, YRDC, ANXA11, IDH2,
FEM1A, ALMS1, ADARB1, DOT1L, GOLGA3, DCBLD1, CCNT2, GAREM1, SBDS, EIF2S1, UROD, VAMP3, EIF3E, PNP, LSM12,
PPP5C, ZNF292, TTLL4, PDE4B, HSPA13, ARHGEF5, RBKS, ITPR3, FKBP15, GBA, SH3RF1, MSH2, TSEN15, MED13L, CDK2,
ANKRD13A, RRBP1, ADAM9, GTF2H4, SH3TC1, LMNA, ANTXR1, KDM4B, HADHA, SLC7A11, NCAPH, SLC39A8, CAV2,
SLC25A38, LARP1, CLCF1, LAPTM4B, PLCD3, MTMR12, RB1, HMGCL, CENPB, MARK4, SMARCA5, HECTD4, HMGN3,
TNRC6C, RTCB, ANKRD10, TTC37, MYCBP2, IRS2, GNAI2, NKRF, WDR19, SETD1A, CDC123, BST1, IRF2BPL, LRRFIP2,
PEPD, PSENEN, TPD52L2, ALDH3A2, SYF2, PER1, NSD2, WDR92, PHLDB2, CARS1, THOC6, DCAF5, MELK, GMEB2, LTBP3,
BLVRA, ZBTB11, CXorf56, SNRPB2, SHROOM4, AKR1C3, RPL27A, NDUFB2, NFKB1E, IER5L, ELAVL1, MMUT, RNASEH2C,
MRPL40, CXCL2, ZNRF3, DAP3, EVA1A, GPT2, PIGK, HACD3, PPP2RIA, GLG1, TACO1, LSM4, HYOU1, MDM4, PRKD2,
MKKS, EEF1A2, FNIP1, GON4L, Chsa-A, USP9X, HAVCR1, ACAA1, AP5B1, BCL2L2, FOXC1, FAM160B1, ZFHX4, DHX35,
USB1, LACTB2, TSEN34, FHOD3, CNDP2, ZBTB48, ATP5F1B, RNF38, PDLIM7, USP22, MIPEP, WDR83OS, PUS1, GPR37,
SELPLG, ZNF627, DYNC2LI1, ETV3, ZNF106, CSF1, RNF181, MRPL50, NASP, UNC45A, DDX20, PRMT1, TEF, ZNF12, DHX30,
RAB1B, FAM162A, TFAM, ZKSCAN2, KHSRP, CLCN6, GALE, HKDC1, ADAM10, RHBDD1, NIPBL, KDM4C, DDX58, PYURF,
DRAP1, AMBRA1, TBCID8B, SLC4A2, SVIP, NDUFV1, AK3, GMCL1, COX16, DGCR8, ITPA, SUCLG1, HMGN1, ZBTB21,
MTMR3, PXDC1, SEC61A1, NFE2L1, ATP5MD, RPN1, HS3ST3B1, ATXN7, BMP2K, SEC63, TMEM63B, KIF4B, SEC24B,
TMED4, SKIL, RBM15, CALCOCO1, PPARGC1A, BAIAP2L1, UBQLN1, SPTLC3, EOMES, ACTR6, UCKL1, PPP2R5D, TMEM67,
TTC7A, COMMD10, OGDH, GAREM2, TXLNG, ADNP, CDC23, PCDH7, USP14, DYNC1LI2, FKBP8, APOBR, NDUFB7, SETD4,
ZNF441, B3GALT6, ZNF276, TOX4, ZSWIM6, ZNF629, SNW1, NDUFAF3, TGFB1, LPCAT4, VPS35L, TIMM21, YY1AP1, MRPL9,
RASAL2, OTUD7B, WDR5, PMPCA, NREP, SERPINA1, IQGAP1, SLC39A7, ERCC6L, BET1, NEK2, SCYL1, MIER2, PRKACA,
NCKAP5, ATAD2B, RECQL, TRIM8, TRAPPC6A, ZNF473, PON2, RNF168, LRFN4, DCAF13, PCNX3, PBX2, TRA2B, MRPS25,
FOSL1, NUP88, ZNF74, STRADA, RNMT, NELFA, RSC1A1, PLS1, VCPIP1, PPMIG, HNF1B, PRRC1, FLRT3, POLR3A, METTL9,
CNPY2, ZNF484, MCCC2, CLASP1, QSOX1, VAMP7, PARP6, RABL3, ZC3H12C, RABGGTB, TNIP1, SPATA2, CPSF1, STARD9,
ABHD17C, CDK19, ZC3H12A, FBXO9, TNFRSF1B, HSD17B10, AOX1, TMEM168, YBX3, RALY, ZNF764, MAPKAPK2, TRAP1,
SOCS6, PFDN2, TNFAIP6, ZNF496, PAN3, KDM5B, MARCHF7, B4GALNT4, CLU, PHLPP2, RAB11A, ASB7, ATXN2, FIZ1,
TAPBPL, SEC11A, EIF4G3, STT3A, OSGIN2, FBL, PPIF, ABHD17B, CLCC1, MRPS22, NDUFS3, SCAF8, PIGF, ELP2, AKR1A1,
GCH1, GTF2IRD1, AHCTF1, NDUFS1, ECH1, PNPLA8, LPIN1, DPYSL3, IFT80, BUD13, HIBADH, MED9, WDR77, FNDC3A,
CNOT1, ATP6V1F, SNF8, IFIT5, MTREX, SDF2, YARS1, TTYH3, FBXL18, PSMD13, IPMK, PLK3, NPM3, MICU1, UBA3,
ANKRD28, SIPA1L3, RPA1, SLC20A1, HOOK1, SKP2, ZNF689, PMEPA1, MANF, MFN2, FAM3C, ATP5PF, RSPRY1, MICOS13,
FIG4, TRPM7, GRK2, ZNF317, ASPH, TMEM50A, HACD2, CCDC47, TUBA4A, MSL1, PRDX4, MAP3K4, ITPRIPL1, ANKRD33B,
ING5, COMMD9, FKBP5, NCSTN, DVL1, SMS, KLF4, NXN, TIAL1, ADH5, ESPL1, NR1D2, NFAT5, SNAP23, FH, APIG1, IFNL1,
NTN4, PARP12, SNRPD1, ZBTB8OS, FTSJ3, SPDL1, NQO1, KLHDC3, NSF, GLA, GATAD2A, HDLBP, C16orf70, CHD8, MRPL48,
IFNGR2, TBC1D10A, CREB1, ASRGL1, DCBLD2, TSPYL1, ZNF668, PIKFYVE, AURKB, IRF3, TMEM19, FUBP1, CA2, ABCB8,
UBE2R2, ESRRA, GINS3, MCL1, PPP2CA, RPS6KA1, CENPW, EFEMP1, MRPS15, GALNT2, CREB5, WDR54, ZNF768, VEZF1,
BICD2, GRAMD4, RPS6KA4, COL27A1, KIF2C, TGM2, PSMD8, ZNF2, POGK, MAP3K8, RPRD1B, LETMD1, SBF1, WBP2,
PHLPP1, ZNF697, CEP95, RMC1, TSC1, NDUFS4, FGD6, TANC2, SAMD4B, UBE3C, ANAPC5, PARP4, C6orf136, HNRNPH3,
DDRGK1, TBC1D14, KIF13B, TTK, WDR74, UQCC1, ENC1, ERGIC2, TRAF3, POLR3H, LRRC42, PEX19, STK17B, VMP1, GPX8,
MRPS23, FAM13B, LONP1, STX1A, ACTR1A, PCCB, RPUSD3, SYMPK, PRPF6, KCTD20, LRP8, CBX1, RPUSD2, RPL5, KDM6A,
DUSP7, CSNK1G2, USP24, SUN2, TRMT44, RPS6KC1, ZNF654, USP37, KRT17, DPP9, SF1, IGFBP2, CWC25, CLK3, CISH,
CRTC3, STUB1, GART, MICU2, GINS1, SGCE, TAPBP, PRKACB, RARA, ATF7IP2, CAPN2, ADGRG6, LRRC24, MED29,
FBXO45, NR4A3, CARD10, PTGES2, MYNN, YIPF5, SLC36A4, MIGA1, DNMBP, CDK4, SMURF2, RRP8, PHF2, PET100, CRY2,
CAMSAP2, KIAA2026, PREX1, INTS11, MAML1, FAR1, CACTIN, SMARCA1, RCAN1, GEM, KLF7, TRIM52, ZNF202, KIF15,
PDZD11, PCGF1, CKAP5, HMMR, CALM3, PDE6D, C4orf3, MRPS14, ARRDC2, LPCAT3, PLAUR, PICALM, CAPZA2, DPH3,
TGFB2, AJUBA, MET, KANSL2, NMT2, RAB18, HS3ST3A1, ASAP1, DYNLRB1, PATZ1, SERINC3, IL4R, RIPK1, AP2A1, CDT1,
ZNF598, EBP, ACACA, VPS28, ATF4, BPGM, SPOCK3, TBX2, MTF1, CDC26, ERP29, RNF138, HDGF, TYMS, SENP7, WDR48,
IQGAP3, ATP6V1B2, MAN1B1, BCOR, NAA15, ZSWIM4, PLEKHA5, CCNL2, IPPK, BAMBI, KHDRBS1, ELF3, AGO3, KIF14,
ENTPD7, ANKRD18B, CCDC115, PRICKLE2, DDX39A, PGLS, SERAC1, AMFR, EYA3, DSP, ACVR1B, TM2D2, MED6, ZNF281,
RHEB, ZBTB49, ARL1, FAM222B, NDUFV2, MIER3, MAP7, SNRK, STXBP5, GPN2, ACO1, RALBP1, PDCD10, LTA4H, SCG5,
ZMIZ2, FNIP2, TSPAN12, LRIG3, FN1, BICDL1, NFU1, TXNRD1, ALKBH4, CCNG1, MTG2, ELF4, HAX1, PLEKHA4, CDCA5,
ANKRD9, LACTB, FLNA, MBTPS1, IRF9, REXO4, ZNF394, PIH1D1, PLEKHM1, COX7A2, UBAP2L, INO80D, SNRPD3,
RNASEH2A, MARCKS, MEA1, ISOC1, CCNQ, WBP1, TST, SH3D21, QDPR, RAP2B, PRICKLE1, TFPT, ENPP5, TDP2, PRMT2,
AGPS, MAP7D1, ZNF609, NKTR, VPS4B, ZNF250, KNTC1, UBA5, RSBN1, TOMM7, CIT, ANKRD1, DTX2, MTMR4,
TOGARAM1, FCHSD2, CAV1, GJC1, CXCL11, LAMTOR4, CHMP2B, GPCPD1, HNRNPUL1, OPA1, KIF13A, NUP153, ZNF543,
CDC37L1, SPRYD7, ZFYVE26, ADAMTS9, QSOX2, ACTR3B, IPO7, ZHX3, ISYNA1, UBAC1, GAB1, KIF23, DDX41, MIEF2,
SUZ12, TADA2B, CENPN, TRIP12, KLHL11, SLC31A1, ERAP1, YIF1A, COLEC12, DNAJC3, TXNDC15, SPINT2, NOTCH2, EPN1,
ORC3, TSPAN6, DNAJB6, PREB, ZCCHC17, ABAT, CEP63, PEX3, USP6NL, KIAA1549, VPS45, EGR2, EFR3A, ANKRD13B,
ARL3, KIF3C, RAC1, CYB561A3, SHROOM2, PSMC5, ACADVL, FRMD4A, HSPA2, MAD1L1, CYBRD1, CDH6, ACVR2A,
C17orf80, LRRC8A, GPI, DPM3, SETX, NXT1, ANAPC16, ZIC2, FAM234A, NCOA5, PLXNA2, NDUFB10, NCKAP1, FBXO3,
PACSIN2, CACYBP, TUBB2A, COPZ1, LITAF, CRCP, HINT1, RAB6A, PPP1CB, AHCYL1, JMJD1C, DDX42, GGCX, KDELR2,
ZNF207, ZBTB24, EIF3B, TPMT, ALDH16A1, KLF16, SDF4, TP53BP2, SLC8A1, CNIH4, NR2F2, ZBED6, BIRC3, PPHLN1,
RNF169, CORO1B, SNX3, MAP1S, PRPS1, KIF1A, UQCC2, DCTN2, TOP2B, FOLR1, PLEKHA7, TIMM23, TCF25, WDHD1,
EIF2B3, MED12, CEP152, PARP14, MIOS, RNF103, GTF2H1, SLCO5A1, AMZ2, TBC1D15, CLK2, PITPNM2, CMPK2, DENND5A,
CHMP4A, SOS1, CD320, NDUFA2, SNRPA, ZNF282, NAF1, FXR2, KRT10, NIPAL3, BAG3, HLA-DMB, ANKRD11, RHOBTB2,
ANAPC13, EEF1D, ZNF561, RAPGEF1, ATG4C, IDH1, TCF20, ANK3, NPHP1, DENND4B, SEPTIN7, ZNF710, BCAP29, DHX36,
ASAP2, TRIM13, MPV17, FLOT2, NAA38, KAT8, CNPY3, SLC12A4, PPP1R26, TERF1, PKD1, SLC35B1, ALDH1A2, EREG,
KANSL3, LDB1, MLLT6, SP3, ERG28, E4F1, KMT5B, MIEN1, TNFSF15, ATP2A2, R3HDM2, BRD7, PCK2, NFKB1, HSD17B11,
BSDC1, USP11, PHF20L1, SERPINF2, ZNF318, ERI3, STAU2, CDK18, MRPS5, HTT, FNBPIL, MSANTD4, IFI44L, SRSF4, DCTN3,
INAVA, KDM7A, KLHL26, PHF6, VEGFB, SH3PXD2B, PRKAB2, UNK, GBE1, SIK3, ATF6B, ISCA1, DENND5B, UBE2I,
PRDM15, SELENOO, MAPKAP1, IQCC, RHOT2, RAD21L1, DDX17, TRMT1L, PFDN6, SATB2, ZUP1, VAMP8, RFC3, ADGRB3,
HGSNAT, SLC39A14, GTF3C4, ITSN2, RSRC1, AP1M1, ZNF823, SLC23A2, SLC35A1, MRPL16, PTPN21, CKAP2L, CRYBG3,
FLOT1, MLEC, CASP3, NSDHL, CCDC25, INTS4, MOSMO, RELB, FGFRL1, SEC22B, FBXL7, SF3B5, SH3D19, RC3H2, HSPG2,
PRPF39, GLDC, ATXN1L, INPP5E, RICTOR, SOBP, MTCL1, YTHDF2, TCP1, TMEM33, RIPK2, SEC62, COPS2, C5orf15,
CHAMP1, TOMM6, AP3B1, HP1BP3, AKIRIN2, ASCC1, DPP4, ACTR1B, GDI2, REPIN1, CPPED1, TENT5A, ZNF672, CYP17A1,
SRC, CINP, CD109, FKBP10, HIRA, GCSH, SENP5, EFHD2, CMSS1, GID8, NT5C2, TRIM44, TM9SF2, HPRT1, TIPIN, SEMA4C,
ZC3H7B, AP3D1, MAST3, IPO5, KIF21A, PFKP, PLA2G4C, BICRA, HECA, ITGB3BP, PPARGC1B, MAFG, TTF1, SCAF1,
TMEM201, CTH, RALB, NUCB2, CENPK, TASOR2, AGPAT2, GLB1, GMPR2, POC1A, SESN1, HMCES, LBR, RBX1, CCT3,
LRRC40, MRPL51, G6PC3, H2AX, SCAMP1, NSL1, PLCG1, CTTNBP2, ANKH, C2CD2, SIPA1L1, PCNX1, GDAP1, DIPK2A,
SEL1L, OTUB2, FAM171B, GRB2, DCAF12, TSPAN14, NUDC, BIRC6, PODXL, NUDT22, DCP1A, MTX2, ARHGEF17, CBLB,
CDC42, WEE1, PGAM4, SLC25A24, COQ8A, OGT, RIN1, APOBEC3C, PRKAG1, TMX1, NCF2, TAF1, PRDX6, PSMD9, DAB2IP,
GADD45GIP1, IL17RA, NAGA, PPIE, UHRF2, PDE7A, RNF121, RFC5, SNX10, RAP1GAP, KSR1, LRP1, CPA4, MTFR1, ACOT13,
MCM9, SNX18, RPLP0, UVRAG, TFAP2A, ATP5MC2, ALKBH3, RILPL2, UAP1, ZFYVE9, CNTFR, SRP68, SIN3B, SMARCE1,
LSM6, FAHD1, ARVCF, SLC13A1, BUD31, MMP14, WWTR1, MRPS24, UGGT2, TBC1D23, CISD3, FAM114A2, MAZ, ABHD15,
SCARF2, SPOCK1, USF1, MLXIP, SLC38A2, SRR, MMD, ASAH1, PROSER2, MLH1, MARF1, LANCL1, VCL, BCAT1, IFRD1,
TONSL, SLC25A30, NDUFB11, NOB1, HSPBP1, VSIR, URGCP, TMEM258, HBS1L, PLXNB2, VPS37B, KANSL1L, OGDHL,
TOPBP1, DUSP10, PGM5, DNAJC19, MAML2, TNS2, LAMTOR2, SALL2, CDKN2AIP, PFDN1, SNRNP48, SPPL3, CNOT7, RNF5,
NBL1, KNL1, POR, HDGFL2, RLIM, C15orf61, MYBBP1A, MGAT1, SGK1, MAU2, MACROH2A2, HERC2, ALG8, PMVK, ECPAS,
CPSF4, AEBP2, MKI67, SNRPC, ZNF367, SH3BGRL, ECT2, ATF6, LAMB3, ARHGAP35, PUM1, SOCS3, TUT7, ZCCHC8, PSME4,
EZR, SF3B2, KPNA1, LAMTOR5, SEC11C, LARP7, CYP27B1, SRRM1, MOCS3, BBS2, SLC38A9, DARS1, PIGT, TMEM170A,
ABHD17A, FAM53C, KIF22, SLC39A6, SSNA1, MEX3D, ZNF263, XPO6, RACGAP1, RFC4, GSPT2, COPG2, COX17, MCU,
CEP250, PDIK1L, CDV3, ADAM15, FKBP9, SF3B1, NOSIP, SLC9A3R1, ANXA3, RNFT1, MMP7, TRMT10B, TBC1D22B, ZBTB39,
CNST, BRWD1, NEPRO, BCAR3, HCN2, KLHL28, ANKRD52, DNAAF5, NBR1, CENPI, CTSC, GPKOW, SIMC1, ZNF565,
DGUOK, ZSCAN22, KLHL22, ERBB2, TMEM189-UBE2V1, GCN1, ATG16L1, ITM2B, MFSD2A, BYSL, SEC23A, METAP2,
ANKRD13D, CDC6, GIT2, KIN, BCL3, ZNF526, CHMP2A, DCUN1D3, ADAM19, HMGA1, LCMT2, CSNK2B, PRMT3, WRN,
CNOT2, STAT6, TNPO3, E2F6, TLK1, HELZ, FAM135A, CAP2, GLRX2, ELF2, SLC39A3, WIPI2, CHSY1, ZNF180, CLIP2, CDON,
METTL7B, NFS1, CCDC174, BPTF, SLC38A10, FMC1, CBX6, DSTYK, PLEC, FAM32A, CTNNBL1, MECR, M6PR, TIMM44,
HAT1, REPS1, WLS, DDR2, SLC6A13, CSPP1, GATA6, ZNF732, ING1, SNRPN, CENPA, GRPEL1, GNG11, SCAMP4, VPS13B,
TGFBR3, ARHGAP11A, TNPO1, PDK2, SUPT4H1, TRIR, PCDHGB7, CSNKIG1, GFPT1, TFB2M, ARF1, CD82, DDX51, FZD5,
RASL11A, SMAP1, ACE2, GCFC2, RER1, BTD, TNRC18, BRF2, LRCHI, PSMD3, EPHX1, NFX1, TOP3A, IMPAD1, RDH13,
PTPN23, RTTN, NEMP1, GPATCH2L, SERINC1, SNX17, MGAT2, FST, HOMEZ, PHKA2, OGFOD1, LLPH, AP1M2, ATXN7L2,
IKBIP, FOPNL, PPIH, ABHD10, FBXO38, CIZ1, MSX1, CKLF, PGM3, PI4KB, KRI1, RIPOR1, RSL24D1, FOXO3, NAA16, VSIG10,
ERCC6, TMEM98, DIP2B, SEC61B, CCDC167, USP3, NAA50, RANBP9, LUZP1, MTSS2, RASA2, WDR11, TRAF3IP2, PRPF4B,
SWI5, IGF2BP3, ZWILCH, GOLGA7, PAXBP1, CPNE3, MAT2B, RRPIB, RAD51AP1, NAPSA, GINS4, WBP11, ZFP36L1, PLA2R1,
TTC38, KARS1, KAT2A, PPP1R3F, RILPL1, TRIM33, NDUFS6, RCOR3, MPZL1, ATG2B, EIF3K, HDDC2, MTRR, MBD5,
CYSTM1, ERLIN2, GAPVD1, PNPT1, ZNF622, SIVA1, SSR1, BRF1, ARF6, TENT4B, C19orf48, CPSF6, NOTUM, OAS1, ZBTB7B,
ZNF16, TESK1, COP1, UHMK1, CASP6, WDR43, GTF3C1, GJB4, TJAP1, RBM26, VPS39, PARP10, BSN, SKIDA1, TBX20,
TOMM34, CASP8AP2, SMIM30, MRPL3, ABCC9, CD3EAP, COPS4, GCC1, PPIP5K2, RBFA, SMARCA2, C15orf40, HEXB,
SERPINB1, DNM2, FPGT, ZNF500, RAB2B, VEGFC, TMEM248, SKA1, COMMD6, SLC35F2, ITPKB, PHPT1, PBLD, AKTIP,
SIGMAR1, FSTL1, EEFSEC, ORMDL2, AAGAB, STMN3, USP15, CPNE8, TOPORS, CDKN1A, KLHL15, COPS3, MCRS1, NXT2,
COX11, SIRT1, CEMIP, MIER1, GRINA, TADA3, FAM131A, ZSCAN2, EIF2B4, DHRS1, UBA6, MTHFD1, PRDM2, GPS1, IPO9,
CSTF1, C12orf57, IP6K2, RNASEK, TMEM69, TRAFD1, GSE1, SEPTIN11, H2AJ, MGST2, TSPYL4, GLB1L2, ZBTB40, ZNF121,
TMEM167A, MAP3K13, GALNT7, SOCS2, THRAP3, STX12, LRR1, STXBP2, GATA2, TGS1, LYAR, DDX24, ALOXE3,
SELENOT, COPG1, SIRT3, CIS, HMGN5, FLNC, XPO5, RNF214, GRB7, TSFM, TAF10, UBE2NL, ANKRD46, FITM2, TECTA,
JMJD8, PLRG1, SAMD8, KCTD9, DCAF11, YEATS2, NDUFABI, GASI, CCT4, JUP, ZNF507, PARP2, RPAP3, IRF2, NSFL1C,
NDUFA10, HTATIP2, PMS2, PTK7, NOL9, MGMEI, CAPN15, ARHGAP39, DTX3L, ITFG1, TGFBR2, MEGF8, PGP, PARD3,
MTX1, C2CD2L, NAA40, GON7, FSCNI, SNCA, SNX1, TACC3, CREBZF, PFKFB4, ARAF, GSTCD, CLPTMIL, RBM6, PHACTR2,
CTIF, SLC38A1, ALKBH5, MPHOSPH8, OSBPL5, RAB24, TIAMI, RAD51C, TSG101, RAB21, MBTPS2, TLE3, ITGAV, NF1,
METTL21A, MRPL22, LAMP1, PARP9, SNRPA1, DEAF1, IWS1, SRF, APOBEC3F, ASF1B, SUCLA2, AVL9, MORC3, ATRIP,
GULP1, COL8A1, TTC26, PROS1, HSPH1, SOWAHC, SPTLC2, ZBTB37, BCAT2, KIFC1, DIS3, NACC2, CEP76, MTHFD2, PSMB8,
TRIP10, IFFO1, ATXN7L1, FAM174C, GMPPA, GNAI3, CPNE1, ZNF687, SCAP, ITPRIPL2, BASP1, GATD1, TMEM14A, HAUS1,
NANP, CAMSAP3, PIP5K1A, AHI1, FBXL6, ITGB1BP1, CBX2, SPNS1, SRPK2, RSBN1L, UBR4, EXT2, ISG20L2, STAMBPL1,
RAD51, DUSP2, TMEM205, UBR1, FAM98B, GMEB1, TAF13, IFI16, GLRX3, BCS1L, PRKCE, RBMS2, UBXN1, FAM118A,
TRRAP, FJX1, TAF5L, PPCS, SPCS1, SPRYD3, CD68, C20orf194, UPP1, TAOK2, CCDC124, SDHB, CDK8, STYXL1, ATXN7L3,
ERLEC1, PYCR3, IPO11, GAR1, KNSTRN, TBC1D1, DCAF6, FBLN1, ADD1, MCUB, RYK, GOLPH3, MCPH1, HMGXB4, DCDC2,
SNRNP200, TFIP11, BNIP1, CTSB, CBR4, AIF1L, HLF, RPS7, NDUFC1, RING1, TRMT12, HBQ1, IMPDH2, RNF215, SMAD6,
ALDH4A1, SPIRE1, EDRF1, DGLUCY, JMJD6, THAP12, PLCXD2, IGSF3, CBX4, PFKL, FBXL5, RIMS2, TBCD, FRZB, GLS,
FIGN, COMMD7, BCL2L13, MAP3K2, OSTM1, ALDH18A1, NUS1, PSMG1, MAPK3, ZNF8, AKAP10, UQCRFS1, CREBRF,
ZNF236, RNF144A, ITPKC, PDCD11, ZNF800, TMEM254, DENND4C, SRSF1, MRPS33, FXN, B3GLCT, HDAC2, SIAE, NAPA,
NEU3, ASXL2, MSH6, LSR, EMSY, CCND1, IFT52, AJM1, LRP11, KCTD2, POLR3E, IRF2BP1, ADSS1, RND3, PTPN2, RBCK1,
NCOA3, RGMB, CCDC85B, CUL9, PSMB10, MYO18A, TP53, NCAPD2, SNPH, PDCD5, HFE, MMACHC, JKAMP, GTPBP4,
TPD52L1, C16orf58, METTL22, NBEAL2, POLE3, MAP3K9, TIPRL, ASB2, INCENP, MMS22L, PSMD11, B2M, IRAK2, MAPKBP1,
RAB5B, BAZ2B, INO80E, PYCR1, ARHGEF10L, ARHGEF39, CCHCR1, PTPN4, PRR5, LIG1, UBXN4, RSL1D1, FECH, GALM,
MAP4K3, PSMC4, TOR4A, MCM10, TMEM214, MAP4K4, PTRH1, RPS6KA3, AMOTL1, CWC27, VAC14, AHDC1, COMMD1,
PHF23, BMP1, NSMCE1, SRSF2, LYPLA2, CYB5D2, EMP3, ZCRB1, CRYZL1, TSPAN4, WDR91, EPC2, PACS2, ST6GALNAC4,
EMC10, CUL4A, ALDH5A1, TENT5B, SGCB, SPSB1, KDSR, NUDT9, ZNF114, CLDN15, OGFRL1, UCHL5, ZBTB34, THEM4,
LRRC14, CXCL1, ZNF805, TRAPPC12, SMAGP, RBMS1, TIGD2, SMNDC1, CDCA2, RNF167, MORN2, CCDC142, ALG5, ITPK1,
CDC16, TMEM236, EXOSC1, NOL7, TICRR, BUD23, STOML2, HMG20B, TTC28, SZRD1, IDE, GPATCH1, CD151, LEPROTL1,
CPD, PPP2R3C, BRD8, SMO, OAZ2, SFMBT1, CIDEC, ABHD4, SH3BP5, NT5DC2, CLIP4, RPGRIP1L, TTC5, SPCS3, MPP3,
TMED7, MED17, PDCD6IP, TRAPPC3, ATG14, METRNL, CTSO, MESD, RBM19, AGA, CCDC22, SH3BGRL3, RGP1, UTP3,
AMACR, NPC1, ZNF330, SLC25A5, PTAR1, RRN3, RSF1, PRPF19, SLC25A28, EPB41L3, MCAT, WDR41, FHL2, CERS2,
FAM120A, USP34, TRAMI, CS, MED12L, KAT14, CAND1, DCDC1, DICER1, ZNF614, MCRIP1, GNPTG, HDAC7, INSIG1,
PTCH1, RMND5B, ADGRL1, LRP4, TBX3, S100PBP, COAI, KBTBD2, MBD1, KPNA6, WTAP, RWDD2B, DONSON, ZNF555,
PIAS4, IFI27L2, ZSCAN20, TXN2, NAE1, YIPF1, CSRNP2, PAIP2B, CCDC34, TPGS1, EFNA5, LAMP2, SLC2A2, FAM20B,
CSKMT, TPST2, SORT1, ZNF581, IFT81, BROX, KEAP1, ZMYND8, TNIP2, CAMTA2, NEKI, SEMA4B, TOMM70, ALYREF,
NAGLU, PRPF8, GSTZ1, HOXA9, CHAC1, DSEL, GABRA5, WASHC3, COL5A1, TXNDC17, MKRN2, FBXW8, D2HGDH, PHC2,
GFM2, MRPS12, DYRK2, IGHMBP2, TRAPPC8, TM4SF4, GTF2I, ADSS2, HOXC10, SUMF2, SULF2, NPEPPS, DNPEP, ARHGEF9,
ANO5, ITGB2, ATG101, NR6A1, PRR36, RANBP2, BRSK1, RHOA, RETREG3, PHYH, EXOSC4, KRT80, PLBD2, ALG11,
MOSPD3, AP2B1, MRPL53, OSBPL9, RHPN2, MAP3K5, SLU7, EIF3I, NUDCD3, MED15, TRAF5, PSMD12, CIC, ASH2L, ZNF14,
RNH1, TARBP1, SPR, PPP6R3, P4HA2, CLUAP1, ZNF547, ARID5A, NEK7, ZNF777, PTPRK, DHX33, RPP38, TRIM26, GIPC1,
CHCHD6, CD55, POLR3B, CCP110, NTPCR, PHF20, TUBA1C, UTP6, MAP2K4, GALNT10, FAM216A, MECP2, SH3GLB1,
SLC9A1, NUP62, PAPSS1, TTC39C, SLC19A1, CBARP, FAM20C, DDB2, TOR1AIP2, APBB2, LIPA, MRPL2, CHD9, VPS18, GPN3,
ACER2, NR2C2, JARID2, ZNF304, WAC, SERTAD3, MRPL39, ZNF513, GSKIP, BMPRIA, SLC7A6, RAD23A, SECISBP2L,
TWSG1, ZNF529, FAT1, SNRNP27, DENND1B, SHLD2, IRGQ, PLEKHN1, MLYCD, MAPK8IP3, SPRY1, GPBP1, CSTF2,
AURKAIP1, RCC1L, SH2B1, DCTPP1, KPNB1, APOBEC3G, PDCD7, ABCC5, BSCL2, PIK3CD, MRPS34, ZSCAN21, UNC119B,
PRADC1, DZIP1, GLRX5, COX15, BEND7, SPAG1, GTF2A1, EXOSC9, PLEKHA6, CCDC103, ACAT1, TCEAL4, SKA3, PPDPF,
ZNF652, REEP5, ZNF621, ZNF579, IRF7, EEF2K, TCEAL9, APIB1, CCDC58, ANO10, ZFP3, GLBIL, MYEF2, NUDT5, PLPPR5,
CBR1, ZNF703, PATL1, CMTM6, RMI2, HERC1, DOK4, PLEKHF2, ATP5F1C, TMOD4, CCNA1, HEATR9, KIF7, ZNF70, RBM42,
USP53, ATP6VOC, GPN1, EDC3, NDUFA12, RASSF1, DIAPH1, NUBPL, PJA2, UGT8, NCK1, TJP1, RIN2, CARD19, CIGALT1,
PLGRKT, FEZ2, MXD3, MOSPD2, TMEM185A, FILIP1L, MAD2L2, RHOC, ZNF620, CFH, ZHX2, POLB, NRDE2, PARN, MRPL36,
MTF2, RBBP8, CACNB1, NUP42, FABP4, HERC4, REEP2, ACOT7, DIPK1B, IFT27, NELFCD, CIR, MRGBP, AGBL5, MTFR2,
CTNNAL1, MGMT, DCTN5, NF2, TCEA1, SYDE2, COMMD8, HIC2, SCARA3, STRAP, B3GNT2, ARHGAP12, KCTD6, FAM8A1,
NME7, SGMS1, RBM17, RBM23, ATG12, TBCK, ECD, EMG1, NDFIP1, OXA1L, KIFC3, ZNF205, MTMR6, ENO3, ASH1L,
RNF19A, GMFB, STAM, WWC3, ORAI2, BLVRB, TMEM140, DNER, SS18, XPOT, UBE2B, DBF4, AUNIP, PER3, PRR14L,
ACAP3, TIGD1, TXNRD2, WDR26, NUBP1, ATP5ME, AGK, CDHR2, INTS6L, SLC29A1, LGALSL, DELE1, CLPB, ADAMTSL3,
GOLT1B, PPP6R2, ZNF436, WDCP, WSB2, NPTX1, UCHL3, TPK1, SLC12A2, TOB2, CCDC157, ADAP2, ZEB2, SLF2, EIF3L,
ARFGEF1, NANS, ZFP1, LAP3, FANCE, MTR, CYLD, NUFIP2, ILK, ABLIM1, PANK3, NCBP3, PLAAT3, SCO2, NUFIP1, NCOA7,
COPS7A, XPA, RPA3, KLF15, PRKCZ, STRN4, AIMP2, MYL6B, SRSF7, CMTM3, TRAPPC1, ZER1, TERF2, FARSB, LIMD1,
PRR3, CFAP36, GFER, XRCC4, PABPC4, TMEM63A, NSUN5, ZNF639, NEK3, TCTNI, TRMT61A, NR2F6, C3orf38, ESYT1,
SMCR8, TBC1D20, CHST3, CXCL3, SEMA4D, DDA1, PPP4R3B, ZNF333, ABRACL, ZNF41, FUT4, SF3A1, RBSN, UNC5D, DBT,
C7orf26, CCDC62, CAPRIN2, NIBAN1, ENOX2, SPTLC1, PXMP4, CYFIP2, CLDN12, TNFRSF11A, LCAT, N6AMT1, PPP1R14C,
MED1, CRYAB, SIAH2, CDR2, STOX1, NEMF, PARPBP, DAPK3, KLHL36, ARHGAP30, CFDP1, SCRN2, TNFSF10, ZNF77,
IRAK1, AUTS2, NR4A1, ZNF554, GSN, ANAPC1, DUS4L, ELOF1, UBE2J2, ZFAND3, ZMYM4, MAGI1, FAM126B, FNBP1, CHD5,
KPNA5, CHMP4B, BABAM1, DGKH, UBE2K, CPM, UTP11, TTC17, DUS3L, TUBE1, FAM229B, CEACAM19, ARVI, TNFRSF9,
STAM2, MINPP1, COQ10B, GTF2E1, DNAJC11, PBDC1, TUBD1, TIRAP, DUS1L, DPY19L4, INPP5B, SFT2D1, INTS3, VCPKMT,
CREB3L4, JAK1, IFI30, CPT1A, PRPF40B, TRIM56, STK24, VPS72, DEPDC1, GSK3B, USP1, TRIP13, ETNK2, PCGF6, PNO1,
APBB1IP, SESTD1, CARS2, TFDP2, SASHI, SWAP70, RABGAP1, ATP6VOA1, INTU, H1-0, SUGT1, NCK2, NEIL3, AACS,
ABCA4, GTSE1, TRIM23, PWWP3A, MAN2B2, OXSR1, REEP4, CBX5, NTS, SPTAN1, CCDC82, SURF2, GLMN, TRIM11,
TECPR2, EIF2B1, SNX25, KLHDC10, LMO7, PRKCI, TYW5, UNC119, NKAPD1, MGAM, NOP53, ZNF184, RAVER2, TRIQK,
CNOT11, USF2, DENND6B, LTO1, B4GALNT2, TTBK2, H2AW, GSDMD, COQ8B, FBXO21, MUL1, WDR37, TFG, PMM2,
ACTR3, NAPRT, WHAMM, PLCE1, TBPL1, C2orf42, ZSWIM3, FDXR, SOCS7, PIGG, USP21, EGR3, MRPS2, IST1, NGRN, PDXP,
APRT, DKC1, AFTPH, USP7, CMTR2, CALML4, BTAF1, SLC17A1, ERI2, CC2D1B, DYM, MYO5A, PIK3R4, UBALD2, INTS10,
S100A13, TMEM182, GFM1, CILK1, TMED1, ABHD6, DCTN6, MAPK1, TTC39A, PAAF1, VPS36, RPRD1, SLC6A12, PAFAH2,
ANKS1A, CNKSR3, TRPM4, PRKAA1, COPB1, ISCU, PTPN3, S100A6, ABCA2, ZBTB9, APPBP2, FAM102A, SALL1, CSK, HCN3,
KLHL18, PTPRA, FAU, HK1, MOB2, RCC2, AAK1, N4BP3, THAP4, ZDHHC20, PSMF1, TLNRD1, TFAP4, SF3B3, TMEM126B,
AATF, ETFDH, CCNY, BAZ2A, KLHL12, MNAT1, CCDC117, TLR3, DZIP3, LIPE, BRWD3, GLCE, TCTN3, GNE, ZMYM2,
CEP192, NDUFS7, PLEKHG2, KLHL24, ARFRP1, IMP4, LDOC1, MFSD3, KCTD3, NRDC, GOT1, WDR61, POLE, PTGR1, JAK2,
TMED5, FBXO34, SPG7, CEP55, TMEM11, BBS9, UTP14C, TMEM222, UBP1, PTPA, PTPRE, TRIP6, NABP1, VHL, TRAF2, LYN,
BACH1, NAP1L3, UHRF1, SRGAP1, SUMO3, LGR4, KYNU, OTUD3, TMEM54, SFXN3, EMC3, AQR, TMTC4, MRPS10,
B4GALT7, EBPL, THSD4, PLEKHH2, ARHGAP29, CTU1, RPUSD4, ELP1, SDHAF2, LAMB1, HBP1, NFRKB, AGPAT1, YIPF4,
HYAL1, ARMC7, USP47, PDP1, FRA10AC1, MMADHC, SDF2L1, HIBCH, SGSM1, NEFL, AXIN2, C18orf54, MTHFS, NDUFV3,
LZTS1, CCDC198, MBLAC2, ORC5, AGRN, SERTAD1, AP3M1, ALDH7A1, OTUB1, PRKAR2A, TSTD1, TRAPPC4, WDR81,
RBM10, JRK, BBS4, ZNF597, ARHGAP21, C11orf49, JOSD1, CCDC91, ANP32B, ZNF821, ABI1, CYP1B1, SLC12A9, HTRA1,
RAB29, C11orf54, PEX2, MAMLD1, SLC25A22, TRNT1, THUMPD2, ELAC2, MSL3, PDE4A, CCDC120, POP1, XPO4, DNAJB2,
IDNK, KRTCAP2, B4GALT3, HIPK2, DDX27, RTN4IP1, CLASRP, SETDIB, SLC45A4, CEBPB, BTC, TPM4, COBL, GBA2,
HECTD2, ARHGEF3, UPK1B, PABPN1, ZNF107, MTO1, GNB1, ING3, GPD2, MED21, KIAA0895L, CHP1, EML4, SNX5, CHDH,
IP6K1, RETSAT, TMOD3, ZBTB5, FUCA2, MRPL41, KPNA3, HSDL2, PLEKHH1, ZBED5, C8orf33, CYP2R1, TORIA, PDCD6,
C19orf53, PBXIP1, KIF26A, PORCN, NSMCE4A, UBL4A, RTCA, GFAP, RNASE4, MADD, PSMC6, BCL9L, STT3B, SH3BP2,
CLNS1A, HARS1, SMPD4, SESN2, TRPM5, ARHGAP26, XPO7, HSPE1-MOB4, SLC5A11, FBXO30, EEF1G, KDELR1, ASFIA,
C5orf22, IGF2R, ZNF341, PIK3CB, LRRCC1, PCMTD1, SMARCB1, FAM83D, FBRS, MRE11, TCTN2, TWNK, UBR3, ELK3, TPP2,
RIOK1, NT5C3B, NEO1, ZNF628, ZC3H18, ZNF219, LRRN4, FAM184A, MANBAL, ANXA4, CBX8, REXIBD, RAD52, SLC6A15,
C5orf51, NFKBID, ZDHHC3, SLC7A13, TNS3, CYFIP1, MAPK8, DACT1, TSKU, SBF2, VGLL3, PUDP, ILKAP, ECM1, ANXA7,
FCAR, ZFC3H1, NPR2, STAT2, DESI2, FAM210A, DERA, CCT5, UBE2Z, SERPINB6, BTG2, MFAP3L, EVI5, SNX6, BCR, SAXO1,
NSG1, PSMG3, RETREG1, TMEM51, WTIP, KIAA1143, NCBP2, HIVEP3, LRRC49, GOLGA1, HCFC1, RFWD3, RFXANK, BEX4,
SAFB2, GLTP, TAF12, SPTY2D1, SLC35A5, DCK, KRT83, PURA, TPGS2, TP53RK, FTCD, DISP2, FTO, CIAO2B, MAPK1IP1L,
SPATS2, MAPK7, HDAC4, TLR2, HASPIN, ZRANB1, ADK, ANO3, MISP, GRSF1, TBL1XR1, GBP1, ERI1, ZNF445, ASB1, SNX16,
CTTNBP2NL, REV3L, TNFSF12-TNFSF13, TMCO3, KDM5C, SYT17, VAPB, GALNT18, TAGLN, GPSM1, HROB, POLR1A,
SURF4, TMBIM1, LAMC2, DAP, GPATCH3, CCS, HPF1, BRD9, STX8, ATP6VOB, LLGLI, CHRNA5, ZNF451, KIAA1671,
MPV17L2, GIGYF2, SOS2, PHF13, PHOSPHO1, ZNF813, IFNGR1, PIGU, AKAP7, BZW2, TRAF7, SMIM19, LY6E, APBB3,
SUMF1, GSTM4, CCT6B, LRRC25, CYB5R1, SSR4, CCNEI, HOMER1, WDYHVI, ASL, MSANTD2, SPTSSB, PCDH9, IGFIR,
MON1B, ARHGAP5, SLC25A12, NDFIP2, RMND5A, MANEA, F2RL2, LEPR, YKT6, FCGRT, RAB3GAP1, NENF, SCFD1, ACSF3,
HSBP1L1, TMTC3, CASC3, ANGEL1, ZFYVE27, SMAD1, SCAPER, RAB33B, CENPF, GMNN, FAM160A1, NEDD4, CKAP4,
SMC1A, CYTH3, NOX4, BTN2A1, HSPB11, TEX10, FAM91A1, PA2G4, MYLIP, DOCK9, PSMC3IP, FBXO15, TNNI3, RPL15,
TNFRSF10A, BAHD1, BABAM2, FASTKD2, TRIM35, NLRX1, CD99L2, NCAM1, KLHL3, BMPR2, DEDD, ISCA2, ATXN1, ADI1,
ZNF706, ETAA1, CDK5R1, AKAP1, GCA, USP4, MRM2, AK1, TMTC2, LYSMD4, H1-10, ADCY10, PLEKHG4, AGFG1, CDC40,
KCNN1, CTPS2, AKAP12, EXOC5, MND1, MIGA2, NDUFS8, DGKE, ZNF277, TMX2, CCDC86, EFCAB2, SLC9A7, PALB2,
KLHL17, ADCY3, CAD, NTHL1, SMG7, DHX38, MITD1, TSPO, DUSP11, CTCF, ITPRIP, ATG13, CHST10, MTM1, C4orf48,
MRPL38, UGGT1, BDKRB1, CALCOCO2, PTPN12, PGBD1, ERCC4, F2, INO80C, TMEM160, ZNF664, PAFAH1B2, C12orf29,
CUTA, PWWP2B, GLMP, ZNF143, TARS2, GARNL3, XPR1, BMT2, GALT, DCLK2, CCDC112, IMPACT, PITPNC1, GIGYF1,
PID1, LDLRAP1, AIMP1, NDRG2, RBBP5, FGFR1OP2, TTLL11, AMPD2, ENTPD6, INTS7, MFNG, RPL39L, C1GALT1C1,
CCNG2, RCL1, ERCC8, HABP2, DNMT3B, MAPRE2, C17orf113, CLIC5, FER, CCDC102A, TAF4, ZNF81, SMIM1, ALAS1, LNX2,
GASK1B, RBM18, GNPTAB, GSPT1, UFM1, ZMAT1, RNF217, MRRF, DCUN1D1, C4orf33, PRR5L, NOC4L, TMEM243, NGDN,
ST6GAL1, MPP4, MAPK11, TCERG1, DUS2, ZNF83, MRPL55, ANAPC4, SLC13A3, ACADS, KIAA1958, MAST2, FBXL12,
TAF9B, BTBD2, ACP2, GFOD2, HPS4, LHPP, HINT3, NDUFAF1, LIMS1, DHRS13, RNASE10, CTNNBIP1, CDK5RAP1,
CYB561D2, CLSTN3, LAYN, BCL6B, EMC7, APEH, IFT20, CERCAM, DLG4, MTMR1, ATP6VOD1, ZFPL1, HIP1, GLIPR2, RDM1,
ZNF576, KMT5A, XAB2, ACACB, EMB, KDM2B, KIF12, GNPDA2, MYDGF, CDC42EP4, TINF2, MBD2, RANGRF, OAS3,
KCNN3, RPL22L1, UBE2T, CIAPIN1, CCNDBP1, ADCY9, SLC1A3, LSM14B, RAB20, DDX55, MAGI3, ZNHIT6, TXNL4A,
NISCH, USP39, EXOC4, VAMP5, TMEM50B, PSMD5, EPS8, TWISTNB, AMER1, ARMC10, KDM3A, SFI1, CDH2, GEMIN7,
FARS2, IPO4, ZNF415, ZSWIM8, FKBP11, PRKAR1B, SPRY4, NDUFAF5, SLC3A2, GAMT, ZNF695, PIK3CA, WDR5B, ABCB10,
NIM1K, C1orf109, HACL1, TMEM245, CCNA2, NHS, TBC1D4, CFAP44, DHX8, MBOAT7, TMEM38B, EAF2, QSER1, HIKESHI,
PNPO, RBPMS, EFNB1, NCOR1, IFT57, MX1, KIFC2, EIF2A, PWWP2A, MEX3C, UBE3B, ZFP62, PKDCC, OLA1, TEX30, MAP1A,
PNPLA6, B9D2, LSM8, SMG9, IGF2BP2, DIP2A, DRAM2, AGTPBP1, GDAP2, OMA1, MSI2, ENO2, DNAJB4, MIEF1, MAN2C1,
NRTN, ERCC5, SATB1, HAPLN3, SCAND1, IL18RAP, CITED2, COA6, AMH, ATG5, BLOC1S1, IGDCC4, POLE2, INTS8, GDF11,
TRIP4, MLF1, TXNDC11, CDK16, CRYBG1, PHACTR1, POLDIP2, EFCAB6, ATP2B4, MTMR14, INPPL1, NPY1R, DFFA, NIF3L1,
AHSA1, HEXD, B3GALNT2, ERLIN1, SMAD3, CELF1, PCIF1, TCN2, MBD3, SHF, ACOT8, ARHGAP31, DVL2, PDSS2, NVL,
NRAS, CUL1, NRF1, GNPAT, SPNS2, RASGEF1B, USP46, DHX16, CPT2, PPPIR12B, ARMCX4, ATG9A, C1orf74, SPA17, PIGX,
DCAKD, LMBRD2, ZXDC, TMEM141, IGBP1, CNP, EIF4G1, CELSR3, CASP2, DPYSL2, KIAA1324, MRPL21, SCG3, STK38,
DRG1, CYBC1, EXOC7, ELOVL4, THBS3, ABCB7, GPR155, TUSC3, PDGFC, CDKL5, NAGK, SLC35C2, SNX7, SAR1B, GTF2H3,
E2F8, RNF146, ATP8B3, ABCD4, ZNF18, CENPP, PUF60, CYB5A, F8, HAUS2, DYNLL2, DAAM2, SYDE1, ZBED1, ADD3,
SCN8A, EFNB2, SNX33, RDH10, PPP4C, GLYR1, MRPL44, LENG8, NUMB, PPARD, RITA1, MVK, SPATA2L, TMEM184C,
GPRIN1, LFNG, MAST1, TMEM115, ZNF830, VAPA, ENOSF1, ILF3, MAB21L3, UBE4A, CAMLG, PLOD3, LRATD2, MEX3A,
LMF2, ARHGAP32, TRIM39, ECI2, TMEM185B, GBF1, PCBD2, PPP1R37, RO60, C8orf76, RPL32, TMEM18, PQBP1, FAM168B,
TIGAR, IQUB, FOXC2, CHCHD10, EMC1, DYNLRB2, ORMDL3, CACNA1B, CYP39A1, WDR33, PNRC2, CCDC90B, SYAP1,
TEX101, DPH1, GPSM3, AKIP1, PPIC, DLST, TDG, ARHGEF26, TAB2, MRPL18, WASHC5, CDKN3, CNNM2, SEC23B, NUP85,
CARM1L2, COX14, KCTD15, IFRD2, LIN37, RECQL5, GPR85, TRIM3, MACO1, VAV2, MEIS1, RHOG, RPP30, PDGFA,
TMEM147, RNF19B, DNAJC16, TMEM208, PTPN11, EEF1AKNMT, BBX, UBE2E3, TIGD6, OSTF1, CMPK1, GOSR1, MDP1,
UBAP1, MYLK, MORC4, JCAD, ZNF707, MRPS31, NNT, STK4, SNX24, DENND1A, CLCN2, WDR24, SPATA5L1, ITGA5, USP25,
ANKRD23, ZNF512, RPLP2, CBL, TENM3, MICALL1, FIS1, UTP23, MRPS7, TWF1, E2F1, ATPAF2, CCDC181, CBFA2T2,
ZNF564, POLD1, TM9SF4, TIMM13, FAM171A1, IFTAP, PPP1R8, FGFR1, TECPR1, STPG4, ARL10, ZKSCAN1, ATP1B1, PAMR1,
ORAI3, TNPO2, PTCD2, MTBP, MYO1D, CCDC50, DVL3, XXYLT1, CCDC51, ICAM1, CNIH1, ZDHHC13, NQO2, SOX15,
SLC7A1, VWA1, IL6, PLOD2, KBTBD4, DCAF8, MYOF, CELSR2, HOXB7, F1IR, CASKIN2, MMP13, ZNF133, TLE1, RBM45,
C9orf78, NUDT16L1, TEX2, RABEPK, ATM, ATP6V1G1, EXOSC10, PIK3C2B, PTX3, THUMPD1, FAM149B1, FOXRED2,
ZNF786, VEZT, P2RX4, ZNF34, TMEM138, IBTK, CDC7, FBXW4, BMF, VKORC1, MAFK, PBRM1, SLIT2, IL15, TBC1D12,
BAG4, MPI, ERC1, TAF3, NUDCD1, YPEL5, BACE1, ARPP19, TAF1B, TLN1, SFTPB, FAP, OTULIN, TXNRD3, BCL2, CNOT6,
KDM1A, LIN7C, ENPP1, TRANK1, CREB3, AK8, ALS2CL, GPR176, ASPM, CHORDC1, ZNF280C, CHST11, EPHA4, PLD1,
USP35, LPXN, MED31, USP33, PTPRH, THOC2, UBAC2, HUS1, RABGAP1L, HELQ, ATG4A, CNOT3, VAT1, LEMD3, SLC35B3,
FBXO46, TRO, PMM1, YTHDF3, DNASE1L1, TMEM200A, TMEM102, TAZ, CARM1, SRA1, FBXO6, TAB1, ARL13B, ZFP82,
TALDO1, PEX5, SKAP2, TRIM14, GXYLT2, CLTB, KHDC4, PTS, DLX5, COL4A3, STAT4, ERCC2, MFSD1, LRRC8E, H1-3,
PLCB4, FOXRED1, SLC26A2, MNS1, AVIL, ECHDC3, RRP15, AP1AR, PPP1R12C, IFNLR1, CHERP, PIK3R1, BTG1, AP1S3,
ARIH1, MOB3A, UHRF1BP1, THAP3, MFGE8, ULK4, HS6ST2, GDE1, EDEM2, TRAK1, DDX31, CPT1B, SLC28A1, SC5D,
TMEM267, CRTAP, PIP5K1B, SPRTN, CFAP20, PKIG, YPEL2, ZNF35, DUSP14, ZNF692, JOSD2, LIG4, ING4, DDAH1, FTSJ1,
WDTC1, PISD, PTCD3, TMCC2, CLPTM1, CENPU, BICD1, DNAJC21, NR2F1, NAMPT, SLC46A3, PTGFRN, GALNT12, NRM,
JADE2, EVC2, THAP6, IRAK1BP1, PLXND1, GALK1, SACM1L, MED7, EGR4, SLC43A3, SH3BP5L, CDC37, ZNF189, MAP2K7,
SLC22A5, XRCC1, DAZAP1, RASSF2, CDAN1, RMDN1, LEO1, MAGED1, WRNIP1, SECISBP2, NDUFAF8, FRS3, RFC1,
SERINC2, SLC35A4, GREB1L, TLN2, TM2D1, RPL19, ETV6, RND1, NME6, TMEM181, CEP41, ZFAND2A, NOD1, TBC1D19,
BNIP3, CDK7, MYO6, METTL17, ZGLP1, TEP1, SLC4A7, PDIA5, TBRG4, BFAR, DEPP1, TMEM186, LRWD1, CCDC8, HAGH,
CDCA4, NEDD1, GNB2, PODXL2, MPG, ATP2A1, GAB2, NGF, POLR2I, RETREG2, WDR62, RBBP9, FAM234B, SHFL, PRRC2B,
RARS2, BORCS6, NCDN, PARVA, VAV3, RPS23, APLF, MCM7, INTS9, MYO19, OSBPL7, USP31, FBXO48, ZNF559, EXOC3L2,
ADORA2A, C14orf28, GORASP2, STBD1, NET1, ELP4, MTARC2, LNPK, DDIAS, SLC17A3, CDSN, NOP14, METTL14, CZIB,
YIF1B, ATP6AP1L, PYGO2, GPSM2, PDP2, EBAG9, SREBF2, R3HDM4, ELF1, PPP3R1, C18orf32, PTGER4, SHISA5, BEX2,
RHBDF1, PRXL2A, CNOT10, SVOP, MAEA, ZNF319, UBASH3B, IPO13, LANCL2, BRAF, SCAI, RANBP17, SLC16A1, HMGN4,
HMBS, SMIM14, ATP13A2, PGS1, PMF1, IFT122, LTBP4, ADPGK, WDR35, UBXN11, RNF20, TRMT2A, YAP1, AGER, LARP1B,
CNNM3, RAB8B, SSU72, BID, HTR2A, WDR90, PLEKHG4B, C2orf69, SLTM, FAIM, MVP, GAA, METTL6, VARS2, ACSL3,
NEXMIF, TMEM30A, UBTD2, TMEM179B, FAM120B, GNPDA1, VDR, RPL10A, TEDC1, TCF19, EDEM3, ADAMTS3,
ARHGEF11, PRKG1, L2HGDH, DCLRE1A, ODR4, ZNF280B, PRKCA, COL6A2, ELMOD2, PRKRIP1, SMARCD1, EPHB2,
NDUFAF4, NXPH4, MTFR1L, GNL2, ZNF677, SMARCD3, SLC25A39, NDUFAF2, POLI, PRMT6, USF3, SLC6A8, OLFML2B,
SLC24A2, SRSF9, TBC1D8, PUM2, TMEM131L, DTWD2, LPCAT1, DNLZ, MRPS18A, CC2D2A, C1D, RP2, NCEH1, NKX3-1,
CUL7, DGCR2, SYNM, ZNF32, C1orf122, DOCK1, PIPOX, NCAPD3, C1orf112, PAIP1, RNF7, H2BC21, TEX261, TTC30A, TUSC2,
FAM104B, RND2, FMNL3, ORC4, ZSWIM5, NIPSNAP1, PARD3B, PPP1R11, PPP1R9A, SOCS4, SUPT5H, PKMYT1, KDM5A,
LMNB2, MICAL1, TRAPPC11, ACOX1, ZBTB3, PRIMPOL, CNTRL, C11orf95, CLN8, ADCK5, SHQ1, SRRT, SSBP4, ZYG11B,
ATP8A1, KANK2, SP1, LINS1, PPP1R13B, TBCB, CLYBL, RTL6, GFOD1, AMN, ALDOC, FAM76B, EPS15L1, SLC30A3, RAB23,
PDHA2, ZBTB25, HAUS7, SLC16A9, ZFYVE28, MBD6, MYOM3, PCSK7, ANAPC2, ORC2, CCDC191, WNT5A, RBM48, LRRC32,
BTBD10, DROSHA, IFT43, NUBP2, CNOT6L, ACAD11, ZNF174, CCDC113, C6orf89, TLL1, AP4E1, TMEM260, PHB2, ELL,
SCRIB, TGFB3, ZNF414, PLA1A, FAM3A, ABCE1, C12orf66, PMFBP1, RNF123, BAD, ADAMTS13, ARIH2, KCTD12, ZNF606,
DDX19B, CACHD1, TMEM129, DNAJC25, ALG13, ELP5, LIMA1, ARPC1B, PRR14, QPCTL, LCMT1, ZNF783, VPS25, SLC2A10,
BTBD11, CCDC28A, ICE2, SEC23IP, TUBB6, THAP7, GNA12, SUV39H2, WBP4, KIF24, UBTF, APOL5, THEM6, CLDN10,
ARFGAP1, E2F7, PGAP3, KIAA0100, GPR19, RUNX1T1, HCCS, TRMT5, DGKA, NIT2, TRAPPC13, MPND, PELI2, ZNF84, PAX6,
F2R, SYCE3, UPRT, RIOK3, FDFT1, USP30, KBTBD8, KIF20B, DPYSL5, DBNL, ALG6, PAK4, NKAIN1, THNSL1, RASL11B,
CLIP3, GDPD1, LRRC39, ITGB6, ARRB2, CD300LG, DHDDS, ALG3, ELOVL2, FAM160A2, CCDC30, KHNYN, ZNF385A,
C8orf37, IFT46, TRIM25, COL21A1, CYP1A1, SLC39A11, CDKN2D, TRPC1, DPH7, ALDH8A1, CPEB3, GLI3, EMP2, WDR18,
DNAH5, TIAM2, TMEM184B, TIMP3, BRD3, MTMR2, TRABD, ZDHHC5, GTF2F2, TEPSIN, PEAR1, GRASP, E2F5, ZC3H6,
CENPT, GGNBP2, NT5DC3, TMEM161A, ATG7, MCIDAS, CNR1, BOD1L1, MED19, MRPS27, NIBAN2, CLGN, PIP4K2C,
FAM13A, ZNF584, MOCS2, C2orf74, DNAL4, ZNF430, CRABP1, ECSIT, TRIM2, ORC6, ITPR2, KIF25, EHMT2, MATN2,
TMEM132A, TIMM8B, GJB3, PDS5B, SPRED3, SMG6, SMARCAL1, GNB5, MYBL2, NUP50, SPATA20, NIP7, STIL, GPATCH11,
ZGRF1, KIF18A, NSMCE2, KIF5B, PRPS2, MAP3K21, MAPK14, MGAT5, TBL1X, PUS7L, TRPC4AP, GPRIN3, ADGRG1, GRK3,
TESC, OBI1, CHD6, MUC20, NFE2L2, HAUS3, RAB5IF, ZBTB1, RERG, RWDD4, BPHL, RAD54L, TTLL5, IMPDH1, INF2, SACS,
DLL3, PARD6A, CROCC, SMPD2, CDC42EP1, CD101, ATP11B, INTS13, NOP9, TYK2, CDC42EP2, TPBG, ZMAT5, URM1,
PSME3, IPP, ZNF322, AXL, TRMT61B, LMX1B, CLEC16A, FERMT2, PCOLCE2, PRPS1L1, GJA1, FMNL1, SLC7A10, ALDH6A1,
OBSL1, BCL2L1, ABRAXAS2, LRRK2, SPEG, XYLT1, HNF4G, PPP1CC, STAG1, MCCC1, THYN1, MPDZ, PPWD1, PLCL2,
FAM204A, UBE2E1, EHD4, SLC6A9, IL17RC, PIMREG, C16orf87, SCX, TSPAN1, ARMC1, ABCF3, DYRK3, GPHN, PIK3C3, LCT,
NECTIN2, HSPA4L, GTPBP1, CD160, TMEM184A, DDX3X, TIMM8A, IKBKB, RGCC, BCL7A, FOXK1, KBTBD6, MRPS30,
SH3GLB2, HCN1, DCLRE1C, SLFN5, PPL, CHST12, CIAO1, SNX8, CA11, ST3GAL4, IL12A, DHRS9, BBS10, SHANK3, SNX27,
CCDC43, ROR1, UPF3B, CACNA1H, MYH15, POGLUT1, GATB, HDHD2, ERMP1, NARS2, FLVCR1, PLPP1, LPAR1, NPLOC4,
RPAIN, CRY1, SART1, SLC35E2B, VPS50, CNEP1R1, MGLL, PLOD1, VPS53, ZNF234, FOXP4, FMR1, ZNF197, KRTCAP3,
NUP35, UBE2Q2, ST6GALNAC2, BOLA3, MAN2A2, MTRF1, RABEP1, RPS6KA5, TAB3, ZNF43, SEC13, INTS2, NRG1,
SLC25A11, MSLN, OSBPL8, PIP4K2B, CXCL16, CDKL1, DCAF17, FAM168A, NUMBL, HS2ST1, ANKS6, KIF6, TP53I13, ZNF784,
SNX9, NIT1, TYMP, DMAC2L, MAJIN, TRUB1, HEATR5B, STX3, HERPUD2, TGOLN2, OCIAD1, COL7A1, FUT1, ABHD16A,
VARS1, FAM200A, GUCD1, AAR2, MARVELD2, PDE3A, FAM210B, HSP90B1, ATG3, CYTH1, DLL1, TBC1D25, EIF5B, TUBG2,
SART3, A4GALT, ZNF132, SLC37A1, SCLT1, ISOC2, ACTR10, NETO1, NDRG4, SLC20A2, SIN3A, DNAH3, INTS12, POLDIP3,
APAF1, CORO1C, RAP1GDS1, HYAL2, RNF223, DMPK, ANKS3, ZNF134, ARPC5L, IMP3, LONRF1, CCNC, FAH, SPIN1, SFR1,
BRINP1, CHTF18, AP5Z1, ENG, NUP62CL, RPTOR, BLOC1S2, COQ3, DNAJB1, SLC48A1, TSC2, PHC3, TMEM218, MRPS26,
ESD, ALG12, BEND3, SOX13, PRSS16, UPK3B, RFX3, EMX2, SEMA3A, SLAIN2, TSNARE1, TWF2, ISM1, EFCAB7, SQLE,
ATF5, GNAI1, MAPRE3, H4-16, IL9R, ATP11A, PGAP4, ACTN2, WASL, EXTL2, PRKD1, CIDEB, NSRP1, PAFAH1B3, STIM2,
HELB, PHF5A, VAMP4, KCNAB2, MRPL4, TRIM28, IKZF5, LHX2, MYO5C, SNRNP70, PNISR, PPMIH, PDXDCI, SASS6,
CXorf38, GLOD4, RNF32, TSSK6, DZIP1L, PPP1R35, CCDC28B, SIRT7, CLSTN1, ROCK1, GABARAP, ARRDC4, SMYD2,
SCN2B, ZFP92, IGFLR1, DAAM1, ELL3, MFN1, RFX6, SLC30A1, TEAD3, BACE2, KIAA0753, ADTRP, PEG10, EPB41L4A,
AFAP1, SUGP2, SERPINA6, CASKIN1, CNKSR1, LSG1, SMARCAD1, NKIRAS2, ACSS2, BDNF, DNAJC22, HSD11B1L, DDAH2,
PRKAR2B, UBE2G1, LRCH3, CSNK1A1, LTBP1, METAP1, ZNF618, WDR34, RPAP1, TUBB4A, TMLHE, FANCM, TFCP2L1,
NPDC1, MED24, MRS2, TTC21B, CDC45, EPOP, CRTC1, RBM25, DDX56, IL17RD, TRIM72, CCDC61, LONP2, THOC5, GATAD1,
EVA1C, PHF11, C1orf174, PROCR, SLC3A1, P4HA1, RAB22A, PICK1, ANKRD39, SCD5, EML1, JADE3, ADPRH, DNASE2,
CEP128, TSNAX, PRDM4, EXOC8, ZCCHC3, ZNF446, TMC7, ETV4, ZNF146, PEX14, RAPGEF3, TMEM128, SNAP25, ZNF326,
HEBP1, FGFR4, DOLK, ZNF546, GPR68, PDIA4, SHC1, RELT, RMDN3, FGD4, SEMA3C, C11orf1, GNL3L, THADA, ZIC5,
ANXA6, TSTA3, CEP78, FBXL20, CAMK2N2, PGAM5, EVC, MTOR, MVB12B, CDK9, OGA, RAB2A, RALA, TTC14, STK25,
CD274, TMEM39A, LRIF1, TMEM101, CPEB2, MEGF9, KAT6B, SENP6, SLC9A8, EXOC2, ARF5, MEF2B, NR1H2, AP5M1,
ALKBH6, RBM4B, OPLAH, OGFOD3, PTP4A2, RPL8, SRRD, SMARCD2, INSR, TBP, BAIAP2L2, NPRL2, ZNF551, DKK1,
HEBP2, VPS13D, CCDC88A, IGSF9B, NSMCE3, CCR7, ANKRA2, CYP2U1, NECAB3, C2CD4C, DBF4B, FBXO8, PNKD,
SLC35F6, ADRM1, CCNK, LAMA3, TBC1D32, FKBP14, SMC3, MRM1, IBA57, NRBP2, CPTP, BANP, ZNF766, FLII, PCBD1,
OCEL1, SELENOI, SLC22A15, BET1L, ARHGEF19, CHST15, SEC31A, DPF1, ALG1, MRPL54, SLC26A7, WASF3, DNTTIP1,
CLSTN2, TSPAN9, NR1H4, ARL2BP, RAB4A, ZNF679, DPP7, FPGS, ILRUN, B4GALT4, EFCAB14, REXO2, SOX12, CDCA8,
KLHL8, UFSP1, SNAPC2, LIMK2, ANKRD12, NHEJ1, ZNF567, MAP6D1, NOC3L, GRHL1, GRIK2, KCNE4, LGR5, ERBB3,
FLYWCH1, IDUA, STRN3, UBE2H, ALKBH8, ARHGAP24, MRFAP1, BTBD6, PPP2R5B, KYAT3, TDP1, ARFIP1, HARBI1,
FBXO31, SNX30, DTNBP1, ELMO2, MEGF11, NAV1, PHF1, GOLGA5, ADGRE5, EML6, PPM1A, TMEM198, HEYL, ITFG2,
ARNT, OSBPL11, NXF3, UBE2M, C15orf41, SEMA3F, DGKQ, GABARAPL1, ERGIC1, EIF5, EXOC6B, UNC79, CXXC5, UXT,
TMEM159, ARL6IP4, PAXX, TTL, COX18, FAM169A, RHBDL2, CHTF8, DNAJB12, C19orf54, RAD18, UBA52, ERCC3, GSTA4,
PHF8, ZNF573, HSPA9, ZBTB6, MYPOP, STAB2, CNBD2, KIAA0895, DNAJB14, QPRT, MORC2, C19orf33, MAPK9, ZCCHC10,
CCDC171, MSRB3, FLNB, METTL8, SRFBP1, CABIN1, TSPAN8, ZNF354A, TET1, WFDC3, COG5, CROT, EBF1, TSEN54, NIPA2,
HELLS, CIART, MKNK2, TMEM139, NABP2, TOR3A, DPM2, ANKIB1, ERAL1, CAMKMT, ABCA5, PCED1A, ANKLE1, ZNF76,
GLT8D1, MECOM, FAM53B, FUZ, INSM2, POLG2, AP3B2, NFXL1, ZNF658, ADCY7, CUL4B, SMIM12, TLCD5, NME3, ZBTB46,
STON2, LAMB2, ETHE1, NRP1, ACBD5, CUX1, KIAA0586, TKFC, DHTKD1, TCF12, ZDHHC18, TMEM45A, ITGA2, ARHGAP45,
ADO, CCSER1, CTC1, TMC5, CANT1, IL1RAP, ANO8, OSGEP, HNRNPLL, U2AF2, CEBPA, AHNAK, MAPKAPK5, ELP6,
TNKS1BP1, SMAD2, PEAK1, DCAF4, LRRTM2, NR2C1, TSPAN19, ZFP64, MYO9A, ATPAF1, MPHOSPH9, STK11, LRCH2,
PTK2, GPR3, SHE, LGI2, SLC16A5, UCP2, SLC6A17, SNX11, NFE2L3, SLC35A3, TRPM3, PDLIM5, UNG, CDKAL1, NLRC5,
RAD17, CCDC57, ZNF572, AKAP8, KALRN, RNF130, CAMKK1, FSD1L, NLK, P3H1, POLR1C, PADI1, EDA2R, FUNDC2,
PAQR8, PPP3CC, RAB14, KIAA2013, C4orf19, ACTL8, KIAA1217, PTPRS, YJU2, TCOF1, ZBED3, PRR11, ANKDD1A, BARD1,
COG6, TFEB, ZNF160, ZNF684, MNX1, RPUSD1, EFCAB3, ZNRF2, VPS9D1, HS1BP3, RAPH1, SUOX, IMMP1L, SPAG4,
CEBPZOS, DEPDC1B, INPP5A, PRPSAP1, GOLM1, WDR44, WFS1, PSRC1, PCM1, PIK3IP1, TXNDC5, ACBD3, AKR1E2, JAM3,
LRRC8D, BACH2, SIAH1, PPFIA3, DCPS, SORBS3, ZNF644, KHDRBS3, TUBGCP4, NSUN7, FBH1, ZNF284, PATJ, DESI1,
DEDD2, NRBF2, MAP7D3, TUT4, ATP23, PPP1R21, CDC25B, SMC4, IRF5, ABCC10, USP13, ID2, ABCC4, WDR1, DNAJC12,
FHAD1, LIMD2, IQSEC1, TMEM120B, C12orf45, CACNG7, ACSS3, SSX2IP, C3, FANCL, CCDC85C, ESCO1, PNPLA7, PPP1R12A,
SLC44A1, TCF7L1, UFL1, PAX8, UBQLN2, VPS41, TRIOBP, PIH1D2, MFAP3, TP53BP1, TOGARAM2, RNF114, LCOR, TFAP2C,
SIRT5, TTLL7, ADIPOR1, INTS1, UPF2, SIPR2, SYNRG, CTBP1, LOXL3, SREBF1, NELFB, ARID4A, C1orf216, POMT2, HOXA1,
GLRX, MAGEH1, KHK, ETV1, FAF2, KIF5C, PDHX, AAMDC, OARD1, METTL15, FAM118B, TMEM199, AMBP, ARID3A,
FAM171A2, BCL7B, ZNF26, ACLY, ARMCX3, MCMBP, SH3KBP1, ARL6, MELTF, LYST, PRELID2, ZNF471, PFKFB1, SLC17A9,
RRM2B, TATDN1, CTDP1, OLFM4, SUCO, ZDHHC21, ATP13A4, CMTM4, DDC, FKBP7, SFXN5, POLR3F, MAP4K5, PDF,
GCDH, KIAA0930, TMEM242, LYRM7, ATXN7L3B, FAF1, TEDC2, AIFM2, EEA1, RPP21, WDR73, ZFPM1, ZMIZ1, BLNK,
ZC4H2, ZNF641, UBE2G2, FLACC1, STARD8, RAE1, PURB, RAB11FIP1, MEAF6, MLLT1, BORA, H2BC11, NUDCD2, CTDSPL2,
DPF2, B4GALT2, LSS, TRPM6, TAF7, GNG12, CAST, GEN1, PDXK, ARFGEF2, NAT9, PRELP, FOXO4, TMEM44, UBXN6,
NME4, STRIP1, CHCHD5, ITPR1, SPAST, POC1B, STX10, SUPT7L, UNC5A, CYP3A4, PRTG, MIS18BP1, LRRC6, RASGEF1A,
C2orf49, LHFPL2, NBEAL1, SRSF6, CEP104, DNALI1, LXN, CCDC93, WNK1, AP3S1, RAB35, C7orf25, WDFY1, INSIG2, EXOC3,
B4GALNT3, RCCD1, GAN, MTCH1, SEMA4F, USP16, NEURL2, RAB9B, MSANTD3-TMEFF1, PLEKHG5, PIK3C2A, PRRG1,
PABPC1L, CENPL, AGMAT, PSMB9, CEL, OSBPL1A, IQCD, C1orf53, PIGZ, PHKB, WDR55, ATP1B2, ATG4D, SYNGR1, YDJC,
FMO2, SHOX2, SEPTIN10, MEGF10, RFTN2, LEF1, TRMT13, NAGPA, AP1G2, DNA2, ADAMTS4, XPNPEP1, ZDHHC7, SNX19,
NOVA1, FBXO7, HNRNPH1, NAA80, TNFAIP2, ABHD11, GALNT11, EXOC3L4, TULP3, ZMYND19, STPG1, DTWD1, SLC37A4,
RAI14, ZCCHC12, TM4SF1, ENGASE, CREBL2, USP48, IL10RB, SCML2, ZNF586, DHX57, TIMM10, BNIP2, CADM4, EXOSC7,
LONRF3, C6orf120, RIPOR2, SDR42E1, AMZ1, LMCD1, ASAP3, AGPAT3, DNASE1, APLP1, FN3K, F2RL1, GLCCI1, ZNF226,
RASA1, ARAP3, EIF5A2, TFDP1, SLC39A9, SLC6A16, YPEL1, SNX15, SYNJ2BP, GEMIN5, SIL1, MYADM, PROB1, ASIC1,
FKTN, SYTL1, COL20A1, CDS2, LAT2, PSEN2, ENKD1, RAP2C, KBTBD3, FBXO39, EXD2, NEMP2, CDK14, ANXA10, APH1A,
PPARA, ZNF512B, KCNQ1, FGF7, ADAMTS15, ARMH3, GPC3, THAP10, CACNA1D, KLHL5, THSD1, FBXL14, CMIP, RAD51B,
CBFB, NUDT14, ZSCAN30, ZNF613, SYPL1, DALRD3, RCAN3, CCNF, FBXO33, GOLGB1, UBTD1, IKBKG, WWC1, FZD6,
LRRC4B, GRAMD1C, PHIP, TENM4, KCTD13, DNMT3A, GRK4, PPP1R16A, FGFR3, UMAD1, ADAMTS2, PYGB, MYBL1,
HOXA5, WDR17, CIQTNF2, FOXJ2, NEB, FAM83B, SAPCD2, ZC2HC1A, GGACT, CAPN7, FASTKD3, PITPNM1, YARS2,
UTP25, ARFIP2, CORT, ELK4, IL1R2, APBA2, GRIPAP1, OTX1, ICAM5, TNNT1, RASGRP2, ZEB1, TREM1, CDIP1, STN1,
ZNF273, ELOVL5, CABCOCO1, TMEM107, ARL16, CREB3L2, FDX1, TBL3, CLDN1, PHF3, OSR2, TOP1, PDLIM4, SUSD6,
ALPK2, B4GALT1, LARP4B, SRSF5, DIXDC1, PFDN4, DUSP28, COA7, NFIC, PIGP, ACVR1C, FZD7, INCA1, CLP1, TMEM150A,
TCP11L1, MBNL3, SPEF2, MKNK1, MROH1, SCEL, GUF1, GRIN3B, RFTN1, SPRYD4, TTC27, SPEF1, FAM241A, AARS2,
FBXO25, ZBTB47, ZNF346, ACBD6, TNK2, DNMT1, HMGCS1, SLC43A2, DHCR24, C1orf116, STYX, SHISA2, PLXDC2,
HSPA12A, SLC25A20, BCAS1, RECK, MTTP, COL4A5, CORO2B, KDM8, PPP2R1B, RIOK2, KLF2, SYT13, FAM53A, PGBD4,
ZPR1, DHX32, CASP9, RAD9B, ZNF214, SLC2A6, PITX2, LAPTM5, MAX, NR4A2, PPCDC, SLC37A2, SYNGAP1, JMY, PARG,
ABCB11, CYREN, PRSS36, VRK3, SMYD4, PAWR, PNMA1, DIS3L, TRPM1, TRIM27, TRAPPC5, TNFRSF19, RNF141, SLC4A8,
ATL2, ZNF791, MIF4GD, SLC8A2, EBF3, PSME1, GRB14, INPP5F, KDELR3, ZGPAT, LATS1, DR1, NLGN1, CTF1, RHOU, RHOV,
DCAF10, GTF3C2, ZDHHC1, MRAS, METTL4, HAVCR2, POMGNT1, RAB3GAP2, ELMOD3, ZNF232, EPM2AIP1, STX5, AMD1,
CUTC, FGFBP3, MAP3K3, PERP, CPNE7, TSHZ1, TXNDC9, CLEC12A, PCNX4, TMEM104, B3GAT1, EHBP1, CRYGS, SEC14L2,
LURAP1L, WDR59, ZNF334, LPIN2, SLC17A2, TMEM52B, ZNF432, TIMM17B, COQ9, KCNK13, SLC29A2, GPAA1, PKP4,
ADIPOQ, TMCO1, TRAPPC2B, CLPX, C10orf143, NXF1, SARM1, QKI, SCML1, SLC19A2, BAX, BCO2, B3GALT5, RP9, BLM,
SBNO1, ABCA8, ERCC1, RAPGEF5, HEATR1, RNF40, STK10, MOCS1, SLC25A19, MAFA, CHEK1, EPB41L5, THUMPD3,
MARK1, CNTNAP1, ATF7, MAN2B1, UTP4, IFT22, KLHDC2, RTKN2, DOK1, ULK1, ADPRS, INPP1, PYGO1, TBX19, RGS12,
SIKE1, MGA, GPS2, SEC24D, RAB3A, MBNL2, TRADD, CLIP1, FAM160B2, ZNF391, H2BC5, ZNRD1, PNMA2, EGFR, C1orf194,
SIDT2, GPRC5A, MAFF, SLC22A12, PRMT7, SERP1, LSM14A, MAGIX, POLR2M, GPATCH4, STX7, ZNF251, MTFP1, MTIF3,
SEMA3E, ZNF57, GINM1, AMIGO1, SLC25A13, PSMD7, HOMER2, ZDHHC4, BTBD3, PNPLA3, PTPRB, MYO1C, MAP2K5,
CLHC1, SLC2A13, SGTB, TOR1AIP1, ATOH8, RELL1, UQCC3, ZSCAN16, RPS20, IL22RA1, ANAPC7, ADM, KLC2, DNAJC13,
LGALS2, ARHGAP10, ASMTL, TERF2IP, DOLPP1, SH3RF2, PTGIS, RPS6KA2, C3orf62, COX7A1, ACCS, SCAF11, ZNF696,
HLA-DMA, TEFM, ZNF165, TAF1A, MPP5, LRP6, MXD4, ARGLU1, ZNF175, LYRM2, NOL3, PFKM, YEATS4, SLC52A2,
KCTD11, CATSPER2, PKP2, CHMP7, UNC93B1, SAP30L, CORO6, KCTD19, GID4, VWA5A, KIF26B, CDADC1, MCF2L, RESF1,
TXNL1, BRI3BP, C21orf91, MRFAP1L1, SLCO4A1, RASD1, ORAI1, EMC8, NR2C2AP, PLAU, CHM, CEP170, ODF2L, ENOPH1,
ZMAT3, MAP4K2, RHOQ, RFK, AMN1, RAPGEF4, PLPPR2, GEMIN6, CPEB4, ERAP2, HDAC6, LSM1, SIRT2, IQCE, S100A3,
LRRC8B, DNAH7, TSPAN10, CDC5L, TRMT10C, TOP1MT, FAM161A, TMEM41A, VCAM1, TRMU, CENPS, CREM, UQCRC2,
SLC41A2, TNS1, EML3, CCNH, C8G, CERS5, FAHD2A, CADM2, PHF21A, NAA25, HGD, EDEM1, KLHL7, MPLKIP, KHDC1,
SPIN3, TAF1C, ATP11C, SOCS1, SLC25A6, TAF5, COQ7, GAS2L1, CCNB3, SUV39H1, HMBOX1, KIFAP3, PLEK, RHOBTB3,
FRYL, ARHGAP40, RSPH4A, TMEM39B, FUOM, PPFIBP2, SLC41A3, POLL, CIP2A, HOXA7, STRBP, CCDC81, ORMDL1,
MKLN1, CACNB3, RIN3, EEF1AKMT4, PDE8A, MCMDC2, PDGFD, INIP, PPME1, PARVB, TLR6, ACHE, STK26, CYP51A1,
CYTIP, GM2A, KCTD7, RHBDD3, TMEM126A, PEX7, SAV1, ATPSCKMT, RBPMS2, CCDC6, KDM3B, BAP1, TNFAIP1,
CWF19L2, DDHD1, FRMD8, KMT5C, ZNF671, DCUN1D2, FAM43A, LSM2, DACH1, SLC66A3, RPL27, FAM214A, UFD1,
CAPN8, SMIM8, C14orf93, FURIN, SCYL3, BDH1, PYGL, ZNF721, TUBG1, MYO16, ZNF449, RAB27A, SULF1, C2CD5, ACSF2,
SUSD5, C8orf82, TM4SF18, MACROD1, NPC1L1, SFXN2, NECAP2, NOL10, SLC25A14, ATRN, TCAF1, TNFAIP8, LARP6,
CATSPERE, CACUL1, DDX49, CAPN3, ARSK, CCDC9, TTC8, MLST8, RRP7A, MED20, TNFRSF14, CCDC130, CYP24A1, SYNC,
WAS, EFNA2, ACER3, VASP, KRBA2, KRAS, SHC3, KRBA1, DLX1, MTERF4, SLC25A21, NAIF1, GGA1, SH2D4A, ZNF358,
PRR7, DSN1, KLHDC9, C3orf67, SLC35E1, CLINT1, C3orf14, MRPL12, HEMK1, LLGL2, ZSWIM7, AP3M2, BMP7, GMPR,
BEGAIN, EIF4EBP2, MAPT, STK32B, USH1G, GTF3A, PINLYP, HNMT, PLA2G2F, ANAPC15, SPART, SLC35F5, TBXA2R,
CMBL, LMAN2L, STAC3, ST7L, SLC35B4, PCP2, SULT2A1, TTC9C, DERL2, GTF3C3, SLC44A4, CCDC107, TDRD3, TRDMT1,
GDPD5, FYN, WWP2, TOM1, BDKRB2, SLC35E4, TSPAN7, DCLRE1B, P2RY2, CNRIP1, SPIRE2, MINDY2, JAKMIP2, SLC4A3,
BICRAL, PAPPA, SIX5, ZNF407, MYOZ3, EGLN2, PLIN4, EIF4E2, PAH, ABCA6, DOCK3, ENTPD4, VPS51, SH2B2, FERMT3,
ANKRD44, GNB1L, ITGA4, C9orf116, CD70, SYNGR2, FBXO27, ZNF566, NDC1, OXLD1, H6PD, FOXM1, MCF2, FBXO4, SAFB,
CFLAR, COX19, EBF4, UTP18, DISP1, PIR, LYRM1, RFX5, NFKBIB, MAD2L1BP, HAUS6, NINJ1, PTRHD1, TMEM25, CCNO,
IRX5, COPS7B, ARG2, TGFA, KTN1, THAP5, VEPH1, C12orf10, KIF2A, TTLL1, TBRG1, SLC26A6, CCPG1, CEP57L1, SYNGR4,
THAP8, ZBTB45, CCDC121, PEA15, SLC25A35, PLCD1, CFAP65, FAM241B, KREMEN1, WNT3, RAP2A, RUFY2, EPHX2,
RASSF9, ZNF549, CACNA1G, SEMA3B, SLBP, TMEM134, UMPS, RNF2, MRPL57, RADX, ZNF485, EYA4, SLC25A16, OSCP1,
PFKFB2, MTERF2, ZFR2, C16orf71, BCAM, ANXA2R, ECHDC2, TP53INP1, ARMC5, CD52, RAB11B, C17orf75, HEY1, PTPN18,
RPS6KL1, CEP170B, NFYC, PCDHB14, PEX6, NUDT12, NAA10, XRRA1, SIRT6, PAK3, TEX22, ABHD5, NGLY1, SLC22A18,
WDR83, CMTM8, KLF13, PLA2G12A, PRAF2, KDF1, CGREF1, HEATR6, R3HCC1L, DNTT, PARP8, DDX59, PANX2, TPRKB,
NME5, ROR2, SHANK1, ZBTB20, DPYD, TSR1, ANKRD26, CDKN2C, KIF27, STKLD1, ZNF266, CNIH3, STXBP1, PDE5A,
GPR15, SGO1, NEK6, KLC4, ATAD5, TPR, FGFR1OP, ING2, ADGRE1, CDC42SE1, HACE1, MZF1, NDST1, E2F2, OXSM, PIGO,
DTX1, HDHD3, SMPDL3A, BOLA1, CHCHD1, NUDT6, TOMM40L, CRLF1, RAB11FIP2, MGAT3, UBE2Q1, KRCC1, MTERF3,
AOC2, AASDH, BLOC1S3, CEP70, VNN2, VWCE, GREM1, C17orf98, TIGD4, CSAD, ILVBL, PCDH1, LOXL2, TRAPPC6B,
CALHM5, MTUS1, TUFM, MCTS1, IL18BP, LIPI, CCDC92, AGPAT4, GRHPR, IGSF8, MVB12A, BBOF1, CRNKL1, LMTK3,
XYLB, UBE2S, TARS3, ADCY6, PANK1, APPL1, MAP3K12, TOB1, CTXN1, SIPR3, HOGA1, SRSF12, TIGD3, XKR9, DDX6,
CHUK, ATE1, TMEM132D, PIGQ, PLAGL1, PROSER1, MRPL52, BEND6, RHNO1, ZNF792, BBS5, WIPI1, RNF13, TRAK2,
YPEL4, CHST2, SETD7, ADAR, TEKT5, NRCAM, UBE3A, SMARCC2, LGALS12, DHX40, SLC9A6, TTC23, XRN2, WDR31,
CCDC146, MICU3, DDX52, FGF12, SAMHD1, NSMF, MME, SYNE1, INPP4B, RAB42, FETUB, BOP1, FAM136A, KIF1B, ATP2C1,
ABCA7, NCF1, MDH1B, PPM1L, SLC37A3, TAF1L, RARB, GABARAPL2, ITSN1, ZC3HC1, ZNF85, RALGAPA2, BAAT, BIN1,
LIX1L, NECTIN3, MED22, TLR1, CCNJL, ST7, SPOCK2, DNAJC18, GPC1, RPL9, CEP68, RNGTT, KIAA1109, TMEM165,
CSNK1G3, CD47, BLZF1, ARHGEF10, QRSL1, TBR1, EIPR1, TMEM74, CXCR4, CEP295, PACC1, GGN, WDR70, RASD2, ROBO2,
ITGB8, RSRP1, CA5B, ZNF416, TSPOAP1, RGS17, RTRAF, CAPN1, FZD2, C12orf4, FEN1, GNRH1, KIF3A, PPIL2, TSPAN31,
TSPYL2, ICA1, RASIP1, FBXL16, SYT14, TOM1L2, WRAP73, SMCO4, PHF24, CRELD1, NTNG2, ZNF253, FAM117A, SCN3A,
COGI, SNTA1, MRPL34, CFAP43, UBE3D, PIDD1, CENPC, RNF122, THNSL2, TLCD2, IL15RA, CASD1, FYTTD1, BEST1,
SPON1, DENND3, PDZD4, SH3RF3, ARSJ, ATRNL1, RASGRP1, APPL2, ZNF518B, LARS2, SH3GL2, KCNH2, AKT1, C19orf47,
GAS2L3, LOX, RHBDD2, NRARP, FAM189B, GCHFR, NPFF, ACTA2, YAF2, ANKRD16, APOBEC3H, MINDY3, SORCS3,
BCORL1, PJA1, AK9, ACE, TSTD2, ARFGEF3, PPM1K, NUP205, SON, ATF1, RALGAPA1, RHOBTB1, SMCHD1, SRPX2, FSTL4,
MARVELD3, PCYOX1L, DHRS7, MINK1, RAPSN, SFT2D2, RBM28, SAMD13, FSIP1, DIAPH3, DYNLT3, WDR20, BTBD9,
AKT2, CPN2, ABTB1, NUP160, TMEM207, GLI4, SERPINB8, VPS16, STK31, ALOX12B, SUGP1, BEST2, FDXACB1, SLC25A44,
EFCAB11, GTF3C5, NEDD4L, RNF185, EIF2B5, NCR3LG1, DUSP19, APEX2, RBM12B, GPIHBP1, PRDX2, C4orf46, HHEX,
HDHD5, LIFR, AVEN, ELFN2, POLN, SLC18B1, SUPT3H, ERO1A, FGD3, TBL2, TTC22, ITIH5, KIAA0408, MCTP2, MTHFD1L,
HUNK, PRDM10, LRP2BP, RABAC1, EPAS1, ZNF425, LDB3, OTUD6B, ZNF653, ZFYVE16, HOXA6, SNAPC1, LCN2, ARMC9,
TAOK3, B4GALT6, MROH6, DDX3Y, ELP3, RAB3IL1, EDN2, ABLIM2, CDCA7, HCFC2, SYCP2, PHF10, CHMP6, DDX54,
FAM78B, NCALD, HOXB3, SLC66A1, MPP6, TRMT6, ADAP1, WASF2, ZNF469, TTI2, ZFHX2, CSRP2, JAZF1, SLC16A10, NR1I2,
RAB9A, MAN1C1, LMBR1, LRRC4C, PLEKHG6, MS4A8, RNF166, ACOX2, SLC25A23, SETBP1, PDSS1, SPATA7, WNT11,
LDLRAD4, MTDH, PIGA, ZRANB3, TCIRG1, ADA2, METAP1D, DPM1, YIPF6, TAP1, TOE1, LAMB4, STX18, NPTXR, ABCD3,
NADSYN1, SLC12A5, TEC, SLC44A2, SMAD4, TRIM47, STK17A, PHAX, FSBP, MYH7B, AGBL2, SCARF1, KDM1B, MAP3K6,
RAD1, WDR76, RPP40, HIC1, SLC2A3, NDUFAF6, LCP1, APBB1, BATF3, EIF1AD, VANGL1, SPON2, DISC1, TRAPPC9, TRIM38,
ARMCX1, GPLD1, EGLN3, EIF3F, TBKBP1, POLR2D, CHGA, AIM2, BTF3L4, CFAP97, DNAJC7, GTF2H5, KIF21B, POLR2C,
TMEM88B, LIMCH1, ARFGAP2, CAPS2, CDK5RAP2, ABHD3, KBTBD7, RFESD, CTPS1, GLRB, DBNI, GNL3, HOXA3,
SLC35B2, TPRA1, GSTO2, TM6SF2, SPATA6, ZNF880, CYB561, RALGPS2, LRRC56, STAG2, AKAP3, APOO, KIF5A, MFAP2,
RAD54B, THAP1, C11orf52, KCNJ11, HARS2, USP20, WDPCP, FHDC1, STAMBP, EEF1AKMT1, UMODL1, KRR1, PLAG1,
ETFRF1, CEP162, FUT8, OSMR, AZGP1, RNF182, FAM126A, POLR3K, SLCO4C1, ZFYVE21, CSNK1A1L, CYP20A1, LRRC10B,
KCNMA1, EARS2, SLC29A3, MBD4, NYAP1, CDK3, DNAJB9, PIFO, SDHAF4, TMEM86A, UBE2L6, TNRC6B, IQCB1, MOB3C,
BEST4, ANO2, MRC2, TGFBR1, DTD2, LAMTOR1, ZDHHC14, NOL4L, ALDH1B1, MID2, MYRF, PAQR9, PGGT1B, DIMT1,
FOXA3, TCF3, FAM120AOS, BFSP1, EFHC1, ARMH1, MYCL, MPV17L, ZC2HC1C, OPA3, YBEY, IFT140, C7orf31, NSUN4,
KISS1, S100A14, ME2, TMEM9, NUDT3, CAPRIN1, L1CAM, RDX, SLX4IP, TINAGL1, DNAJC27, NCOA1, MORN4, TMEM41B,
P2RX6, SNED1, ABI3, GET1, SIX4, PTPN13, FBXL2, LIPT1, GABRA2, HSD17B1, PPOX, ZFP37, ZNF345, ZNF789, SPAG9,
MTPAP, MRPL43, TMEM80, GPR160, STIM1, SLC35G1, FGF13, UBE2J1, ENTR1, LIN9, FBXO16, SDCCAG8, SH3GL1, NCBP1,
SP4, DHODH, TMEM68, B9D1, EPB41L4B, CDK2AP1, SLK, SLC10A3, MAMDC2, AKAP14, NHLRC2, PIP5K1C, ZNHIT2,
PHETA1, PCDHB2, GPR107, HOOK2, CDKN1C, INTS14, STRADB, ZNF740, EXOSC3, RASSF6, NSD3, CSRNP3, GPR180, PEX1,
TRIM21, LRRC46, ZNF662, GOT2, PINK1, TMEM183A, CHTOP, C6orf223, MMAB, ITPKA, TMEM234, TCTA, PAGR1, RAB40C,
BVES, SCFD2, NUP54, SEPTIN6, ESF1, TMEM132B, BAK1, RGS19, CCDC138, FAM50A, FAAP24, PEX13, ZNF836, EPB41L1,
SMTN, SLC35C1, PTX4, ARL4C, PIGH, ADAT2, AGT, IQCG, PASK, C12orf73, HRAS, TMEM62, RUNX2, CHAC2, BIN3, NDEL1,
TRMT10A, ACBD4, FCHO1, PUS3, WDFY2, GPC2, RAB5A, CD83, DCP2, KLC1, DEF8, WIPF2, EXO1, SEMA6A, CUL2,
ETFBKMT, NOL8, PAFAH1B1, POFUT2, STAG3, SEC22C, SURF1, SSC4D, CEPT1, RXRB, USE1, RASGEF1C, STK16, ACIN1,
TK2, C19orf44, ZNRF1, CRYGC, GCNT2, RBM20, STX2, TFCP2, BICC1, RBMX2, HTD2, CCDC186, DAG1, DMRT1, STX6,
PLEKHD1, CNOT8, ZFP30, NXPE3, CD2BP2, MTMR8, ABCB6, SEMA4G, LYNX1-SLURP2, KISS1R, ETS1, PDE12, RCBTB1,
PPM1M, LMNTD2, CAMK2G, LIG3, KIAA1328, RBM41, HDAC5, EXOG, B3GALNT1, LMO1, RYR3, SHPRH, ADAL, HTRA2,
SDC3, SPIDR, NADK, UBA7, SAP30, CCDC102B, ZDBF2, COBLL1, MDGA1, SARDH, TM4SF20, EHHADH, DTNB, PANX1,
REXO5, GLIS1, EIF2AK2, CRYL1, STK39, HES2, CLCN4, CYP2J2, GRIP1, CSGALNACT1, VPS37A, DECR2, VANGL2, N4BP2,
FCHO2, STARD3, PEF1, GAK, NMD3, SAPCD1, FREM2, SORBS1, ACP6, FANCA, KCNV1, HPS3, C9orf40, AKAP6, CEP135,
NEK11, ATP5F1D, IDI1, TRAPPC2L, ADGRA2, SMCO2, TRIB2, APOL6, OXNAD1, RAB39A, ERICH2, PLPP5, ROM1, NAT1,
PLXNA3, ADGRG2, KIAA1211L, SHANK2, WSCD1, RRAGD, SDR39U1, AGR3, FZD3, RGS10, SLC25A45, UBXN7, ADGRV1,
SELL, TMPRSS2, ZNF329, MOB1B, USP28, PDCD2, GPR108, EGLN1, TUBB2B, NUP155, TPCN1, ARHGAP28, CRISPLD2,
PCYT2, QARS1, NETO2, PMEL, SFRP4, TRNP1, CNPY4, NOL6, PRRG4, ANGEL2, KXD1, RAP1GAP2, SHROOM1, TBK1,
DIPK1A, NAT10, FAM76A, TMEM35B, TBC1D22A, RASGRP3, KIF3B, WDFY3, NPAT, PIP4P2, C1RL, FANCB, TNKS, TASP1,
AFDN, RASSF7, CIR1, NPHP3, ATF2, IQCH, NPL, CDR2L, BCL7C, RTL1, STX17, ITGA1, STING1, STOX2, ATP9A, VIPAS39,
ARHGAP9, IL6ST, ARMT1, APOC1, COA5, DNM3, PKN2, ESRP1, MFSD6, RBPJ, ABCC2, DNAJC2, EIF1AX, SLC9A5, TATDN3,
THOC7, KCTD17, METTL25, LIPC, MMP16, SFPQ, CBFA2T3, LRFN3, ACAD10, TYSND1, PLSCR3, C9orf64, ZNF593, COPRS,
MANEAL, MIPOL1, RNPEPL1, DPP8, COA8, TENT2, DLX6, MFSD11, ADAMTS5, POU2AF1, EPHB4, NFKBIL1, UROS, TMA16,
GFRA4, PHTF2, STX4, TMEM120A, ARMC8, CBLL1, LEMD2, OR2C1, MAP2K1, DPH2, SLF1, ZFAND2B, CDKL3, NAPEPLD,
C2CD3, BTG4, LRATD1, CWC22, UACA, SUPT20H, EME2, GHR, ANKRD61, MKRN1, PIAS2, ZNF23, FUT9, SEMA6C, ACTN3,
ARHGEF6, ZNF550, AGGF1, STXBP6, YIPF2, CCNE2, DISP3, TMEM187, YY1, IL6R, NMI, PGPEP1, PLTP, SLC26A4, NEIL2,
EIF4EBP3, MMP15, ACTA1, DDX28, PTBP3, PNPLA2, TG, BTN3A2, SLC22A23, EIF3D, F12, VRK2, CFAP52, BTBD8, RHOF,
CCDC65, C3orf18, IGFBPL1, SFT2D3, CCSER2, TRNAU1AP, FAM124A, FMO5, RBMS3, TMEM150B, PAPLN, HES7, ULK3,
SCMH1, ADSL, PIEZO2, AGFG2, SEC14L1, PTPRG, WNT7A, MAOA, CFAP410, PDGFRA, TMEM192, ZNF770, HOXA2, USP44,
PHKG1, STARD5, FERMT1, MBLAC1, THRB, LPAR3, N4BP2L2, RCOR2, RAB11FIP4, NDUFB9, FGR, PDE1C, ATP7A, KCNK5,
DCLK1, HAGHL, BRIP1, SPACA9, HEATR3, DMXL2, SYT10, KLHDC4, PRXL2B, WASHC4, EMC6, SPG21, ECRG4, POLR1B,
LRRC45, STAT3, TCF7, CEP290, RIF1, SLC25A29, LTN1, PHETA2, RAB43, WDR25, DMBX1, RFXAP, ZSCAN23, ZSWIM9,
HIPK1, PARP3, WAPL, LYSMD1, TMEM169, C9orf85, IL18R1, SGMS2, C3orf20, BCL2L11, CUL5, LRRC18, TMC6, CDK20,
PHEX, OAF, NKPD1, SRSF11, FBXO41, BGN, NIPSNAP3A, GOLT1A, RTBDN, SEH1L, TRIM41, TMX4, CHPT1, UBR5,
LRRC74B, CCDC134, CRB3, CHI3L1, GSDME, DNAJC28, GALNS, VMA21, ARMC2, ZNF182, FAM177A1, MRNIP, ICA1L,
C2orf68, CRB1, KCNC3, TRIM62, ZNF558, KITLG, NLGN2, NRG2, SLC35E3, UCK1, EPG5, TSPAN2, SH2D5, ZNF200, DERL3,
FAM185A, HOXD9, ZSCAN10, JRKL, MED4, ZDHHC17, ARHGEF1, KAT2B, EDC4, IL13RA1, LEMD1, ACAD8, DUSP18,
FAM78A, KLHL29, ZNF557, RINL, GCNT4, TANK, CASP7, ZNF615, COLEC10, FARP2, GABBR2, TBC1D30, PKHDIL1, FHOD1,
RFFL, DND1, SUGCT, CAMK2A, MKRN2OS, HABP4, PIM2, ITGA2B, IAH1, CACFD1, ECI1, PTPN6, SETMAR, EZH1, PTPN9,
CEACAM20, ABHD13, KBTBD11, Clorf35, AMMECR1, RAB40B, FAM219B, CYRIB, ANKRD29, ANK2, QRFPR, TOR2A,
AFAPIL1, FOXI1, SGF29, EPGN, DCAF15, PRICKLE3, WWP1, MCUR1, PPP4R4, LIN54, ZNF438, ATG4B, TMEM204, RYR2,
PHKA1, ANKMY2, ZNF846, ZNF302, BAG2, OXR1, SOX8, HSD17B4, BTNL8, PIP5KL1, CXADR, BLOCIS4, THTPA, URI1,
CDCA7L, RIMS4, JAGN1, NAPG, ATG9B, SMTNL2, TLE6, RIT1, IZUMO4, SGO2, SPSB3, CCDC88B, EPS8L2, MEN1, ABCA3,
FAM200B, SLFN12, SYVNI, MAP3K11, ZMYND10, CHD3, AKAP11, EMID1, GOLPH3L, FBXL4, ICAM2, LRRC41, CCDC15,
POLRMT, SAMM50, ZNRD2, TNNI2, DMTF1, NCKIPSD, H2BC3, TEX14, FRY, CERS6, WDR53, IFI35, PRSS58, PCDH10,
CHRNB4, HLA-DOA, CYP2C19, SAMD1, CLCN3, KLHL6, CPLANE1, AFG1L, ATP6V1E2, CCDC84, PYM1, AGPAT5, CAB39,
ABITRAM, NFATC1, PEMT, SLC12A3, MSRA, ARHGEF40, AHCYL2, SOAT1, VLDLR, DYRK1B, ST3GAL2, UCN2, ATP13A1,
SYT7, CRNN, ZFP14, ADCK1, RHBDF2, PHLDA3, SEC31B, ZNF410, ACVR2B, CDYL2, LZTS3, KIRREL1, MARCHF3, BATF2,
HDGFL3, AMPD3, PIGW, LUC7L3, TADA1, C9orf43, CBY1, ZNF101, KAZALD1, NUDT21, PLA2G7, LURAP1, UTP20, UBXN8,
BNC2, SLC11A2, POLM, SLC19A3, ATF7IP, TMEM203, LRRIQ1, CDH3, ARL11, C10orf88, RAD9A, PLIN2, ASB6, BNIPL,
CCDC12, MFF, PKNOX1, ARL5B, AMIGO2, BCKDK, SMDT1, LETM2, NRGN, ZC3HAV1L, AP4M1, EZH2, RTN4RL2, USP19,
COMT, ZMYND12, DDX47, SLC25A51, ZC3H8, GCLM, ITCH, CTR9, WWOX, PGM2L1, STARD3NL, MSI1, NECTIN1, MPP2,
MRTO4, TRIM58, ABT1, PAX9, SLC16A2, ITGB1BP2, FBN1, PHOSPHO2, PGBD2, TBCID2, PLCG2, CD2AP, MAP9, MMAA,
MAP4K1, TCAIM, ACD, TRIAP1, LDHC, SRGAP3, MIIP, NOA1, CDC42BPB, TRAF3IP1, MRVI1, HAUS5, CPSF2, RPS6KA6,
SRGAP2, KLHL30, JPH3, DNAAF2, CTSZ, METTL1, SREK1IP1, CEP131, NUDT19, EFHB, TSR3, POLR3C, MBOAT1, TICAM2,
ANO6, SV2A, SLC17A5, NCOA2, SIGLEC15, IQGAP2, RNF14, MRPL17, GPD1L, CLDN16, ZNF366, ABR, PTPRU, TMEM230,
DNAH17, PEX10, SLC25A18, ATP2B1, RPS19BP1, RGS7, SLIT1, RASSF4, NME2, NSUN3, STRIP2, SEC16B, PC, ENTPD5,
ADAM23, CNGA3, ACSL1, ARL8B, FGGY, TCEANC2, TRMT2B, RGS14, PRRT3, ZBED2, WDR13, TRAM2, VWA7, SCAMP2,
PELI3, CCR6, ARSB, PDLIM1, ABHD8, PALD1, SLITRK5, FGD1, PKP3, KCNJ1, LSP1, VPS54, R3HCC1, HECW2, HES4, CD3D,
PPIL4, ZNF93, CCAR1, SDSL, KCTD21, POLG, TMEM175, KCND1, RAB26, DQX1, GPAM, SYT5, ACAP2, UBL3, CMTR1,
GLIPR1L1, FRMPD2, PDE2A, RAB36, RTL8B, DCUN1D5, RPP14, PPFIBP1, FKBPL, LZTR1, NDOR1, TSPAN15, SMPD1,
B3GNT5, MRO, SNTB2, LDLRAD3, TMEM79, FAM117B, RIC8A, TCEA2, ZBTB26, CIB2, AZIN2, PNPLA4, ANKRD6, TROAP,
ATL1, LRRC34, FYB2, PLB1, SCAMP3, ADIPOR2, ZNF575, SLC47A1, FAM92A, FAM222A, ATMIN, ATP9B, LZTS2, HSPA14,
RMI1, KIZ, FAM102B, SLC2A5, PDE9A, HADH, IQCA1, TMEM87B, MYOZ1, CEP72, ALAD, ZDHHC12, EID2B, SLPI,
SLC9A3R2, CGNL1, CYS1, ERC2, VGLL4, ARFGAP3, NUP210L, R3HDMI, MRTFB, MTMR9, MOK, ZNF530, TMEM209,
LMBRD1, KAT7, CIQTNF1, HRH4, COQ2, ASB8, SPATA6L, SLC27A2, RIPOR3, DPY19L1, PAQR5, RCHY1, GUK1, XAF1,
NSUN6, FABP3, PWP1, PXYLP1, CALHM6, CYTH2, LZIC, RNF24, CEP19, TUBGCP6, GCSAM, TM6SF1, EMC9, ABCC6, CNTF,
RRS1, C5orf58, H2AC7, ZFYVE1, HEXIM2, AKAP9, FLYWCH2, ZNF365, CHDIL, FBXO22, ZNF221, ALKBH7, HOXB13, KCNJ4,
SIGIRR, KRT7, PLPP6, LSMEM2, PHYKPL, KANK3, HOXB5, SAAL1, CCT6A, DNAH6, ECE1, LPCAT2, WDR93, PSTPIP1,
RNF26, RXRA, GABPB1, CDCP1, TTC39B, GMPS, PPP1R1A, SLC27A1, PTP4A3, IL7, SLC25A4, STX11, CD180, HYAL3, PGAP6,
WDR75, WDR45, RSAD1, RNF6, CCDC126, NMT1, FAN1, NAXD, RCBTB2, SNAP47, TCF4, LIN52, ZDHHC15, WBP2NL, ERN2,
METTL7A, FZD9, C15orf65, JTB, ZNF44, ZNF155, GNA11, RPS16, WDR36, MANIA2, WWC2, TTYH1, RANBP3, MAB21L4,
LRRC2, CHKA, SGSM3, PSD, HPS1, LRBA, TRARG1, EMILIN1, CDC34, FBXO43, SMIM29, CCDC160, UTP14A, GYS1,
TSC22D1, TMCC3, TOR1B, ENKUR, AGXT2, CCM2L, NEK8, BOK, PLA2G4A, OSBPL3, ZBTB38, RPS6KB2, TRMT9B, WASF1,
HINT2, EPOR, TMEM220, CFAP45, GGT5, HM13, MDFIC, RTN2, C3orf33, DTX3, NAALADL1, RNF157, SPATA1, SGCA,
WNT2B, METTL18, ZFAT, GDPD3, METTL16, FRMD1, SLC2A12, SEPTIN8, MYORG, PARS2, SMIM15, ZNF440, EVA1B,
DNAAF4, GXYLT1, TULP4, CARNMT1, SCARB1, PSPC1, PPP3CB, C9orf135, SLC6A3, ZNF444, CC2D1A, IRX3, USP2, LIPT2,
TMED8, MLH3, P3H4, PNMASA, TESMIN, ALB, JHY, DNAL1, PEX26, RUSC1, FBXO5, POP5, CCDC127, DPEP1, RAPGEFL1,
TTC19, CDKN2AIPNL, TRAF6, ID3, SP140L, ZSCAN26, SPTBN1, DDX11, CYP27A1, SLC27A4, CACNA2D2, PEX11G, MGAT5B,
TIMM22, BAHCC1, PAX2, AMDHD1, C16orf91, GALNT13, BGLAP, TRABD2A, ARSA, PIANP, CHIC1, PCGF3, UXS1, DOK3,
STXBP4, DHX34, CCND3, NDE1, RGL3, TMEM60, EIF2AK1, SLCO2A1, DNAJC24, BRMS1, UEVLD, ANKRD54, RAD51AP2,
TNFSF4, PTDSS2, C12orf65, NHLRC1, MBNL1, CENPQ, DLGAP1, MSRB2, NUB1, ZSCAN18, GALNT1, CDC14B, LRRC47,
METTL5, GPR63, HINFP, PCGF5, GATA5, RNF11, ADPRM, MR1, XRN1, KCTD10, PCSK6, TELO2, LRG1, IQSEC2, TLE2,
FAM178B, SEC22A, JPH1, ZNF483, NPHP4, HECTD3, CCDC114, TPST1, IL411, ERO1B, SYT3, MTSS1, PJVK, MMS19,
ARHGAP33, DPH5, HDDC3, HSD17B13, TMEM164, RHPN1, SLC38A7, MARCHF5, RPAP2, PIGC, MVD, RABL6, DBR1, BTRC,
ZNF260, CEBPG, CYHR1, PANK4, PUSL1, RFLNB, BLCAP, CASQ2, OVCA2, SRP72, SRD5A3, ZNF511, NALCN, SYNPO2L,
ABCA13, GABPB2, PSD3, CDK10, HES6, ISG20, LTBP2, MANSC4, NT5C, ARHGAP6, PAK6, NUDT7, ARHGAP27, NAA30,
DTX4, NIFK, CCT8, CRTC2, DHRS11, LZTFL1, NMRK1, CYP4V2, CHRD, MN1, NEK4, PRKCB, GUCY2C, STXBP5L, ASPHD2,
STC2, GFPT2, BMP4, MYH14, SLC15A4, CPLX1, AEBP1, KCNC4, RWDD2A, ANTXR2, TDRKH, SPINK5, RNF170, C1orf131,
FBXO36, ADCK2, PHF19, SPHK2, FAM219A, GCNT1, ENTPD2, GCAT, NAP1L4, PDE1B, TRIM45, AFMID, ENAH, PUS10,
EFNA1, TMEM53, LACC1, AGAP2, BMPR1B, AP1S2, TAF6L, SGPL1, FAM110A, SHC4, PITPNB, UNC50, CCDC149, DENR,
USH2A, RPH3AL, ZAN, RUFY1, DNAJC30, RASL12, SLC2A8, CPNE4, TLR5, MEF2C, CNTLN, PYGM, SLC44A3, SYT1, MCRIP2,
PCDHAC2, JDP2, ADAT1, KLHDC8A, DCP1B, LPGAT1, MED11, L3MBTL3, MIB2, EI24, TMEM8B, CEP44, XYLT2, SLC26A1,
GPR78, TBC1D2B, DMXL1, FKRP, NUMA1, GGT7, TDRD6, STARD4, STAU1, CLEC11A, NAA35, ANGPTL6, GK5, TEAD2,
OSBPL6, P4HB, TEAD4, SEPTIN14, IDH3A, LRRC15, PBX4, FIGNL1, PDLIM2, LRRC23, SLC4A1AP, XRCC3, DEGS1, ITGA8,
CNTN4, ARSG, CTSK, PPP2R5A, TOLLIP, ZNF222, CFAP61, ZNF527, BAZ1A, CABLES1, CDK6, COX20, FLT3LG, MPP7,
OBSCN, P2RY11, PLEK2, RFX2, FILIP1, HOXC13, TTC13, NAP1L1, SLCO3A1, ATG16L2, SUSD4, LRRK1, BRAP, MLX,
TNFRSF13C, ZSCAN32, ZNF724, DNM1, POP7, PTK2B, GIT1, SIX1, FBXO17, GYG2, AOPEP, PAK1IP1, PI4KA, C17orf97,
CLOCK, LETM1, ACSM3, ADAMTS17, PHLDA2, SERPINA5, FADS1, RCC1, DXO, RSPH9, TEX264, HS3ST5, BRSK2, BBS12,
GOLGA4, GABBR1, MFSD14B, ANKAR, GPR153, MT1X, PNN, PRKCD, TAF2, DDX10, LRRC75B, SOX10, MTFMT, MMP24,
HSD11B2, GSR, TMC4, TMEM170B, ASB11, CNNM4, PRSS23, RNF10, MTMR11, FBF1, ENDOD1, SWT1, HGH1, ERF, TRIM37,
MASTL, CMC2, HCLS1, TEX9, PES1, TRIM66, POLR3D, C11orf91, ELOVL7, HDAC10, PLEKHG7, PPP2R5E, MTHFR, ANKRD24,
IKZF4, VPS11, NAT14, RAB11FIP3, ONECUT2, FARSA, RRAGB, BORCS5, DHRS3, APC, HRCT1, ZMYM1, PPIG, TRIM36,
MINDY4, CHEK2, GPRC5C, SLC26A11, ISL2, C6orf62, ALOX15, TMEM81, POP4, AMOT, MTX3, ADAMTS10, ZCWPW1,
PPP1R3D, SLC25A15, H2AC14, MS4A2, HAPLN4, SPOPL, TTI1, RIC8B, GRID2IP, PCTP, TXLNA, NOXO1, FNDC4, DHRS7B,
HOXC5, TMEM251, VPS8, POFUT1, DNAH9, MAP2K6, MTA2, C6orf52, SLC2A4, DDX50, NLE1, MMP19, LEAP2, MEIG1,
FKBP4, QRICH1, FADS3, INSC, CCAR2, SNAPC5, SOX9, CLN6, MED27, RNPC3, ZBTB42, DNAJC6, TUBA3D, PCOLCE, SCN5A,
DRC3, KIAA0513, SERGEF, AVPI1, ZNF638, CDYL, GUCY1B1, PRSS56, ZNF25, ATR, GIN1, TTC4, CDKL2, BRCA2, VMAC,
KIAA0319, TOM1L1, TKT, SDE2, SEPTIN9, GNG4, RBM34, SLC25A42, CCDC148, CDC42BPA, GMPPB, TUBB3, YWHAB, RAF1,
L3MBTL2, CSF1R, SEC14L5, GHDC, WHRN, GMIP, LAX1, SMAP2, DPAGT1, DUSP9, DYNC2H1, XRCC2, HPCAL1, KRT28,
SORD, RNF25, LAT, ZNF682, WDR64, DLL4, CRBN, FER1L6, IMPA1, TCP11L2, KPTN, NT5C3A, MFSD13A, ALG2, KLC3,
SAP130, DOK7, FAM172A, NPHS1, UCN, PKIA, KATNA1, ACVRL1, ARRDC1, SOX7, PDE7B, KCNH3, C18orf21, MKS1, WNK2,
SLC11A1, CYP7B1, LRRC9, CCL22, CFD, KSR2, ADRB2, ATP6VOA2, FAM50B, FBP1, HORMAD2, OCLN, PACSIN1, PYROXD1,
YPEL3, C20orf27, COIL, GET4, SCN2A, SYTL3, ZDHHC23, NFYA, MEDAG, GTPBP8, DUSP26, EPHX3, PHC1, STRA6, ANXA9,
BCLAF1, GRK6, CAMK1, ARHGAP18, GRB10, RAB6B, ALG14, CCDC78, ST3GAL3, ABHD14B, NELFE, ZNF227, DRP2, HK2,
KIF9, RTL9, TCAP, SHBG, CCDC80, PLXNA1, BAG5, CARD14, LMLN, LCN12, VPS52, DCTD, GRIK5, TSPAN33, GGA2, NAB1,
MTG1, ZNF30, ABCA1, COTL1, WDR6, HTR5A, SNAI2, ZNF879, DPY19L3, OCRL, BTBD16, SRSF10, CCDC40, PWWP3B,
QTRT1, RAB3B, ROPN1L, C16orf46, CSDC2, NME9, TMEM145, USP27X, PTDSS1, VILL, ITGA10, KIAA1324L, TMEM190,
TMEM143, DERL1, SHISA4, PRXL2C, FBXO10, CARF, MOCOS, NES, PFDN5, OPTN, PEX12, ZBTB22, UNC13D, MFSD4A,
TMEM177, APBA1, RPS6KB1, CNTN3, WRAP53, GYG1, KCNJ14, CCM2, KCNS3, PLAC1, ARHGAP44, ADRA2C, MEIS2,
CFAP58, MARCHF4, ABCB9, MGAT4B, ANO4, AKT3, ASTN2, BMP3, SSC5D, EXTL3, DUSP12, ZNF699, FANCF,
CSGALNACT2, F3, TFB1M, MRPL10, SMIM6, SPTBN5, PDRG1, ODF2, CD37, ZBTB7C, RIBC1, EPHA10, UNC13B, ALKAL2,
CXXC4, ZNF268, TMEM253, AKAP5, REP15, ZNF720, PIAS3, SARAF, SLC6A4, TMEM250, LRRN3, ZC3H13, ZC3H15, GPR27,
HS6ST1, RHOD, INPP5J, ZNF383, RTF1, RRNAD1, WSCD2, PAG1, CFAP46, ARHGDIG, LRRC28, MAP3K10, HMOX1, STXBP3,
VNN1, CGGBP1, RBFOX2, INVS, MID1IP1, MLLT11, DDR1, AASDHPPT, ABCC8, DOCK11, MAVS, ZFP90, NPRL3, SLC16A11,
HFM1, HECW1, CALB1, PIH1D3, ANKEF1, ARRB1, BHMT2, RNF135, SHARPIN, MOAP1, COA4, GSTK1, USP51, SIRT4,
ZNF169, FASTKD1, HSCB, FAM114A1, FRG1, TTC21A, PUS7, RNF34, TBC1D5, VWA8, DIAPH2, LPIN3, SPATA33, FCSK,
OSBPL2, PAQR4, RABEP2, HOXD8, ZKSCAN3, TTC12, CRLF3, TIMELESS, TACC2, KANK1, SAMD9, GPR161, PCDHA1, SLIT3,
YLPM1, CCDC24, RPE65, SLC5A6, NR1H3, PCBP4, SLC25A52, TMEM106A, ARMC6, GABPA, CRAT, PHF7, H2BC13, HGS,
MFSD9, F11, KLHL2, TADA2A, CENPO, LCORL, HOXD4, GTPBP2, RRP9, ANKFY1, DENND11, NRROS, ABCC3, KCNK1,
PLK4, SNAPIN, UST, PCLO, ZNF808, TACC1, TMEM42, GJA3, SYT11, DST, ZC3H7A, PKN1, STRN, LRRC31, PSMB4, MOV10,
TMEM63C, CYB561D1, SH2D3C, NUDT16, CLDN7, B4GALNT1, L3HYPDH, SEMA5A, SERTAD4, NACAD, PLAT, FAM120C,
TBC1D9B, ARPC4, MPHOSPH10, HPS5, OPHN1, SLC25A40, ETV5, PCMTD2, MAPK6, CORO1A, ZNF616, TEKT2, DBP,
ABHD14A, TSSC4, CYB5D1, PDK3, CYP26B1, ELMO3, MON2, PRDM8, USP8, ADORA1, PPP4R2, GLI2, SYSI, COQ10A,
MARK3, RHBDL3, IFT172, ACOT6, UBXN2B, MUC1, PCDHB15, BDP1, RGS20, FKBPIB, TNFRSF21, ASCC2, RECQL4, TYRO3,
CORO2A, SYTL4, MRI1, ZBTB8A, APTX, FAM122B, HOXC8, ATP7B, FAM227A, ZCCHC7, SS18L1, FAXC, HIF1A, ATAD3A,
TRAIP, HACD4, BAIAP3, NOL4, RHBDL1, DENND2C, NEURLIB, MXRA8, TCTE3, POLK, PLEKHO1, PDPR, TPHI, TMCO4,
TRMT11, ANO1, TGFBI, IFITM10, PANK2, SLC17A7, ACTG2, JAK3, CHN1, TDRD7, ZSCAN31, FMO4, N4BP2L1, FZD1, MFSD8,
OPRL1, RASL10B, TMEM268, WDR97, GPR75, PIGV, FAM89B, SCRN3, FUT11, LRP10, INPP4A, EEF1AKMT3, APBA3,
TMEM45B, STMN4, GNPNAT1, OPN3, PCDHB8, INSYN1, XPC, UBQLN4, STAC, ANGPTL4, MAP3K7, STK11IP, HPN, ZNF419,
ART3, IL17D, HILPDA, SFXN4, USP43, C1orf115, TMEM223, ZNF516, P2RX7, ASPA, SSBP2, RELA, IGDCC3, SLC66A2,
GOLGA2, C22orf39, ATAT1, MEX3B, PTPRM, SH3BGRL2, SLC12A6, ATP4A, GABRA1, LRRC1, ZNF382, OTOF, PPAN, ZNF536,
GNB4, PRUNE1, LRRC75A, IKZF2, RNFT2, SHTN1, ANO7, MIOX, ADM5, MANIA1, CASC1, C5AR1, LSM10, PLXNB1, RPP25L,
FRMD5, HOXC9, LIN7B, ZRSR2, AIP, EXOC6, ARHGEF7, CYP2E1, FBXW5, SLC34A1, USO1, FBLIM1, ZNF235, RAB3C, BST2,
COLGALT1, RAB3D, SHOC2, STAC2, PPP1R32, COG7, NFIX, ELOVL6, SEZ6L2, FBXW2, GPR39, PRCC, RGS9BP, SLC22A11,
ZDHHC16, PGM1, LRRC61, ZFAND4, FOXD2, TMEM241, ESPN, ENDOG, LNP1, TIMP4, ZNF583, NOXA1, ZNF397, PTPRN,
CAMK2N1, SOGA1, HNF1A, SLC25A17, ESAM, SAMD12, ALDH3B2, NPPB, NCR1, EFR3B, FCHSD1, SCAMP5, NAT8L, ROCK2,
TRMT1, ARL15, SRPRA, ABHD2, CALR3, CDK2AP2, JMJD4, KCNK3, NUDT15, SLC44A5, BRAT1, EHD3, LRCH4, BTG3,
MED14, PSMG4, MID1, PLA2G12B, NKAP, ZNF350, SAT2, EHD2, EPN2, KCNIP3, FAM151B, GINS2, RUFY3, EDF1, HDX,
ASPSCR1, KLF9, ENOX1, UBLCP1, SYCE2, ADGRF5, CENPH, FNDC3B, MAPK12, XKR5, ALDH1L1, FAT4, GNAZ, MOXD1,
SCD, FARP1, SRD5A1, DNAJB13, KNOP1, MOGAT1, CEP164, AP5S1, C20orf96, GALNT17, PTGES, SLC16A13, TERT, TMX3,
METTL26, TSPAN5, ANKRD49, C6orf118, IMPA2, DRG2, NCAPH2, FXYD6, ZNF362, ZFR, FAM111A, NNMT, EME1, GLYATL1,
OSBPL10, TMEM87A, IRF8, TESPA1, KLHL42, SFTA2, STARD10, IL23A, TRPT1, KCNT1, CDK5R2, ZFP69, PRRT4, DHCR7,
HYLS1, MALT1, ENHO, EFNA4, MYB, FAM161B, ZNF24, CSF2, ODF3L2, SPTBN2, HYKK, DOCK4, SP5, AP2A2, ALDH1L2,
FICD, MAP7D2, SEPSECS, SMKR1, TAT, FAM110B, OTULINL, KCTD5, FZD4, KLHL25, PDZK1IP1, SGTA, DMAP1, KCND2,
NUP58, ZNF215, KIAA0319L, THRA, RDH12, TCTEX1D2, BAG1, LRRC8C, MTURN, TJP3, KIAA1586, MOGS, OTUD7A,
PPP2R2B, HOXC4, RNASEL, HOXD3, ARHGEF37, OSBP, TANGO6, ZNF649, CERKL, CHFR, CDPF1, FADS2, PPP2R2C,
ZNF780B, C1orf21, CEP83, THAP11, GPBP1L1, COL12A1, MONIA, EIF3M, GPBAR1, INPP5K, DDHD2, NFYB, ZNF467, HSF2BP,
AADAT, CFL2, QTRT2, CMTM7, PELP1, RANBP6, DHDH, L3MBTL4, BRIX1, DLG3, PROSER3, TMEM238, ZNF45, CHRNA7,
PSMD6, TNNC1, UBXN2A, CNTN2, NLN, SLC2A1, ATL3, RAC3, SFN, RNF187, PRSS12, ERMARD, C1orf159, MAPK8IP1,
ARMH4, PHLDB1, TYW3, CD58, PACRGL, TNFRSF11B, MICAL2, TMEM9B, DHPS, FAM149A, DHX9, DMTN, PDK1, PRODH2,
RGS11, SPAG6, SLC8B1, TGDS, ZSWIM1, MISP3, CTRL, CHST7, CHRAC1, ELMOD1, ARHGEF12, ANKRD34A, LRRC36,
CCDC97, PPP3CA, DEUP1, CHMP1A, VAV1, MAGEE1, ARCN1, CD3G, EFEMP2, NUDT13, CEP57, ROS1, CYB5R2, FNTA,
CAMK2B, DNAJC1, DZANK1, TBC1D7, EVI5L, FAM24A, TMEM263, FAM124B, CCDC39, DAXX, KCTD14, WDR47, ABLIM3,
KATNB1, TPRG1L, FLVCR2, LHFPL5, SPIN4, FLRT2, PPP2CB, HMCN1, TMEM59L, TSGA10, ATRX, SCN1B, DNAH10, UFSP2,
ADGRF4, IL1R1, ZC3H3, ZNF225, MCF2L2, MBIP, ADGRB2, MED28, NEGR1, SPPL2A, CDH24, GFRA3, GPRASP2, YES1,
B3GAT3, ERFE, CASS4, GPR137B, CASP4, PGAP2, AGBL3, MAP3K15, SNX13, TGM4, GRK5, PRIM2, C19orf57, DNHD1, RTL5,
ZNF385C, GRWD1, PABPC5, LPAR2, HIP1R, MMP17, MFSD10, TMF1, LMO4, TSPAN17, VGLL2, MED30, RCOR1, VOPP1,
NIPA1, WNT4, STK19, PEX11B, GNA15, RTN4R, TSNAXIP1, MED10, ROGDI, SLC5A1, MSH3, HOXA4, MAN2A1, PMS1, ITGA7,
TLCD1, DDX39B, RHOT1, SLC45A3, ZC3H14, FLAD1, TMEM127, DIS3L2, ALX1, CITED4, CPXM1, TPPP3, ZHX1, TIA1,
BRMS1L, TMEM37, DCUN1D4, UTRN, PGF, RIMKLB, SCN9A, SPATA24, SULT1C4, PCID2, C17orf64, CD209, SHLD1, TERB1,
ELAC1, ESRRG, SWSAP1, TMEM219, ALKBH2, CA12, MFSD5, PKN3, GLT8D2, ANKRD37, MYO1A, FSD1, FAM155B, KLHL32,
PGM2, PECR, SGSH, MYD88, PPTC7, TULP2, CADPS2, CLMN, NFATC4, ASGR1, KDM4D, WNT7B, ZNF426, CARM1L3, RAB28,
ETNK1, FAM89A, RNF44, CCDC66, PRR22, RIMKLA, BTBD1, FAM207A, HOOK3, ZNF347, TRIM32, LIMS2, ARL8A, EPS15,
TMUB2, TENM1, CSPG5, SPHK1, PITPNM3, GATM, CIPC, RCAN2, PSD2, C2orf76, SMIM13, DYNC1LI1, PLAC8, POMT1,
SNRNP40, TMUB1, UBALD1, CCDC87, SCLY, UBE2D4, CAPN10, KAZN, EED, USP54, MIB1, STRC, PKHD1, FN3KRP, SHD,
CFAP70, PI3, PROCA1, CYP2B6, FASTK, GLYCTK, ZNF862, CA13, CIQL1, PTRH2, CAMK1D, ZNF428, ATG2A, BCAS4,
C11orf71, CCL27, SRCIN1, FAM110C, CABLES2, VWF, APIP, POLR2H, TXNL4B, FOXJ1, ANGPTL3, LARGE2, PROM2,
CFAP298, SAP30BP, ZBTB41, CD96, ADRB1, CARM1L1, ENDOV, ERICH3, RABIF, AP4B1, BCDIN3D, MYL3, C1QTNF6,
SLC4A4, MREG, SLC6A6, ZNF688, PRNP, NFIB, KLHDC7A, OSGEPL1, SLC26A10, SFRP1, PBX1, ZNF71, MFSD4B, SSTR5,
CARD6, GAS6, SNTB1, CEP120, ACTR5, RXYLT1, SNAI3, CSPG4, NTAN1, SAMD14, ADH1B, MFAP5, RCE1, KATNBL1,
RASAL1, TIMM29, DSC2, HJURP, LCLAT1, NECAB1, PHKG2, SPESP1, VPS13C, LIPG, LYPLAL1, MANSC1, PLPP2, TRIM22,
CHGB, DCAF1, MACIR, ZNF518A, PGAP1, AREL1, CXXC1, DUSP22, PHF14, SIPA1L2, C5orf34, CLCN5, TMEM130, BCLAF3,
SFMBT2, LARP4, JAM2, CPNE2, ZNF891, GNG7, PTPRD, WDR7, COX10, SUFU, NAT16, LRRN4CL, GDF9, PCSK1N, NBN,
CEP97, EXOC3L1, KIAA1841, RUNDC3B, MAPILC3A, TUBGCP2, TBCID13, ZCCHC24, RARG, EHD1, PCSK5, NAALADL2,
NOS3, BBS7, SNAP29, SOX18, TCEAL1, TENM2, RBM38, LNPEP, MTRES1, AUP1, GORASP1, LCA5L, PLCXD1, DRAM1,
ISLR2, PAF1, ST13, DES, SLC36A2, SMIM4, FYCO1, MOSPD1, SPATA17, CTDSP1, DOHH, FHL3, VPS37C, C16orf74, PTPN1,
RNF145, ZFYVE19, CASTOR1, GTPBP10, TRUB2, SELENOS, BEAN1, PRKAB1, SNN, P3H2, PIF1, MEMO1, ANKZF1, ERMAP,
HSD17B8, PLCH2, SFXN1, LPAR5, SF3A2, ST3GAL6, NID1, PI4K2A, TMEM121, TBC1D16, TTC16, ZW10, KCNH4, HMGCR,
PHF21B, PXK, DGAT1, HOXB6, TUBGCP5, PIK3R3, PRCP, RHEBL1, SPPL2B, SRM, SLFNL1, TUBGCP3, GP2, KCNMB4,
MBOAT2, NFASC, NUDT18, MTHFD2L, ZYX, RIMS3, DPH6, MPST, PIK3R5, PILRA, SAMD10, PCDHB6, FBXL13, SERINC5,
SSRP1, USP10, CHRNB2, FAM122A, ZNF585B, MNT, SLC7A8, HSD17B14, PPPIR3E, SEC61A2, VWA5B2, HOXB8, WDR78,
FBXO32, NAB2, SLC46A1, AIG1, EID2, PLEKHA8, AFAP1L2, ANKRD22, WASHC1, ZNF850, C11orf80, HRH1, GPR83, PTGFR,
RPIA, GUCY1A2, PRMT9, HSPA1L, LUC7L2, TAF6, TMOD2, VTIIB, ZNF185, ALPK1, RNF8, CRELD2, ZKSCAN8, TLCD3A,
SNCG, TMEM144, SGSM2, ACTRT3, IGIP, HYDIN, UBE4B, PLA2G6, PUM3, FAM221A, DENND4A, MEIS3, SMYD5, HUWE1,
TIGD7, CEP85, KLHL20, S100A5, C1orf94, SNX4, TSEN2, TP53111, TNS4, CCDC151, ZNF845, EFCAB13, KCNN2, PRELID3A,
ASB13, PPM1J, HTR1D, TMEM216, STAP2, TASOR, AMT, CTSD, CTNS, FAM167A, ADAMTSL5, CCDC110, ZNF354B, PPFIA4,
SGK2, XPNPEP3, ARNT2, DBNDD2, DNAJC4, FRRS1, HLA-F, DEPTOR, PALM3, STK38L, ZNF256, A1CF, ALPK3, PRKD3,
CTSH, PGR, ZBTB44, ASB3, CDC27, FAM104A, NKX3-2, CTSW, UHRF1BP1L, GPAT4, CD40, CDS1, FBXO24, GRAMD2B,
PHYHIPL, PLPPR3, FHL1, CASR, WDR4, TMCO6, DENND2B, NADK2, YIPF3, ZNF605, SLC9A2, LYPD1, SDHAF1, CNPPD1,
RAB34, SMAD5, USP49, LYPD6, WDR3, SKIV2L, PXMP2, CFAP69, COL4A4, ZNF793, KLHL13, LRFN5, HDAC11, NKIRAS1,
CREG2, PPP1R14A, SLC47A2, TMEM91, DNAJC9, MMRN2, CLBA1, GMFG, TMEM167B, TTBK1, H2BC18, DNAH2, CDO1,
MPZL3, H1-2, DSCC1, FAM81A, ZNF181, PPAT, HAUS8, PSTK, PARVG, RILP, ARID3C, SETD6, PLXDC1, USP40, SLAIN1,
MYO1B, RNF115, MAP2, GTDC1, CLTCL1, C1orf127, EPM2A, CRPPA, FANCC, MTHFSD, FOXN3, MZT1, NFATC2IP, AMIGO3,
ARNTL, MED18, CHAF1B, FAM131C, PEX11A, HR, OTOA, SBK1, TBC1D24, ARHGEF2, CCDC184, SNRNP35, SORBS2, FBXL3,
MED23, C5, TDO2, DACT3, RYR1, BCO1, CASC4, TRABD2B, RIOX2, PRSS35, CCDC17, FRK, NUP214, TRMO, CAMKV, APC2,
TMEM151B, SLC38A3, LMBR1L, ANKRD30B, EIF2D, CDC42BPG, TFPI2, CYB5R4, PLCD4, POC5, CCDC173, SLC10A7,
ADAMTS14, GORAB, PALLD, CRACD, STYK1, SLC2A14, GDI1, ASCC3, LHFPL6, NPEPL1, CRTAC1, LRIG1, SP100, CEP112,
ANKRD42, EIF1AY, MCTP1, PDE4DIP, ASB5, PSKHI, RGL2, SPSB4, CLTA, ZNF233, CBY3, FAM174A, SNX29, EVL, DNM1L,
TXK, GLI1, PECAM1, SLC27A5, ZDHHC2, NEIL1, EPHB3, ANKMY1, SAC3D1, AZI2, CBX7, CPLANE2, MYBPH, HPSE, ZNF667,
BAIAP2, DENND1C, MYOM2, CDC42SE2, FBXL19, OGFOD2, PPFIA2, RTKN, RALGPS1, DOCK7, AFG3L2, SLC35D1, WBP1L,
DUSP23, GPR137, ZNF248, PM20D2, CAPN5, TAF4B, ZNF300, AGL, DOP1B, INKA1, LRRC3, LRRC58, TMEM231, ALPL,
CAB39L, PAEP, SCN3B, EIF3A, GALNT6, IL18, NMNAT3, TMEM40, TRIP11, KCNK6, KCTD18, PRPF40A, TNFSF9, ZNF213,
SYNE2, CUL3, DPYSL4, MYBPC2, UBE2E2, ZNF704, AQP11, CNTN1, SETD9, CSNK1D, DGKZ, SYT8, TSPAN13, COG2,
CFAP53, MTIF2, SLC7A9, ZNF224, CCDC9B, FAM13C, JCHAIN, THEGL, ARMCX5, EEPD1, VWA3A, ZNF22, EMP1, TSHZ3,
RWDD3, H2BC15, BEND5, STK32A, C4orf47, CRTAM, ANKRD13C, FTCDNL1, ARHGAP42, EBI3, NARF, POLH, TMEM17,
JAG2, KIAA1755, MTCP1, EIF2B2, PACSIN3, CHST6, ST8SIA4, GALNT9, IKBKE, NCAM2, NECAP1, TTC9, KLHDC8B,
EHBP1L1, FA2H, SLC38A5, SLC50A1, TMEM259, WNT9A, ZBTB14, PDE6B, ZNF148, CHRNB1, PAN2, GCC2, OGG1, ACTC1,
VSIG10L, STEAP2, CASP8, ATP6V0A4, ATP6V1H, CCDC18, LGALS8, LTB4R2, FXR1, TTLL12, NOVA2, ARL9, SCNM1, CDC73,
MINDY1, C17orf67, FBN3, LCA5, C1orf50, RAB30, ZFAND1, SAMD9L, NHLRC3, HEATRSA, KIAA0556, PINX1, ZNF577,
ERICH6, RWDD1, SLC51A, PLEKHG1, ADAM12, CMTM1, DRC7, IGF2BP1, VASH2, GTF2B, TWIST1, MLKL, FES, COQ6,
CWF19L1, SLC25A46, IFT88, GSAP, DDX23, MDC1, PACS1, C12orf75, CHST14, GKAP1, NPAS2, TEAD1, TMEM117, KAT5,
POU1F1, TESK2, TM2D3, CERT1, CXorf58, HLA-DQB1, VGF, FRAT1, KIAA0355, EXOSC2, C16orf86, ZNF691, TRIM29,
ST3GAL5, USH1C, RABGGTA, DNAJC17, DENND6A, FLT3, SIPA1, SCRN1, ABCA9, TRERF1, PLEKHA1, TBCEL, HMG20A,
PRKCG, RYBP, PIP4K2A, FANCG, LRRC20, METTL3, ATG10, METRN, FBX02, AGO4, DAGLB, TANGO2, GLE1, SENP8, GPT,
ADAM22, STK3, DHRSX, HOXB4, SLC31A2, TTC25, CACNA2D1, CPQ, NT5E, CEP126, CCDC69, SGPP1, TMEM135, IL27RA,
ACOT11, PXN, SLC49A3, MPZ, NUDT17, CACNA2D4, H2BC17, MC3R, PNKP, SSH3, ZNF570, MYO9B, ACTR8, TMEM38A,
AK7, PAK1, PARP16, PDCL, STARD6, UBOX5, KLRG2, EXD3, FAM189A2, GABRG3, GNAQ, AUH, MBTD1, FMN1, SF3B4,
SYNJ2, TTLL6, ACSBG1, CYP4B1, CPOX, CCN5, PI4K2B, FER1L5, FAAH, RAB31, ZNF841, FUT10, ZNF285, GCM1, NT5M,
RAB7B, RASSF5, CCDC106, TMEM217, NIN, OLFM2, FCER2, RALGAPB, FGL2, STK36, U2SURP, SLC30A5, PLEKHA3, BBOX1,
FBXL15, RNF144B, NMRAL1, TMEM225B, COASY, GNL1, LRP5, PIGB, PPT2, TPM2, ME1, SLC25A26, CCDC159, SMG8,
BLMH, CDHR1, ICMT, KIF18B, ASPHD1, MCOLN1, NTMT1, SOX6, PLXNB3, MCC, TNFAIP8L1, FBXL17, RNF175, SLC29A4,
SP2, STK32C, MANBA, OSGIN1, SLC39A13, MXI1, ZFP28, RPS14, ADAM11, B3GNT4, PIGR, PPM1F, ACP3, B4GAT1, MYLK3,
RAD51D, SMPD3, SOWAHA, LRSAM1, FOXN4, POLQ, ABRA, CCL24, MGAT4A, SYT4, ZNF395, C19orf12, CTU2, CACNAIA,
PDS5A, FAM98C, REEP3, TMEM270, ZMYM3, VPS13A, CMC4, NRP2, RELCH, SLC16A6, SP140, TMEM94, STAT5B, LYSMD3,
FAM199X, ILDR2, CPA5, INPP5D, TC2N, LARGE1, OCA2, SLC4A11, ZC3H10, THAP2, RDH14, LPP, H1-4, MAIP1, IRAK3,
HNRNPAO, POMK, RINT1, ABI2, RRP1, SVBP, NGEF, ANGPTL7, IGLON5, PCCA, AGAP1, CCDC150, UNC13A, AKT1S1, PPIL1,
TTPAL, MCAM, NMNAT1, GPRC5B, SLC24A1, ZBED8, MPP1, TP73, THG1L, ACP5, IL1RL1, SETDB2, ZDHHC9, ZNF28, GLS2,
WDSUB1, PLA2G15, C1orf198, HEXIM1, KLF12, GAS8, SYNGR3, MGRN1, SH3PXD2A, NIPSNAP2, RRAS, MEF2A, PTOV1,
ZMYND11, USP45, C1orf210, MATK, MYLK4, PREP, NDUFAF7, WDR66, EPB41, VPS26B, MDM1, ESS2, UNC80, COMMD5,
CCER2, HID1, MRM3, PTPMT1, RAB27B, TAL2, LRRC73, PCED1B, RAB15, ALKAL1, RNF149, UAP1L1, CUBN, DOP1A,
ERICH1, CDNF, TRIM63, BMS1, GALR2, VPS26C, CTDSP2, NOL11, ESYT2, BLOC1S6, RAP1A, RSPH3, WARS2, TMEM70,
SLC36A1, TAMM41, ADGRF1, FMN2, GCNT7, KATNAL1, MAPK8IP2, RGS9, AASS, MTPN, NOTCH4, WNT9B, SVEP1, PAQR3,
ABRAXAS1, ACSM5, CCDC136, FBXO40, HACD1, IGSF9, KCNC1, KIAA1191, LDAH, LDHD, LRP3, MED8, PCSK4, PIGM,
PTEN, SAYSD1, TBCCD1, HPCAL4, SLC30A7, EML5, GFRA1, WT1, ZBTB33, ACOT9, CHMP4C, LRGUK, SLC35A2, HYI,
EPHB6, CTNND1, FOXO6, TTC31, GPR137C, APH1B, TAPT1, CATSPERZ, ZNF75D, NWD1, GCLC, YAE1, B3GNTL1, CCDC71L,
GSTM5, TSC22D4, PCDHGA1, RRAS2, OSER1, TMEM106B, SLC30A4, CENPV, ANGPT2, SMIM3, RRAGC, AGO1, GCNT3,
LRP2, MEAK7, ZCCHC4, MST1, CENPJ, MTERF1, PITX1, PALM, ARMCX2, CNOT9, EPS8L1, ANKRD27, LRRC27, SNAPC3,
RMND1, ROBO3, TNN, CCSAP, GOLGA7B, RNF208, PARP11, RUNDC1, CAVIN1, YTHDC2, NAA60, MED16, RBM7, TOMM40,
TNIK, CYP26A1, MAT2A, JAKMIP1, NEK9, TMOD1, PLIN3, LIPH, MAP1B, SEPHS1, SLC41A1, RNF112, CHCHD4, DCAF16,
C2orf72, C3orf52, STS, POU2F1, SPTB, GYPC, LILRB5, SMAD9, DYNC1I1, ST8SIA1, ZNF580, HCFC1R1, CCDC189, SYT9,
POMGNT2, FAM214B, SPNS3, LIN7A, ZNF780A, EOGT, SYT12, RUNDC3A, SNX14, SPTBN4, TRA2A, ZNF487, SCRG1, EDA,
PLCB1, RGS22, TRIT1, ZNF771, DHX29, PYROXD2, DTNA, COL24A1, SAMD15, HOXA13, CLPP, VTI1A, ADAMTSL4,
ANKRD40, THOC1, MSMP, DYSF, EMD, HSPBAP1, SMC6, HHAT, PITHD1, TNFRSF18, GAL3ST4, PRKAA2, SLC2A11,
PLEKHA2, ZNF283, ARMCX6, ZNF582, DBNDD1, LRMDA, DAB1, TRIM9, LYSMD2, CLDN23, TNFRSF12A, FOXF2, C19orf81,
HSF2, ZNF736, PLEKHF1, SNX21, RRP12, ARAP1, CNKSR2, DGKG, LAMC1, PLSCR1, PTAFR, SLC15A2, TWIST2, ZNF517,
PSMG2, ENTPD3, MAGEF1, TIMMDC1, ZNF311, MFSD12, SLC22A4, TBC1D17, CILP2, CREB3L3, STOML1, ZNF140, SORL1,
SYNPO, TXNDC16, CCDC77, UPF3A, EXT1, LMF1, LRRC7, LSM11, SLC7A6OS, SYP, OFD1, CELSR1, CPZ, CIAO3, COL1A1,
C19orf25, TP53INP2, KCNG1, TMEM232, FOXH1, THOP1, MPPE1, EXOC1, MUC12, CADM1, CAMKK2, TTYH2, ZNF610,
COL6A3, MIA3, RGS16, RB1CC1, CEP89, FOLH1, ITGAX, BRI3, CHPF2, CLCN7, MAML3, COMTD1, IL1RAPL1, SEL1L3,
HSDL1, VPS37D, P2RY6, RADIL, SLC23A1, DLGAP4, HOMER3, RRP36, SH3BP1, LGI3, RNF180, FZR1, MOG, DAO, MMP9,
SLC49A4, SLMAP, ARL14, ASTE1, PDIA2, ANKRD65, ARNTL2, PLEKHM3, RP1, C18orf25, HSD3B7, TAF8, UBFD1, MRGPRF,
ADAM8, LONRF2, PIAS1, JAG1, P4HTM, CHODL, FBLL1, HIF1AN, TMEM237, HOXC6, HSF1, METTL23, PNLDC1, H2AC20,
SLC4A1, CCDC59, NPR3, LIAS, MTRF1L, DOCK2, HSD17B7, LTK, NIPAL1, KRT12, NUDT1, SLC35G2, SMUG1, TMED3, MAL2,
RELL2, RSKR, DAB2, MTA3, CCDC122, GNAL, PLPBP, SLAMF7, TBX15, AKNA, RBM43, VASH1, ZADH2, CD163, NUP43,
DEPDC7, DLX4, WNT10B, DYNC1H1, NAP1L5, PLD2, TMIGD3, CNTROB, MED25, FUBP3, C12orf43, ZNF837, TRIM65, HLCS,
TTC32, COQ5, EMX1, TARBP2, GPANK1, SIK2, IRAK4, FBXO44, NOSTRIN, STEAP3, ZNF540, CDC14C, DNTTIP2, DOCK8,
UVSSA, OTUD5, ANGPTL8, ATP6VOD2, SELENBP1, FRAS1, ENPP4, FBXW9, KCNMB1, SH3YL1, ABCF1, ATP2A3, ADGRA3,
FAM187A, PGGHG, PRPF4, NOG, CCDC32, PLCB3, SLC30A6, SPOCD1, FNDC5, SLC25A27, C7orf50, FLI1, NBAS, TFE3, TIFA,
VWA3B, CHID1, GRTP1, PEX16, ETV2, ITGA3, MRPS6, MUC13, RASGRF1, TNFRSF25, ZIK1, SLC2A4RG, INHBE

TABLE 4B
Genes and Proteins from RNAseq Analysis.

NFKBIA, PPP1R15A, TNFAIP3, ARRDC3, JUN, NFKBIZ, IFIT2, HSPA5, DUSP1, IL11, EEF1A1, TIMP2, KANSLI, IFIT3, SQSTM1,
TIPARP, ZC3HAV1, KLF6, ENO1, GADD45B, NPC2, ANXA1, IGFBP3, DDIT3, PSAP, SPP1, CSRNP1, IFIH1, ADAMTS1, GPNMB,
CTSL, BHLHE40, ARID5B, PRDM1, TPT1, TUBA1B, IER3, TMBIM6, HES1, OAZ1, EGR1, GHITM, COX3, APLP2, KPNA2, VIM,
HNRNPA1, YWHAQ, NUAK2, AMOTL2, GRN, PRKDC, CCNL1, CHD2, MYC, ALDH1A1, CALM2, PFKFB3, LGMN, COX1,
CALM1, ACTR2, DYRK1A, PLK2, SUPT16H, PDGFB, TPI1, CCN1, SLC39A1, COX2, IFIT1, STMN1, PFN1, CHRDL1, DUSP8,
CST3, NOCT, ATF3, SPEN, NEURL3, RHOB, RGS2, CRYZ, PHGDH, IRF1, CLK1, CXCL10, LAPTM4A, RUNX1, MYH9, SMAD7,
EPC1, AEN, AP2M1, TUBA1A, CDC42EP3, RPS4X, ZFAND5, MT-ND4, CTNNB1, FOS, POGLUT2, MSN, RRM1, GANAB,
FAM83G, TXNIP, HSP90AB1, BRD2, CYTB, EPRS1, PDZD8, ARHGEF28, LGALS1, FOSL2, HMGB2, HSPA8, CD63, ARL6IP5,
TMED9, CHD7, ND3, IER2, EIF1, DHX58, MMP1, TUFT1, IL1A, PDZK1, UQCRC1, HNRNPM, PABPC1, CCN2, ATP6, HIVEP2,
SPRY2, CCL2, ZFP36L2, LGALS3, ATP5F1A, ERRFI1, EPHA2, SLC12A7, LRPAP1, SETD5, PPT1, AKAP17A, EEF2, ND4L,
PDIA3, NHSL1, PPARG, ATPIA1, DUSP5, SLC25A25, ZNF503, HSPD1, XRCC5, SBNO2, TNFRSF10B, PCNA, PTTG1IP, PELI1,
TPX2, PGRMC1, STARD13, HNRNPU, CALU, CLIC4, PPPIR18, PPM1D, MAP3K1, OAT, TSC22D2, TPM1, WSB1, EPCAM, OS9,
EDN1, MIDN, ND1, KLF3, S100A4, NPM1, KMT2C, CLDN2, RPN2, EPB41L2, CD9, PIM1, AKR1B1, MDH1, URB1, SERPINE1,
NARS1, NDUFA4, ZMPSTE24, NCKAP5L, SKP1, PPRC1, MIF, PSMB1, ATP6AP2, CTNNA1, GSTP1, SPCS2, ND5, PAIP2, ZFP36,
DDB1, FRS2, ANP32A, MCFD2, STK35, PSMA5, MMP10, BCL10, IDH3B, MCM3, ATP5MC1, DDX1, SFSWAP, CETN2, BCAR1,
CALR, MT-ND2, ATP6VOE1, MYL12A, EPHA7, SERPINH1, UQCR10, NOLC1, CAP1, KAT6A, NCL, PRC1, PSMB7, CERK, SND1,
ARHGAP23, NR1D1, ANP32E, YTHDF1, FAM107B, PALM2AKAP2, PIM3, TMED2, SEPTIN2, PDIA6, IGFBP6, PTPN14, PRDX3,
INTS5, TNRC6A, SH3BP4, ZBTB7A, MYL12B, THBS1, TAGLN2, BCAP31, CSDE1, RPS12, NDUFA13, COL4A1, UQCRQ, GPC4,
EDARADD, HMGXB3, CRIP2, LAMA5, FASN, POLR2L, PSMB3, UGDH, MDH2, ABL2, ACO2, JUND, PSMD1, SOX4, PSME2,
NUAK1, TENT4A, DYNC1I2, ZNF608, SDC2, KLF10, COL5A2, STIP1, WARS1, GDF15, NDUFA5, RPS6, PARP1, MDM2, NOP56,
CCL5, ZNF592, PGD, JUNB, MYH10, GADD45A, ERH, ZNF217, DUSP6, EIF4G2, FAM71A, WDR45B, RBM3, CREBBP, DLD,
RPS19, BUB1, NCOA6, MARS1, NACCI, LMTK2, JPT2, NUP98, ACADM, CD44, ANKRD50, TRIO, SRRM2, COX5B, TMED10,
USP36, FRMD6, ELL2, DUSP16, SPAG5, IREB2, G3BP2, KDM6B, TK1, ENY2, MTMR10, AGO2, POLR2B, COX8A, PELO,
FBXO42, STAT1, CSNK2A2, KMT2E, TET3, KMT2D, COX411, PGRMC2, KIF20A, POLD2, STOM, CYP3A5, MRPL13, SETD2,
ATN1, MED13, HELZ2, ASXL1, KMT2B, GPATCH8, DLGAP5, FOXN2, MORF4L2, RIPK4, OTUD1, VDAC3, NOP10, COPE,
FAM193A, MAP2K3, XRCC6, DSTN, TMEM59, IFFO2, RUSC2, ATP6AP1, NCOR2, NUTF2, RNF111, PARD6B, FANCD2, EWSR1,
ITGB1, RIC1, RPL30, ATP5F1E, ITPRID2, PSMB2, SERF2, URB2, INHBA, SAMD4A, BTF3, PSMA4, C16orf72, SRP14, TMCC1,
B4GALT5, MAGED2, AP1S1, DAZAP2, PCGF2, CIRBP, ZWINT, CEP85L, HNRNPH2, ARIDIA, RNF41, SOCS5, PPP4R3A, IK,
SSBP1, PHF12, SRSF3, LARS1, PLK1, TNFRSFIA, GBX2, PSMB6, DDX5, DIDO1, TLK2, NFIL3, CCNB1, MARS2, UGCG, VPS29,
EIF2AK4, FGF5, PPPIR3C, ARAP2, SEC24A, ZNF316, SPATS2L, ISG15, SCAF4, ETFA, SLC7A5, TMEM123, SEMA7A, TUBB4B,
RBBP6, PREPL, REXO1, ARHGAP1, DLG5, ETS2, PSMB5, DPP3, RAD54L2, GARS1, ALCAM, ABL1, POLA1, POGZ, ARID2,
NDUFB5, CSE1L, CIB1, CKAP2, NDUFA8, YTHDC1, CTSA, CAPZB, ACTN4, KPNA4, SPRED2, HNRNPC, SSR2, HBEGF, AHCY,
PRPF31, ECHS1, TRAF1, CRAMP1, COPA, RLF, KCNJ8, GPX1, NFKB2, RPLP1, FBXO11, API5, BRPF1, ZNF462, EXPH5, UBA2,
SNRPF, DUT, MGST3, TAF1D, ARPC5, EAF1, CAMSAP1, SSH2, NAA20, PDHB, ANLN, CDH17, IRF2BP2, CDH16, MLLT10,
ZCCHC14, CAPG, SDC4, COPS5, MAP3K14, SKI, SUPV3L1, NCAPG2, USP42, BDH2, BANF1, MSL2, ATP1B3, CDC20, DUSP3,
DDOST, HS3ST1, MCM6, PRKAG2, AIFM1, PBK, BEX3, COG3, AMMECR1L, IVD, NDUFB3, RASSF3, LDLR, FRMD4B, DAD1,
POLR2G, RGL1, ACAA2, NDUFA9, N4BP1, NUCKS1, PSMA7, VCP, TPP1, SRP54, G3BP1, MEF2D, TET2, ATP5PB, MITF, BSG,
ATP10D, CD81, RBM33, HSPA4, ANKRD17, CNN3, ID1, CETN3, GLUL, NUCB1, ZNF398, APOE, PSMD10, SPARC, TRAF4,
POMP, IFI44, BCCIP, CHMP5, SCARB2, NDUFS2, HEG1, GALK2, VCAN, SERBP1, PRDX5, TECR, HLTF, TES, S100A1, IPO8,
SLIRP, KIAA1522, UBN1, VPS26A, RREB1, CCNB1IP1, TSPAN3, OCIAD2, PLEKHM2, HERPUD1, NEDD9, HSPB1, SHMT2,
KDM2A, BRPF3, SMURF1, TMEM109, FOXJ3, ANXA5, ATP6VIA, PTPRF, SNRPB, ND6, RPS3, RERE, DHX15, AFF1, TRIB1,
YOD1, GATAD2B, CLUH, UBL5, UIMC1, PCYOX1, NUF2, SETDB1, DAPK1, CYB5B, KLF11, UQCRB, COPS6, BTBD7, TTC7B,
IER5, FNBP4, PLEKHG3, PPFIA1, ST3GAL1, MCM2, PMPCB, ITM2C, GNS, FEM1C, SCP2, RAB10, ZKSCAN5, RUVBL1,
HECTD1, SLC30A9, ABTB2, RBM39, PSMA6, SLC39A10, PPP1R9B, CRKL, PSMD2, USP5, NME1, RBAK, LAMA4, SLC25A37,
STARD7, TANC1, DSG2, RBM8A, MARCHF6, ZCCHC2, POLE4, CDK12, ZNF7, SHCBP1, CBX3, MB21D2, IFNAR1, PPP1R15B,
RBBP7, PSMD14, EFTUD2, DDX60, ROMO1, MRPL15, G6PD, HADHB, AKAP13, MIDEAS, DYNLT1, INO80, ERN1, NDRG1,
FBXO28, BUB3, TM9SF3, CTTN, RAB12, ARPC2, LASP1, PRKCSH, FSTL3, CPSF7, NPNT, FBXW11, EIF4A1, RUBCN, CLDN4,
CNBP, NPTN, NRIP1, MARK2, TRPS1, BCAS2, E2F4, RACK1, ZDHHC6, RSPO3, MYO10, CHD4, RBM15B, DOCK5, REV1, SRI,
PPP2R2A, CYBA, FOXA1, BAZ1B, PIGN, NUP107, MRPS9, DOCK10, PIBF1, PSIP1, VRK1, PLD3, PCF11, RANBP1, UPF1,
SRBD1, SUN1, RPS2, HNRNPDL, BRD4, TGFBRAP1, RAB7A, ARID3B, EVPL, YWHAG, LMNB1, CLK4, PCBP1, SERPINE2,
ZZEF1, ZFHX3, ZNF267, CHD1, APP, CCT2, ZBTB4, ZFY, IMMT, C1QBP, KIFBP, ETFB, NOTCH1, EP300, AKAP8L, DYNLL1,
ADNP2, CLIC1, MGST1, TM7SF3, ZSCAN12, MICALL2, GET3, BNIP3L, CAPNS1, MAGOH, C11orf58, MAPRE1, CCDC88C,
TBC1D10B, CANX, PTPRJ, ZNF746, MRPL27, ZNF655, ZNF574, ZNF853, EIF1B, SAT1, FANCI, POLR2K, PER2, KIF11, CNOT4,
IVNS1ABP, ZBTB43, PIEZO1, UBIAD1, SDC1, RAB11FIP5, MRTFA, PTBP2, MCM5, RAB8A, NRBP1, CD164, GPAT3, NOC2L,
POLR2E, ZNF787, TICAM1, COL4A2, VEGFA, LRIG2, CMAS, ATP5MPL, LIF, KLHL21, TGIF2, CREG1, SLC12A1, PIN1, IP6K3,
MYSM1, TOP2A, PCNT, ARHGDIA, EXOSC5, ADGRL2, HIRIP3, FSTL5, DGKD, DNAJA3, SYNCRIP, RFX7, RFNG, CDC25A,
PITRM1, CHCHD7, VTA1, XBP1, MFHAS1, RBM47, GLO1, ACTN1, BZW1, RGS3, PSMC2, LAS1L, CDCA3, CDK13, GZF1,
ZDHHC8, DHX37, PARK7, CHURCH, ACAD9, SMG1, SNIP1, UBR2, EMC2, H2AC6, DAGLA, TPM3, PAK2, RASSF8, ATP13A3,
PSMA3, SLC1A4, EIF2AK3, CHAF1A, ARID4B, SAE1, BECN1, ATXN10, PLIN1, GOLIM4, ZNF131, ENSA, TFRC, RAI1, CRLS1,
FBXW7, PPP1CA, PIGS, ASNS, ZNF142, GEMIN4, TBCID9, CYC1, CSRP1, PKD2, SLC25A33, LGALS3BP, PAXIP1, ZNHIT1,
ELOA, NAV2, SARNP, SUPT6H, LHX1, RAPGEF2, CAPZA1, FAM83H, ZNF335, RBM5, MACF1, COMMD2, IARS2, JADE1,
NDUFB6, IDH3G, ZNF827, CHPF, EIF4A2, SSB, NDC80, SH2B3, RPA2, S100A16, PLS3, ZC3H4, SNAPC4, RSAD2, SHMT1, RCN2,
LEPROT, ZNF3, RTF2, ITGA6, IFI6, SZT2, VPS35, KIDINS220, PAPOLA, ATXN2L, FOXO1, FUS, PAPSS2, CALD1, MAGT1,
CDKN1B, RAB5C, MRPL46, UBA1, AURKA, NHP2, PTH1R, TAX1BP1, COX5A, NDUFA6, INTS6, CD46, RAD23B, ARID1B,
EBNA1BP2, POLR1E, ZSCAN29, PHACTR4, POLR2F, PCMT1, C11orf68, SLC25A3, DMAC2, RSU1, GNA13, DDIT4, SELENOW,
RNF128, EP400, DPCD, PPP6C, EIF6, TPRN, PRMT5, SERPINI1, ZSCAN25, AXIN1, PPP1R10, MAST4, PPP4R1, RNF139, LMAN2,
LIMK1, ZBTB18, PRAG1, OASL, SERTAD2, LOXL1, SEC16A, MTA1, HNRNPD, UTP15, TMEM43, AFF4, DHFR, ACTL6A, BCL6,
DTYMK, ICE1, EHMT1, SCIN, ATP8B2, FOXK2, TRIM4, CLTC, H2AZ2, GLIS2, ZNF778, ECHDC1, ABCC1, BMP2, CEMIP2,
EAPP, OST4, KRT81, LRRC59, PRKAR1A, RBL2, VIRMA, UCK2, SCPEP1, MRPS35, LUC7L, CCNB2, CUEDC2, TAF15, ARL6IP6,
RRM2, STK40, UGP2, RNPEP, ERP44, PNRC1, DMAC1, CAMTA1, AZIN1, TARS1, MIS18A, DCXR, CWC15, HIPK3, NUP37,
RAD21, EIF4E, CPE, NEU1, TOP3B, PTER, NOP58, TXNDC12, HSD17B12, GSS, PHRF1, MAF1, ATP8B1, FAM193B, GTF3C6,
RPS21, BEX1, CRIM1, RSRC2, MPDU1, CCNJ, PML, HERC6, CLDND1, XPO1, ZMAT2, ZNF385B, FLCN, HNRNPL, UBN2,
EIF4H, ATP6V1C1, RNF213, DNAJB5, ATAD2, CCNT1, USPL1, SYNJ1, RPS13, ARHGAP17, HNRNPR, TBX18, SLC16A3, RPS15,
ERBIN, COMMD4, TAFA4, MAP11, MRPL11, SAP18, MRPS11, TNFRSF10D, BPNT1, DHRS2, PSPH, DARS2, OGFR, SPC25,
RASA3, TRIM24, PDCD4, NSUN2, GGA3, SETD3, UBE2D1, RARS1, TCEAL8, YME1L1, USP38, UBAP2, ZBTB10, TNKS2,
LMAN1, SPECC1, SAR1A, GRAMD1B, ZNF79, CTDSPL, CDC25C, ZBTB2, CDC14A, RPS5, CDK5RAP3, KIAA0232, LRP12,
DDX18, RELN, TMEM106C, MCM3AP, NUP93, AGTRAP, TIMP1, G2E3, ATRAID, PSMC3, MICAL3, NCOA4, ARF4, COG4,
BCKDHB, DECR1, MARCHF9, DTL, MCM4, PFAS, MLF2, GNG10, RUVBL2, CRK, ZNF48, MRPL58, HPS6, EXOSC8, DNAJC10,
UBE2A, SF3A3, SMARCC1, LYZ, MFSD14A, PSEN1, CTSF, SCCPDH, LSM7, SPINDOC, CEBPZ, NCLN, NUP210, C15orf39,
YWHAZ, MSMO1, PRR13, EPDR1, ESCO2, PARL, NAXE, NCAPG, ZNF408, BCL9, SYBU, EMC4, MFAP1, SRPRB, SMC2, REL,
DLG2, HLX, RPS8, SF3B6, AARS1, ATP6V1E1, TIGD5, TRIB3, CEP350, MYO1E, NR3C1, SHROOM3, HTATSF1, SNX2, RFC2,
EDIL3, ACADSB, SMG5, POT1, SPTSSA, RNF216, S100A2, CPSF3, PLEKHH3, OTUD4, PRDX1, PLP2, PAPOLG, PDE4D,
MACROH2A1, PTBP1, ARL14EP, PLAGL2, DMWD, ACVR1, WDR46, CCT7, PPP2R2D, IARS1, RANGAP1, EID1, ANKLE2,
ZFAND6, HSD17B2, DIP2C, AKIRIN1, TMEM131, RC3H1, THOC3, EIF4ENIF1, TSN, TFPI, TTC1, WIZ, RTL8C, SPRED1, PAM,
ABCB1, PPP1R3B, SEC61G, CENPM, ADAM17, ITGB5, AREG, VDAC2, CISD2, LRFN1, ZBED4, GRAMD1A, ZNF384, ACSL5,
LRRC17, MRPL42, MEPCE, MRPL37, NSD1, TM7SF2, AKR7L, YWHAH, RAN, ARL2, MXDI, CAT, MALSU1, COPB2, AIDA,
PRR12, TGIF1, CYB5R3, PMAIP1, DUSP4, CDK17, BRD1, ORC1, REST, MRPL28, LTBR, UBE20, YRDC, ANXA11, IDH2,
FEM1A, ALMS1, ADARB1, DOT1L, GOLGA3, DCBLD1, CCNT2, GAREM1, SBDS, EIF2S1, UROD, VAMP3, EIF3E, PNP, LSM12,
PPP5C, ZNF292, TTLL4, PDE4B, HSPA13, ARHGEF5, RBKS, ITPR3, FKBP15, GBA, SH3RF1, MSH2, TSEN15, MED13L, CDK2,
ANKRD13A, RRBP1, ADAM9, GTF2H4, SH3TC1, LMNA, ANTXR1, KDM4B, HADHA, SLC7A11, NCAPH, SLC39A8, CAV2,
SLC25A38, LARP1, CLCF1, LAPTM4B, PLCD3, MTMR12, RB1, HMGCL, CENPB, MARK4, SMARCA5, HECTD4, HMGN3,
TNRC6C, RTCB, ANKRD10, TTC37, MYCBP2, IRS2, GNAI2, NKRF, WDR19, SETD1A, CDC123, BST1, IRF2BPL, LRRFIP2,
PEPD, PSENEN, TPD52L2, ALDH3A2, SYF2, PER1, NSD2, WDR92, PHLDB2, CARS1, THOC6, DCAF5, MELK, GMEB2, LTBP3,
BLVRA, ZBTB11, CXorf56, SNRPB2, SHROOM4, AKR1C3, RPL27A, NDUFB2, NFKBIE, IER5L, ELAVL1, MMUT, RNASEH2C,
MRPL40, CXCL2, ZNRF3, DAP3, EVA1A, GPT2, PIGK, HACD3, PPP2R1A, GLG1, TACO1, LSM4, HYOU1, MDM4, PRKD2,
MKKS, EEF1A2, FNIP1, GON4L, Chsa-A, USP9X, HAVCR1, ACAA1, AP5B1, BCL2L2, FOXC1, FAM160B1, ZFHX4, DHX35,
USB1, LACTB2, TSEN34, FHOD3, CNDP2, ZBTB48, ATP5F1B, RNF38, PDLIM7, USP22, MIPEP, WDR83OS, PUS1, GPR37,
SELPLG, ZNF627, DYNC2LI1, ETV3, ZNF106, CSF1, RNF181, MRPL50, NASP, UNC45A, DDX20, PRMT1, TEF, ZNF12, DHX30,
RAB1B, FAM162A, TFAM, ZKSCAN2, KHSRP, CLCN6, GALE, HKDC1, ADAM10, RHBDD1, NIPBL, KDM4C, DDX58, PYURF,
DRAP1, AMBRA1, TBC1D8B, SLC4A2, SVIP, NDUFV1, AK3, GMCL1, COX16, DGCR8, ITPA, SUCLG1, HMGN1, ZBTB21,
MTMR3, PXDC1, SEC61A1, NFE2L1, ATP5MD, RPN1, HS3ST3B1, ATXN7, BMP2K, SEC63, TMEM63B, KIF4B, SEC24B,
TMED4, SKIL, RBM15, CALCOCO1, PPARGC1A, BAIAP2L1, UBQLN1, SPTLC3, EOMES, ACTR6, UCKL1, PPP2R5D, TMEM67,
TTC7A, COMMD10, OGDH, GAREM2, TXLNG, ADNP, CDC23, PCDH7, USP14, DYNC1LI2, FKBP8, APOBR, NDUFB7, SETD4,
ZNF441, B3GALT6, ZNF276, TOX4, ZSWIM6, ZNF629, SNW1, NDUFAF3, TGFB1, LPCAT4, VPS35L, TIMM21, YY1AP1, MRPL9,
RASAL2, OTUD7B, WDR5, PMPCA, NREP, SERPINA1, IQGAP1, SLC39A7, ERCC6L, BET1, NEK2, SCYL1, MIER2, PRKACA,
NCKAP5, ATAD2B, RECQL, TRIM8, TRAPPC6A, ZNF473, PON2, RNF168, LRFN4, DCAF13, PCNX3, PBX2, TRA2B, MRPS25,
FOSL1, NUP88, ZNF74, STRADA, RNMT, NELFA, RSC1A1, PLS1, VCPIP1, PPM1G, HNF1B, PRRC1, FLRT3, POLR3A, METTL9,
CNPY2, ZNF484, MCCC2, CLASP1, QSOX1, VAMP7, PARP6, RABL3, ZC3H12C, RABGGTB, TNIP1, SPATA2, CPSF1, STARD9,
ABHD17C, CDK19, ZC3H12A, FBXO9, TNFRSF1B, HSD17B10, AOX1, TMEM168, YBX3, RALY, ZNF764, MAPKAPK2, TRAP1,
SOCS6, PFDN2, TNFAIP6, ZNF496, PAN3, KDM5B, MARCHF7, B4GALNT4, CLU, PHLPP2, RAB11A, ASB7, ATXN2, FIZ1,
TAPBPL, SEC11A, EIF4G3, STT3A, OSGIN2, FBL, PPIF, ABHD17B, CLCC1, MRPS22, NDUFS3, SCAF8, PIGF, ELP2, AKR1A1,
GCH1, GTF2IRD1, AHCTF1, NDUFS1, ECH1, PNPLA8, LPIN1, DPYSL3, IFT80, BUD13, HIBADH, MED9, WDR77, FNDC3A,
CNOT1, ATP6V1F, SNF8, IFIT5, MTREX, SDF2, YARS1, TTYH3, FBXL18, PSMD13, IPMK, PLK3, NPM3, MICU1, UBA3,
ANKRD28, SIPAIL3, RPA1, SLC20A1, HOOK1, SKP2, ZNF689, PMEPA1, MANF, MFN2, FAM3C, ATP5PF, RSPRY1, MICOS13,
FIG4, TRPM7, GRK2, ZNF317, ASPH, TMEM50A, HACD2, CCDC47, TUBA4A, MSL1, PRDX4, MAP3K4, ITPRIPL1, ANKRD33B,
ING5, COMMD9, FKBP5, NCSTN, DVL1, SMS, KLF4, NXN, TIAL1, ADH5, ESPL1, NR1D2, NFAT5, SNAP23, FH, AP1G1, IFNL1,
NTN4, PARP12, SNRPD1, ZBTB8OS, FTSJ3, SPDL1, NQO1, KLHDC3, NSF, GLA, GATAD2A, HDLBP, C16orf70, CHD8, MRPL48,
IFNGR2, TBC1D10A, CREB1, ASRGL1, DCBLD2, TSPYL1, ZNF668, PIKFYVE, AURKB, IRF3, TMEM19, FUBP1, CA2, ABCB8,
UBE2R2, ESRRA, GINS3, MCL1, PPP2CA, RPS6KA1, CENPW, EFEMP1, MRPS15, GALNT2, CREB5, WDR54, ZNF768, VEZF1,
BICD2, GRAMD4, RPS6KA4, COL27A1, KIF2C, TGM2, PSMD8, ZNF2, POGK, MAP3K8, RPRD1B, LETMD1, SBF1, WBP2,
PHLPP1, ZNF697, CEP95, RMC1, TSC1, NDUFS4, FGD6, TANC2, SAMD4B, UBE3C, ANAPC5, PARP4, C6orf136, HNRNPH3,
DDRGK1, TBC1D14, KIF13B, TTK, WDR74, UQCC1, ENC1, ERGIC2, TRAF3, POLR3H, LRRC42, PEX19, STK17B, VMP1, GPX8,
MRPS23, FAM13B, LONP1, STX1A, ACTR1A, PCCB, RPUSD3, SYMPK, PRPF6, KCTD20, LRP8, CBX1, RPUSD2, RPL5, KDM6A,
DUSP7, CSNK1G2, USP24, SUN2, TRMT44, RPS6KC1, ZNF654, USP37, KRT17, DPP9, SF1, IGFBP2, CWC25, CLK3, CISH,
CRTC3, STUB1, GART, MICU2, GINS1, SGCE, TAPBP, PRKACB, RARA, ATF7IP2, CAPN2, ADGRG6, LRRC24, MED29,
FBXO45, NR4A3, CARD10, PTGES2, MYNN, YIPF5, SLC36A4, MIGA1, DNMBP, CDK4, SMURF2, RRP8, PHF2, PET100, CRY2,
CAMSAP2, KIAA2026, PREX1, INTS11, MAML1, FAR1, CACTIN, SMARCA1, RCAN1, GEM, KLF7, TRIM52, ZNF202, KIF15,
PDZD11, PCGF1, CKAP5, HMMR, CALM3, PDE6D, C4orf3, MRPS14, ARRDC2, LPCAT3, PLAUR, PICALM, CAPZA2, DPH3,
TGFB2, AJUBA, MET, KANSL2, NMT2, RAB18, HS3ST3A1, ASAP1, DYNLRB1, PATZ1, SERINC3, IL4R, RIPK1, AP2A1, CDT1,
ZNF598, EBP, ACACA, VPS28, ATF4, BPGM, SPOCK3, TBX2, MTF1, CDC26, ERP29, RNF138, HDGF, TYMS, SENP7, WDR48,
IQGAP3, ATP6VIB2, MAN1B1, BCOR, NAA15, ZSWIM4, PLEKHA5, CCNL2, IPPK, BAMBI, KHDRBS1, ELF3, AGO3, KIF14,
ENTPD7, ANKRD18B, CCDC115, PRICKLE2, DDX39A, PGLS, SERAC1, AMFR, EYA3, DSP, ACVR1B, TM2D2, MED6, ZNF281,
RHEB, ZBTB49, ARL1, FAM222B, NDUFV2, MIER3, MAP7, SNRK, STXBP5, GPN2, ACO1, RALBP1, PDCD10, LTA4H, SCG5,
ZMIZ2, FNIP2, TSPAN12, LRIG3, FN1, BICDL1, NFU1, TXNRD1, ALKBH4, CCNG1, MTG2, ELF4, HAX1, PLEKHA4, CDCA5,
ANKRD9, LACTB, FLNA, MBTPS1, IRF9, REXO4, ZNF394, PIH1D1, PLEKHM1, COX7A2, UBAP2L, INO80D, SNRPD3,
RNASEH2A, MARCKS, MEA1, ISOC1, CCNQ, WBP1, TST, SH3D21, QDPR, RAP2B, PRICKLE1, TFPT, ENPP5, TDP2, PRMT2,
AGPS, MAP7D1, ZNF609, NKTR, VPS4B, ZNF250, KNTC1, UBA5, RSBN1, TOMM7, CIT, ANKRD1, DTX2, MTMR4,
TOGARAM1, FCHSD2, CAV1, GJC1, CXCL11, LAMTOR4, CHMP2B, GPCPD1, HNRNPUL1, OPA1, KIF13A, NUP153, ZNF543,
CDC37L1, SPRYD7, ZFYVE26, ADAMTS9, QSOX2, ACTR3B, IPO7, ZHX3, ISYNA1, UBAC1, GAB1, KIF23, DDX41, MIEF2,
SUZ12, TADA2B, CENPN, TRIP12, KLHL11, SLC31A1, ERAP1, YIF1A, COLEC12, DNAJC3, TXNDC15, SPINT2, NOTCH2, EPN1,
ORC3, TSPAN6, DNAJB6, PREB, ZCCHC17, ABAT, CEP63, PEX3, USP6NL, KIAA1549, VPS45, EGR2, EFR3A, ANKRD13B,
ARL3, KIF3C, RAC1, CYB561A3, SHROOM2, PSMC5, ACADVL, FRMD4A, HSPA2, MAD1L1, CYBRD1, CDH6, ACVR2A,
C17orf80, LRRC8A, GPI, DPM3, SETX, NXT1, ANAPC16, ZIC2, FAM234A, NCOA5, PLXNA2, NDUFB10, NCKAP1, FBXO3,
PACSIN2, CACYBP, TUBB2A, COPZ1, LITAF, CRCP, HINT1, RAB6A, PPP1CB, AHCYL1, JMJD1C, DDX42, GGCX, KDELR2,
ZNF207, ZBTB24, EIF3B, TPMT, ALDH16A1, KLF16, SDF4, TP53BP2, SLC8A1, CNIH4, NR2F2, ZBED6, BIRC3, PPHLN1,
RNF169, CORO1B, SNX3, MAP1S, PRPS1, KIF1A, UQCC2, DCTN2, TOP2B, FOLR1, PLEKHA7, TIMM23, TCF25, WDHD1,
EIF2B3, MED12, CEP152, PARP14, MIOS, RNF103, GTF2H1, SLCO5A1, AMZ2, TBCID15, CLK2, PITPNM2, CMPK2, DENND5A,
CHMP4A, SOS1, CD320, NDUFA2, SNRPA, ZNF282, NAF1, FXR2, KRT10, NIPAL3, BAG3, HLA-DMB, ANKRD11, RHOBTB2,
ANAPC13, EEF1D, ZNF561, RAPGEF1, ATG4C, IDH1, TCF20, ANK3, NPHP1, DENND4B, SEPTIN7, ZNF710, BCAP29, DHX36,
ASAP2, TRIM13, MPV17, FLOT2, NAA38, KAT8, CNPY3, SLC12A4, PPP1R26, TERF1, PKD1, SLC35B1, ALDH1A2, EREG,
KANSL3, LDB1, MLLT6, SP3, ERG28, E4F1, KMT5B, MIEN1, TNFSF15, ATP2A2, R3HDM2, BRD7, PCK2, NFKB1, HSD17B11,
BSDC1, USP11, PHF20L1, SERPINF2, ZNF318, ERI3, STAU2, CDK18, MRPS5, HTT, FNBP1L, MSANTD4, IFI44L, SRSF4, DCTN3,
INAVA, KDM7A, KLHL26, PHF6, VEGFB, SH3PXD2B, PRKAB2, UNK, GBE1, SIK3, ATF6B, ISCA1, DENND5B, UBE2I,
PRDM15, SELENOO, MAPKAP1, IQCC, RHOT2, RAD21L1, DDX17, TRMT1L, PFDN6, SATB2, ZUP1, VAMP8, RFC3, ADGRB3,
HGSNAT, SLC39A14, GTF3C4, ITSN2, RSRC1, AP1M1, ZNF823, SLC23A2, SLC35A1, MRPL16, PTPN21, CKAP2L, CRYBG3,
FLOT1, MLEC, CASP3, NSDHL, CCDC25, INTS4, MOSMO, RELB, FGFRL1, SEC22B, FBXL7, SF3B5, SH3D19, RC3H2, HSPG2,
PRPF39, GLDC, ATXN1L, INPP5E, RICTOR, SOBP, MTCL1, YTHDF2, TCP1, TMEM33, RIPK2, SEC62, COPS2, C5orf15,
CHAMP1, TOMM6, AP3B1, HP1BP3, AKIRIN2, ASCC1, DPP4, ACTR1B, GDI2, REPIN1, CPPED1, TENT5A, ZNF672, CYP17A1,
SRC, CINP, CD109, FKBP10, HIRA, GCSH, SENP5, EFHD2, CMSS1, GID8, NT5C2, TRIM44, TM9SF2, HPRT1, TIPIN, SEMA4C,
ZC3H7B, AP3D1, MAST3, IPO5, KIF21A, PFKP, PLA2G4C, BICRA, HECA, ITGB3BP, PPARGC1B, MAFG, TTF1, SCAF1,
TMEM201, CTH, RALB, NUCB2, CENPK, TASOR2, AGPAT2, GLB1, GMPR2, POC1A, SESN1, HMCES, LBR, RBX1, CCT3,
LRRC40, MRPL51, G6PC3, H2AX, SCAMP1, NSL1, PLCG1, CTTNBP2, ANKH, C2CD2, SIPA1L1, PCNX1, GDAP1, DIPK2A,
SEL1L, OTUB2, FAM171B, GRB2, DCAF12, TSPAN14, NUDC, BIRC6, PODXL, NUDT22, DCP1A, MTX2, ARHGEF17, CBLB,
CDC42, WEE1, PGAM4, SLC25A24, COQ8A, OGT, RIN1, APOBEC3C, PRKAG1, TMX1, NCF2, TAF1, PRDX6, PSMD9, DAB2IP,
GADD45GIP1, IL17RA, NAGA, PPIE, UHRF2, PDE7A, RNF121, RFC5, SNX10, RAPIGAP, KSR1, LRP1, CPA4, MTFR1, ACOT13,
MCM9, SNX18, RPLPO, UVRAG, TFAP2A, ATP5MC2, ALKBH3, RILPL2, UAP1, ZFYVE9, CNTFR, SRP68, SIN3B, SMARCE1,
LSM6, FAHD1, ARVCF, SLC13A1, BUD31, MMP14, WWTR1, MRPS24, UGGT2, TBC1D23, CISD3, FAM114A2, MAZ, ABHD15,
SCARF2, SPOCK1, USF1, MLXIP, SLC38A2, SRR, MMD, ASAH1, PROSER2, MLH1, MARF1, LANCL1, VCL, BCAT1, IFRD1,
TONSL, SLC25A30, NDUFB11, NOB1, HSPBP1, VSIR, URGCP, TMEM258, HBS1L, PLXNB2, VPS37B, KANSL1L, OGDHL,
TOPBP1, DUSP10, PGM5, DNAJC19, MAML2, TNS2, LAMTOR2, SALL2, CDKN2AIP, PFDN1, SNRNP48, SPPL3, CNOT7, RNF5,
NBL1, KNL1, POR, HDGFL2, RLIM, C15orf61, MYBBP1A, MGAT1, SGK1, MAU2, MACROH2A2, HERC2, ALG8, PMVK, ECPAS,
CPSF4, AEBP2, MKI67, SNRPC, ZNF367, SH3BGRL, ECT2, ATF6, LAMB3, ARHGAP35, PUM1, SOCS3, TUT7, ZCCHC8, PSME4,
EZR, SF3B2, KPNA1, LAMTOR5, SEC11C, LARP7, CYP27B1, SRRM1, MOCS3, BBS2, SLC38A9, DARS1, PIGT, TMEM170A,
ABHD17A, FAM53C, KIF22, SLC39A6, SSNA1, MEX3D, ZNF263, XPO6, RACGAP1, RFC4, GSPT2, COPG2, COX17, MCU,
CEP250, PDIK1L, CDV3, ADAM15, FKBP9, SF3B1, NOSIP, SLC9A3R1, ANXA3, RNFT1, MMP7, TRMT10B, TBC1D22B, ZBTB39,
CNST, BRWD1, NEPRO, BCAR3, HCN2, KLHL28, ANKRD52, DNAAF5, NBR1, CENPI, CTSC, GPKOW, SIMC1, ZNF565,
DGUOK, ZSCAN22, KLHL22, ERBB2, TMEM189-UBE2V1, GCN1, ATG16L1, ITM2B, MFSD2A, BYSL, SEC23A, METAP2,
ANKRD13D, CDC6, GIT2, KIN, BCL3, ZNF526, CHMP2A, DCUN1D3, ADAM19, HMGAI, LCMT2, CSNK2B, PRMT3, WRN,
CNOT2, STAT6, TNPO3, E2F6, TLK1, HELZ, FAM135A, CAP2, GLRX2, ELF2, SLC39A3, WIPI2, CHSY1, ZNF180, CLIP2, CDON,
METTL7B, NFS1, CCDC174, BPTF, SLC38A10, FMC1, CBX6, DSTYK, PLEC, FAM32A, CTNNBL1, MECR, M6PR, TIMM44,
HAT1, REPS1, WLS, DDR2, SLC6A13, CSPP1, GATA6, ZNF732, ING1, SNRPN, CENPA, GRPEL1, GNG11, SCAMP4, VPS13B,
TGFBR3, ARHGAP11A, TNPO1, PDK2, SUPT4H1, TRIR, PCDHGB7, CSNKIG1, GFPT1, TFB2M, ARF1, CD82, DDX51, FZD5,
RASL11A, SMAP1, ACE2, GCFC2, RER1, BTD, TNRC18, BRF2, LRCH1, PSMD3, EPHX1, NFX1, TOP3A, IMPAD1, RDH13,
PTPN23, RTTN, NEMP1, GPATCH2L, SERINC1, SNX17, MGAT2, FST, HOMEZ, PHKA2, OGFOD1, LLPH, AP1M2, ATXN7L2,
IKBIP, FOPNL, PPIH, ABHD10, FBXO38, CIZ1, MSX1, CKLF, PGM3, PI4KB, KRI1, RIPOR1, RSL24D1, FOXO3, NAA16, VSIG10,
ERCC6, TMEM98, DIP2B, SEC61B, CCDC167, USP3, NAA50, RANBP9, LUZP1, MTSS2, RASA2, WDR11, TRAF3IP2, PRPF4B,
SWI5, IGF2BP3, ZWILCH, GOLGA7, PAXBP1, CPNE3, MAT2B, RRP1B, RAD51AP1, NAPSA, GINS4, WBP11, ZFP36L1, PLA2R1,
TTC38, KARS1, KAT2A, PPP1R3F, RILPL1, TRIM33, NDUFS6, RCOR3, MPZL1, ATG2B, EIF3K, HDDC2, MTRR, MBD5,
CYSTM1, ERLIN2, GAPVD1, PNPT1, ZNF622, SIVA1, SSR1, BRF1, ARF6, TENT4B, C19orf48, CPSF6, NOTUM, OAS1, ZBTB7B,
ZNF16, TESK1, COP1, UHMKI, CASP6, WDR43, GTF3C1, GJB4, TJAP1, RBM26, VPS39, PARP10, BSN, SKIDA1, TBX20,
TOMM34, CASP8AP2, SMIM30, MRPL3, ABCC9, CD3EAP, COPS4, GCC1, PPIP5K2, RBFA, SMARCA2, C15orf40, HEXB,
SERPINB1, DNM2, FPGT, ZNF500, RAB2B, VEGFC, TMEM248, SKA1, COMMD6, SLC35F2, ITPKB, PHPTI, PBLD, AKTIP,
SIGMAR1, FSTL1, EEFSEC, ORMDL2, AAGAB, STMN3, USP15, CPNE8, TOPORS, CDKNIA, KLHL15, COPS3, MCRS1, NXT2,
COX11, SIRT1, CEMIP, MIER1, GRINA, TADA3, FAM131A, ZSCAN2, EIF2B4, DHRS1, UBA6, MTHFD1, PRDM2, GPS1, IPO9,
CSTF1, C12orf57, IP6K2, RNASEK, TMEM69, TRAFD1, GSE1, SEPTIN11, H2AJ, MGST2, TSPYL4, GLBIL2, ZBTB40, ZNF121,
TMEM167A, MAP3K13, GALNT7, SOCS2, THRAP3, STX12, LRR1, STXBP2, GATA2, TGS1, LYAR, DDX24, ALOXE3,
SELENOT, COPG1, SIRT3, CIS, HMGN5, FLNC, XPO5, RNF214, GRB7, TSFM, TAF10, UBE2NL, ANKRD46, FITM2, TECTA,
JMJD8, PLRG1, SAMD8, KCTD9, DCAF11, YEATS2, NDUFAB1, GAS1, CCT4, JUP, ZNF507, PARP2, RPAP3, IRF2, NSFL1C,
NDUFA10, HTATIP2, PMS2, PTK7, NOL9, MGME1, CAPN15, ARHGAP39, DTX3L, ITFG1, TGFBR2, MEGF8, PGP, PARD3,
MTX1, C2CD2L, NAA40, GON7, FSCN1, SNCA, SNX1, TACC3, CREBZF, PFKFB4, ARAF, GSTCD, CLPTM1L, RBM6, PHACTR2,
CTIF, SLC38A1, ALKBH5, MPHOSPH8, OSBPL5, RAB24, TIAM1, RAD51C, TSG101, RAB21, MBTPS2, TLE3, ITGAV, NF1,
METTL21A, MRPL22, LAMP1, PARP9, SNRPA1, DEAF1, IWS1, SRF, APOBEC3F, ASF1B, SUCLA2, AVL9, MORC3, ATRIP,
GULP1, COL8A1, TTC26, PROS1, HSPH1, SOWAHC, SPTLC2, ZBTB37, BCAT2, KIFC1, DIS3, NACC2, CEP76, MTHFD2, PSMB8,
TRIP10, IFFO1, ATXN7L1, FAM174C, GMPPA, GNAI3, CPNE1, ZNF687, SCAP, ITPRIPL2, BASP1, GATD1, TMEM14A, HAUS1,
NANP, CAMSAP3, PIP5KIA, AHI1, FBXL6, ITGB1BP1, CBX2, SPNS1, SRPK2, RSBN1L, UBR4, EXT2, ISG20L2, STAMBPL1,
RAD51, DUSP2, TMEM205, UBR1, FAM98B, GMEB1, TAF13, IFI16, GLRX3, BCS1L, PRKCE, RBMS2, UBXN1, FAM118A,
TRRAP, FJX1, TAF5L, PPCS, SPCS1, SPRYD3, CD68, C20orf194, UPP1, TAOK2, CCDC124, SDHB, CDK8, STYXL1, ATXN7L3,
ERLEC1, PYCR3, IPO11, GAR1, KNSTRN, TBC1D1, DCAF6, FBLN1, ADD1, MCUB, RYK, GOLPH3, MCPH1, HMGXB4, DCDC2,
SNRNP200, TFIP11, BNIP1, CTSB, CBR4, AIF1L, HLF, RPS7, NDUFC1, RING1, TRMT12, HBQ1, IMPDH2, RNF215, SMAD6,
ALDH4A1, SPIRE1, EDRF1, DGLUCY, JMJD6, THAP12, PLCXD2, IGSF3, CBX4, PFKL, FBXL5, RIMS2, TBCD, FRZB, GLS,
FIGN, COMMD7, BCL2L13, MAP3K2, OSTM1, ALDH18A1, NUS1, PSMG1, MAPK3, ZNF8, AKAP10, UQCRFS1, CREBRF,
ZNF236, RNF144A, ITPKC, PDCD11, ZNF800, TMEM254, DENND4C, SRSF1, MRPS33, FXN, B3GLCT, HDAC2, SIAE, NAPA,
NEU3, ASXL2, MSH6, LSR, EMSY, CCND1, IFT52, AJM1, LRP11, KCTD2, POLR3E, IRF2BP1, ADSS1, RND3, PTPN2, RBCK1,
NCOA3, RGMB, CCDC85B, CUL9, PSMB10, MYO18A, TP53, NCAPD2, SNPH, PDCD5, HFE, MMACHC, JKAMP, GTPBP4,
TPD52L1, C16orf58, METTL22, NBEAL2, POLE3, MAP3K9, TIPRL, ASB2, INCENP, MMS22L, PSMD11, B2M, IRAK2, MAPKBP1,
RAB5B, BAZ2B, INO80E, PYCR1, ARHGEF10L, ARHGEF39, CCHCR1, PTPN4, PRR5, LIG1, UBXN4, RSL1D1, FECH, GALM,
MAP4K3, PSMC4, TOR4A, MCM10, TMEM214, MAP4K4, PTRH1, RPS6KA3, AMOTL1, CWC27, VAC14, AHDC1, COMMD1,
PHF23, BMP1, NSMCE1, SRSF2, LYPLA2, CYB5D2, EMP3, ZCRB1, CRYZL1, TSPAN4, WDR91, EPC2, PACS2, ST6GALNAC4,
EMC10, CUL4A, ALDH5A1, TENT5B, SGCB, SPSB1, KDSR, NUDT9, ZNF114, CLDN15, OGFRL1, UCHL5, ZBTB34, THEM4,
LRRC14, CXCL1, ZNF805, TRAPPC12, SMAGP, RBMS1, TIGD2, SMNDC1, CDCA2, RNF167, MORN2, CCDC142, ALG5, ITPK1,
CDC16, TMEM236, EXOSC1, NOL7, TICRR, BUD23, STOML2, HMG20B, TTC28, SZRD1, IDE, GPATCH1, CD151, LEPROTL1,
CPD, PPP2R3C, BRD8, SMO, OAZ2, SFMBT1, CIDEC, ABHD4, SH3BP5, NT5DC2, CLIP4, RPGRIP1L, TTC5, SPCS3, MPP3,
TMED7, MED17, PDCD6IP, TRAPPC3, ATG14, METRNL, CTSO, MESD, RBM19, AGA, CCDC22, SH3BGRL3, RGP1, UTP3,
AMACR, NPC1, ZNF330, SLC25A5, PTAR1, RRN3, RSF1, PRPF19, SLC25A28, EPB41L3, MCAT, WDR41, FHL2, CERS2,
FAM120A, USP34, TRAM1, CS, MED12L, KAT14, CAND1, DCDC1, DICER1, ZNF614, MCRIP1, GNPTG, HDAC7, INSIG1,
PTCH1, RMND5B, ADGRL1, LRP4, TBX3, S100PBP, COA1, KBTBD2, MBD1, KPNA6, WTAP, RWDD2B, DONSON, ZNF555,
PIAS4, IFI27L2, ZSCAN20, TXN2, NAE1, YIPF1, CSRNP2, PAIP2B, CCDC34, TPGS1, EFNA5, LAMP2, SLC2A2, FAM20B,
CSKMT, TPST2, SORT1, ZNF581, IFT81, BROX, KEAP1, ZMYND8, TNIP2, CAMTA2, NEK1, SEMA4B, TOMM70, ALYREF,
NAGLU, PRPF8, GSTZ1, HOXA9, CHAC1, DSEL, GABRA5, WASHC3, COL5A1, TXNDC17, MKRN2, FBXW8, D2HGDH, PHC2,
GFM2, MRPS12, DYRK2, IGHMBP2, TRAPPC8, TM4SF4, GTF2I, ADSS2, HOXC10, SUMF2, SULF2, NPEPPS, DNPEP, ARHGEF9,
ANO5, ITGB2, ATG101, NR6A1, PRR36, RANBP2, BRSK1, RHOA, RETREG3, PHYH, EXOSC4, KRT80, PLBD2, ALG11,
MOSPD3, AP2B1, MRPL53, OSBPL9, RHPN2, MAP3K5, SLU7, EIF3I, NUDCD3, MED15, TRAF5, PSMD12, CIC, ASH2L, ZNF14,
RNH1, TARBP1, SPR, PPP6R3, P4HA2, CLUAP1, ZNF547, ARID5A, NEK7, ZNF777, PTPRK, DHX33, RPP38, TRIM26, GIPC1,
CHCHD6, CD55, POLR3B, CCP110, NTPCR, PHF20, TUBAIC, UTP6, MAP2K4, GALNT10, FAM216A, MECP2, SH3GLB1,
SLC9A1, NUP62, PAPSS1, TTC39C, SLC19A1, CBARP, FAM20C, DDB2, TOR1AIP2, APBB2, LIPA, MRPL2, CHD9, VPS18, GPN3,
ACER2, NR2C2, JARID2, ZNF304, WAC, SERTAD3, MRPL39, ZNF513, GSKIP, BMPRIA, SLC7A6, RAD23A, SECISBP2L,
TWSG1, ZNF529, FAT1, SNRNP27, DENND1B, SHLD2, IRGQ, PLEKHN1, MLYCD, MAPK8IP3, SPRY1, GPBP1, CSTF2,
AURKAIP1, RCC1L, SH2B1, DCTPP1, KPNB1, APOBEC3G, PDCD7, ABCC5, BSCL2, PIK3CD, MRPS34, ZSCAN21, UNC119B,
PRADC1, DZIP1, GLRX5, COX15, BEND7, SPAG1, GTF2A1, EXOSC9, PLEKHA6, CCDC103, ACAT1, TCEAL4, SKA3, PPDPF,
ZNF652, REEP5, ZNF621, ZNF579, IRF7, EEF2K, TCEAL9, AP1B1, CCDC58, ANO10, ZFP3, GLB1L, MYEF2, NUDT5, PLPPR5,
CBR1, ZNF703, PATL1, CMTM6, RMI2, HERC1, DOK4, PLEKHF2, ATP5F1C, TMOD4, CCNA1, HEATR9, KIF7, ZNF70, RBM42,
USP53, ATP6VOC, GPN1, EDC3, NDUFA12, RASSF1, DIAPH1, NUBPL, PJA2, UGT8, NCK1, TJP1, RIN2, CARD19, C1GALT1,
PLGRKT, FEZ2, MXD3, MOSPD2, TMEM185A, FILIP1L, MAD2L2, RHOC, ZNF620, CFH, ZHX2, POLB, NRDE2, PARN, MRPL36,
MTF2, RBBP8, CACNB1, NUP42, FABP4, HERC4, REEP2, ACOT7, DIPK1B, IFT27, NELFCD, C1R, MRGBP, AGBL5, MTFR2,
CTNNAL1, MGMT, DCTN5, NF2, TCEA1, SYDE2, COMMD8, HIC2, SCARA3, STRAP, B3GNT2, ARHGAP12, KCTD6, FAM8A1,
NME7, SGMS1, RBM17, RBM23, ATG12, TBCK, ECD, EMG1, NDFIP1, OXA1L, KIFC3, ZNF205, MTMR6, ENO3, ASH1L,
RNF19A, GMFB, STAM, WWC3, ORAI2, BLVRB, TMEM140, DNER, SS18, XPOT, UBE2B, DBF4, AUNIP, PER3, PRR14L,
ACAP3, TIGD1, TXNRD2, WDR26, NUBP1, ATP5ME, AGK, CDHR2, INTS6L, SLC29A1, LGALSL, DELE1, CLPB, ADAMTSL3,
GOLT1B, PPP6R2, ZNF436, WDCP, WSB2, NPTX1, UCHL3, TPK1, SLC12A2, TOB2, CCDC157, ADAP2, ZEB2, SLF2, EIF3L,
ARFGEF1, NANS, ZFP1, LAP3, FANCE, MTR, CYLD, NUFIP2, ILK, ABLIM1, PANK3, NCBP3, PLAAT3, SCO2, NUFIP1, NCOA7,
COPS7A, XPA, RPA3, KLF15, PRKCZ, STRN4, AIMP2, MYL6B, SRSF7, CMTM3, TRAPPC1, ZER1, TERF2, FARSB, LIMD1,
PRR3, CFAP36, GFER, XRCC4, PABPC4, TMEM63A, NSUN5, ZNF639, NEK3, TCTN1, TRMT61A, NR2F6, C3orf38, ESYT1,
SMCR8, TBC1D20, CHST3, CXCL3, SEMA4D, DDA1, PPP4R3B, ZNF333, ABRACL, ZNF41, FUT4, SF3A1, RBSN, UNC5D, DBT,
C7orf26, CCDC62, CAPRIN2, NIBAN1, ENOX2, SPTLC1, PXMP4, CYFIP2, CLDN12, TNFRSF11A, LCAT, N6AMT1, PPP1R14C,
MED1, CRYAB, SIAH2, CDR2, STOX1, NEMF, PARPBP, DAPK3, KLHL36, ARHGAP30, CFDP1, SCRN2, TNFSF10, ZNF77,
IRAK1, AUTS2, NR4A1, ZNF554, GSN, ANAPC1, DUS4L, ELOF1, UBE2J2, ZFAND3, ZMYM4, MAGI1, FAM126B, FNBP1, CHD5,
KPNA5, CHMP4B, BABAM1, DGKH, UBE2K, CPM, UTP11, TTC17, DUS3L, TUBE1, FAM229B, CEACAM19, ARV1, TNFRSF9,
STAM2, MINPP1, COQ10B, GTF2E1, DNAJC11, PBDC1, TUBD1, TIRAP, DUS1L, DPY19L4, INPP5B, SFT2D1, INTS3, VCPKMT,
CREB3L4, JAK1, IFI30, CPT1A, PRPF40B, TRIM56, STK24, VPS72, DEPDC1, GSK3B, USP1, TRIP13, ETNK2, PCGF6, PNO1,
APBB1IP, SESTD1, CARS2, TFDP2, SASH1, SWAP70, RABGAP1, ATP6VOA1, INTU, H1-0, SUGT1, NCK2, NEIL3, AACS,
ABCA4, GTSE1, TRIM23, PWWP3A, MAN2B2, OXSR1, REEP4, CBX5, NTS, SPTAN1, CCDC82, SURF2, GLMN, TRIM11,
TECPR2, EIF2B1, SNX25, KLHDC10, LMO7, PRKCI, TYW5, UNC119, NKAPD1, MGAM, NOP53, ZNF184, RAVER2, TRIQK,
CNOT11, USF2, DENND6B, LTO1, B4GALNT2, TTBK2, H2AW, GSDMD, COQ8B, FBXO21, MUL1, WDR37, TFG, PMM2,
ACTR3, NAPRT, WHAMM, PLCE1, TBPL1, C2orf42, ZSWIM3, FDXR, SOCS7, PIGG, USP21, EGR3, MRPS2, IST1, NGRN, PDXP,
APRT, DKC1, AFTPH, USP7, CMTR2, CALML4, BTAF1, SLC17A1, ERI2, CC2D1B, DYM, MYO5A, PIK3R4, UBALD2, INTS10,
S100A13, TMEM182, GFM1, CILK1, TMED1, ABHD6, DCTN6, MAPK1, TTC39A, PAAF1, VPS36, RPRD1A, SLC6A12, PAFAH2,
ANKS1A, CNKSR3, TRPM4, PRKAA1, COPB1, ISCU, PTPN3, S100A6, ABCA2, ZBTB9, APPBP2, FAM102A, SALL1, CSK, HCN3,
KLHL18, PTPRA, FAU, HK1, MOB2, RCC2, AAK1, N4BP3, THAP4, ZDHHC20, PSMF1, TLNRD1, TFAP4, SF3B3, TMEM126B,
AATF, ETFDH, CCNY, BAZ2A, KLHL12, MNAT1, CCDC117, TLR3, DZIP3, LIPE, BRWD3, GLCE, TCTN3, GNE, ZMYM2,
CEP192, NDUFS7, PLEKHG2, KLHL24, ARFRP1, IMP4, LDOC1, MFSD3, KCTD3, NRDC, GOT1, WDR61, POLE, PTGR1, JAK2,
TMED5, FBXO34, SPG7, CEP55, TMEM11, BBS9, UTP14C, TMEM222, UBP1, PTPA, PTPRE, TRIP6, NABP1, VHL, TRAF2, LYN,
BACH1, NAP1L3, UHRF1, SRGAP1, SUMO3, LGR4, KYNU, OTUD3, TMEM54, SFXN3, EMC3, AQR, TMTC4, MRPS10,
B4GALT7, EBPL, THSD4, PLEKHH2, ARHGAP29, CTU1, RPUSD4, ELP1, SDHAF2, LAMB1, HBP1, NFRKB, AGPAT1, YIPF4,
HYAL1, ARMC7, USP47, PDP1, FRA10AC1, MMADHC, SDF2L1, HIBCH, SGSM1, NEFL, AXIN2, C18orf54, MTHFS, NDUFV3,
LZTS1, CCDC198, MBLAC2, ORC5, AGRN, SERTAD1, AP3M1, ALDH7A1, OTUB1, PRKAR2A, TSTD1, TRAPPC4, WDR81,
RBM10, JRK, BBS4, ZNF597, ARHGAP21, C11orf49, JOSD1, CCDC91, ANP32B, ZNF821, ABI1, CYP1B1, SLC12A9, HTRA1,
RAB29, C11orf54, PEX2, MAMLD1, SLC25A22, TRNT1, THUMPD2, ELAC2, MSL3, PDE4A, CCDC120, POP1, XPO4, DNAJB2,
IDNK, KRTCAP2, B4GALT3, HIPK2, DDX27, RTN4IP1, CLASRP, SETD1B, SLC45A4, CEBPB, BTC, TPM4, COBL, GBA2,
HECTD2, ARHGEF3, UPK1B, PABPN1, ZNF107, MTO1, GNB1, ING3, GPD2, MED21, KIAA0895L, CHP1, EML4, SNX5, CHDH,
IP6K1, RETSAT, TMOD3, ZBTB5, FUCA2, MRPL41, KPNA3, HSDL2, PLEKHH1, ZBED5, C8orf33, CYP2R1, TOR1A, PDCD6,
C19orf53, PBXIP1, KIF26A, PORCN, NSMCE4A, UBL4A, RTCA, GFAP, RNASE4, MADD, PSMC6, BCL9L, STT3B, SH3BP2,
CLNS1A, HARS1, SMPD4, SESN2, TRPM5, ARHGAP26, XPO7, HSPE1-MOB4, SLC5A11, FBXO30, EEF1G, KDELR1, ASF1A,
C5orf22, IGF2R, ZNF341, PIK3CB, LRRCC1, PCMTD1, SMARCB1, FAM83D, FBRS, MRE11, TCTN2, TWNK, UBR3, ELK3, TPP2,
RIOK1, NT5C3B, NEO1, ZNF628, ZC3H18, ZNF219, LRRN4, FAM184A, MANBAL, ANXA4, CBX8, REX1BD, RAD52, SLC6A15,
C5orf51, NFKBID, ZDHHC3, SLC7A13, TNS3, CYFIP1, MAPK8, DACT1, TSKU, SBF2, VGLL3, PUDP, ILKAP, ECM1, ANXA7,
FCAR, ZFC3H1, NPR2, STAT2, DESI2, FAM210A, DERA, CCT5, UBE2Z, SERPINB6, BTG2, MFAP3L, EVI5, SNX6, BCR, SAXO1,
NSG1, PSMG3, RETREG1, TMEM51, WTIP, KIAA1143, NCBP2, HIVEP3, LRRC49, GOLGA1, HCFC1, RFWD3, RFXANK, BEX4,
SAFB2, GLTP, TAF12, SPTY2D1, SLC35A5, DCK, KRT83, PURA, TPGS2, TP53RK, FTCD, DISP2, FTO, CIAO2B, MAPK1IP1L,
SPATS2, MAPK7, HDAC4, TLR2, HASPIN, ZRANB1, ADK, ANO3, MISP, GRSF1, TBL1XR1, GBP1, ERI1, ZNF445, ASB1, SNX16,
CTTNBP2NL, REV3L, TNFSF12-TNFSF13, TMCO3, KDM5C, SYT17, VAPB, GALNT18, TAGLN, GPSM1, HROB, POLR1A,
SURF4, TMBIM1, LAMC2, DAP, GPATCH3, CCS, HPF1, BRD9, STX8, ATP6VOB, LLGL1, CHRNA5, ZNF451, KIAA1671,
MPV17L2, GIGYF2, SOS2, PHF13, PHOSPHO1, ZNF813, IFNGR1, PIGU, AKAP7, BZW2, TRAF7, SMIM19, LY6E, APBB3,
SUMF1, GSTM4, CCT6B, LRRC25, CYB5R1, SSR4, CCNE1, HOMER1, WDYHV1, ASL, MSANTD2, SPTSSB, PCDH9, IGF1R,
MON1B, ARHGAP5, SLC25A12, NDFIP2, RMND5A, MANEA, F2RL2, LEPR, YKT6, FCGRT, RAB3GAP1, NENF, SCFD1, ACSF3,
HSBP1L1, TMTC3, CASC3, ANGEL1, ZFYVE27, SMAD1, SCAPER, RAB33B, CENPF, GMNN, FAM160A1, NEDD4, CKAP4,
SMC1A, CYTH3, NOX4, BTN2A1, HSPB11, TEX10, FAM91A1, PA2G4, MYLIP, DOCK9, PSMC3IP, FBXO15, TNNI3, RPL15,
TNFRSF10A, BAHD1, BABAM2, FASTKD2, TRIM35, NLRX1, CD99L2, NCAMI, KLHL3, BMPR2, DEDD, ISCA2, ATXN1, ADI1,
ZNF706, ETAA1, CDK5R1, AKAP1, GCA, USP4, MRM2, AK1, TMTC2, LYSMD4, H1-10, ADCY10, PLEKHG4, AGFG1, CDC40,
KCNN1, CTPS2, AKAP12, EXOC5, MND1, MIGA2, NDUFS8, DGKE, ZNF277, TMX2, CCDC86, EFCAB2, SLC9A7, PALB2,
KLHL17, ADCY3, CAD, NTHL1, SMG7, DHX38, MITD1, TSPO, DUSP11, CTCF, ITPRIP, ATG13, CHST10, MTM1, C4orf48,
MRPL38, UGGT1, BDKRB1, CALCOCO2, PTPN12, PGBD1, ERCC4, F2, INO80C, TMEM160, ZNF664, PAFAH1B2, C12orf29,
CUTA, PWWP2B, GLMP, ZNF143, TARS2, GARNL3, XPR1, BMT2, GALT, DCLK2, CCDC112, IMPACT, PITPNC1, GIGYF1,
PID1, LDLRAP1, AIMP1, NDRG2, RBBP5, FGFR1OP2, TTLL11, AMPD2, ENTPD6, INTS7, MFNG, RPL39L, C1GALT1C1,
CCNG2, RCL1, ERCC8, HABP2, DNMT3B, MAPRE2, C17orf113, CLIC5, FER, CCDC102A, TAF4, ZNF81, SMIM1, ALAS1, LNX2,
GASK1B, RBM18, GNPTAB, GSPT1, UFM1, ZMAT1, RNF217, MRRF, DCUN1D1, C4orf33, PRR5L, NOC4L, TMEM243, NGDN,
ST6GAL1, MPP4, MAPK11, TCERG1, DUS2, ZNF83, MRPL55, ANAPC4, SLC13A3, ACADS, KIAA1958, MAST2, FBXL12,
TAF9B, BTBD2, ACP2, GFOD2, HPS4, LHPP, HINT3, NDUFAF1, LIMS1, DHRS13, RNASE10, CTNNBIP1, CDK5RAP1,
CYB561D2, CLSTN3, LAYN, BCL6B, EMC7, APEH, IFT20, CERCAM, DLG4, MTMR1, ATP6VOD1, ZFPL1, HIP1, GLIPR2, RDM1,
ZNF576, KMT5A, XAB2, ACACB, EMB, KDM2B, KIF12, GNPDA2, MYDGF, CDC42EP4, TINF2, MBD2, RANGRF, OAS3,
KCNN3, RPL22L1, UBE2T, CIAPIN1, CCNDBP1, ADCY9, SLCIA3, LSM14B, RAB20, DDX55, MAGI3, ZNHIT6, TXNL4A,
NISCH, USP39, EXOC4, VAMP5, TMEM50B, PSMD5, EPS8, TWISTNB, AMER1, ARMC10, KDM3A, SFI1, CDH2, GEMIN7,
FARS2, IPO4, ZNF415, ZSWIM8, FKBP11, PRKARIB, SPRY4, NDUFAF5, SLC3A2, GAMT, ZNF695, PIK3CA, WDR5B, ABCB10,
NIM1K, C1orf109, HACL1, TMEM245, CCNA2, NHS, TBC1D4, CFAP44, DHX8, MBOAT7, TMEM38B, EAF2, QSER1, HIKESHI,
PNPO, RBPMS, EFNB1, NCOR1, IFT57, MX1, KIFC2, EIF2A, PWWP2A, MEX3C, UBE3B, ZFP62, PKDCC, OLA1, TEX30, MAP1A,
PNPLA6, B9D2, LSM8, SMG9, IGF2BP2, DIP2A, DRAM2, AGTPBP1, GDAP2, OMA1, MSI2, ENO2, DNAJB4, MIEF1, MAN2C1,
NRTN, ERCC5, SATB1, HAPLN3, SCAND1, IL18RAP, CITED2, COA6, AMH, ATG5, BLOC1S1, IGDCC4, POLE2, INTS8, GDF11,
TRIP4, MLF1, TXNDC11, CDK16, CRYBG1, PHACTR1, POLDIP2, EFCAB6, ATP2B4, MTMR14, INPPL1, NPY1R, DFFA, NIF3L1,
AHSA1, HEXD, B3GALNT2, ERLIN1, SMAD3, CELF1, PCIF1, TCN2, MBD3, SHF, ACOT8, ARHGAP31, DVL2, PDSS2, NVL,
NRAS, CUL1, NRF1, GNPAT, SPNS2, RASGEF1B, USP46, DHX16, CPT2, PPP1R12B, ARMCX4, ATG9A, C1orf74, SPA17, PIGX,
DCAKD, LMBRD2, ZXDC, TMEM141, IGBP1, CNP, EIF4G1, CELSR3, CASP2, DPYSL2, KIAA1324, MRPL21, SCG3, STK38,
DRG1, CYBC1, EXOC7, ELOVL4, THBS3, ABCB7, GPR155, TUSC3, PDGFC, CDKL5, NAGK, SLC35C2, SNX7, SAR1B, GTF2H3,
E2F8, RNF146, ATP8B3, ABCD4, ZNF18, CENPP, PUF60, CYB5A, F8, HAUS2, DYNLL2, DAAM2, SYDE1, ZBED1, ADD3,
SCN8A, EFNB2, SNX33, RDH10, PPP4C, GLYR1, MRPL44, LENG8, NUMB, PPARD, RITA1, MVK, SPATA2L, TMEM184C,
GPRIN1, LFNG, MAST1, TMEM115, ZNF830, VAPA, ENOSF1, ILF3, MAB21L3, UBE4A, CAMLG, PLOD3, LRATD2, MEX3A,
LMF2, ARHGAP32, TRIM39, ECI2, TMEM185B, GBF1, PCBD2, PPP1R37, RO60, C8orf76, RPL32, TMEM18, PQBP1, FAM168B,
TIGAR, IQUB, FOXC2, CHCHD10, EMC1, DYNLRB2, ORMDL3, CACNA1B, CYP39A1, WDR33, PNRC2, CCDC90B, SYAP1,
TEX101, DPH1, GPSM3, AKIP1, PPIC, DLST, TDG, ARHGEF26, TAB2, MRPL18, WASHC5, CDKN3, CNNM2, SEC23B, NUP85,
CARM1L2, COX14, KCTD15, IFRD2, LIN37, RECQL5, GPR85, TRIM3, MACO1, VAV2, MEIS1, RHOG, RPP30, PDGFA,
TMEM147, RNF19B, DNAJC16, TMEM208, PTPN11, EEFIAKNMT, BBX, UBE2E3, TIGD6, OSTF1, CMPK1, GOSR1, MDP1,
UBAP1, MYLK, MORC4, JCAD, ZNF707, MRPS31, NNT, STK4, SNX24, DENND1A, CLCN2, WDR24, SPATA5L1, ITGA5, USP25,
ANKRD23, ZNF512, RPLP2, CBL, TENM3, MICALL1, FIS1, UTP23, MRPS7, TWF1, E2F1, ATPAF2, CCDC181, CBFA2T2,
ZNF564, POLD1, TM9SF4, TIMM13, FAM171A1, IFTAP, PPP1R8, FGFR1, TECPR1, STPG4, ARL10, ZKSCAN1, ATP1B1, PAMR1,
ORAI3, TNPO2, PTCD2, MTBP, MYO1D, CCDC50, DVL3, XXYLT1, CCDC51, ICAM1, CNIH1, ZDHHC13, NQO2, SOX15,
SLC7A1, VWA1, IL6, PLOD2, KBTBD4, DCAF8, MYOF, CELSR2, HOXB7, F1IR, CASKIN2, MMP13, ZNF133, TLE1, RBM45,
C9orf78, NUDT16L1, TEX2, RABEPK, ATM, ATP6V1G1, EXOSC10, PIK3C2B, PTX3, THUMPD1, FAM149B1, FOXRED2,
ZNF786, VEZT, P2RX4, ZNF34, TMEM138, IBTK, CDC7, FBXW4, BMF, VKORC1, MAFK, PBRM1, SLIT2, IL15, TBC1D12,
BAG4, MPI, ERC1, TAF3, NUDCD1, YPEL5, BACE1, ARPP19, TAF1B, TLN1, SFTPB, FAP, OTULIN, TXNRD3, BCL2, CNOT6,
KDM1A, LIN7C, ENPP1, TRANK1, CREB3, AK8, ALS2CL, GPR176, ASPM, CHORDC1, ZNF280C, CHST11, EPHA4, PLD1,
USP35, LPXN, MED31, USP33, PTPRH, THOC2, UBAC2, HUS1, RABGAP1L, HELQ, ATG4A, CNOT3, VAT1, LEMD3, SLC35B3,
FBXO46, TRO, PMM1, YTHDF3, DNASE1L1, TMEM200A, TMEM102, TAZ, CARM1, SRA1, FBXO6, TAB1, ARL13B, ZFP82,
TALDO1, PEX5, SKAP2, TRIM14, GXYLT2, CLTB, KHDC4, PTS, DLX5, COL4A3, STAT4, ERCC2, MFSD1, LRRC8E, H1-3,
PLCB4, FOXRED1, SLC26A2, MNS1, AVIL, ECHDC3, RRP15, AP1AR, PPP1R12C, IFNLRI, CHERP, PIK3R1, BTG1, AP1S3,
ARIH1, MOB3A, UHRF1BP1, THAP3, MFGE8, ULK4, HS6ST2, GDE1, EDEM2, TRAK1, DDX31, CPT1B, SLC28A1, SC5D,
TMEM267, CRTAP, PIP5K1B, SPRTN, CFAP20, PKIG, YPEL2, ZNF35, DUSP14, ZNF692, JOSD2, LIG4, ING4, DDAH1, FTSJ1,
WDTC1, PISD, PTCD3, TMCC2, CLPTM1, CENPU, BICD1, DNAJC21, NR2F1, NAMPT, SLC46A3, PTGFRN, GALNT12, NRM,
JADE2, EVC2, THAP6, IRAK1BP1, PLXND1, GALK1, SACM1L, MED7, EGR4, SLC43A3, SH3BP5L, CDC37, ZNF189, MAP2K7,
SLC22A5, XRCC1, DAZAP1, RASSF2, CDAN1, RMDN1, LEO1, MAGED1, WRNIP1, SECISBP2, NDUFAF8, FRS3, RFC1,
SERINC2, SLC35A4, GREB1L, TLN2, TM2D1, RPL19, ETV6, RND1, NME6, TMEM181, CEP41, ZFAND2A, NOD1, TBC1D19,
BNIP3, CDK7, MYO6, METTL17, ZGLP1, TEP1, SLC4A7, PDIA5, TBRG4, BFAR, DEPP1, TMEM186, LRWD1, CCDC8, HAGH,
CDCA4, NEDD1, GNB2, PODXL2, MPG, ATP2A1, GAB2, NGF, POLR2I, RETREG2, WDR62, RBBP9, FAM234B, SHFL, PRRC2B,
RARS2, BORCS6, NCDN, PARVA, VAV3, RPS23, APLF, MCM7, INTS9, MYO19, OSBPL7, USP31, FBXO48, ZNF559, EXOC3L2,
ADORA2A, C14orf28, GORASP2, STBD1, NET1, ELP4, MTARC2, LNPK, DDIAS, SLC17A3, CDSN, NOP14, METTL14, CZIB,
YIF1B, ATP6AP1L, PYGO2, GPSM2, PDP2, EBAG9, SREBF2, R3HDM4, ELF1, PPP3R1, C18orf32, PTGER4, SHISA5, BEX2,
RHBDF1, PRXL2A, CNOT10, SVOP, MAEA, ZNF319, UBASH3B, IPO13, LANCL2, BRAF, SCAI, RANBP17, SLC16A1, HMGN4,
HMBS, SMIM14, ATP13A2, PGS1, PMF1, IFT122, LTBP4, ADPGK, WDR35, UBXN11, RNF20, TRMT2A, YAP1, AGER, LARP1B,
CNNM3, RAB8B, SSU72, BID, HTR2A, WDR90, PLEKHG4B, C2orf69, SLTM, FAIM, MVP, GAA, METTL6, VARS2, ACSL3,
NEXMIF, TMEM30A, UBTD2, TMEM179B, FAM120B, GNPDA1, VDR, RPL10A, TEDC1, TCF19, EDEM3, ADAMTS3,
ARHGEF11, PRKG1, L2HGDH, DCLRE1A, ODR4, ZNF280B, PRKCA, COL6A2, ELMOD2, PRKRIP1, SMARCD1, EPHB2,
NDUFAF4, NXPH4, MTFR1L, GNL2, ZNF677, SMARCD3, SLC25A39, NDUFAF2, POLI, PRMT6, USF3, SLC6A8, OLFML2B,
SLC24A2, SRSF9, TBCID8, PUM2, TMEM131L, DTWD2, LPCAT1, DNLZ, MRPS18A, CC2D2A, C1D, RP2, NCEH1, NKX3-1,
CUL7, DGCR2, SYNM, ZNF32, C1orf122, DOCK1, PIPOX, NCAPD3, C1orf112, PAIP1, RNF7, H2BC21, TEX261, TTC30A, TUSC2,
FAM104B, RND2, FMNL3, ORC4, ZSWIM5, NIPSNAP1, PARD3B, PPP1R11, PPP1R9A, SOCS4, SUPT5H, PKMYT1, KDM5A,
LMNB2, MICAL1, TRAPPC11, ACOX1, ZBTB3, PRIMPOL, CNTRL, C11orf95, CLN8, ADCK5, SHQ1, SRRT, SSBP4, ZYG11B,
ATP8A1, KANK2, SP1, LINS1, PPP1R13B, TBCB, CLYBL, RTL6, GFOD1, AMN, ALDOC, FAM76B, EPS15L1, SLC30A3, RAB23,
PDHA2, ZBTB25, HAUS7, SLC16A9, ZFYVE28, MBD6, MYOM3, PCSK7, ANAPC2, ORC2, CCDC191, WNT5A, RBM48, LRRC32,
BTBD10, DROSHA, IFT43, NUBP2, CNOT6L, ACAD11, ZNF174, CCDC113, C6orf89, TLL1, AP4E1, TMEM260, PHB2, ELL,
SCRIB, TGFB3, ZNF414, PLAIA, FAM3A, ABCE1, C12orf66, PMFBP1, RNF123, BAD, ADAMTS13, ARIH2, KCTD12, ZNF606,
DDX19B, CACHD1, TMEM129, DNAJC25, ALG13, ELP5, LIMA1, ARPC1B, PRR14, QPCTL, LCMT1, ZNF783, VPS25, SLC2A10,
BTBD11, CCDC28A, ICE2, SEC23IP, TUBB6, THAP7, GNA12, SUV39H2, WBP4, KIF24, UBTF, APOL5, THEM6, CLDN10,
ARFGAP1, E2F7, PGAP3, KIAA0100, GPR19, RUNX1T1, HCCS, TRMT5, DGKA, NIT2, TRAPPC13, MPND, PELI2, ZNF84, PAX6,
F2R, SYCE3, UPRT, RIOK3, FDFT1, USP30, KBTBD8, KIF20B, DPYSL5, DBNL, ALG6, PAK4, NKAIN1, THNSL1, RASL11B,
CLIP3, GDPD1, LRRC39, ITGB6, ARRB2, CD300LG, DHDDS, ALG3, ELOVL2, FAM160A2, CCDC30, KHNYN, ZNF385A,
C8orf37, IFT46, TRIM25, COL21A1, CYP1A1, SLC39A11, CDKN2D, TRPC1, DPH7, ALDH8A1, CPEB3, GLI3, EMP2, WDR18,
DNAH5, TIAM2, TMEM184B, TIMP3, BRD3, MTMR2, TRABD, ZDHHC5, GTF2F2, TEPSIN, PEAR1, GRASP, E2F5, ZC3H6,
CENPT, GGNBP2, NT5DC3, TMEM161A, ATG7, MCIDAS, CNR1, BOD1L1, MED19, MRPS27, NIBAN2, CLGN, PIP4K2C,
FAM13A, ZNF584, MOCS2, C2orf74, DNAL4, ZNF430, CRABP1, ECSIT, TRIM2, ORC6, ITPR2, KIF25, EHMT2, MATN2,
TMEM132A, TIMM8B, GJB3, PDS5B, SPRED3, SMG6, SMARCAL1, GNB5, MYBL2, NUP50, SPATA20, NIP7, STIL, GPATCH11,
ZGRF1, KIF18A, NSMCE2, KIF5B, PRPS2, MAP3K21, MAPK14, MGAT5, TBL1X, PUS7L, TRPC4AP, GPRIN3, ADGRG1, GRK3,
TESC, OBI1, CHD6, MUC20, NFE2L2, HAUS3, RAB5IF, ZBTB1, RERG, RWDD4, BPHL, RAD54L, TTLL5, IMPDH1, INF2, SACS,
DLL3, PARD6A, CROCC, SMPD2, CDC42EP1, CD101, ATP11B, INTS13, NOP9, TYK2, CDC42EP2, TPBG, ZMAT5, URM1,
PSME3, IPP, ZNF322, AXL, TRMT61B, LMX1B, CLEC16A, FERMT2, PCOLCE2, PRPSIL1, GJA1, FMNL1, SLC7A10, ALDH6A1,
OBSL1, BCL2L1, ABRAXAS2, LRRK2, SPEG, XYLT1, HNF4G, PPPICC, STAGI, MCCC1, THYN1, MPDZ, PPWD1, PLCL2,
FAM204A, UBE2E1, EHD4, SLC6A9, IL17RC, PIMREG, C16orf87, SCX, TSPANI, ARMC1, ABCF3, DYRK3, GPHN, PIK3C3, LCT,
NECTIN2, HSPA4L, GTPBP1, CD160, TMEM184A, DDX3X, TIMM8A, IKBKB, RGCC, BCL7A, FOXK1, KBTBD6, MRPS30,
SH3GLB2, HCN1, DCLREIC, SLFN5, PPL, CHST12, CIAO1, SNX8, CA11, ST3GAL4, IL12A, DHRS9, BBS10, SHANK3, SNX27,
CCDC43, ROR1, UPF3B, CACNA1H, MYH15, POGLUT1, GATB, HDHD2, ERMP1, NARS2, FLVCR1, PLPP1, LPAR1, NPLOC4,
RPAIN, CRY1, SART1, SLC35E2B, VPS50, CNEP1R1, MGLL, PLOD1, VPS53, ZNF234, FOXP4, FMR1, ZNF197, KRTCAP3,
NUP35, UBE2Q2, ST6GALNAC2, BOLA3, MAN2A2, MTRF1, RABEP1, RPS6KA5, TAB3, ZNF43, SEC13, INTS2, NRG1,
SLC25A11, MSLN, OSBPL8, PIP4K2B, CXCL16, CDKL1, DCAF17, FAM168A, NUMBL, HS2ST1, ANKS6, KIF6, TP53I13, ZNF784,
SNX9, NIT1, TYMP, DMAC2L, MAJIN, TRUB1, HEATR5B, STX3, HERPUD2, TGOLN2, OCIAD1, COL7A1, FUT1, ABHD16A,
VARS1, FAM200A, GUCD1, AAR2, MARVELD2, PDE3A, FAM210B, HSP90B1, ATG3, CYTH1, DLL1, TBC1D25, EIF5B, TUBG2,
SART3, A4GALT, ZNF132, SLC37A1, SCLT1, ISOC2, ACTR10, NETO1, NDRG4, SLC20A2, SIN3A, DNAH3, INTS12, POLDIP3,
APAF1, CORO1C, RAP1GDS1, HYAL2, RNF223, DMPK, ANKS3, ZNF134, ARPC5L, IMP3, LONRF1, CCNC, FAH, SPIN1, SFR1,
BRINP1, CHTF18, AP5Z1, ENG, NUP62CL, RPTOR, BLOC1S2, COQ3, DNAJB1, SLC48A1, TSC2, PHC3, TMEM218, MRPS26,
ESD, ALG12, BEND3, SOX13, PRSS16, UPK3B, RFX3, EMX2, SEMA3A, SLAIN2, TSNARE1, TWF2, ISM1, EFCAB7, SQLE,
ATF5, GNAI1, MAPRE3, H4-16, IL9R, ATP11A, PGAP4, ACTN2, WASL, EXTL2, PRKD1, CIDEB, NSRP1, PAFAH1B3, STIM2,
HELB, PHF5A, VAMP4, KCNAB2, MRPL4, TRIM28, IKZF5, LHX2, MYO5C, SNRNP70, PNISR, PPM1H, PDXDC1, SASS6,
CXorf38, GLOD4, RNF32, TSSK6, DZIP1L, PPP1R35, CCDC28B, SIRT7, CLSTN1

TABLE 5
Quantitative spent media analyses.

| Valine | 688.3106 | 666.3276 | 687.1486 | 726.6834 | 742.2259 | 734.6735 | 713.4117 | 723.6366 | 724.1721 |
|---|---|---|---|---|---|---|---|---|---|
| Tyrosine | 338.1831 | 327.7677 | 326.5834 | 343.8425 | 352.2437 | 347.5299 | 345.4387 | 347.0065 | 344.3643 |
| Tryptopha | 41.5253 | 30.4119 | 30.7293 | 32.1466 | 35.2669 | 34.0157 | 33.4861 | 35.9654 | 33.4315 |
| Threonine | 670.812 | 645.6735 | 675.3672 | 695.9072 | 717.343 | 706.3706 | 699.0744 | 704.1109 | 690.685 |
| Serine | 328.2095 | 311.9989 | 324.9122 | 338.8552 | 352.5299 | 340.9182 | 342.7516 | 344.2997 | 347.3304 |
| Pyruvate | 514.4112 | 521.016 | 527.7019 | 581.9079 | 555.1826 | 561.5174 | 490.2091 | 503.302 | 496.3023 |
| Pyridoxine | 14.2512 | 13.9288 | 14.1272 | 14.4386 | 15.0325 | 14.743 | 14.317 | 14.5991 | 14.1872 |
| Proline | 29.3359 | 28.666 | 28.4093 | 30.7583 | 31.7469 | 31.3161 | 30.9258 | 32.2871 | 32.1999 |
| Phenylalan | 336.3792 | 331.4149 | 334.1316 | 348.1533 | 356.9122 | 353.6332 | 342.593 | 350.6099 | 340.5258 |
| Nicotinami | 27.0354 | 25.2945 | 23.99 | 25.9326 | 25.916 | 26.1311 | 25.6508 | 26.4979 | 25.624 |
| Methionine | 165.8587 | 160.8794 | 161.9876 | 168.822 | 175.4045 | 173.2925 | 170.4041 | 171.8375 | 166.9656 |
| Lysine | 660.3905 | 614.3841 | 653.896 | 678.4309 | 699.4027 | 706.8744 | 672.973 | 697.1506 | 691.8935 |
| Leucine | 610.6567 | 586.1636 | 607.5055 | 620.8797 | 645.3635 | 634.5085 | 621.6727 | 640.1201 | 615.008 |
| Lactate | 8252.184 | 7999.382 | 7917.546 | 8659.138 | 8823.292 | 8955.436 | 7948.947 | 8180.459 | 8501.741 |
| Isoleucine | 604.8306 | 595.0797 | 597.8336 | 630.1736 | 651.2711 | 639.9193 | 611.6153 | 633.5023 | 623.7919 |
| Histidine | 164.5193 | 150.4069 | 159.4787 | 164.2636 | 169.0722 | 169.2057 | 163.177 | 167.6862 | 165.8815 |
| Glycine | 375.9867 | 353.4097 | 367.459 | 390.9086 | 389.0506 | 386.1501 | 383.52 | 377 | 397.0287 |
| Glutamine | 1915.277 | 1793.907 | 1849.628 | 1953.742 | 2012.845 | 2024.954 | 1935.288 | 1961.141 | 1912.672 |
| Glutamate | 152.9766 | 140.6193 | 155.3132 | 158.0707 | 162.6397 | 157.7599 | 149.5142 | 157.999 | 161.8075 |
| Glucose | 17310.59 | 16663.31 | 17095.22 | 18226.77 | 18309.13 | 18528.01 | 17940.45 | 17727.19 | 17503.59 |
| Cystine | 134.5638 | 118.9751 | 128.759 | 139.6829 | 141.8095 | 131.2034 | 129.6339 | 127.3886 | 131.598 |
| Aspartate | 8.2442 | 7.2312 | 7.3839 | 6.5953 | 7.0671 | 8.6349 | 8.1665 | 9.1974 | 9.3164 |
| Asparagine | 1.1128 | 0.5423 | 0.5394 | 0.5485 | 0.6149 | 0.621 | 0.7236 | 0.786 | 0.783 |
| Arginine | 305.6195 | 289.1805 | 301.3199 | 312.6565 | 326.6335 | 323.2981 | 314.6084 | 320.3649 | 321.0473 |
| Alanine | 181.4841 | 167.0675 | 171.6259 | 188.3103 | 188.3831 | 182.4606 | 176.6541 | 191.8497 | 192.0047 |
| Group | Mock_01 | Mock_02 | Mock_03 | Mock_04 | Mock_05 | Mock_06 | SARS- | SARS- | SARS- |
| Valine | 697.854 | 702.6601 | 697.6449 | 740.914 | 719.0609 | 716.4532 | 739.5663 | 733.9569 | 716.0338 |
| Tyrosine | 338.8138 | 325.1803 | 333.4291 | 350.5504 | 353.5139 | 347.7998 | 352.271 | 350.7283 | 346.9765 |
| Tryptopha | 36.0356 | 34.4187 | 38.3342 | 31.071 | 43.304 | 35.5644 | 41.9661 | 34.7557 | 36.5499 |
| Threonine | 680.5361 | 664.0692 | 677.0097 | 703.5036 | 697.3614 | 693.2714 | 721.0861 | 695.9777 | 702.7659 |
| Serine | 337.9984 | 331.3212 | 327.0949 | 361.0976 | 352.8384 | 357.1358 | 374.9639 | 342.2636 | 352.7439 |
| Pyruvate | 508.6787 | 514.5746 | 514.3263 | 716.1481 | 719.2055 | 743.4019 | 732.8229 | 744.0779 | 730.443 |
| Pyridoxine | 14.2022 | 14.0419 | 13.9613 | 15.3551 | 14.823 | 15.0563 | 15.4529 | 14.8529 | 14.8799 |
| Proline | 31.5095 | 31.9404 | 30.7331 | 23.7502 | 23.2226 | 24.197 | 26.3419 | 24.6052 | 24.7625 |
| Phenylalan | 336.2901 | 334.8096 | 336.7183 | 353.6766 | 352.1438 | 349.2638 | 357.7247 | 353.5397 | 344.5926 |
| Nicotinami | 26.5294 | 25.5376 | 27.5993 | 24.5862 | 26.1125 | 26.4855 | 27.8193 | 25.8229 | 25.2423 |
| Methionine | 166.5752 | 163.7207 | 164.3622 | 181.7806 | 178.2943 | 180.3027 | 182.0367 | 180.5628 | 178.7472 |
| Lysine | 663.9269 | 673.3521 | 661.4339 | 708.7324 | 688.3134 | 685.0556 | 703.3017 | 688.3739 | 683.3224 |
| Leucine | 614.4304 | 607.9456 | 609.5201 | 663.8281 | 677.2435 | 662.2538 | 669.993 | 655.9925 | 650.6457 |
| Lactate | 8107.693 | 8204.871 | 7717.772 | 3482.446 | 3482.862 | 3531.593 | 3560.167 | 3592.547 | 3489.728 |
| Isoleucine | 607.1721 | 603.1587 | 602.8182 | 640.1822 | 663.442 | 644.2385 | 654.6233 | 649.2324 | 642.3362 |
| Histidine | 161.9115 | 162.7722 | 161.7064 | 169.2857 | 169.0971 | 169.2185 | 174.9683 | 168.7505 | 167.7429 |
| Glycine | 390.5837 | 377.727 | 377.0872 | 388.775 | 398.6231 | 389.4699 | 397.217 | 370.5411 | 381.6646 |
| Glutamine | 1930.268 | 1869.513 | 1905.072 | 2180.2 | 2156.665 | 2156.886 | 2196.817 | 2160.606 | 2114.655 |
| Glutamate | 155.117 | 161.7982 | 151.1189 | 99.3716 | 90.4827 | 93.6585 | 97.545 | 92.7312 | 94.4931 |
| Glucose | 17492.75 | 17226.44 | 17693.54 | 20150.1 | 18992.94 | 19509.07 | 19572.08 | 19085.27 | 19165.31 |
| Cystine | 133.8875 | 129.9225 | 124.6643 | 143.1917 | 138.1728 | 134.9933 | 142.8779 | 133.8882 | 130.6608 |
| Aspartate | 10.4536 | 8.5166 | 9.515 | 5.4013 | 7.3601 | 7.4542 | 6.8552 | 7.6457 | 7.1351 |
| Asparagine | 0.6494 | 0.7706 | 0.7224 | 0.3085 | 0.5466 | 0.4762 | 0.5613 | 0.3636 | 0.3559 |
| Arginine | 312.0188 | 306.4439 | 308.7624 | 330.2071 | 324.2448 | 325.8255 | 335.3413 | 324.3771 | 321.2249 |
| Alanine | 178.8378 | 186.2924 | 171.0025 | 105.3891 | 102.5718 | 105.0699 | 109.0977 | 106.0558 | 109.3688 |
| Group | SARS- | SARS- | SARS- | Mock_medi | Mock_medi | Mock_medi | SARS- | SARS- | SARS- |

TABLE 6
Intracellular metabolite profiling.

| pvalue | 0.013559 | 0.007332 | 0.005387 | 0.424352 | 0.139608 | 0.76E−06 | 0.127537 | 0.062496 | 0.258246 |
|---|---|---|---|---|---|---|---|---|---|
| log2Foldcha [symbol] | 0.147133 | −0.40498 | −0.26416 | −0.04908 | −0.13581 | 1.252754 | −0.13817 | −0.19914 | −0.14228 |
| ttest | 0.013559 | 0.007332 | 0.005387 | 0.424352 | 0.139608 | 1.76E−06 | 0.127537 | 0.062496 | 0.258246 |
| SARS-COV- | 9722881541 | 351501395.8 | 13239684755 | 7768636461 | 184137388.5 | 9990994 | 1865115009 | 34019741.08 | 81489025.27 |
| [symbol] | | | | | | | | | |
| SARS-COV- | 11550823978 | 316785264.1 | 11732895775 | 8393465936 | 186873676.5 | 13228368 | 1903049284 | 35160919.86 | 129706047.9 |
| [symbol] | | | | | | | | | |
| SARS-COV- | 10153962734 | 349259587 | 14311613794 | 7861699897 | 188134944.5 | 10465250 | 1899715072 | 33765491.96 | 108861306.2 |
| [symbol] | | | | | | | | | |
| SARS-COV- | 11114120927 | 429903518 | 13752531842 | 8136405756 | 223308427.7 | 11528096 | 2242621208 | 37653541.4 | 103952030.8 |
| [symbol] | | | | | | | | | |
| SARS-COV- | 11061377320 | 490253805 | 16235926891 | 8708233425 | 241533721.3 | 10318928 | 2426560434 | 45828161.74 | 99738045.27 |
| [symbol] | | | | | | | | | |
| SARS-COV- | 10454058218 | 304524871.8 | 12691630761 | 7021417688 | 214800733.9 | 11407491 | 2234970346 | 32320252.67 | 75503120.46 |
| [symbol] | | | | | | | | | |
| Mock_06 | 10346358739 | 519770488 | 15414637459 | 7774750730 | 232544330.3 | 5860663 | 2378694070 | 39189850.11 | 103364566.9 |
| Mock_05 | 10025321905 | 522346542.8 | 17007219905 | 8364652478 | 238668306.6 | 5563646 | 2398782423 | 41798795.21 | 107527142.3 |
| Mock_04 | 9417596045 | 463378488.5 | 15869259469 | 8530291683 | 206426576.6 | 4878667 | 2059539939 | 44510302.11 | 111457598.1 |
| Mock_03 | 9170388200 | 504635128.2 | 17425769184 | 7983520635 | 246979245.1 | 4862164 | 2480166210 | 45809529.37 | 111584587.2 |
| Mock_02 | 9604810276 | 502353329.8 | 16536858720 | 9198545293 | 239089387.2 | 3842764 | 2472554561 | 44239535.84 | 110064193.9 |
| Mock_01 | 9281974689 | 456378561.5 | 16180336694 | 7695323924 | 197356982.8 | 3082849 | 2045917682 | 35578215.87 | 117363003 |
| Metabolite | In house | In house | In house | In house | In house | In house | In house | In house | In house |
| annotation | RT + MSMS | RT + MSMS | RT + MSMS | RT + MSMS | RT + MSMS | RT + MSMS | RT + MSMS | RT + MSMS | RT + MSMS |
| source | library | library | library | library | library | library | library | library | library |
| Method_ionization | philic_Neg | philic_Pos | philic_Pos | philic_Neg | philic_Neg | philic_Neg | philic_Neg | philic_Neg | philic_Neg |
| mode | | | | | | | | | |
| Metabolite | Glutamate | N6,N6,N6- | Glutamine | Taurine | Fumarate | FGAR | Malate | 2- | Creatine |
| | | Trimethyl-L- | | | | | | Hydroxyglutarate | |
| | | lysine | | | | | | | |
| pvalue | 0.196825 | 0.003748 | 0.007415 | 0.467233 | 0.002367 | 2.37E−05 | 0.002471 | 0.011292 | 0.271506 |
| log2Foldcha [symbol] | 0.379047 | −0.60489 | −0.60671 | −0.05354 | −0.59993 | 0.368383 | −0.4873 | 1.681291 | 0.053114 |
| ttest | 0.196825 | 0.003748 | 0.007415 | 0.467233 | 0.002367 | 2.37E−05 | 0.002471 | 0.011292 | 0.271506 |
| SARS-COV- | 77730164.22 | 138451661 | 376012155 | 788335730.8 | 1511129488 | 427604113.3 | 133480633.4 | 86454285.87 | 744502729.2 |
| [symbol] | | | | | | | | | |
| SARS-COV- | 126930388.9 | 121302219.7 | 337555058 | 973276857 | 1338687581 | 492844600.3 | 156631557.5 | 55550887.16 | 859287063.9 |
| [symbol] | | | | | | | | | |
| SARS-COV- | 193060070 | 144950554.6 | 380536076 | 982627327.2 | 1555533025 | 451135618.3 | 156570060.8 | 41178145.65 | 834070817.4 |
| [symbol] | | | | | | | | | |
| SARS-COV- | 231126297.9 | 160659457.2 | 471118900 | 941004486.7 | 1810265312 | 460073688.7 | 124834058 | 62254205.8 | 880409420.2 |
| [symbol] | | | | | | | | | |
| SARS-COV- | 146613756.6 | 247738665.2 | 611424477 | 949329823.4 | 2563173418 | 469583881.5 | 175740404 | 35389952.69 | 841308723.9 |
| [symbol] | | | | | | | | | |
| SARS-COV- | 114327720.5 | 189974059.3 | 616550787 | 780061945.1 | 2026806544 | 424919300.4 | 127502202.4 | 24829221.9 | 816822335.9 |
| [symbol] | | | | | | | | | |
| Mock_06 | 107976432.5 | 246457179.6 | 705031402 | 971897372.8 | 2637264463 | 369948214.4 | 189871337.5 | 18536150.18 | 864521382.6 |
| Mock_05 | 91331619.88 | 292867707.8 | 824521173 | 948282538 | 3116746474 | 317679526.5 | 195812658.4 | 17284527.25 | 829977416.9 |
| Mock_04 | 133896360.2 | 248540535.4 | 703634745 | 970790435.9 | 2531263836 | 354167710.1 | 211254267.3 | 16176967.69 | 797720683.6 |
| Mock_03 | 119257927.1 | 242790927.8 | 571410381 | 828883967.7 | 2566045797 | 348726650.3 | 220386786.3 | 14668078.55 | 752102807.3 |
| Mock_02 | 112836522.9 | 275033570.7 | 882567926 | 986074251.7 | 3119210264 | 371946704.2 | 245496657.1 | 15592990.99 | 789378977.3 |
| Mock_01 | 118899590.9 | 219851045 | 566269094 | 913442175.4 | 2406858066 | 349352628.8 | 163435834.2 | 13046130.04 | 762821915 |
| Metabolite | In house | In house | In house | In house | In house | In house | In house | In house | In house |
| annotation | RT + MSMS | RT + MSMS | RT + MSMS | RT + MSMS | RT + MSMS | RT + MSMS | RT + MSMS | RT + MSMS | RT + MSMS |
| source | library | library | library | library | library | library | library | library | library |
| Method_ionization | philic_Pos | philic_Neg | amide_acidic | philic_Neg | philic_Neg | philic_Neg | philic_Neg | philic_Pos | philic_Neg |
| mode | | | | | | | | | |
| Metabolite | S- | Succinate | Nicotinamide | UDP | Valine | Glycerol-3- | Glycine | 5- | Phosphocreatine |
| | Adenosyl- | | | Glucuronate | | Phosphate | | Aminoimidazole | |
| | homocysteine | | | | | | | ribotide | |
| pvalue | 0.241864 | 0.004518 | 0.002933 | 0.002673 | 0.047603 | 0.055238 | 0.000263 | 0.001964 | 0.977506 |
| log2Foldcha [symbol] | −0.21307 | −0.50734 | −0.19772 | −0.58618 | 0.355531 | −0.18541 | −0.84754 | −0.70194 | −0.0097 |
| ttest | 0.241864 | 0.004518 | 0.002933 | 0.002673 | 0.047603 | 0.055238 | 0.000263 | 0.001964 | 0.977506 |
| SARS-COV- | 110234027.5 | 737979177.6 | 471545996.4 | 2497114488 | 4693313946 | 6774364.782 | 9513168.19 | 2926215.394 | 143986058 |
| [symbol] | | | | | | | | | |
| SARS-COV- | 77044476.97 | 611805856.3 | 457285979.8 | 2089738947 | 5005509744 | 7197167.084 | 10788918.47 | 2644449.073 | 162362164 |
| [symbol] | | | | | | | | | |
| SARS-COV- | 72251286.5 | 766218453.9 | 506361330.5 | 2557546734 | 4239795109 | 8002516.764 | 9334904.211 | 2600735.887 | 177626756 |
| [symbol] | | | | | | | | | |
| SARS-COV- | 91267031.83 | 828863072.5 | 511845707.7 | 2814034524 | 5036849896 | 8841529.435 | 13090186.56 | 3189237.409 | 195560151 |
| [symbol] | | | | | | | | | |
| SARS-COV- | 137013592.9 | 1143685230 | 560315457.5 | 4094989143 | 3858693922 | 9445645.915 | 17160239.97 | 3620047.126 | 148802003 |
| [symbol] | | | | | | | | | |
| SARS-COV- | 68514904.3 | 863779658.9 | 524486589 | 3375446313 | 4635594076 | 7405329.571 | 9018796.855 | 2320736.499 | 399513618 |
| [symbol] | | | | | | | | | |
| Mock_06 | 103920286.8 | 1177927965 | 587874344.8 | 4303320444 | 3702952711 | 8310799.61 | 17912919.38 | 4302799.952 | 249835416 |
| Mock_05 | 102310695.4 | 1406042040 | 603515720.5 | 4697877174 | 3310519564 | 9327994.841 | 22056216.77 | 5318580.435 | 206978015 |
| Mock_04 | 120697856.7 | 1070346934 | 575240076.4 | 4015443619 | 2374529677 | 9862534.575 | 20285844.84 | 4100293.57 | 198056587 |
| Mock_03 | 104057123.9 | 1036670703 | 551952607.5 | 4025038252 | 3973294470 | 8960307.041 | 20571017.69 | 5211608.953 | 87257492 |
| Mock_02 | 114919175.9 | 1286583020 | 584337386.1 | 5048263357 | 3024740578 | 9359333.151 | 23374944.89 | 5705568.22 | 270607126 |
| Mock_01 | 98959868.91 | 1061818014 | 574254906.3 | 4075443840 | 5083829074 | 8382533.202 | 19791106.14 | 3505261.709 | 223402123 |
| Metabolite | In house | In house | In house | In house | In house | In house | In house | In house | In house |
| annotation | RT + MSMS | RT + MSMS | RT + MSMS | RT + MSMS | RT + MSMS | RT + MSMS | RT + MSMS | RT + MSMS | RT + MSMS |
| source | library | library | library | library | library | library | library | library | library |
| Method_ionization | philic_Neg | philic_Neg | philic_Pos | philic_Neg | amide_acidic | philic_Neg | philic_Neg | philic_Neg | amide_acidic |
| mode | | | | | | | | | |
| Metabolite | Mannose-6- | Arginine | Serine | Isoleucine | Lactate | Glucosamine- | Saccharate/ | Saccharate/ | Pyruvate |
| | Phosphate/ | | | | | 6-Phosphate | Galactarate_01 | Galactarate_02 | |
| | Fructose-1- | | | | | | | | |
| | phosphate | | | | | | | | |
| pvalue | 0.01846 | 0.02646 | 0.980284 | 0.050662 | 0.041405 | 0.780473 | 6.86E−05 | 0.001166 | 0.943234 |
| log2Foldcha [symbol] | 0.324605 | −0.55689 | 0.001387 | −0.50792 | −0.75904 | −0.04533 | 0.798309 | 0.363787 | 0.015777 |
| ttest | 0.01846 | 0.02646 | 0.980284 | 0.050662 | 0.041405 | 0.780473 | 6.86E−05 | 0.001166 | 0.943234 |
| SARS-COV- | 670916304.8 | 598578208 | 2246823153 | 11480493 | 807040649 | 8433602909 | 330298760.2 | 614252773 | 185979617 |
| [symbol] | | | | | | | | | |
| SARS-COV- | 849746757.2 | 491320046 | 2714859095 | 14811332 | 720677688 | 10462498626 | 287954898.4 | 736241196.1 | 235476059 |
| [symbol] | | | | | | | | | |
| SARS-COV- | 629977282.6 | 605065266 | 2147679262 | 13782863 | 841353391 | 9741168721 | 322936061.5 | 677208137.1 | 183239395 |
| [symbol] | | | | | | | | | |
| SARS-COV- | 690561247 | 755684063 | 2248311483 | 17956310 | 1179791431 | 11258523467 | 392714165.2 | 729413889 | 268066643 |
| [symbol] | | | | | | | | | |
| SARS-COV- | 555013206.1 | 913525599 | 2376865521 | 16134305 | 1484101848 | 12268141226 | 365113735 | 768413073.4 | 255094318 |
| [symbol] | | | | | | | | | |
| SARS-COV- | 745087075.9 | 1000093953 | 2286048187 | 22125935 | 1677787898 | 9877775871 | 338840136.2 | 611285216 | 317017724 |
| [symbol] | | | | | | | | | |
| Mock_06 | 615215291.4 | 1254418315 | 2271375262 | 27914237 | 2185667664 | 12283524293 | 202390329.2 | 555504506 | 305863620 |
| Mock_05 | 599397388.8 | 1198169431 | 2417023424 | 22253016 | 2288886851 | 10518604933 | 189508056.3 | 595923572.8 | 234849280 |
| Mock_04 | 464552000.8 | 1115571868 | 2335144403 | 26296761 | 2140129584 | 13034707769 | 177206429.8 | 525812266.1 | 282491628 |
| Mock_03 | 545906030.2 | 646327228 | 2433908872 | 11885651 | 808782307 | 9150107176 | 215826953.3 | 527977927.8 | 128975229 |
| Mock_02 | 551591746.6 | 1303290925 | 2313190058 | 28270376 | 2596032938 | 6580492257 | 189374034.3 | 544650162.4 | 296891244 |
| Mock_01 | 530237922.8 | 902488735 | 2236468088 | 20305652 | 1337523205 | 12454802180 | 197508631.7 | 464941727.6 | 180088301 |
| Metabolite | In house | In house | In house | In house | In house | In house | In house | In house | In house |
| annotation | RT + MSMS | RT + MSMS | RT + MSMS | RT + MSMS | RT + MSMS | RT + MSMS | RT + MSMS | RT + MSMS | RT + MSMS |
| source | library | library | library | library | library | library | library | library | library |
| Method_ionization | philic_Neg | amide_acidic | philic_Neg | amide_acidic | amide_acidic | philic_Neg | philic_Pos | philic_Neg | amide_acidic |
| mode | | | | | | | | | |
| Metabolite | Threonine | Glucose | N- | Cystathionine | Pyridoxine | Galactitol / | Y- | Proline | S- |
| | | | Acetylaspartate | | | Mannitol/ | Aminobutyric | | Adenosyl- |
| | | | | | | Sorbitol | acid | | methionine |
| pvalue | 0.345986 | 9.9E−05 | 0.000651 | 0.001235 | 0.000844 | 8.56E−06 | 0.20687 | 0.57122 | 0.532139 |
| log2Foldcha [symbol] | 0.068493 | 0.420237 | 1.819275 | 0.770951 | 0.305742 | 0.630486 | −0.06469 | −0.08697 | 0.089958 |
| ttest | 0.345986 | 9.9E−05 | 0.000651 | 0.001235 | 0.000844 | 8.56E−06 | 0.20687 | 0.57122 | 0.532139 |
| SARS-COV- | 360890992.2 | 235898062.9 | 89452252 | 4792449436 | 2365809085 | 35246804.03 | 348660110.3 | 7799730.01 | 167401635 |
| [symbol] | | | | | | | | | |
| SARS-COV- | 414625828 | 237454356.3 | 112164204 | 6880257111 | 2950204965 | 38479654.37 | 396606787.1 | 7117473.329 | 172110473.4 |
| [symbol] | | | | | | | | | |
| SARS-COV- | 391057824.4 | 255396516.2 | 103381162 | 6258987690 | 2716519264 | 38512275.43 | 334696374.9 | 7594640.684 | 176762031.6 |
| [symbol] | | | | | | | | | |
| SARS-COV- | 399860141.6 | 272882039.4 | 156368684 | 6099900334 | 2848770863 | 41541852.51 | 369351203.7 | 9335637.122 | 166737993.3 |
| [symbol] | | | | | | | | | |
| SARS-COV- | 373157559.8 | 276996832.4 | 167442389 | 4183048398 | 2910258809 | 41268214.19 | 350585486.6 | 11207935.94 | 226857720.9 |
| [symbol] | | | | | | | | | |
| SARS-COV- | 459506416.6 | 235246583.7 | 166296213 | 5425736494 | 2665592696 | 35402753.71 | 357363105.3 | 12672048.3 | 158982947.5 |
| [symbol] | | | | | | | | | |
| Mock_06 | 380801401.5 | 191688280.6 | 38676881 | 3863922866 | 2379586612 | 23847620.59 | 372064934.4 | 9229800.672 | 219294188.4 |
| Mock_05 | 405153061.6 | 194438831.2 | 41032964 | 2528449981 | 2376116911 | 26064588.73 | 392071084.5 | 10519742.63 | 187447269.3 |
| Mock_04 | 332859683.7 | 188009533.9 | 50569877 | 3438383051 | 2163845236 | 26205433.53 | 388374498.6 | 9498565.929 | 143599047.1 |
| Mock_03 | 384498055.3 | 202903785.3 | 16506903 | 2942151498 | 2229420146 | 25610777.92 | 336491647.9 | 10173676.51 | 131848819.9 |
| Mock_02 | 417420258.8 | 185807291 | 46187698 | 3209201232 | 2110421480 | 24195435.43 | 377145892.5 | 10878199.84 | 171870448.5 |
| Mock_01 | 367129554.6 | 168473649 | 32329207 | 3732201598 | 2054873273 | 22938319.67 | 390050066.5 | 8890263.609 | 150180844.6 |
| Metabolite | In house | In house | In house | In house | In house | In house | In house | In house | In house |
| annotation | RT + MSMS | RT + MSMS | RT + MSMS | RT + MSMS | RT + MSMS | RT + MSMS | RT + MSMS | RT + MSMS | RT + MSMS |
| source | library | library | library | library | library | library | library | library | library |
| Method_ionization | philic_Neg | philic_Pos | amide_acidic | philic_Pos | philic_Neg | philic_Neg | philic_Neg | philic_Neg | philic_Pos |
| mode | | | | | | | | | |
| Metabolite | Alpha- | 5- | Putrescine | L-Glutathione | Aspartate | Asparagine | Phosphorylcholine | Inosine | methionine |
| | ketoglutarate | Aminovaleric | | reduced | | | | monophosphate | sulfoxide |
| | | acid | | | | | | | |
| pvalue | 0.001409 | 0.000205 | 0.114351 | 0.60823 | 0.000117 | 0.002041 | 0.377101 | 0.001961 | 0.128438 |
| log2Foldcha [symbol] | 0.407066 | −0.70963 | −0.12917 | −0.03858 | 0.486849 | −0.55038 | 0.061837 | −0.68773 | −0.24356 |
| ttest | 0.001409 | 0.000205 | 0.114351 | 0.60823 | 0.000117 | 0.002041 | 0.377101 | 0.001961 | 0.128438 |
| SARS-COV- | 13954523827 | 77364929.42 | 4636030100 | 728995464.2 | 285279815.2 | 318701235.1 | 595970009.8 | 25968620.69 | 62279469.28 |
| [symbol] | | | | | | | | | |
| SARS-COV- | 14799653733 | 49021569.68 | 5454945804 | 660660245.7 | 329953017.8 | 266171443.6 | 567595272.3 | 19105452.15 | 86854823.32 |
| [symbol] | | | | | | | | | |
| SARS-COV- | 12131588380 | 70150965.34 | 4607276455 | 699990487.3 | 290433005.8 | 326239912.9 | 564912790.3 | 25995612.85 | 61467242.15 |
| [symbol] | | | | | | | | | |
| SARS-COV- | 13024467946 | 70354292.58 | 5171268478 | 706091737 | 354016419.4 | 368958745.4 | 524106452.8 | 28680583.46 | 75758262.35 |
| [symbol] | | | | | | | | | |
| SARS-COV- | 11425172580 | 96508418.64 | 5791501292 | 808750282.1 | 338878305 | 497162395.7 | 529705021 | 42161209.9 | 58730378.59 |
| [symbol] | | | | | | | | | |
| SARS-COV- | 14950774251 | 70121557.09 | 5483416174 | 760110710.5 | 303980146.8 | 354841657.9 | 457936571.6 | 25798218.47 | 90417705.83 |
| [symbol] | | | | | | | | | |
| Mock_06 | 9764509301 | 111544227.2 | 5324126380 | 771168280.9 | 207790659.6 | 492941199.3 | 581163238.9 | 47585692.9 | 81955263.11 |
| Mock_05 | 9661057757 | 136642407.4 | 5854918030 | 878865948.4 | 243588809.2 | 566256068.6 | 513692610 | 52708367.87 | 71915310.34 |
| Mock_04 | 9303999983 | 117859733.3 | 5733101726 | 668269075.9 | 231355194.3 | 513067369.9 | 479411183 | 45870650.09 | 93605917.75 |
| Mock_03 | 10128612812 | 104343883.2 | 6252370537 | 745456171.6 | 201851682.8 | 482636584.8 | 525382806.2 | 41781493.82 | 70873063.39 |
| Mock_02 | 10695002171 | 125019684.9 | 6000977887 | 731650989.3 | 233543368.8 | 590178466.9 | 491579487.4 | 47964204.63 | 89343862.94 |
| Mock_01 | 10995095027 | 113567216.8 | 4896156409 | 687470554.1 | 239488593.4 | 477290909.2 | 513049046.2 | 34227509.99 | 107907850.1 |
| Metabolite | MSMS | In house | In house | In house | In house | In house | In house | In house | In house |
| annotation | | RT + MSMS | RT + MSMS | RT + MSMS | RT + MSMS | RT + MSMS | RT + MSMS | RT + MSMS | RT + MSMS |
| source | | library | library | library | library | library | library | library | library |
| Method_ionization | philic_Pos | philic_Pos | philic_Neg | philic_Pos | philic_Pos | philic_Neg | philic_Pos | philic_Pos | philic_Neg |
| mode | | | | | | | | | |
| Metabolite | Choline | Citrulline | Citrate | L-Glutathione | UDP-N- | Methionine | Alanine | Folate | Glucose-6- |
| | | | | oxidized | acetylglucosamine/ | | | | Phosphate |
| | | | | | UDP-N- | | | | |
| | | | | | acetyl- | | | | |
| | | | | | galactosamine | | | | |
| pvalue | 0.502124 | 0.057274 | 0.269209 | 0.002791 | 0.004231 | 0.000612 | 0.001869 | 0.003412 | 0.005297 |
| log2Foldcha [symbol] | 0.256172 | 0.610469 | −0.1131 | −0.45706 | −0.50872 | −0.44547 | −0.84241 | −0.44929 | −0.41892 |
| ttest | 0.502124 | 0.057274 | 0.269209 | 0.002791 | 0.004231 | 0.000612 | 0.001869 | 0.003412 | 0.005297 |
| SARS-COV- | 3167667 | 807241613.7 | 181123691.9 | 18272187758 | 2730547844 | 3250071223 | 817318544 | 456837033.1 | 304891483.6 |
| [symbol] | | | | | | | | | |
| SARS-COV- | 6710727 | 994027467.7 | 197113499.8 | 11157037875 | 2435906627 | 2540445828 | 565930419.6 | 403697186.9 | 285797895.2 |
| [symbol] | | | | | | | | | |
| SARS-COV- | 8812015 | 453518720.9 | 170990237.5 | 13220909901 | 3038709977 | 3384608852 | 734252893.4 | 474496449.1 | 299086499.8 |
| [symbol] | | | | | | | | | |
| SARS-COV- | 15456624 | 521489093.2 | 203287185 | 14153741372 | 3520809479 | 3230056237 | 805403884.7 | 525438311.4 | 358061311.1 |
| [symbol] | | | | | | | | | |
| SARS-COV- | 8828307 | 620041993.8 | 240040450.5 | 17816818305 | 4523486847 | 3985641476 | 1285910374 | 683300369.5 | 407767311.3 |
| [symbol] | | | | | | | | | |
| SARS-COV- | 13773352 | 441295539 | 158643708.1 | 14164592682 | 3710044434 | 3445251087 | 493338136.1 | 505036061.3 | 241408610.5 |
| [symbol] | | | | | | | | | |
| Mock_06 | 9626839 | 470826040 | 197251939.8 | 20236771991 | 4559939042 | 4940087197 | 1351058562 | 664760190.9 | 438248289.2 |
| Mock_05 | 6484959 | 403266127.5 | 203789849.3 | 22157527229 | 5290313899 | 4530927439 | 1694928895 | 747573496.4 | 488080593.3 |
| Mock_04 | 11941626 | 416353051 | 222389784.9 | 19213182439 | 4748073690 | 4073937462 | 1233247207 | 665600226.4 | 376813031.9 |
| Mock_03 | 3394538 | 387832326.4 | 204500905.2 | 18205636381 | 4391520296 | 4456811823 | 1435304875 | 642565300.3 | 410318280.7 |
| Mock_02 | 8506912 | 437618423.2 | 226611495.1 | 22431933611 | 5211007654 | 4795007662 | 1616204669 | 789294859.3 | 437035988.2 |
| Mock_01 | 7561171 | 397679374.2 | 190535422.8 | 19634105834 | 4197228477 | 4215088997 | 1100411675 | 652945663.4 | 385664243 |
| Metabolite | In house | In house | In house | In house | In house | In house | In house | In house | In house |
| annotation | RT + MSMS | RT + MSMS | RT + MSMS | RT + MSMS | RT + MSMS | RT + MSMS | RT + MSMS | RT + MSMS | RT + MSMS |
| source | library | library | library | library | library | library | library | library | library |
| Method_ionization | amide_acidic | philic_Neg | philic_Neg | philic_Neg | philic_Neg | philic_Pos | philic_Neg | philic_Neg | philic_Pos |
| mode | | | | | | | | | |
| Metabolite | S- | 3- | N- | Pyroglutamate/ | Leucine | L-Histidine | Lysine | Tryptophan | N,N- |
| | adnosyolhomo- | Phosphoglycerate/ | Acetylglutamate | Oxoproline | | | | | Dimethyl-L- |
| | cysteine | 2- | | | | | | | arginine |
| | | Phosphoglycerate | | | | | | | |
| pvalue | 0.021797 | 0.240415 | 0.003202 | 0.005453 | 0.005393 | 0.001147 | 0.194112 | 0.001672 | 0.040927 |
| log2Foldcha [symbol] | 0.73502 | −0.32263 | −0.52986 | 1.020203 | 0.543268 | −0.52318 | 0.155118 | −0.53171 | −0.23452 |
| ttest | 0.021797 | 0.240415 | 0.003202 | 0.005453 | 0.005393 | 0.001147 | 0.194112 | 0.001672 | 0.040927 |
| SARS-COV- | 295629 | 4812445 | 73660070.86 | 5592594 | 15198741.73 | 139899861 | 46039039.55 | 1804268922 | 33241263.12 |
| [symbol] | | | | | | | | | |
| SARS-COV- | 433080 | 4207690 | 56884955.4 | 10702128 | 15319464.51 | 112907416.5 | 46699817.25 | 1610765322 | 35762562.69 |
| [symbol] | | | | | | | | | |
| SARS-COV- | 370494 | 1896571 | 66664277.95 | 8755439 | 10897090.28 | 96032908.52 | 36954109.87 | 1813585380 | 37382542.03 |
| [symbol] | | | | | | | | | |
| SARS-COV- | 274522 | 4055259 | 66472776.19 | 12113437 | 11800206.95 | 133979218.1 | 43686678.64 | 1996463554 | 37422169.12 |
| [symbol] | | | | | | | | | |
| SARS-COV- | 311856 | 4316641 | 93303573.1 | 6380999 | 12614875.26 | 167630678.9 | 38831029.79 | 2759806201 | 43399857.59 |
| [symbol] | | | | | | | | | |
| SARS-COV- | 159274 | 3191076 | 48839343.57 | 8941871 | 10181102.32 | 129883359.8 | 45253574.55 | 1888387834 | 26409286.24 |
| [symbol] | | | | | | | | | |
| Mock_06 | 206590 | 3656960 | 101070725.8 | 7117345 | 7756798.402 | 201160137.8 | 45075473.64 | 2641082694 | 42980239.45 |
| Mock_05 | 195264 | 5098472 | 101901610.1 | 3481888 | 9398919.229 | 204935682.5 | 36206037.27 | 3127534242 | 43962220.64 |
| Mock_04 | 207064 | 6946878 | 94345080.54 | 5200016 | 8238091.744 | 181200492.6 | 47419100.62 | 2795469749 | 41858708.25 |
| Mock_03 | 165011 | 2837041 | 99963600.85 | 3249056 | 8745508.377 | 170889746.5 | 31218644.24 | 2677196092 | 40126021.76 |
| Mock_02 | 179873 | 5637741 | 104162778.8 | 3578527 | 8785301.96 | 178327097 | 37749439.13 | 3231516513 | 44414870.72 |
| Mock_01 | 154605 | 3936209 | 84480662.84 | 3251457 | 9235591.167 | 184916338.4 | 33549397.65 | 2691660829 | 37982379.87 |
| Metabolite | In house | In house | In house | In house | In house | In house | In house | In house | In house |
| annotation | RT + MSMS | RT + MSMS | RT + MSMS | RT + MSMS | RT + MSMS | RT + MSMS | RT + MSMS | RT + MSMS | RT + MSMS |
| source | library | library | library | library | library | library | library | library | library |
| Method_ionization | philic_Neg | amide_acidic | philic_Neg | philic_Neg | philic_Pos | philic_Pos | philic_Neg | philic_Neg | philic_Pos |
| mode | | | | | | | | | |
| Metabolite | SAICAR | Cysteine | Allantoin | PRPP | Argininosuccinic | Ornithine | Fructose 1,6- | Phenylalanine | Kynurenine |
| | | | | | acid | | bisphosphate | | |

| | | | | | | | | | |
|---|---|---|---|---|---|---|---|---|---|
| pvalue | 0.28625 | 0.73689 | 0.002756 | 0.664651 | 0.001008 | 0.136979 | 0.010638 | 0.405113 | 0.267793 |
| log2Foldcha [symbol] | 0.25293 | 0.037323 | −0.56084 | −0.08696 | −0.58066 | 0.183668 | −0.29363 | −0.14109 | −0.12469 |
| ttest | 0.28625 | 0.73689 | 0.002756 | 0.664651 | 0.001008 | 0.136979 | 0.010638 | 0.405113 | 0.267793 |
| SARS-COV- | 13738407576 | 23805805.1 | 92445417.43 | 65567344.71 | 3949416433 | 3797686277 | 10576546.25 | 14483817.67 | 320273567.9 |
| [symbol] | | | | | | | | | |
| SARS-COV- | 15196021144 | 27344828.09 | 93600611.27 | 54355731.72 | 3286067863 | 4426949956 | 13356247.69 | 11976543.65 | 428021411.6 |
| [symbol] | | | | | | | | | |
| SARS-COV- | 15345960262 | 25171865.79 | 107095522.6 | 41796792.05 | 4267501223 | 4539178656 | 14330689.61 | 7385709.765 | 355314749.4 |
| [symbol] | | | | | | | | | |
| SARS-COV- | 15949153232 | 33500618.37 | 117849628.9 | 49310290.23 | 4428684319 | 4589141641 | 14830411.47 | 11557668.89 | 359700424.8 |
| [symbol] | | | | | | | | | |
| SARS-COV- | 14570397047 | 24879802.73 | 165291998.9 | 72127540.16 | 5876727326 | 4398126208 | 13224677.79 | 13469162.19 | 374254886.5 |
| [symbol] | | | | | | | | | |
| SARS-COV- | 13452212184 | 29323629.28 | 110011846.6 | 79955864.03 | 3811409577 | 3572470943 | 11537540.91 | 9532366.96 | 249885460.4 |
| [symbol] | | | | | | | | | |
| Mock_06 | 16892985031 | 27196596.27 | 158852051.7 | 69166202.37 | 6054206718 | 3205970876 | 16325731.32 | 9191730.843 | 393007838.9 |
| Mock_05 | 11485397769 | 29930290.62 | 191982613.6 | 59186104.01 | 6767704572 | 4436597645 | 16236703.13 | 13329531.66 | 397794971.6 |
| Mock_04 | 13333474291 | 26712133.23 | 163838319.6 | 55942679.08 | 6090829965 | 3966970462 | 18678546.51 | 11509827.78 | 364122954.5 |
| Mock_03 | 15869608674 | 21531371.36 | 160133347.6 | 68082642.66 | 5586511092 | 4048494942 | 14004571.84 | 13145223.91 | 359569141.1 |
| Mock_02 | 3406720344 | 30349100.64 | 190102362.1 | 88195907.3 | 7443551764 | 2737575224 | 15077128.59 | 15161966.22 | 405291999.5 |
| Mock_01 | 13072149204 | 24118049.74 | 147466539.1 | 45098902.01 | 6372411718 | 3900814552 | 15107455.71 | 13094836.84 | 356107577.1 |
| Metabolite | In house | In house | In house | In house | In house | In house | In house | In house | In house |
| annotation | RT + MSMS | RT + MSMS | RT + MSMS | RT + MSMS | RT + MSMS | RT + MSMS | RT + MSMS | RT + MSMS | RT + MSMS |
| source | library | library | library | library | library | library | library | library | library |
| Method_ionization | philic_Pos | philic_Neg | philic_Neg | philic_Neg | philic_Pos | philic_Pos | philic_Neg | philic_Neg | philic_Neg |
| mode | | | | | | | | | |
| Metabolite | L-Carnitine | Aminoadipate | Creatinine | 3- | Tyrosine | Deoxycarnitine | GDP-Glucose | Ribose-5- | Xylitol/ |
| | | | | Hydroxymethyl- | | | | Phosphate/ | Ribitol/ |
| | | | | glutarate | | | | Xylulose-5- | Arabitol |
| | | | | | | | | phosphate | |

| pvalue | 0.000913 | 0.003306 | 0.285988 | 0.05013 | 4.96E−06 | 0.038023 | 0.049724 | 0.465446 | 0.545911 |
|---|---|---|---|---|---|---|---|---|---|
| log2Foldcha [symbol] | −0.58296 | 0.413659 | −0.10278 | 0.173539 | −0.67365 | −0.50106 | 0.196102 | −0.0984 | 0.134632 |
| ttest | 0.000913 | 0.003306 | 0.285988 | 0.05013 | 4.96E−06 | 0.038023 | 0.049724 | 0.465446 | 0.545911 |
| SARS-COV- | 53588554.55 | 154445135.4 | 12914522.03 | 21502151978 | 1219104997 | 662943523.7 | 100147275.5 | 799845559.7 | 393851169.7 |
| [symbol] | | | | | | | | | |
| SARS-COV- | 48644144.33 | 140594636.8 | 15630582.78 | 24075056490 | 1248313568 | 553411479 | 122182904.5 | 919034388.1 | 469758637.1 |
| [symbol] | | | | | | | | | |
| SARS-COV- | 49278167.21 | 141616384.9 | 13155076.84 | 26440151578 | 1250528141 | 663351244.9 | 113493786.4 | 1070676965 | 379676222 |
| [symbol] | | | | | | | | | |
| SARS-COV- | 49659127.68 | 117650782 | 13747602.04 | 24155102913 | 1533544610 | 758118679.7 | 115267986.9 | 956254579.5 | 375893973.3 |
| [symbol] | | | | | | | | | |
| SARS-COV- | 68059985.2 | 113667917.4 | 13814461.71 | 23118668912 | 1570353105 | 1191494039 | 101729205.2 | 798825137.5 | 584305984.5 |
| [symbol] | | | | | | | | | |
| SARS-COV- | 35815071.69 | 117798793.3 | 16124869.27 | 19292747645 | 1209098231 | 483912176.4 | 111555703.3 | 712508692.5 | 573195391 |
| [symbol] | | | | | | | | | |
| Mock_06 | 71774697.15 | 106485582.8 | 18805694.63 | 20797448189 | 2073032020 | 959006147.9 | 96979168.84 | 1096542652 | 600686767 |
| Mock_05 | 79490431.26 | 83328308.63 | 14757816.46 | 21038930523 | 2025890669 | 1208225100 | 101444195.7 | 779144556 | 496453067.7 |
| Mock_04 | 72475519.81 | 110400050 | 13631418.6 | 22383994103 | 2076242974 | 822530042.9 | 101959227.8 | 1082694796 | 299314578.8 |
| Mock_03 | 76587325.94 | 97522213.58 | 14833640.3 | 19495470138 | 2069120684 | 1121189246 | 83381226.55 | 748607926.8 | 506559205.2 |
| Mock_02 | 84666661.99 | 98898674.06 | 14154356.54 | 19952184057 | 2382333822 | 1141898718 | 114843427.3 | 905717120.7 | 303385809 |
| Mock_01 | 71938814.25 | 93258699.59 | 15508939.23 | 19209458405 | 2183584324 | 851480601 | 81331154.72 | 1015504640 | 322886276.9 |
| Metabolite | In house | In house | In house | In house | In house | In house | In house | In house | In house |
| annotation | RT + MSMS | RT + MSMS | RT + MSMS | RT + MSMS | RT + MSMS | RT + MSMS | RT + MSMS | RT + MSMS | RT + MSMS |
| source | library | library | library | library | library | library | library | library | library |
| Method_ionization | philic_Pos | philic_Neg | philic_Pos | philic_Pos | philic_Pos | philic_Pos | philic_Neg | philic_Neg | philic_Neg |
| mode | | | | | | | | | |
| Metabolite | 1- | Phosphoenol- | CDP- | Betaine | Acetyl-L- | 1-Methyl-L- | Hypotaurine | UDP Glucose/ | D-Gluconate |
| | Amino- | pyruvate | ethanolamine | | carnitine | Histidine | | UDP | |
| | cyclopropane- | | | | | | | Galactose | |
| | carboxylate | | | | | | | | |
| pvalue | 0.031625 | 0.024282 | 0.080341 | 0.042731 | 0.069432 | 0.006225 | 0.372136 | 0.029137 | 0.187651 |
| log2Foldcha [symbol] | −0.49118 | −1.06828 | −0.44794 | −0.41712 | −1.26337 | 0.624842 | −0.18826 | 2.299494 | −0.17978 |
| ttest | 0.031625 | 0.024282 | 0.080341 | 0.042731 | 0.069432 | 0.006225 | 0.372136 | 0.029137 | 0.187651 |
| SARS-COV- | 5475421.707 | 39416417.68 | 1583409332 | 28551880.82 | 2460976.603 | 15671931.82 | 16066005.12 | 422386878.5 | 245409154.6 |
| [symbol] | | | | | | | | | |
| SARS-COV- | 5159964.926 | 29167228.28 | 1872598886 | 35637758.14 | 3147186.535 | 15776900 | 33334316.38 | 565177037.7 | 350410517.6 |
| [symbol] | | | | | | | | | |
| SARS-COV- | 6843709.949 | 60274455.05 | 1947001960 | 45304545.37 | 2500317.3 | 9353433.192 | 26796456.21 | 140716441 | 241375567.5 |
| [symbol] | | | | | | | | | |
| SARS-COV- | 7399494.582 | 30633924.71 | 1912041653 | 34538651.55 | 2369611.538 | 15107083.28 | 31437502.34 | 45811497.68 | 269294527.6 |
| [symbol] | | | | | | | | | |
| SARS-COV- | 8418263.274 | 79077713.93 | 2067606918 | 56499630.95 | 3946176.899 | 17048622.98 | 33949920.57 | 456683702.7 | 276063600.5 |
| [symbol] | | | | | | | | | |
| SARS-COV- | 2691134.071 | 25798248.44 | 1392639798 | 35400921.72 | 3689538.538 | 17733365.84 | 15613187.75 | 234201428.7 | 259905715.3 |
| [symbol] | | | | | | | | | |
| Mock_06 | 8058168.996 | 73626714.56 | 2114699827 | 60937354.28 | 4298974.78 | 8253271.636 | 31971739.51 | 49585773.74 | 307123774.5 |
| Mock_05 | 9181397.852 | 154195538.9 | 2140342298 | 55261945.3 | 4905709.58 | 9645471.528 | 26772616.18 | 56949122.02 | 277577174.1 |
| Mock_04 | 8463059.354 | 50400631.57 | 1791040751 | 45657482.47 | 15125463.17 | 6589422.198 | 33267646 | 90779549.61 | 241781434.3 |
| Mock_03 | 7760263.374 | 71088889.68 | 2047177933 | 44053209.3 | 4054430.61 | 11174962.28 | 26304383.1 | 31614920.52 | 309050736.9 |
| Mock_02 | 9830115.45 | 90947589.81 | 3728553595 | 66045095.14 | 4861941.89 | 12606040.48 | 35441167.29 | 125202594.7 | 378657726.3 |
| Mock_01 | 7291606.435 | 114100973.5 | 2876662762 | 43076618.79 | 10236684.03 | 10543310.78 | 25350951.96 | 24708835.2 | 346234842.5 |
| Metabolite | In house | In house | In house | In house | In house | In house | In house | MSMS | In house |
| annotation | RT + MSMS | RT + MSMS | RT + MSMS | RT + MSMS | RT + MSMS | RT + MSMS | RT + MSMS | | RT + MSMS |
| source | library | library | library | library | library | library | library | | library |
| Method_ionization | philic_Neg | philic_Neg | philic_Neg | philic_Neg | philic_Neg | philic_Neg | philic_Neg | philic_Neg | philic_Neg |
| mode | | | | | | | | | |
| Metabolite | Cytidine | Hippurate | Isociitrate | 2- | 2- | Ureidosuccinic | N- | L- | Glyceraldehyde |
| | | | | Hydroxybutyrate/ | Quinoline- | acid | Acetylcysteine | Cysteinesulfinic | 3-phosphate |
| | | | | Alpha- | carboxylate/4- | | | acid | |
| | | | | hydroxy- | Quinoline- | | | | |
| | | | | isobutyrate | carboxylate | | | | |

| | | | | | |
|---|---|---|---|---|---|
| pvalue | 0.22673 | 0.003357 | 0.001799 | 0.002518 | 0.117749 |
| log2Foldcha | 0.398657 | −0.7621 | −1.58231 | −1.03496 | −0.51265 |
| [symbol] | | | | | |
| ttest | 0.22673 | 0.003357 | 0.001799 | 0.002518 | 0.117749 |
| SARS-COV- | 32273435.83 | 48147511 | 501389 | 41452964.31 | 1652500430 |
| [symbol] | | | | | |
| SARS-COV- | 15856639.08 | 24403264 | 446446 | 19756373.67 | 1909211645 |
| [symbol] | | | | | |
| SARS-COV- | 11376992.59 | 28167161 | 537733 | 12379919.06 | 1669488907 |
| [symbol] | | | | | |
| SARS-COV- | 15117921.79 | 29504271 | 663792 | 23504245.93 | 2252898050 |
| [symbol] | | | | | |
| SARS-COV- | 15670873.37 | 47629131 | 1844974 | 30289740.8 | 3609040344 |
| [symbol] | | | | | |
| SARS-COV- | 16124399.81 | 21964729 | 44644.6 | 12123201.08 | 2894215969 |
| [symbol] | | | | | |
| Mock_06 | 15897545.74 | 51611918 | 1931691 | 31821127.73 | 3692223275 |
| Mock_05 | 15848347.03 | 72501285 | 2482831 | 54438695.53 | 3371200249 |
| Mock_04 | 10806113.8 | 57358190 | 1700185 | 49967996.24 | 4660801110 |
| Mock_03 | 16090271.44 | 54901115 | 1728778 | 56762290.07 | 1576591006 |
| Mock_02 | 11901337 | 55764458 | 2661593 | 52585509.27 | 4286804216 |
| Mock_01 | 10182972.62 | 46742895 | 1589580 | 40280995.27 | 2367732147 |
| Metabolite annotation | In house | In house | In house | MSMS | MSMS |
| source | RT + MSMS library | RT + MSMS library | RT + MSMS library | | |
| Method_ionization | philic_Neg | philic_Pos | philic_Pos | philic_Neg | amide_acidic |
| mode | | | | | |
| Metabolite | Dihydroxyacetone | Urocanic acid | 5-Formimino- | L-Cysteine- | Spermidine |
| | Phosphate | | tetrahydrofolate | glutathione Disulfide | |

[symbol]

indicates data missing or illegible when filed

TABLE 7

| Probe | | Probe | | | |
|---|---|---|---|---|---|
| # | pp1a_probe set | # | Np-probe set | sgRNAs | |
| 1 | TGAGTTGGACGTGTGTT | 1 | TGATTTTGGGGTCCATT | sgSHMT1_ | CACCGTCCCCATTTGAACACT |
| | TTC | | ATC | SS | GCCA |
| | (SEQ ID NO: 20) | | (SEQ ID NO: 21) | | (SEQ ID NO: 22) |
| 2 | TTGACGTGCCTCTGATA | 2 | AACGTAATGCGGGGTGC | sgSHMT1_ | AAACTGGCAGTGTTCAAATGG |
| | AGA | | ATT | as | GGAC |
| | (SEQ ID NO: 23) | | (SEQ ID NO: 24) | | (SEQ ID NO: 25) |
| 3 | CCACAAGTGCCATCTTT | 3 | AGTTGAATCTGAGGGTC | sgSHMT2_ | CACCGCTACTCACAAGACTCT |
| | AAG | | CAC | SS | TCGA |
| | (SEQ ID NO: 26) | | (SEQ ID NO: 27) | | (SEQ ID NO: 28) |
| 4 | CATAGGGCTGTTCAAGT | 4 | GTTCTCCATTCTGGTTAC | sgSHMT2_ | AAACTCGAAGAGTCTTGTGAG |
| | TGA | | TG | as | TAGC |
| | (SEQ ID NO: 29) | | (SEQ ID NO: 30) | | (SEQ ID NO: 31) |
| 5 | CCATAACATGACCATG | 5 | CGACGTTGTTTTGATCG | | |
| | AGGT | | CGC | | |
| | (SEQ ID NO: 32) | | (SEQ ID NO: 33) | | |
| 6 | TACTGAATGCCTTCGAG | 6 | AGACGCAGTATTATTGG | | |
| | TTC | | GTA | | |
| | (SEQ ID NO: 34) | | (SEQ ID NO: 35) | | |
| 7 | CCTTTATTACCGTTCTT | 7 | TGTTGAGTGAGAGCGGT | | |
| | ACG | | GAA | | |
| | (SEQ ID NO: 36) | | (SEQ ID NO: 37) | | |
| 8 | TGCTATGTTTAGTGTTC | 8 | GGGAATTTAAGGTCTTC | | |
| | CAG | | CTT | | |
| | (SEQ ID NO: 38) | | (SEQ ID NO: 39) | | |
| 9 | GTTATCGACATAGCGA | 9 | AATTGGAACGCCTTGTC | | |
| | GTGT | | CTC | | |
| | (SEQ ID NO: 40) | | (SEQ ID NO: 41) | | |
| 10 | CCAGTTGTTCGGACAAA | 10 | CATCTGGACTGCTATTG | | |
| | GTG | | GTG | | |
| | (SEQ ID NO: 42) | | (SEQ ID NO: 43) | | |
| 11 | TTTCAGAACGTTCCGTG | 11 | CTTCGGTAGTAGCCAAT | | |
| | TAC | | TTG | | |
| | (SEQ ID NO: 44) | | (SEQ ID NO: 45) | | |
| 12 | TCTACCCATAAAGCCAT | 12 | ACCACGAATTCGTCTGG | | |
| | CAA | | TAG | | |
| | (SEQ ID NO: 46) | | (SEQ ID NO: 47) | | |
| 13 | GAAAGGCACATTTGGTT | 13 | GATCTTTCATTTTACCGT | | |
| | GCA | | CA | | |
| | (SEQ ID NO: 48) | | (SEQ ID NO: 49) | | |
| 14 | TGGCTTTAACAAAATCG | 14 | TTTGTTAGCACCATAGG | | |
| | CCC | | GAA | | |
| | (SEQ ID NO: 50) | | SEQ ID NO: 51) | | |
| 15 | AGTAACCACAAGTAGT | 15 | CAGTTGCAACCCATATG | | |
| | GGCA | | ATG | | |
| | (SEQ ID NO: 52) | | (SEQ ID NO: 53) | | |
| 16 | TCAGGTCCTACTTCTGA | 16 | GATTGCAGCATTGTTAG | | |
| | ATT | | CAG | | |
| | (SEQ ID NO: 54) | | (SEQ ID NO: 55) | | |
| 17 | ACAACCTATGTTAGCGC | 17 | TAGAAGCCTTTTGGCAA | | |
| | TAG | | TGT | | |
| | (SEQ ID NO: 56) | | (SEQ ID NO: 57) | | |
| 18 | TTCTCCAACAACACCTG | 18 | ACGAGAAGAGGCTTGAC | | |
| | TAT | | TGC | | |
| | (SEQ ID NO: 58) | | (SEQ ID NO: 59) | | |
| 19 | GGTTGTCATTAAGACCT | 19 | TGTTGCGACTACGTGAT | | |
| | TCG | | GAG | | |
| | (SEQ ID NO: 60) | | (SEQ ID NO: 61) | | |
| 20 | CAAACCTTTCACAGTTT | 20 | GCCTGGAGTTGAATTTC | | |
| | CCA | | TTG | | |
| | (SEQ ID NO: 62) | | (SEQ ID NO: 63) | | |
| 21 | TTATAGCGGCCTTCTGT | 21 | CAGGAGAAGTTCCCCTA | | |
| | AAA | | CTG | | |
| | (SEQ ID NO: 64) | | (SEQ ID NO: 65) | | |
| 22 | ACTGAACAACACCACC | 22 | CAGCAAAGCAAGAGCA | | |
| | TGTA | | GCAT | | |
| | (SEQ ID NO: 66) | | (SEQ ID NO: 67) | | |
| 23 | TCAAGGACGGGTTTGA | 23 | AGCTGGTTCAATCTGTC | | |
| | GTTT | | AAG | | |
| | (SEQ ID NO: 68) | | (SEQ ID NO: 69) | | |
| 24 | ATTTGTCCACCGACAAT | 24 | TTTACCAGACATTTTGCT | | |
| | TTC | | CT | | |
| | (SEQ ID NO: 70) | | (SEQ ID NO: 71) | | |
| 25 | GAATGTCTGAACACTCT | 25 | TAGTGGCAGTACGTTTT | | |
| | CCT | | TGC | | |
| | (SEQ ID NO: 72) | | (SEQ ID NO: 73) | | |
| 26 | CTTTCTGTACAATCCCT | 26 | GCTTGTGTTACATTGTAT | | |
| | TTG | | GC | | |
| | (SEQ ID NO: 74) | | (SEQ ID NO: 75) | | |
| 27 | ACTTCTGTGGGAAGTGT | 27 | TTTGTTCTGGACCACGTC | | |
| | TTC | | TG | | |
| | (SEQ ID NO: 76) | | (SEQ ID NO: 77) | | |
| 28 | ACAGCTTCACTAGTAGG | 28 | TGGTCCCCAAAATTTCC | | |
| | TTG | | TTG | | |
| | (SEQ ID NO: 78) | | (SEQ ID NO: 79) | | |
| 29 | ACAAACTGGTGTACCA | 29 | AGTTCCTTGTCTGATTAG | | |
| | ACCA | | TT | | |
| | (SEQ ID NO: 80) | | (SEQ ID NO: 81) | | |
| 30 | ATCATATTAGGTGCAAG | 30 | CAATTTGCGGCCAATGT | | |
| | GGC | | TTG | | |
| | (SEQ ID NO: 82) | | (SEQ ID NO: 83) | | |
| 31 | AAGTAACCTTTGTTGGT | 31 | GAAGCGCTGGGGGCAA | | |
| | GCA | | ATTG | | |
| | (SEQ ID NO: 84) | | (SEQ ID NO: 85) | | |
| 32 | CTTGCACTTCTATCACA | 32 | ATGCGCGACATTCCGAA | | |
| | GTG | | GAA | | |
| | (SEQ ID NO: 86) | | (SEQ ID NO: 87) | | |
| 33 | CTCATTTACTTCTGTAC | 33 | CGAAGGTGTGACTTCCA | | |
| | CGA | | TGC | | |
| | (SEQ ID NO: 88) | | (SEQ ID NO: 89) | | |
| 34 | TCTAAATCAATGCCCAG | 34 | CTGTGTAGGTCAACCAC | | |
| | TGG | | GTT | | |
| | (SEQ ID NO: 90) | | (SEQ ID NO: 91) | | |
| 35 | CATCTTCAGTACCATAC | 35 | ATTTGGATCTTTGTCATC | | |
| | TCA | | CA | | |
| | (SEQ ID NO: 92) | | (SEQ ID NO: 93) | | |
| 36 | GTCTTGTTGACCAACAG | 36 | TTGTATGCGTCAATATG | | |
| | TTT | | CTT | | |
| | (SEQ ID NO: 94) | | (SEQ ID NO: 95) | | |
| 37 | GTTCCATCTCTAATTGA | 37 | GGTAAGGCTTGAGTTTC | | |
| | GGT | | ATC | | |
| | (SEQ ID NO: 96) | | (SEQ ID NO: 97) | | |
| 38 | TTGGCTGCATTAACAAC | 38 | GAAGAGTCACAGTTTGC | | |
| | CAC | | TGT | | |
| | (SEQ ID NO: 98) | | (SEQ ID NO: 99) | | |
| 39 | CTTATTTAAGGCTCCTG | 39 | ATCATCCAAATCTGCAG | | |
| | CAA | | CAG | | |
| | (SEQ ID NO: 100) | | (SEQ ID NO: 101) | | |
| 40 | ACCTTTGTTAACATTTG | 40 | GGATTGTTGCAATTGTTT | | |
| | GGC | | GG | | |
| | (SEQ ID NO: 102) | | (SEQ ID NO: 103) | | |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of inhibiting reproduction of a virus, comprising administering to a subject in need thereof, an effective amount of one or more inhibitors of folate metabolism and/or one-carbon metabolism in one or more cells of the subject.

2. The method of claim 1, wherein the inhibitor of folate and/or one-carbon metabolism is an inhibitor of cytosolic and/or mitochondrial isoforms of serine hydroxymethyltransferase (SHMT1 and SHMT2, respectively).

3. The method of claim 2, wherein the SHMT1/SHMT2 inhibitor is a compound of Formula (I), 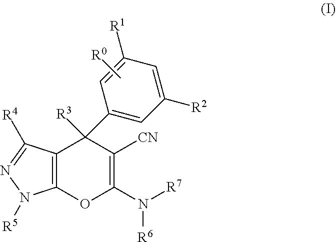
wherein:
R⁰, R¹ and R² are each independently selected from the group consisting of —H, halogen, hydroxyl, nitro, nitrile, —SOR¹¹, —S(O)₂R¹¹, —S(O)₂NR¹⁰R¹², —OR¹¹, —OC(O)R¹²,
C(O)OR¹², —C(O)R¹¹, —C(O)NR¹⁰R¹², —NR¹⁰R¹², —N(R¹²)C(O)R¹¹, NS(O)₂R¹², substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl, and substituted or unsubstituted haloalkoxy; provided that at least one of R⁰, R¹ and R² is selected from the group consisting of substituted or unsubstituted alkenyl, and substituted or unsubstituted alkynyl;
R³, is selected from the group consisting of —H, halogen, hydroxyl, nitro, nitrile, SOR¹¹, —S(O)₂R¹¹, S(O)₂NR¹⁰R¹², —OR¹¹, —OC(O)R¹², —C(O)OR¹², —C(O)R¹¹, C(O)NR¹⁰R¹², —NR¹⁰R¹², N(R¹²)C(O)R¹¹, —NS(O)₂R¹², substituted or unsubstituted alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl, and substituted or unsubstituted haloalkoxy;
R⁴ is selected from the group consisting of —H, substituted or unsubstituted alkyl, substituted or unsubstituted cycloalkyl, substituted or unsubstituted aryl, substituted or unsubstituted heteroaryl, substituted or unsubstituted arylalkyl, and substituted or unsubstituted heteroarylalkyl;
R⁵, R⁶, and R⁷ are each independently selected from the group consisting —H, —C(O)R¹¹, substituted or unsubstituted alkyl, substituted or unsubstituted cycloalkyl, substituted or unsubstituted aryl, substituted or unsubstituted heteroaryl, substituted or unsubstituted arylalkyl, and substituted or unsubstituted heteroarylalkyl; or R⁵ is selected from any of the foregoing and R⁶ and R⁷ taken together with the nitrogen atom to which they are attached form a substituted or unsubstituted 3-6 membered ring;
each occurrence of R¹¹is independently selected from the group consisting of substituted or unsubstituted alkyl, substituted or unsubstituted cycloalkyl, substituted or unsubstituted heterocyclyl, substituted or unsubstituted aryl, and substituted or unsubstituted heteroaryl; and
each occurrence of R¹⁰ and R¹² is independently selected from the group consisting of —H, substituted or unsubstituted alkyl, substituted or unsubstituted cycloalkyl, substituted or unsubstituted heterocyclyl, substituted or unsubstituted aryl, and substituted or unsubstituted heteroaryl.

4. The method of claim 2, wherein the SHMT1/SHMT2 inhibitor is a compound selected from Table 2.

5. The method of claim 2, wherein the SHMT1/SHMT2 inhibitor is a compound of Formula Formula (II), 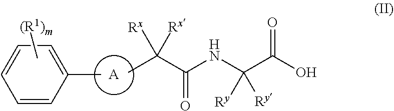
wherein:
Ring A is an optionally substituted bivalent ring;
each R¹ is independently halogen, —CN, —NO₂, —OR, -Cy, or an optionally substituted C₁₋₆ aliphatic group; or
two R¹ groups taken together with their intervening atoms are a 5-8 membered partially unsaturated or aryl fused ring having 0-3 heteroatoms independently selected from nitrogen, oxygen or sulfur; or
each R is independently hydrogen or an optionally substituted group selected from C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
Cy is an optionally substituted group selected from a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
R^{x} and R^{y} are independently hydrogen or an optionally substituted group selected from C₁₋₆ aliphatic or phenyl;
R^{x′} and R^{y′} are independently hydrogen or C₁₋₄ alkyl; or
R^{y} and R^{y′} can be taken together with their intervening atoms to form a 3-6 membered saturated ring having 0-3 heteroatoms independently selected from nitrogen, oxygen or sulfur; and
m is 1, 2, 3, 4 or 5.

6. The method of claim 5 wherein the SHMT1/SHMT2 inhibitor is a compound selected from Table 3.

7. The method of claim 1, wherein the inhibitor of folate and/or one-carbon metabolism is an antifolate agent.

8. A method of inhibiting reproduction of a virus, comprising administering to a subject in need thereof, an effective amount of one or more inhibitors of glucose metabolism.

9. A method of inhibiting reproduction of a virus, comprising administering to a subject in need thereof, an effective amount of one or more inhibitors of oxidative phosphorylation.

10. The method of claim 9, the one or more inhibitors of oxidative phosphorylation is an inhibitor of electron transport chain complex I.

11. The method of claim 10, wherein the inhibitor of electron transport chain complex I is Piericidin A (PierA).

12. The method of claim 9, the one or more inhibitors of oxidative phosphorylation is metformin, phenformin, atovaquone, chloramphenicol, meclizine, or S-meclizine.

13. A method of inhibiting reproduction of a virus, comprising administering to a subject in need thereof, an effective amount of one or more inhibitors of purine synthesis.

14. A method of inhibiting reproduction of a virus, comprising administering to a subject in need thereof, an effective amount of one or more inhibitors of serine synthesis.

15. The method of claim 14, wherein the one or more inhibitors of serine synthesis is one or more inhibitors of phosphoglycerate dehydrogenase (PHGDH).

16. The method of any one of claims 1 to 15, wherein the virus is an RNA virus.

17. The method of claim 16, wherein the RNA virus is a positive-sense single strand RNA virus.

18. The method of claim 17, wherein the positive-sense single strand RNA virus is a coronavirus, flavivirus, or enterovirus.

19. The method of claim 18, wherein the coronavirus is SARS-CoV-1, MERS-CoV or SARS-CoV-2.

20. The method of claim 16, wherein the RNA virus is a negative-sense single strand RNA virus.

21. The method of claim 20, wherein the negative-sense single strand RNA virus is an influenza, paramyxovirus, respiratory syncytial virus, measles, rabies, or Ebola.

22. The method of anyone of claims 1 to 15, wherein the one or more inhibitors is administered in combination with one or more anti-viral therapeutics.

23. The method of claim 22, wherein the one or more anti-viral therapeutics comprises remdesivir, favipiravir, ribavirin, lopinavir/ritonavir, tocilizumab, leronlimab, ivermectin, chloroquine, hydroxychloroquine, colchicine, dexamethasone, prednisone, methylprednisolone, nicotine, vitamin D, spironolactone, casirivimab/imdevimab, bamlanivimab/etesevimab, sotrovimab, convalescent plasma, an mRNA vaccine, a viral vector vaccine, or any combination thereof.

24. The method of anyone of claims 1 to 15, wherein the one or more inhibitors is administered prophylactically.
